(19) United States
(12) Patent Application Publication
(10) Pub. No.: US 2026/0337488 A1
(43) Pub. Date: Oct. 29, 2026

(54) COMPOUND, LIQUID CRYSTAL COMPOSITION, AS WELL AS LIQUID CRYSTAL DISPLAY ELEMENT, SENSOR, LIQUID CRYSTAL LENS, OPTICAL COMMUNICATION EQUIPMENT, AND ANTENNA EACH USING THE LIQUID CRYSTAL COMPOSITION

(71) Applicant: DIC Corporation, Tokyo (JP)

(72) Inventors: Daiki NORO, Saitama (JP); Mika TAKASAKI, Saitama (JP); Junichi MAMIYA, Saitama (JP); Masanao HAYASHI, Saitama (JP); Yutaka KADOMOTO, Saitama (JP); Takaya IKEUCHI, Saitama (JP)

(73) Assignee: DIC Corporation, Tokyo (JP)

(21) Appl. No.: 19/117,086

(22) PCT Filed: Oct. 12, 2023
(86) PCT No.: PCT/JP2023/036950

§ 371 Date: Mar. 31, 2025

(30) Foreign Application Priority Data
Oct. 27, 2022 (JP) 2022-172242

(51) Int. Cl. C09K 19/34; C09K 19/12; C09K 19/18; C09K 19/30; C09K 19/32; G01S 7/481; H01Q 9/04

(52) U.S. Cl. CPC C09K 19/3491; C09K 19/12; C09K 19/18; C09K 19/3003; C09K 19/3059; C09K 19/322; H01Q 9/0442; C09K 2019/122; C09K 2019/123; C09K 2019/181; C09K 2019/183; C09K 2019/185; C09K 2019/3009; C09K 2019/301; C09K 2019/3062; C09K 2019/3063; C09K 2019/3075; G01S 7/4816

(57) ABSTRACT

It is an object of the present invention to provide a compound that can provide a liquid crystal composition with a high Tni, a high Δn, a high Δεr, a low tan δiso, and high storage stability at room temperature, a liquid crystal composition, as well as a liquid crystal display element, a sensor, a liquid crystal lens, optical communication equipment, and an antenna each using the liquid crystal composition.

More specifically, the liquid crystal composition contains one or two or more compounds represented by the general formula (i) having a tetracyclic structure, —C≡C— as a linking group between the ring structures, and an isothiocyanate group (—NCS).

### TECHNICAL FIELD

The present invention relates to a compound, a liquid crystal composition, as well as a liquid crystal display element, a sensor, a liquid crystal lens, optical communication equipment, and an antenna each using the liquid crystal composition.

### BACKGROUND ART

As a new application of a liquid crystal often used for a display application, an antenna using a liquid crystal for transmitting and receiving a radio wave between a moving body, such as an automobile, and a communication satellite has attracted attention. Although a satellite communication has used a parabolic antenna, when the satellite communication is used in a moving body, such as an automobile, the parabolic antenna must be directed toward the satellite at any time and therefore requires a large movable portion. However, an antenna using a liquid crystal can change the transmission and reception direction of a radio wave by the operation of the liquid crystal within a panel, eliminate the need to move the antenna itself, and have a flat shape. Furthermore, to realize global large-capacity and high-speed communications, a low-earth-orbit satellite constellation using a large number of low-earth-orbit satellites is being studied. A liquid crystal antenna that can easily change the direction of transmission and reception of a radio wave is useful for tracking low-earth-orbit satellites that appear from the ground to be continually moving.

In general, autonomous driving of automobiles and the like requires massive data download of high-precision 3D map information. However, an antenna using a liquid crystal can be installed in an automobile to download a large amount of data from a communication satellite without a mechanical movable portion. A frequency band used in satellite communications is an approximately 13 GHz band, which is significantly different from the frequencies used in liquid crystal display applications. Thus, the physical properties required for a liquid crystal are also greatly different, and Δn required for a liquid crystal for an antenna is, for example, approximately 0.4, and the operating temperature ranges from, for example, −20° C. to 120° C.

Furthermore, an infrared laser image recognition and range finder using a liquid crystal has also attracted attention as a sensor for autonomous driving of a moving body, such as an automobile. Δn required for the liquid crystal for this application ranges from, for example, 0.3 to 0.6, and the operating temperature ranges from, for example, 10° C. to 100° C.

Furthermore, it is known that many liquid crystal compounds constituting liquid crystal compositions with a high Δn of 0.2 or more have low compatibility. Thus, it is also important to select a liquid crystal compound with high compatibility.

On the other hand, a technique of a liquid crystal for an antenna is described in, for example, Patent Literature 1.

The use of a liquid crystal material as a constituent of a high-frequency device is proposed in Non-Patent Literature 1.

### CITATION LIST

### Patent Literature

PTL 1: Japanese Unexamined Patent Application Publication No. 2016-37607

### Non Patent Literature

NPL 1: D. Dolfi, “Electronics Letters”, (Great Britain), 1993, Vol. 29, No. 10, pp. 926-928

### SUMMARY OF INVENTION

### Technical Problem

It is an object of the present invention to provide a compound that can provide a liquid crystal composition with a high Tₙᵢ, a high Δn, a high Δεᵣ, a low tan δᵢₛₒ, and high storage stability at room temperature, a liquid crystal composition, as well as a liquid crystal display element, a sensor, a liquid crystal lens, optical communication equipment, and an antenna each using the liquid crystal composition.

### Solution to Problem

As a result of extensive studies, the present inventors have completed the present invention by finding that a liquid crystal composition containing one or two or more compounds represented by the general formula (i) having a tetracyclic structure, —C≡C— as a linking group between the ring structures, and an isothiocyanate group (—NCS) can solve the above problems.

One example of the configuration of the present invention to solve the problems is as follows:
Item 1. A liquid crystal composition containing one or two or more compounds represented by the following general formula (i).

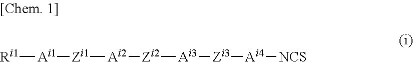

0 0 0 1 0 0 0 2 0 0 0 3 0 0 0 4 (in the general formula (i),
   Rⁱ¹ denotes a hydrogen atom or an alkyl group with 1 to 20 carbon atoms,
   one or two or more —CH₂— groups in the alkyl group are each independently optionally substituted with —O—, —S—, —CO—, and/or —CS—,
   one or two or more —CH₂—CH₂— groups in the alkyl group are each independently optionally substituted with —CO—O—, —O—CO—, —CO—S—, —S—CO—, —CO—NH—, —NH—CO—, —CH═CH—, —CF═CF—, and/or —C≡C—,
   one or two or more hydrogen atoms in the alkyl group are each independently optionally substituted with a halogen atom,
   provided that oxygen atoms are not directly bonded to each other,
   Aⁱ¹, Aⁱ², Aⁱ³, and Aⁱ⁴ each independently denote any one of a hydrocarbon ring with 3 to 16 carbon atoms and a heterocycle with 3 to 16 carbon atoms,
   one or two or more hydrogen atoms in Aⁱ¹, Aⁱ², Aⁱ³, and Aⁱ⁴ are each independently optionally substituted with a substituent Sⁱ¹,
   the substituent Sⁱ¹ denotes any one of a fluorine atom, a chlorine atom, a bromine atom, an iodine atom, a pentafluorosulfanyl group, a nitro group, a cyano group, an isocyano group, an amino group, a hydroxy group, a mercapto group, a methylamino group, a dimethylamino group, a diethylamino group, a diisopropylamino group, a trimethylsilyl group, a dimethylsilyl group, a thioisocyano group, and an alkyl group with 1 to 20 carbon atoms,
   one or two or more —CH₂— groups in the alkyl group are each independently optionally substituted with —O—, —S—, and/or —CO—,
   one or two or more —CH₂—CH₂— groups in the alkyl group are each independently optionally substituted with —CH═CH—, —CF═CF—, —C≡C—, —CO—O—, —O—CO—, —CO—S—, —S—CO—, —CO—NH—, and/or —NH—CO—,
   one or two or more hydrogen atoms in the alkyl group are each independently optionally substituted with a halogen atom,
   provided that oxygen atoms are not directly bonded to each other,
   a plurality of substituents Sⁱ¹, if present, may be the same or different, Zⁱ¹, Zⁱ², and Zⁱ³ each independently denote any one of a single bond and an alkylene group with 1 to 20 carbon atoms,
   at least one of Zⁱ¹, Zⁱ², and Zⁱ³ denotes —C≡C—,
   one or two or more —CH₂— groups in the alkylene group are each independently optionally substituted with —O—, —CF₂—, and/or —CO—, and
   one or two or more —CH₂—CH₂— groups in the alkylene group are each independently optionally substituted with —CH₂—CH(CH₃)—, —CH(CH₃)—CH₂—, —CH═CH—, —CF═CF—, —CH═C(CH₃)—, —C(CH₃)═CH—, —CH═N—, —N═CH—, —N═N—, —C≡C—, —CO—O—, and/or —O—CO—,
   provided that oxygen atoms are not directly bonded to each other)
Item 2. The liquid crystal composition according to Item 1, wherein Rⁱ¹ denotes an alkynyl group with 2 to 20 carbon atoms by substituting one or two or more —CH₂—CH₂— groups in the alkyl group with —C≡C—.
Item 3. The liquid crystal composition according to Item 1 or 2, wherein Zⁱ³ denotes —C≡C—.
Item 4. The liquid crystal composition according to according to any one of Items 1 to 3, further containing one or two or more compounds represented by the following general formula (ii).

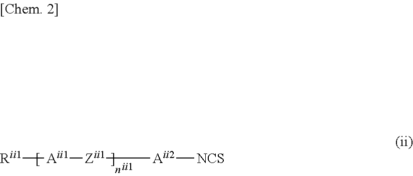

(in the general formula (ii),
   Rⁱⁱ¹ each independently denotes an alkyl group with 1 to 20 carbon atoms,
   one or two or more —CH₂— groups in the alkyl group are each independently optionally substituted with —O—, —S—, —CO—, and/or —CS—,
   one or two or more —CH₂—CH₂— groups in the alkyl group are each independently optionally substituted with —CH═CH—, —CO—O—, —O—CO—, —CO—S—, —S—CO—, —CO—NH—, —NH—CO—, —CH═CH—, —CF═CF—, and/or —C≡C—,
   one or two or more hydrogen atoms in the alkyl group are each independently optionally substituted with a halogen atom,
   provided that oxygen atoms are not directly bonded to each other,
   Aⁱⁱ¹ and Aⁱⁱ² each independently denote a group selected from the group consisting of the following groups (a), (b), (c), and (d):
   (a) a 1,4-cyclohexylene group (one —CH₂— or two or more nonadjacent —CH₂— groups in this group are optionally substituted with —O— and/or —S—),
   (b) a 1,4-phenylene group (one —CH═ or two or more —CH═ groups in this group are optionally substituted with —N═),
   (c) a 1,4-cyclohexenylene group, a bicycle[2.2.2]octane-1,4-diyl group, a naphthalene-2,6-diyl group, a naphthalene-1,4-diyl group, a 1,2,3,4-tetrahydronaphthalene-2,6-diyl group, a 5,6,7,8-tetrahydronaphthalene-1,4-diyl group, a decahydronaphthalene-2,6-diyl group, an anthracene-2,6-diyl group, an anthracene-1,4-diyl group, an anthracene-9,10-diyl group, a phenanthrene-2,7-diyl group, a 2,3-dihydroindene-2,5-diyl group, a 2,3-dihydroindene-1,5-diyl group, or a 2,3-dihydroindene-3,5-diyl group (one —CH═ or two or more —CH═ groups in the naphthalene-2,6-diyl group, the naphthalene-1,4-diyl group, the 1,2,3,4-tetrahydronaphthalene-2,6-diyl group, the 5,6,7,8-tetrahydronaphthalene-1,4-diyl group, the anthracene-2,6-diyl group, the anthracene-1,4-diyl group, the anthracene-9,10-diyl group, or the phenanthrene-2,7-diyl group are optionally substituted with —N═), and
   (d) a thiophene-2,5-diyl group, a benzothiophene-2,5-diyl group, a benzothiophene-2,6-diyl group, a dibenzothiophene-3,7-diyl group, a dibenzothiophene-2,6-diyl group, a thieno[3,2-b]thiophene-2,5-diyl group, or a benzo[1,2-b: 4,5-b′]dithiophene-2,6-diyl group (one —CH═ or two or more —CH═ groups in this group are optionally substituted with —N═),
   one or two or more hydrogen atoms in Aⁱⁱ¹ and Aⁱⁱ² are each independently optionally substituted with a substituent Sⁱⁱ¹,
   the substituent Sⁱⁱ¹ denotes any one of a halogen atom, a pentafluorosulfanyl group, a nitro group, a cyano group, an isocyano group, an amino group, a hydroxy group, a mercapto group, a methylamino group, a dimethylamino group, a diethylamino group, a diisopropylamino group, a trimethylsilyl group, a dimethylsilyl group, a thioisocyano group, and an alkyl group with 1 to 20 carbon atoms,
   one or two or more —CH₂— groups in the alkyl group are each independently optionally substituted with —O—, —S—, —CO—, and/or —CS—,
   one or two or more —CH₂—CH₂— groups in the alkyl group are each independently optionally substituted with —CO—O—, —O—CO—, —CO—S—, —S—CO—, —CO—NH—, —NH—CO—, —CH═CH—, —CF═CF—, and/or —C≡C—,
   one or two or more hydrogen atoms in the alkyl group are each independently optionally substituted with a halogen atom,
   provided that oxygen atoms are not directly bonded to each other,
   a plurality of substituents Sⁱⁱ¹, if present, may be the same or different,
   Zⁱⁱ¹ denotes any one of a single bond and an alkylene group with 1 to 20 carbon atoms,
   one or two or more —CH₂— groups in the alkylene group are each independently optionally substituted with —O—, —CF₂—, and/or —CO—, and
   one or two or more —CH₂—CH₂— groups in the alkylene group are each independently optionally substituted with —CH₂—CH(CH₃)—, —CH(CH₃)—CH₂—, —CH═CH—, —CF═CF—, —CH═C(CH₃)—, —C(CH₃)═CH—, —CH═N—, —N═CH—, —N═N—, —C≡C—, —CO—O—, and/or —O—CO—,
   provided that oxygen atoms are not directly bonded to each other,
   nⁱⁱ¹ denotes an integer in the range of 1 to 4, and
   a plurality of Aⁱⁱ¹, if present, may be the same or different, and a plurality of Zⁱⁱ¹, if present, may be the same or different,
   provided that the compounds represented by the general formula (i) are excluded)
Item 5. The liquid crystal composition according to Item 4, wherein the compound represented by the general formula (ii) is selected from the group consisting of compounds represented by the following general formulae (ii-1) to (ii-8).

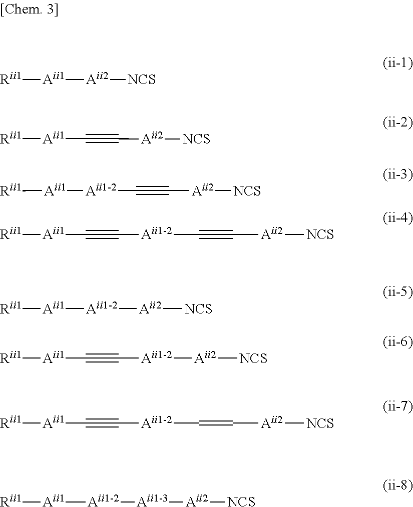

(in the general formulae (ii-1) to (ii-8),
   Rⁱⁱ¹, Aⁱⁱ¹, and Aⁱ¹² denote the same as Rⁱⁱ¹, Aⁱⁱ¹, and Aⁱⁱ², respectively, in the general formula (ii), and
   Aⁱⁱ¹-2 and Aⁱⁱ¹-3 in the general formulae (ii-3) to (ii-8) are each independently defined in the same way as Aⁱⁱ¹ in the general formula (ii))
Item 6. The liquid crystal composition according to any one of Items 1 to 5, wherein Δn at 25° C. and at 589 nm is 0.38 or more.
Item 7. A liquid crystal display element including the liquid crystal composition according to any one of Items 1 to 6.
Item 8. The liquid crystal display element according to Item 7, wherein the liquid crystal display element is driven by an active matrix system or a passive matrix system.
Item 9. A liquid crystal display element that reversibly switches permittivity by reversibly changing an orientation direction of liquid crystal molecules of the liquid crystal composition according to any one of Items 1 to 6.
Item 10. A sensor including the liquid crystal composition according to any one of Items 1 to 6.
Item 11. A liquid crystal lens including the liquid crystal composition according to any one of Items 1 to 6.
Item 12. Optical communication equipment including the liquid crystal composition according to any one of Items 1 to 6.
Item 13. An antenna including the liquid crystal composition according to any one of Items 1 to 6.
Item 14. An antenna according to Item 13, wherein the antenna includes
   a first substrate with a plurality of slots;
   a second substrate facing the first substrate and having a power supply portion;
   a first dielectric layer between the first substrate and the second substrate;
   a plurality of patch electrodes arranged corresponding to the plurality of slots;
   a third substrate on which the patch electrodes are provided; and
   a liquid crystal layer between the first substrate and the third substrate, wherein the liquid crystal layer contains the liquid crystal composition according to any one of Items 1 to 6.
Item 15. A compound represented by the following general formula (i).

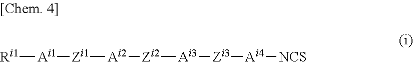

(in the general formula (i),
   Rⁱ¹ denotes a hydrogen atom or an alkyl group with 1 to 20 carbon atoms,
   one or two or more —CH₂— groups in the alkyl group are each independently optionally substituted with —O—, —S—, —CO—, and/or —CS—,
   one or two or more —CH₂—CH₂— groups in the alkyl group are each independently optionally substituted with —CO—O—, —O—CO—, —CO—S—, —S—CO—, —CO—NH—, —NH—CO—, —CH═CH—, —CF═CF—, and/or —C≡C—,
   one or two or more hydrogen atoms in the alkyl group are each independently optionally substituted with a halogen atom,
   provided that oxygen atoms are not directly bonded to each other,
   Aⁱ¹, Aⁱ², Aⁱ³, and Aⁱ⁴ each independently denote any one of a hydrocarbon ring with 3 to 16 carbon atoms and a heterocycle with 3 to 16 carbon atoms,
   one or two or more hydrogen atoms in Aⁱ¹, Aⁱ², Aⁱ³, and Aⁱ⁴ are each independently optionally substituted with a substituent Sⁱ¹,
   the substituent Sⁱ¹ denotes any one of a fluorine atom, a chlorine atom, a bromine atom, an iodine atom, a pentafluorosulfanyl group, a nitro group, a cyano group, an isocyano group, an amino group, a hydroxy group, a mercapto group, a methylamino group, a dimethylamino group, a diethylamino group, a diisopropylamino group, a trimethylsilyl group, a dimethylsilyl group, a thioisocyano group, and an alkyl group with 1 to 20 carbon atoms,
   one or two or more —CH₂— groups in the alkyl group are each independently optionally substituted with —O—, —S—, and/or —CO—,
   one or two or more —CH₂—CH₂— groups in the alkyl group are each independently optionally substituted with —CH═CH—, —CF═CF—, —C≡C—, —CO—O—, —O—CO—, —CO—S—, —S—CO—, —CO—NH—, and/or —NH—CO—,
   one or two or more hydrogen atoms in the alkyl group are each independently optionally substituted with a halogen atom,
   provided that oxygen atoms are not directly bonded to each other,
   a plurality of substituents Sⁱ¹, if present, may be the same or different, Zⁱ¹, Zⁱ², and Zⁱ³ each independently denote any one of a single bond and an alkylene group with 1 to 20 carbon atoms,
   at least one of Zⁱ¹, Zⁱ², and Zⁱ³ denotes —C≡C—,
   one or two or more —CH₂— groups in the alkylene group are each independently optionally substituted with —O—, —CF₂—, and/or —CO—, and
   one or two or more —CH₂—CH₂— groups in the alkylene group are each independently optionally substituted with —CH₂—CH(CH₃)—, —CH(CH₃)—CH₂—, —CH═CH—, —CF═CF—, —CH═C(CH₃)—, —C(CH₃)═CH—, —CH═N—, —N═CH—, —N═N—, —C≡C—, —CO—O—, and/or —O—CO—,
   provided that oxygen atoms are not directly bonded to each other)

### Advantageous Effects of Invention

The present invention can provide a liquid crystal composition containing one or two or more compounds represented by the general formula (i) having a tetracyclic structure, —C≡C— as a linking group between the ring structures, and an isothiocyanate group (—NCS) and thereby provide a liquid crystal composition with a high Tₙᵢ, a high Δn, a high Δεᵣ, a low tan δᵢₛₒ, and high storage stability at room temperature, and the liquid crystal composition is useful for a liquid crystal display element, a sensor, a liquid crystal lens, optical communication equipment, and an antenna.

### DESCRIPTION OF EMBODIMENTS

### (Compound Represented by General Formula (i))

A liquid crystal composition according to the present invention is a compound represented by the general formula (i) having a tetracyclic structure, —C≡C— as a linking group between the ring structures, and an isothiocyanate group (—NCS).

A liquid crystal composition according to the present invention contains one or two or more compounds represented by the general formula (i) having a tetracyclic structure, —C≡C— as a linking group between the ring structures, and an isothiocyanate group (—NCS).

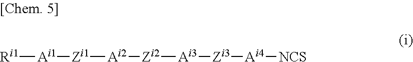

In the general formula (i), Rⁱ¹ denotes a hydrogen atom or an alkyl group with 1 to 20 carbon atoms.

The alkyl group with 1 to 20 carbon atoms is a linear, branched, or cyclic alkyl group, preferably a linear alkyl group.

The number of carbon atoms in the alkyl group with 1 to 20 carbon atoms preferably ranges from 2 to 10 or preferably 2 to 6.

One or two or more —CH₂— groups in the alkyl group are each independently optionally substituted with —O—, —S—, —CO—, and/or —CS—.

One or two or more —CH₂—CH₂— groups in the alkyl group are each independently optionally substituted with —CO—O—, —O—CO—, —CO—S—, —S—CO—, —CO—NH—, —NH—CO—, —CH═CH—, —CF═CF—, and/or —C≡C—.

One or two or more hydrogen atoms in the alkyl group are each independently optionally substituted with a halogen atom.

The halogen atom may be a fluorine atom, a chlorine atom, or a bromine atom.

When the alkyl group is substituted with a predetermined group, oxygen atoms are not directly bonded to each other.

From the perspective of the stability of the compound, sulfur atoms are preferably not directly bonded to each other, and/or an oxygen atom and a sulfur atom are preferably not directly bonded to each other.

For example, Rⁱ¹ can denote an alkoxy group with 1 to 19 carbon atoms by substituting one —CH₂— in the alkyl group with —O—.

The alkoxy group is a linear, branched, or cyclic alkoxy group, preferably a linear alkoxy group.

The number of carbon atoms in the alkoxy group preferably ranges from 2 to 10 or preferably 2 to 6.

Rⁱ¹ can denote an alkylsulfanyl group (alkylthio group) with 1 to 19 carbon atoms by substituting one —CH₂— in the alkyl group with —S—.

The alkylsulfanyl group is a linear, branched, or cyclic alkylsulfanyl group, preferably a linear alkylsulfanyl group.

The number of carbon atoms in the alkylsulfanyl group preferably ranges from 2 to 10 or preferably 2 to 6.

Rⁱ¹ can denote an alkenyl group with 2 to 20 carbon atoms by substituting one or two or more —CH₂—CH₂— groups in the alkyl group with —CH═CH—.

The alkenyl group is a linear, branched, or cyclic alkenyl group, preferably a linear alkenyl group.

The number of carbon atoms in the alkenyl group preferably ranges from 2 to 10 or preferably 2 to 6.

Rⁱ¹ can denote an alkynyl group with 2 to 20 carbon atoms by substituting one or two or more —CH₂—CH₂— groups in the alkyl group with —C≡C—.

The alkynyl group is a linear, branched, or cyclic alkynyl group, preferably a linear alkynyl group.

The number of carbon atoms in the alkynyl group preferably ranges from 2 to 10 or preferably 2 to 6.

The alkynyl group is preferably an alkynyl group represented by the following formula (Rⁱ¹-A) from the perspective of the ease of synthesis and elongation of a conjugated system.

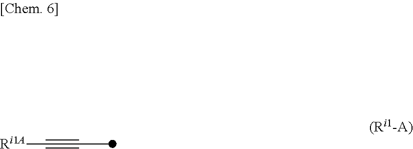

In the formula (Rⁱ¹-A), R^{i1A} denotes an alkyl group with 1 to 18 carbon atoms.

The alkyl group with 1 to 18 carbon atoms is a linear, branched, or cyclic alkyl group, preferably a linear alkyl group.

The number of carbon atoms in the alkyl group with 1 to 18 carbon atoms preferably ranges from 1 to 8.

One or two or more —CH₂— groups in the alkyl group are each independently optionally substituted with —O—, —S—, —CO—, and/or —CS—.

One or two or more —CH₂—CH₂— groups in the alkyl group are each independently optionally substituted with —CO—O—, —O—CO—, —CO—S—, —S—CO—, —CO—NH—, —NH—CO—, —CH═CH—, —CF═CF—, and/or —C≡C—.

One or two or more hydrogen atoms in the alkyl group are each independently optionally substituted with a halogen atom.

The halogen atom may be a fluorine atom, a chlorine atom, a bromine atom, or an iodine atom.

When the alkyl group is substituted with a predetermined group, oxygen atoms are not directly bonded to each other.

From the perspective of the stability of the compound, sulfur atoms are preferably not directly bonded to each other, and/or an oxygen atom and a sulfur atom are preferably not directly bonded to each other.

In the formula (Rⁱ¹-A), a black dot represents a bonding arm to Aⁱ¹.

Rⁱ¹ can denote an alkenyloxy group with 2 to 19 carbon atoms by substituting one —CH₂— in the alkyl group with —O— and substituting one or two or more —CH₂—CH₂— groups with —CH═CH—.

The alkenyloxy group is a linear, branched, or cyclic alkenyloxy group, preferably a linear alkenyloxy group.

The number of carbon atoms in the alkenyloxy group preferably ranges from 2 to 10 or preferably 2 to 6.

Rⁱ¹ can denote an alkyl halide group with 1 to 20 carbon atoms by substituting one or two or more hydrogen atoms in the alkyl group with a halogen atom.

The alkyl halide group is a linear, branched, or cyclic alkyl halide group, preferably a linear alkyl halide group.

The number of carbon atoms in the alkyl halide group preferably ranges from 2 to 10 or preferably 2 to 6.

Rⁱ¹ can denote an alkoxy halide group with 1 to 19 carbon atoms by substituting one —CH₂— in the alkyl group with —O— and substituting one or two or more hydrogen atoms in the alkyl group with a halogen atom.

The alkoxy halide group is a linear, branched, or cyclic alkoxy halide group, preferably a linear alkoxy halide group.

The number of carbon atoms in the alkoxy halide group preferably ranges from 2 to 10 or preferably 2 to 6.

Specific examples of the alkyl group with 1 to 20 carbon atoms (including substituted ones) in Rⁱ¹ include the groups represented by the formulae (Rⁱ¹-1) to (Rⁱ¹-46):

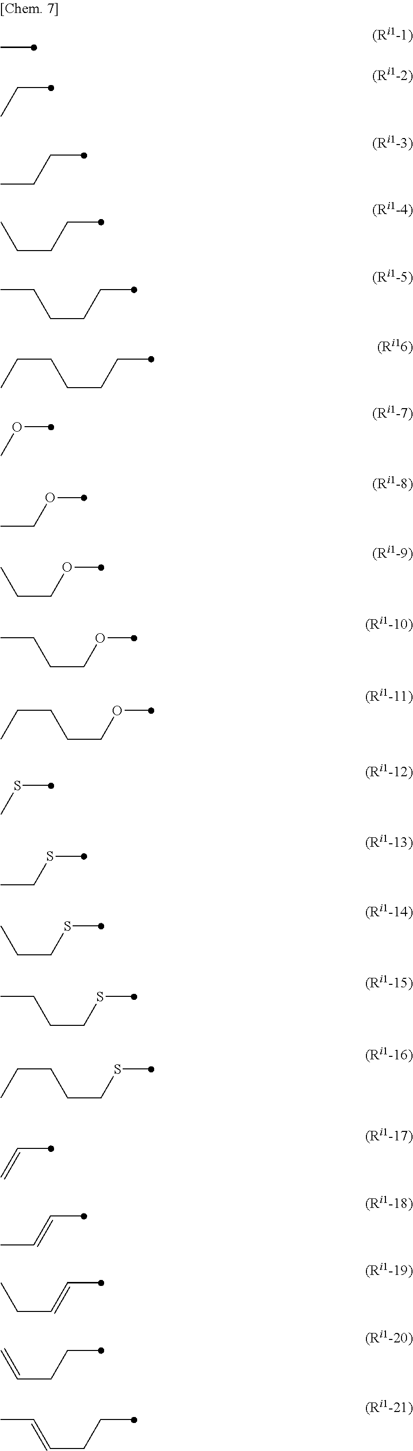

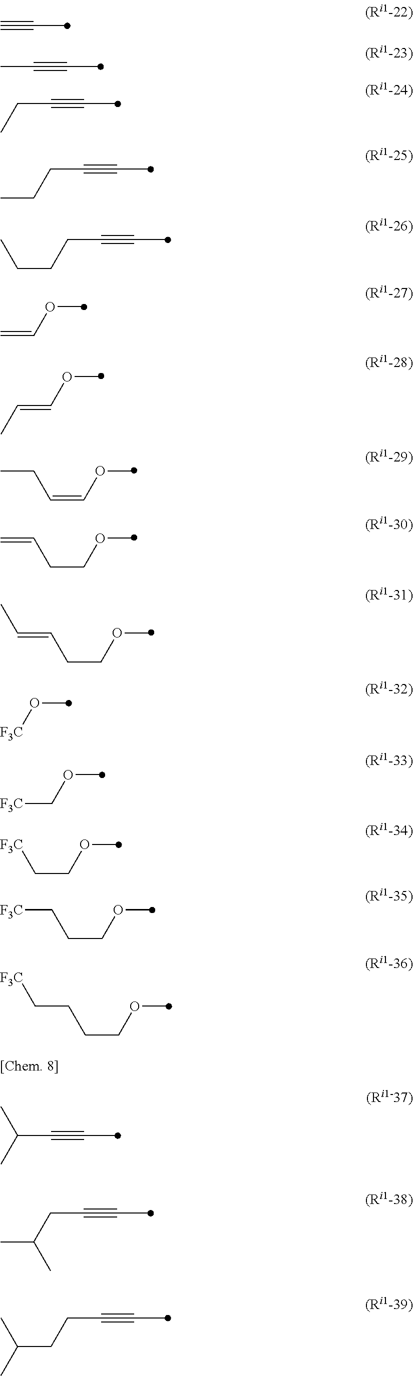

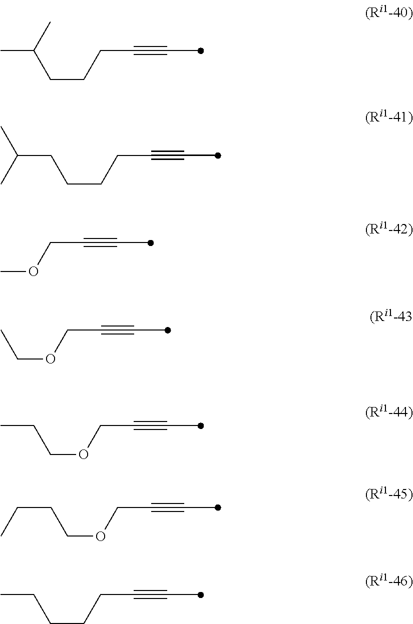

In the formulae (Rⁱ¹-1) to (Rⁱ¹-46), a black dot represents a bonding arm to Aⁱ¹.

Rⁱ¹ is preferably a linear alkyl group with 2 to 6 carbon atoms or a linear alkynyl group with 2 to 8 carbon atoms from the perspective of Δn and compatibility with another liquid crystal compound.

In the general formula (i), Aⁱ¹, Aⁱ², Aⁱ³, and Aⁱ⁴ each independently denote any one of a hydrocarbon ring with 3 to 16 carbon atoms and a heterocycle with 3 to 16 carbon atoms. More specifically, a hydrocarbon ring with 3 to 16 carbon atoms or a heterocycle with 3 to 16 carbon atoms preferably denotes a group selected from the group consisting of the groups (a), (b), (c), and (d):
(a) a 1,4-cyclohexylene group (one —CH₂— or two or more nonadjacent —CH₂— groups in this group are optionally substituted with —O— or —S—),
(b) a 1,4-phenylene group (one —CH═ or two or more nonadjacent —CH═ groups in this group are optionally substituted with —N═),
(c) a 1,4-cyclohexenylene group, a bicycle[2.2.2]octane-1,4-diyl group, a naphthalene-2,6-diyl group, a naphthalene-1,4-diyl group, a 1,2,3,4-tetrahydronaphthalene-2,6-diyl group, a 5,6,7,8-tetrahydronaphthalene-1,4-diyl group, a decahydronaphthalene-2,6-diyl group, an anthracene-2,6-diyl group, an anthracene-1,4-diyl group, an anthracene-9,10-diyl group, a phenanthrene-2,7-diyl group (one —CH═ or two or more —CH═ groups in the naphthalene-2,6-diyl group, the naphthalene-1,4-diyl group, the 1,2,3,4-tetrahydronaphthalene-2,6-diyl group, the 5,6,7,8-tetrahydronaphthalene-1,4-diyl group, the anthracene-2,6-diyl group, the anthracene-1,4-diyl group, the anthracene-9,10-diyl group, or the phenanthrene-2,7-diyl group are optionally substituted with —N═), and
(d) a thiophene-2,5-diyl group, a benzothiophene-2,5-diyl group, a benzothiophene-2,6-diyl group, a dibenzothiophene-3,7-diyl group, a dibenzothiophene-2,6-diyl group, a thieno[3,2-b]thiophene-2,5-diyl group (one —CH═ or two or more nonadjacent —CH═ groups in this group are optionally substituted with —N═).

One or two or more hydrogen atoms in Aⁱ¹, Aⁱ², Aⁱ³, and Aⁱ⁴ are each independently optionally substituted with a substituent Sⁱ¹.

The substituent Sⁱ¹ denotes any one of a fluorine atom, a chlorine atom, a bromine atom, an iodine atom, a pentafluorosulfanyl group, a nitro group, a cyano group, an isocyano group, an amino group, a hydroxy group, a mercapto group, a methylamino group, a dimethylamino group, a diethylamino group, a diisopropylamino group, a trimethylsilyl group, a dimethylsilyl group, a thioisocyano group, and an alkyl group with 1 to 20 carbon atoms.

The alkyl group is a linear, branched, or cyclic alkyl group, preferably a linear alkyl group.

The number of carbon atoms in the alkyl group preferably ranges from 2 to 10 or preferably 3 to 6.

One or two or more —CH₂— groups in the alkyl group are each independently optionally substituted with —O—, —S—, and/or —CO—.

One or two or more —CH₂—CH₂— groups in the alkyl group are each independently optionally substituted with —CH═CH—, —CF═CF—, —C≡C—, —CO—O—, —O—CO—, —CO—S—, —S—CO—, —CO—NH—, and/or —NH—CO—.

One or two or more hydrogen atoms in the alkyl group are each independently optionally substituted with a halogen atom.

The halogen atom may be a fluorine atom, a chlorine atom, a bromine atom, or an iodine atom.

When the alkyl group is substituted with a predetermined group, oxygen atoms are not directly bonded to each other.

From the perspective of the stability of the compound, sulfur atoms are preferably not directly bonded to each other, and/or an oxygen atom and a sulfur atom are preferably not directly bonded to each other.

The substituent Sⁱ¹ is preferably a fluorine atom, a linear alkyl group with 1 to 6 carbon atoms, or a linear alkoxy group with 1 to 6 carbon atoms.

At least one of Aⁱ¹, Aⁱ², Aⁱ³, and Aⁱ⁴ is preferably substituted with at least one substituent Sⁱ¹.

Aⁱ⁴ is preferably substituted with at least one substituent Sⁱ¹.

A plurality of substituents Sⁱ¹, if present, may be the same or different.

The substitution position of the substituent Sⁱ¹ in Aⁱ¹ is preferably any one of the formulae (Aⁱ¹-SP-1) to (Aⁱ¹-SP-4):

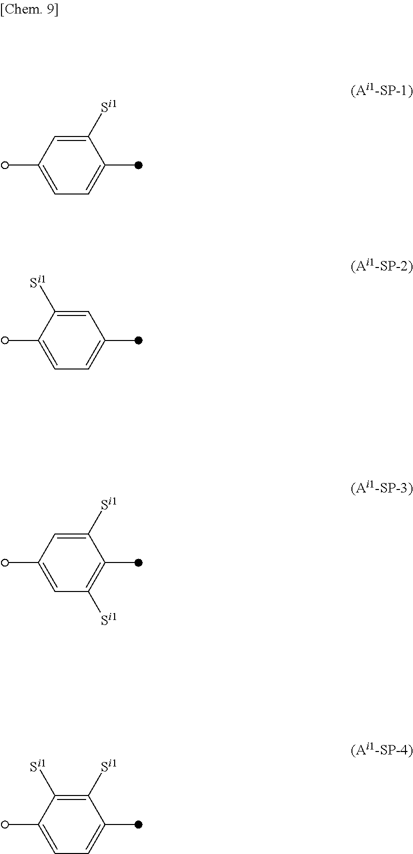

In the formulae (Aⁱ¹-SP-1) to (Aⁱ¹-SP-4), a white dot represents a bonding arm to Rⁱ¹, and a black dot represents a bonding arm to Zⁱ¹.

The substitution position of the substituent Sⁱ¹ in Aⁱ² is preferably any one of the formulae (Aⁱ²-SP-1) to (Aⁱ²-SP-4):

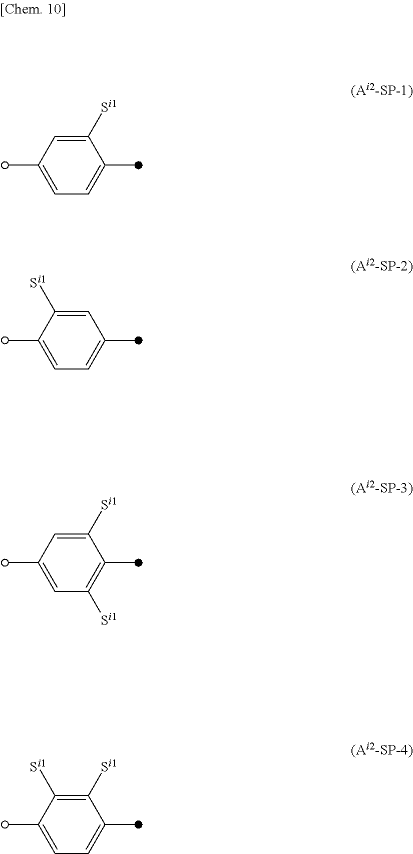

In the formulae (Aⁱ²-SP-1) to (Aⁱ²-SP-4), a white dot represents a bonding arm to Zⁱ¹, and a black dot represents a bonding arm to Zⁱ².

The substitution position of the substituent Sⁱ¹ in Aⁱ³ is preferably any one of the formulae (Aⁱ³-SP-1) to (Aⁱ³-SP-4):

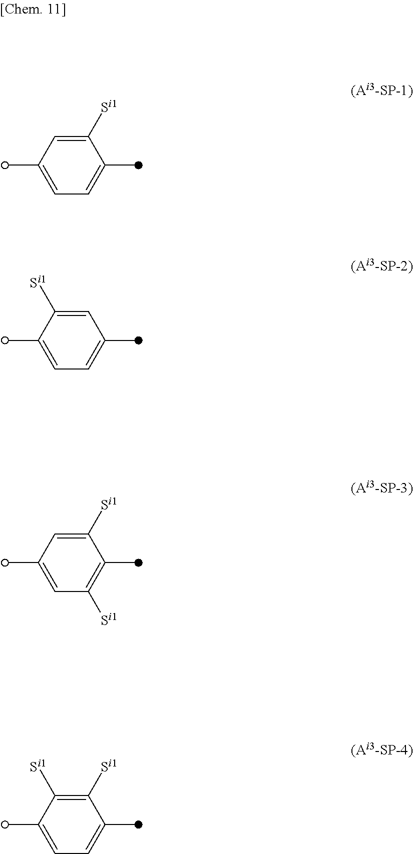

In the formulae (Aⁱ³-SP-1) to (Aⁱ³-SP-4), a white dot represents a bonding arm to Zⁱ², and a black dot represents a bonding arm to Zⁱ³.

The substitution position of the substituent Sⁱ¹ in Aⁱ⁴ is preferably any one of the following formulae (Aⁱ⁴-SP-1) to (Aⁱ⁴-SP-4):

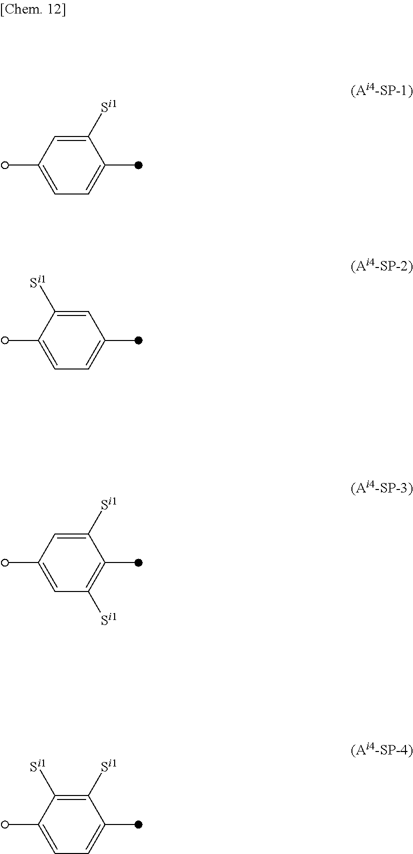

In the formulae (Aⁱ⁴-SP-1) to (Aⁱ⁴-SP-4), a white dot represents a bonding arm to Zⁱ³, and a black dot represents a bonding arm to an isothiocyanate group (—NCS).

More specifically, Aⁱ¹ preferably denotes any one of the formulae (Aⁱ¹-1) to (Aⁱ¹-11):

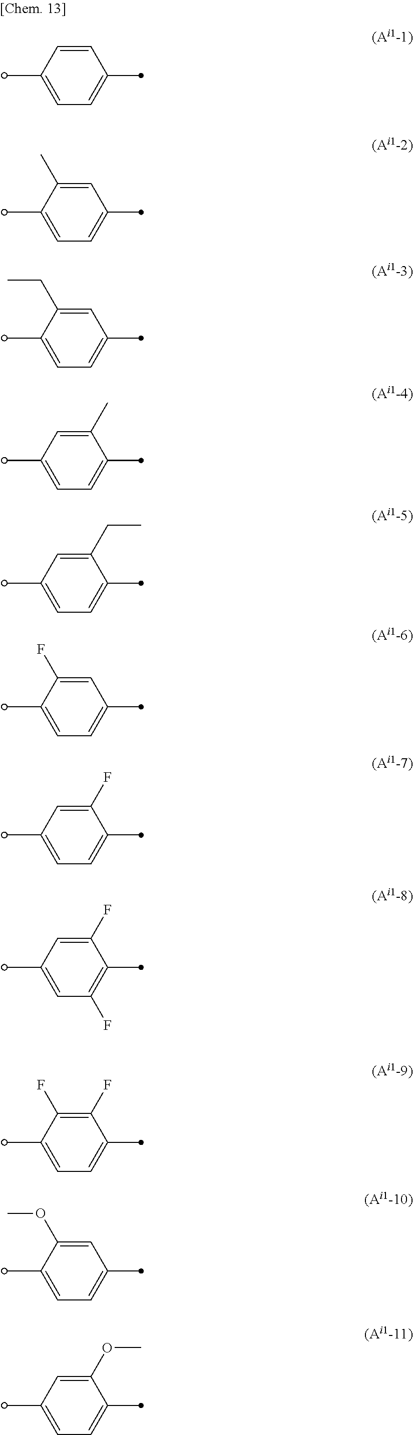

In the formulae (Aⁱ¹-1) to (Aⁱ¹-11), a white dot represents a bonding arm to Rⁱ¹, and a black dot represents a bonding arm to Zⁱ¹.

More specifically, Aⁱ² preferably denotes any one of the formulae (Aⁱ²-1) to (Aⁱ²-11):

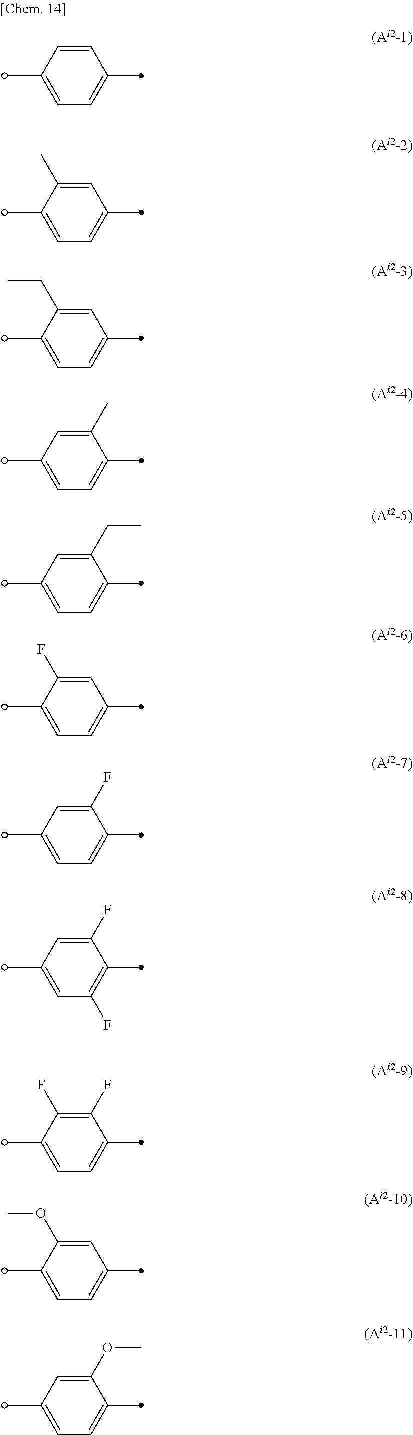

In the formulae (Aⁱ²-1) to (Aⁱ²-11), a white dot represents a bonding arm to Zⁱ¹, and a black dot represents a bonding arm to Zⁱ².

More specifically, Aⁱ³ preferably denotes any one of the formulae (Aⁱ³-1) to (Aⁱ³-11):

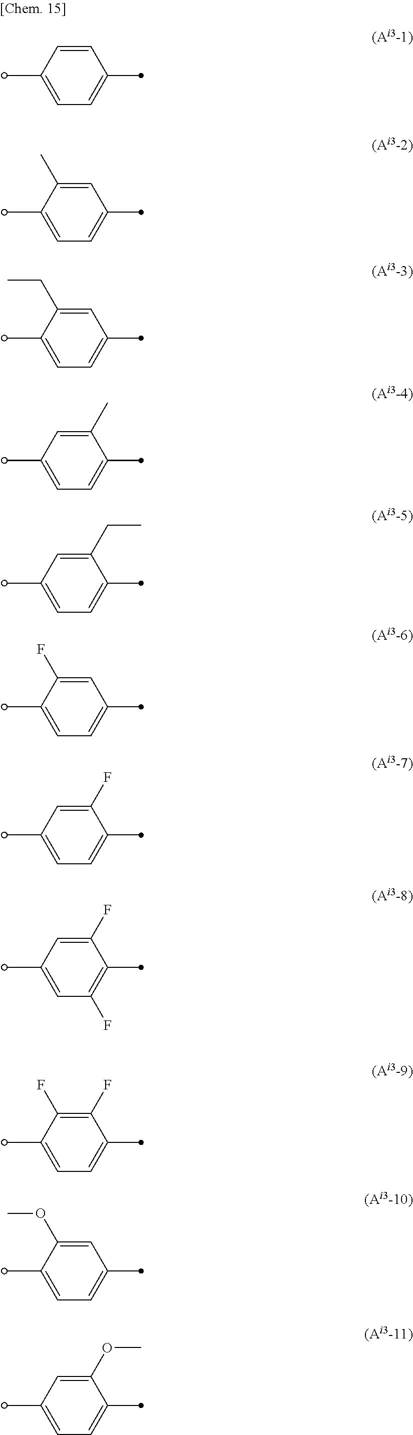

In the formulae (Aⁱ³-1) to (Aⁱ³-11), a white dot represents a bonding arm to Zⁱ², and a black dot represents a bonding arm to Zⁱ³.

More specifically, Aⁱ⁴ preferably denotes any one of the formulae (Aⁱ⁴-1) to (Aⁱ⁴-11):

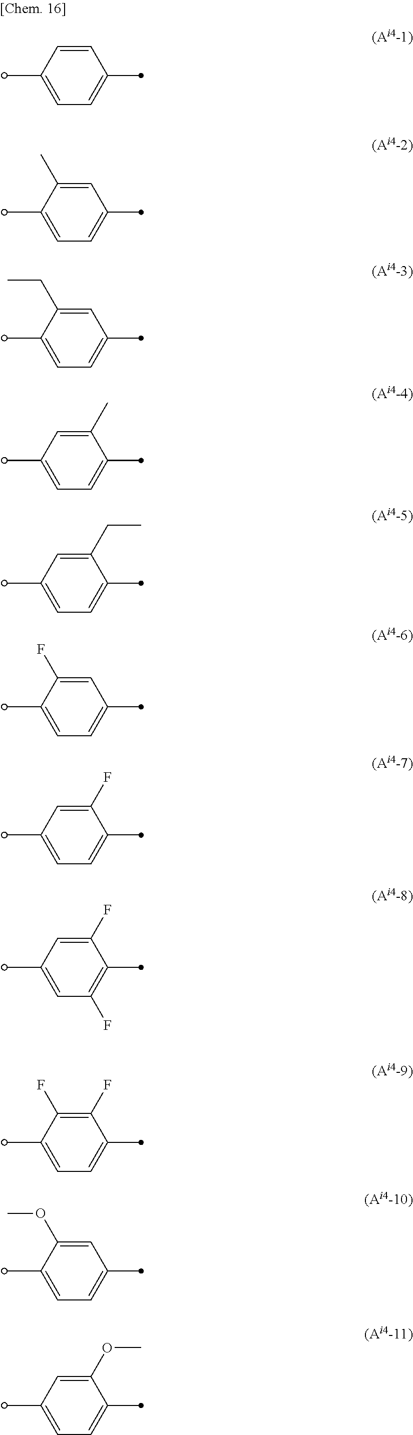

In the formulae (Aⁱ⁴-1) to (Aⁱ⁴-11), a white dot represents a bonding arm to Zⁱ³, and a black dot represents a bonding arm to an isothiocyanate group (—NCS).

Zⁱ¹, Zⁱ², and Zⁱ³ each independently denote any one of a single bond and an alkylene group with 1 to 20 carbon atoms.

At least one of Zⁱ¹, Zⁱ², and Zⁱ³ denotes —C≡C—.

The alkylene group is a linear, branched, or cyclic alkylene group, preferably a linear alkylene group.

The number of carbon atoms in the alkylene group preferably ranges from 2 to 10 or preferably 2 to 6.

One or two or more —CH₂— groups in the alkylene group are each independently optionally substituted with —O—, —CF₂—, and/or —CO—.

One or two or more —CH₂—CH₂— groups in the alkylene group are each independently optionally substituted with —CH₂—CH(CH₃)—, —CH(CH₃)—CH₂—, —CH═CH—, —CF═CF—, —CH═C(CH₃)—, —C(CH₃)═CH—, —CH═N—, —N═CH—, —N═N—, —C≡C—, —CO—O—, and/or —O—CO—.

When the alkylene group is substituted with a predetermined group, oxygen atoms are not directly bonded to each other.

Specific examples of the alkylene group with 2 to 20 carbon atoms (including substituted ones) include the groups represented by the formulae (Z^{i1/2/3}-1) to (Z^{i1/2/3}-24):

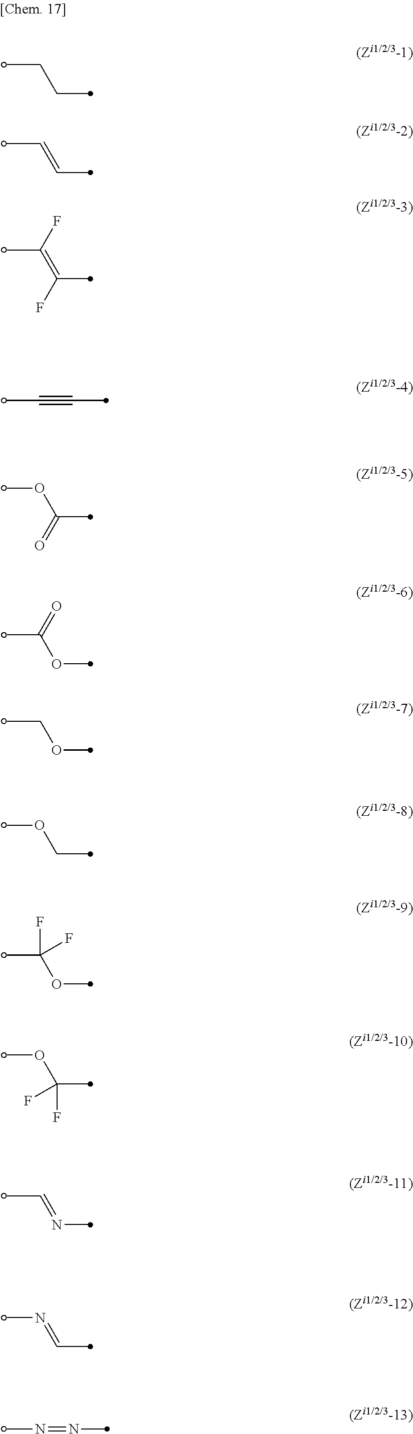

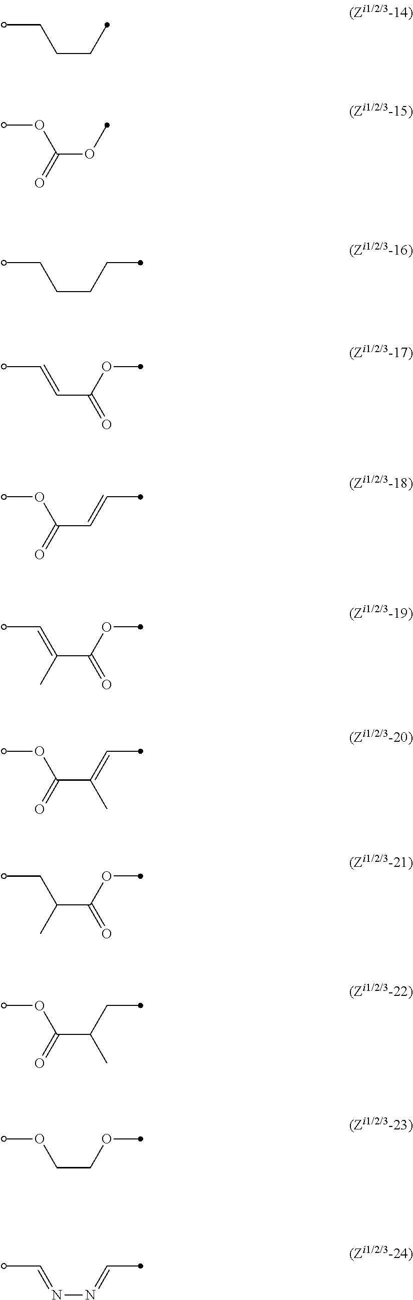

In the formulae (Z^{i1/2/3}-1) to (Z^{i1/2/3}-24), a white dot represents a bonding arm to Aⁱ¹, Aⁱ², or Aⁱ³, and a black dot represents a bonding arm to Aⁱ², Aⁱ³, or Aⁱ⁴.

From the perspective of improving Δn, Zⁱ³ is preferably represented by the formula (Z^{i1/2/3}-4) (—C≡C—).

Compounds represented by the general formula (i) are preferably the compounds represented by the following general formulae (i-1) to (i-4):

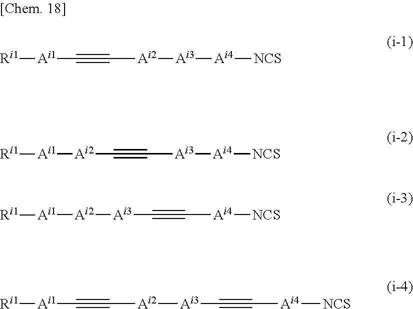

In the general formulae (i-1) to (i-4), Rⁱ¹, Aⁱ¹, Aⁱ², Aⁱ³, and Aⁱ⁴ each independently denote the same as Rⁱ¹, Aⁱ¹, Aⁱ², Aⁱ³, and Aⁱ⁴ in the general formula (i).

Compounds represented by the general formula (i-1) are preferably the compounds represented by the following general formulae (i-1-1) to (i-1-20):

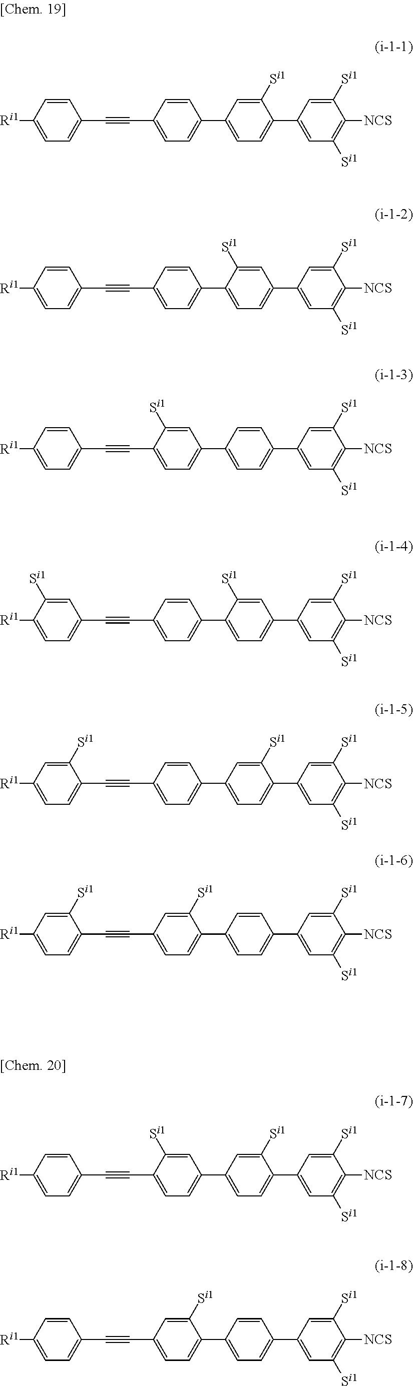

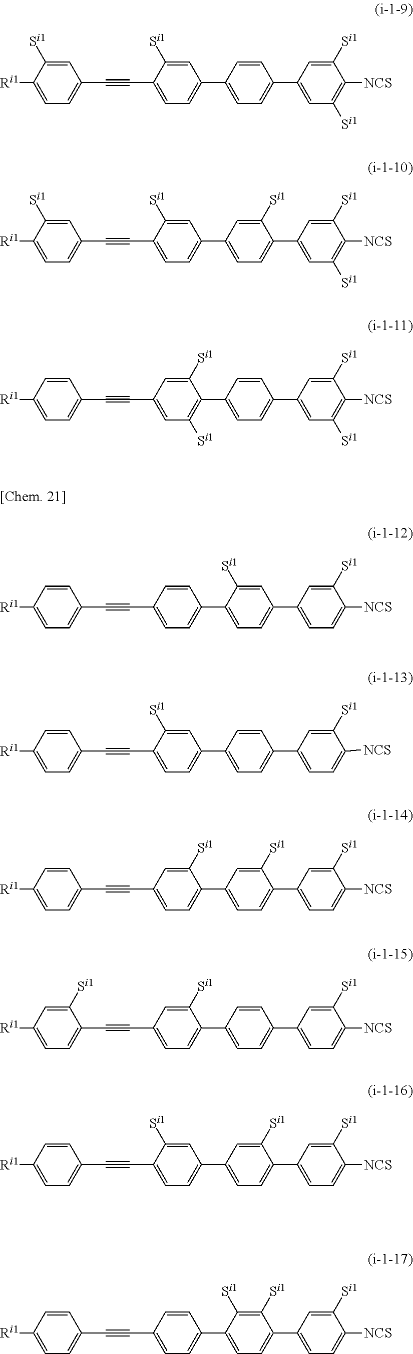

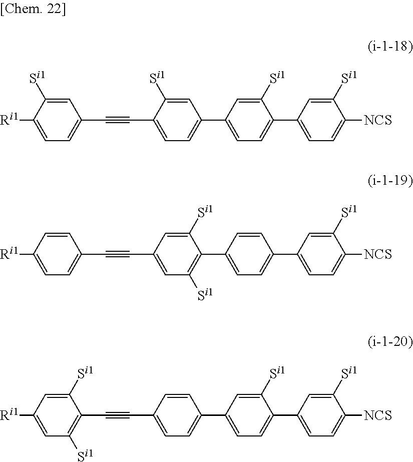

In the general formulae (i-1-1) to (i-1-20), Rⁱ¹ and Sⁱ¹ each independently denote the same as Rⁱ¹ and Sⁱ¹ in the general formula (i).

Specific examples of the compounds represented by the general formula (i-1-1) include the compounds represented by the following structural formulae (i-1-1.1) to (i-1-1.8):

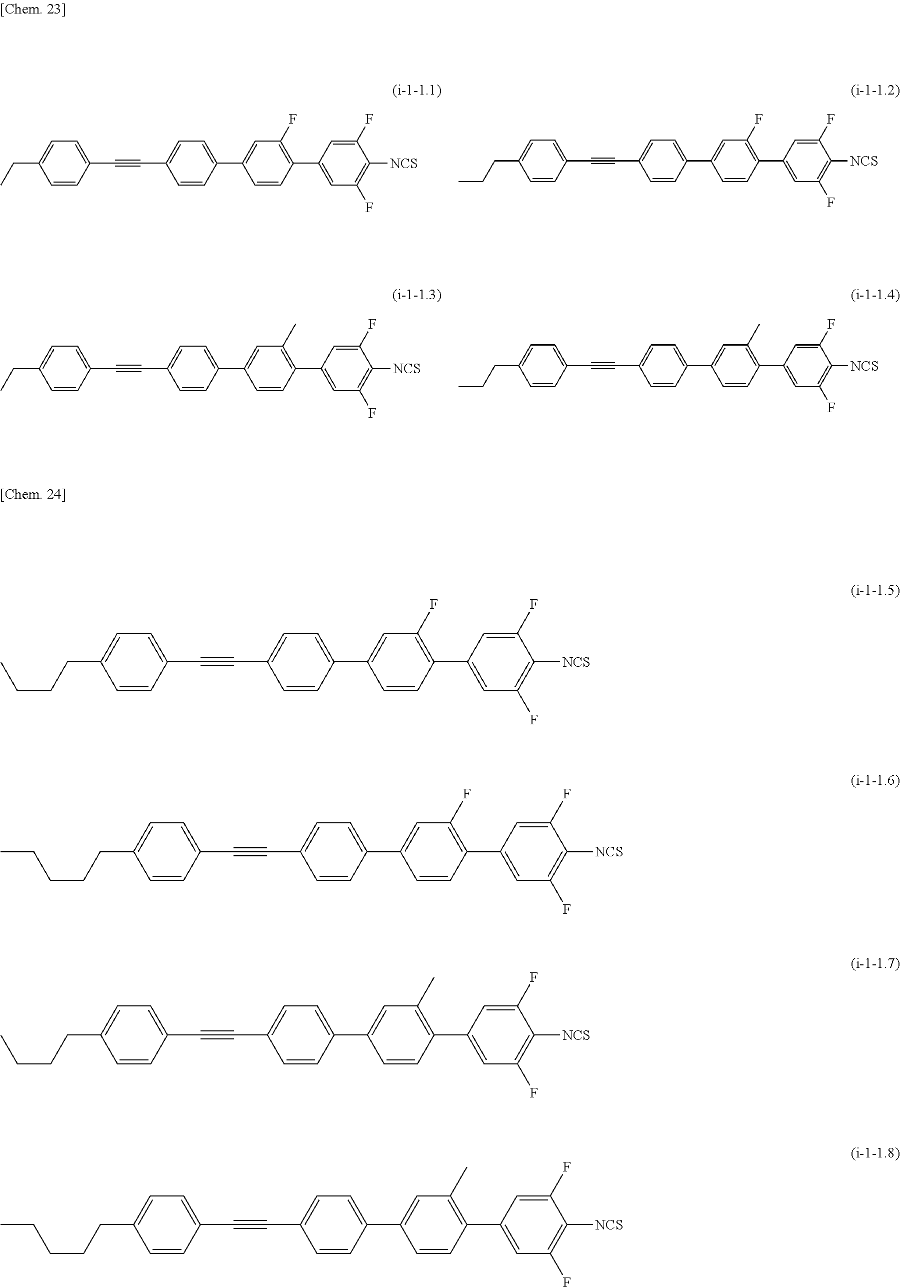

Specific examples of the compounds represented by the general formula (i-1-2) include the compounds represented by the following structural formulae (i-1-2.1) to (i-1-2.4):

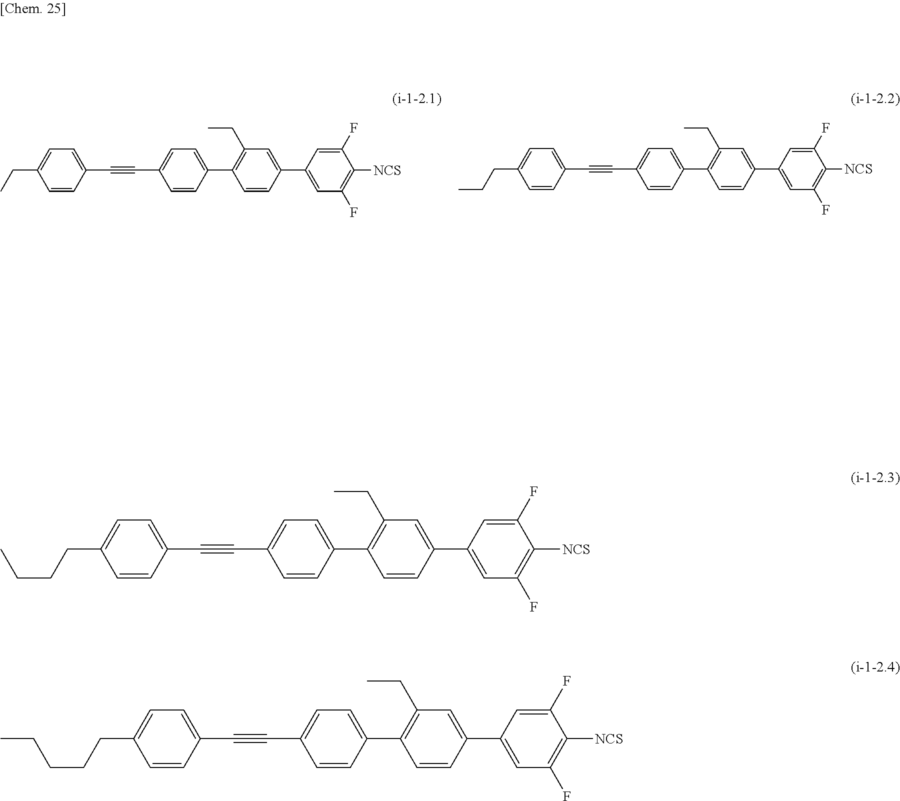

Specific examples of the compounds represented by the general formula (i-1-3) include the compounds represented by the following structural formulae (i-1-3.1) to (i-1-3.4):

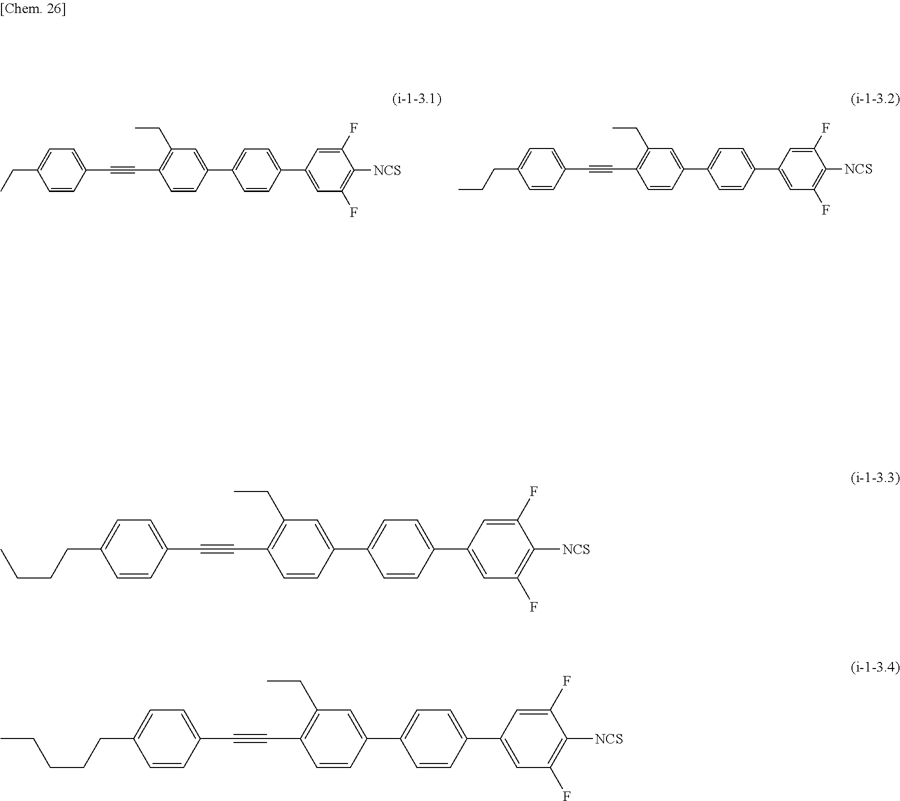

Specific examples of the compounds represented by the general formula (i-1-4) include the compounds represented by the following structural formulae (i-1-4.1) to (i-1-4.4):

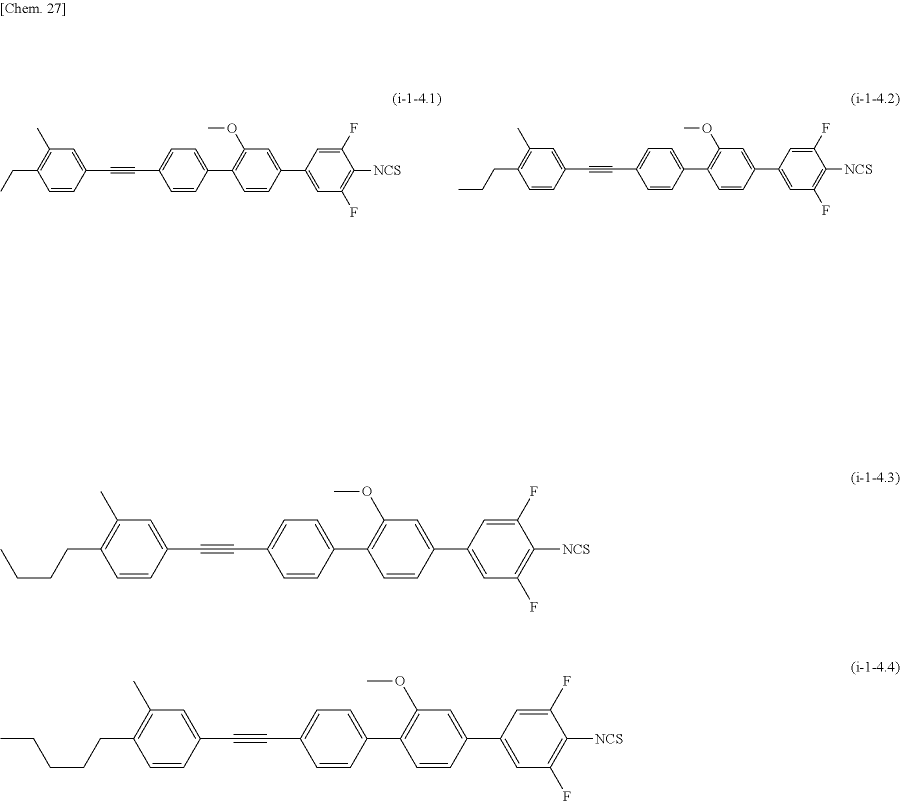

Specific examples of the compounds represented by the general formula (i-1-5) include the compounds represented by the following structural formulae (i-1-5.1) to (i-1-5.4):

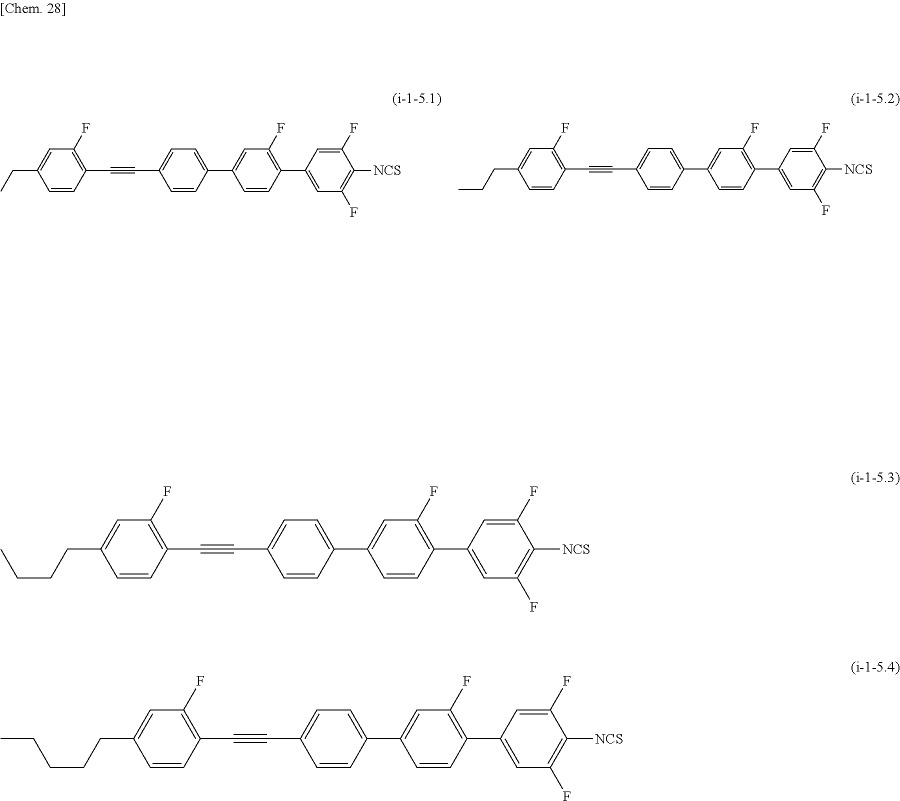

Specific examples of the compounds represented by the general formula (i-1-6) include the compounds represented by the following structural formulae (i-1-6.1) to (i-1-6.4):

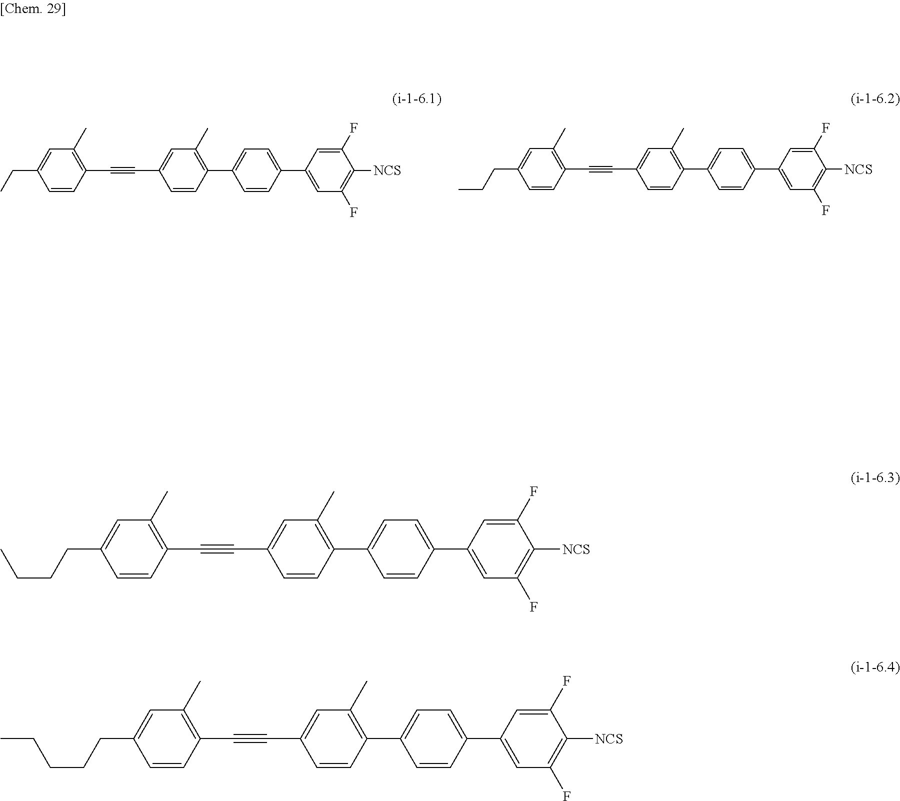

Specific examples of the compounds represented by the general formula (i-1-7) include the compounds represented by the following structural formulae (i-1-7.1) to (i-1-7.8):

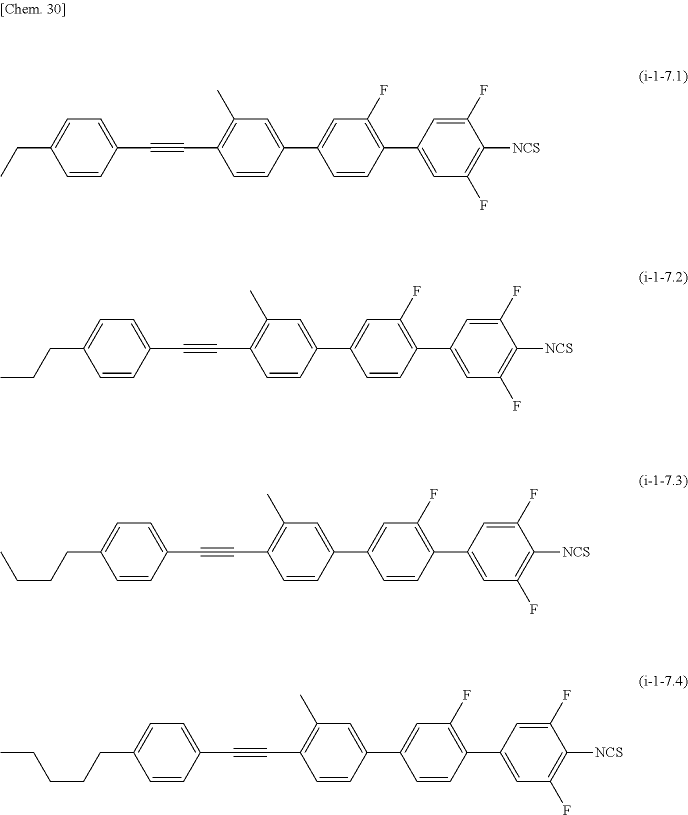

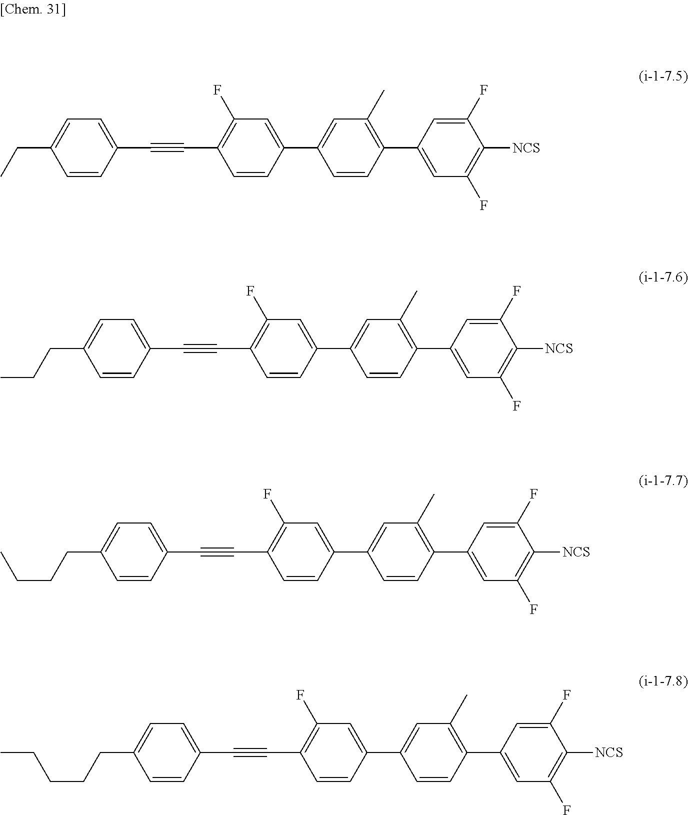

Specific examples of the compounds represented by the general formula (i-1-8) include the compounds represented by the following structural formulae (i-1-8.1) to (i-1-8.8):

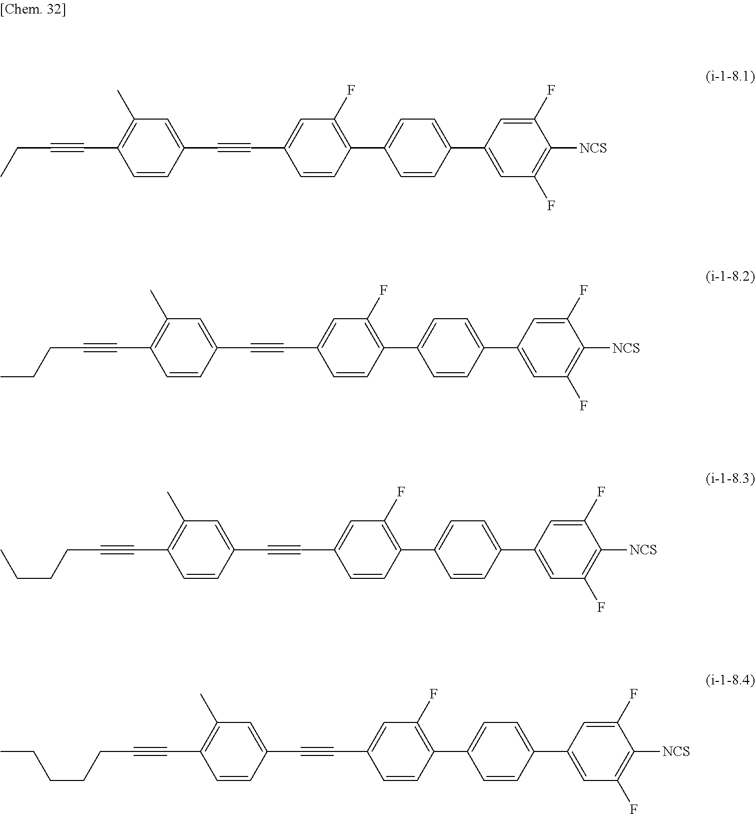

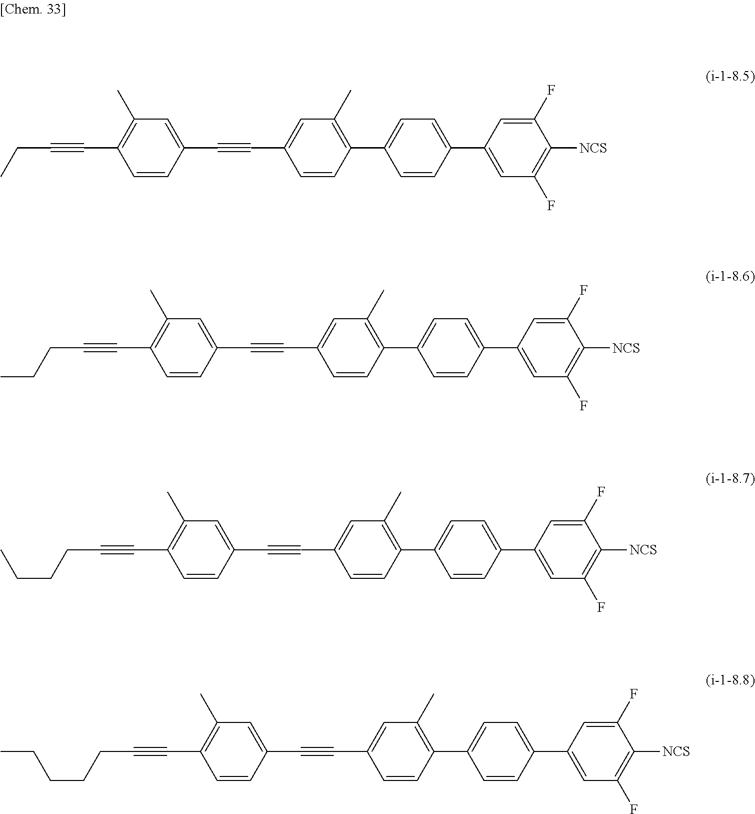

Specific examples of the compounds represented by the general formula (i-1-9) include the compounds represented by the following structural formulae (i-1-9.1) to (i-1-9.8):

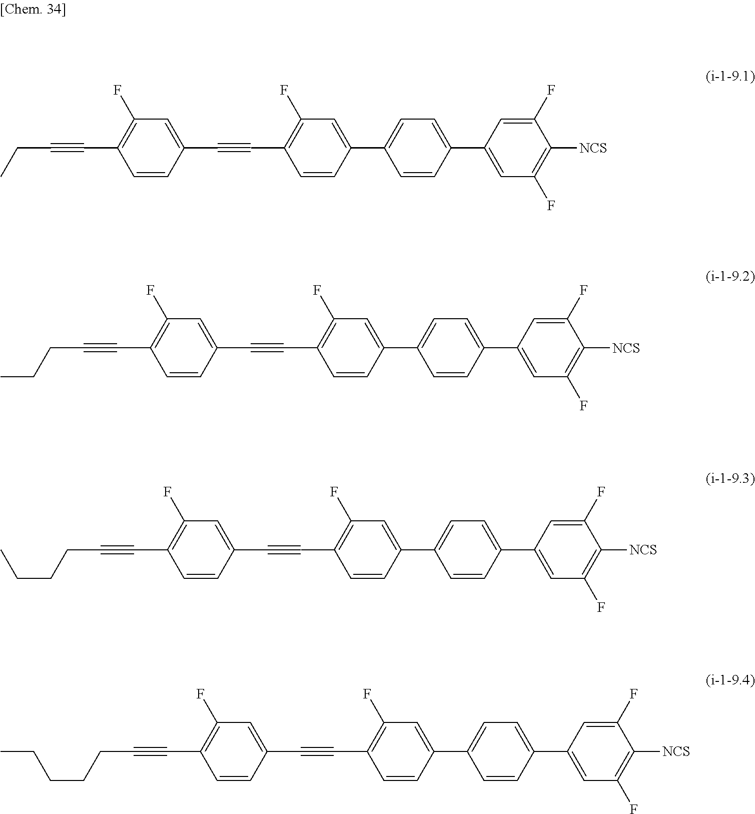

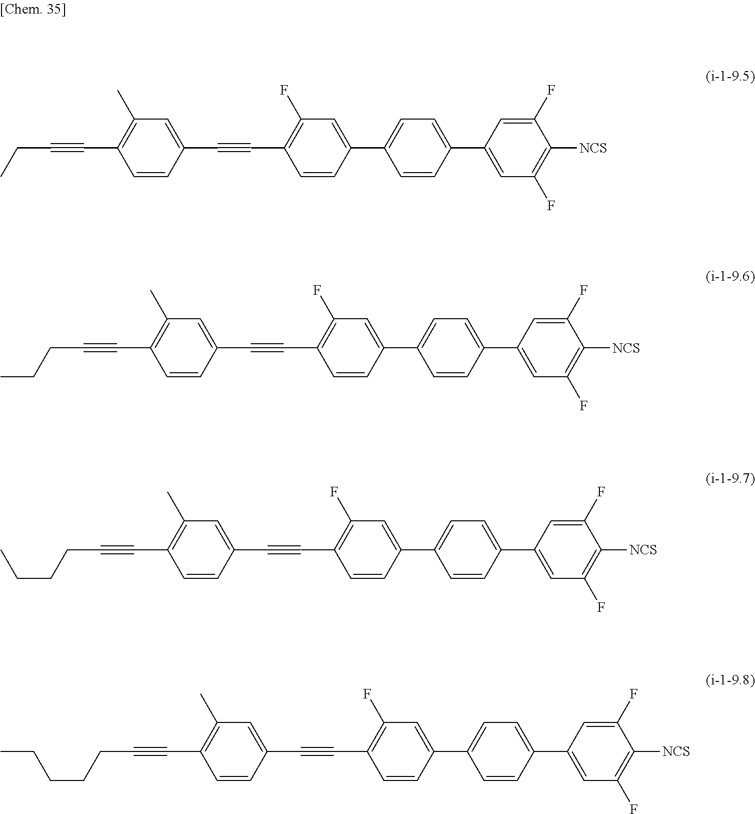

Specific examples of the compounds represented by the general formula (i-1-10) include the compounds represented by the following structural formulae (i-1-10.1) to (i-1-10.4):

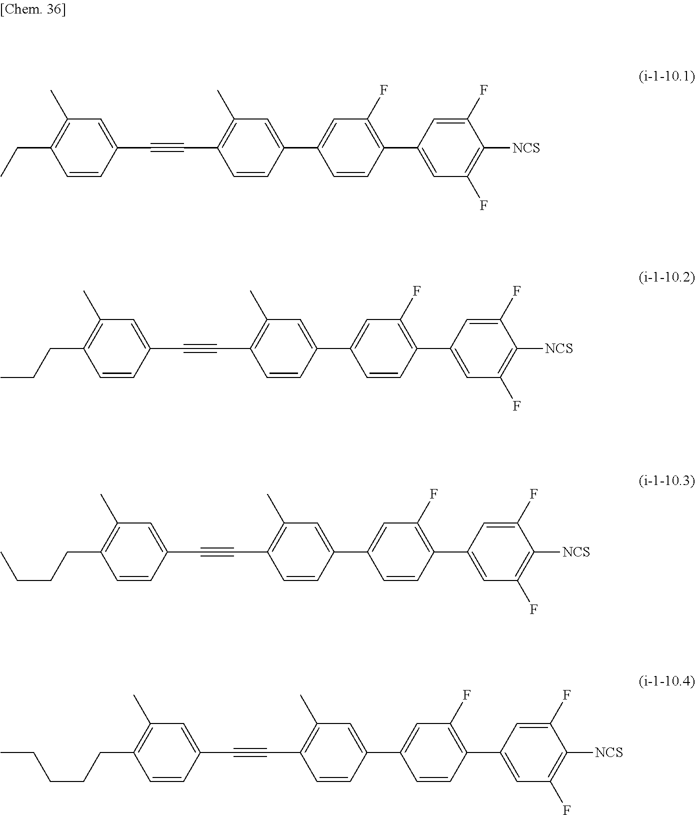

Specific examples of the compounds represented by the general formula (i-1-11) include the compounds represented by the following structural formulae (i-1-11.1) to (i-1-11.4):

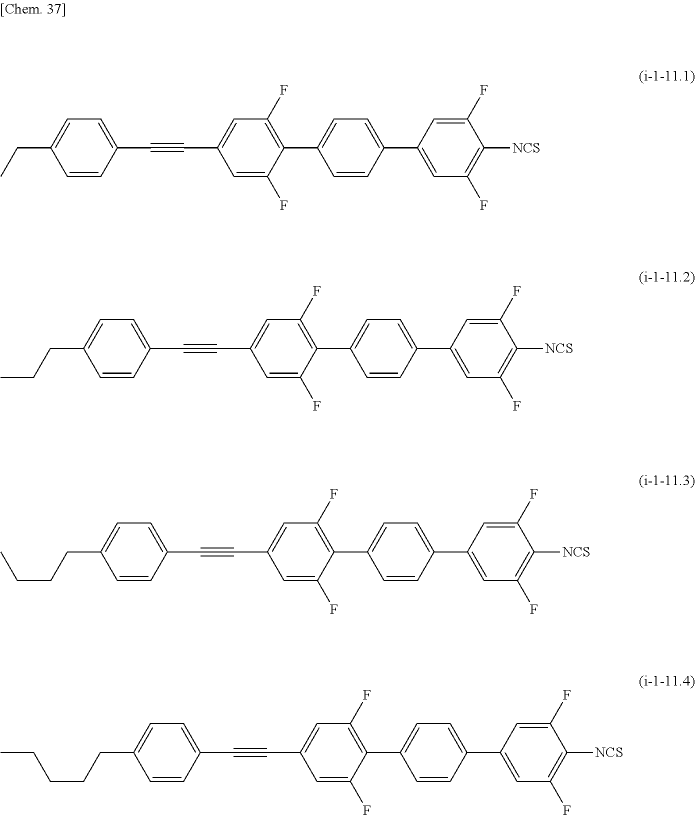

Specific examples of the compounds represented by the general formula (i-1-12) include the compounds represented by the following structural formulae (i-1-12.1) to (i-1-12.8):

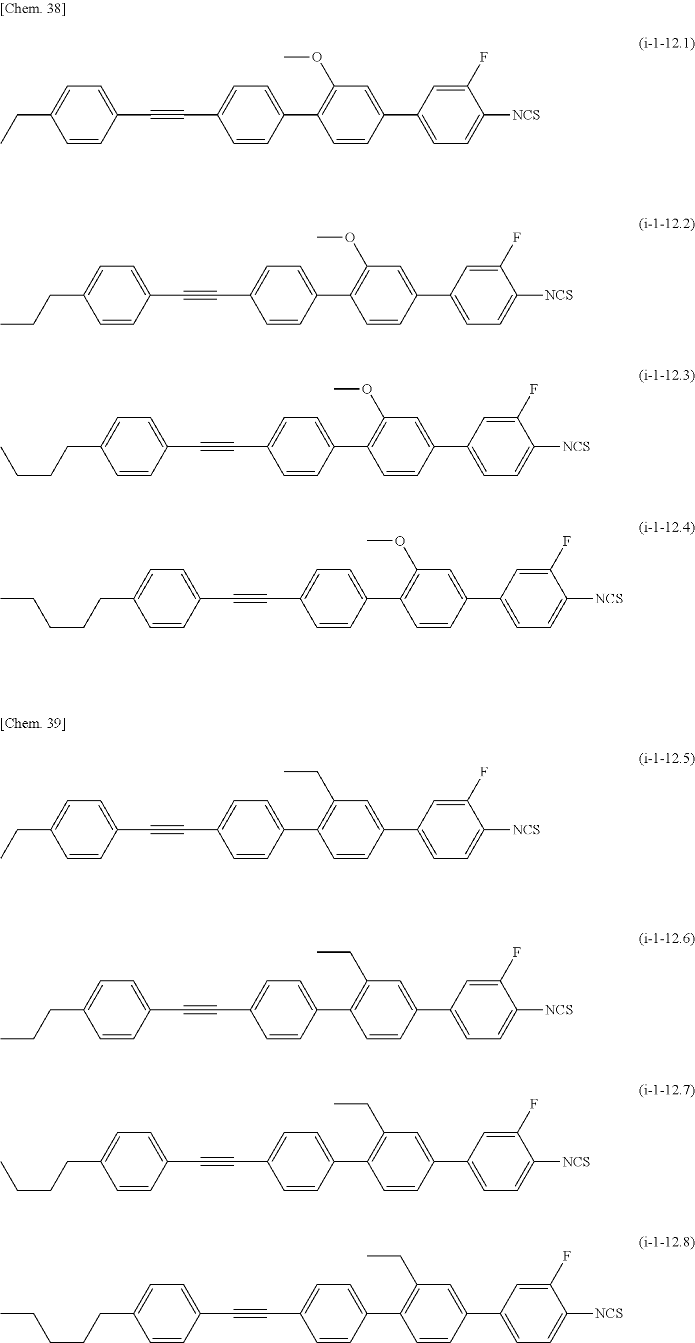

Specific examples of the compounds represented by the general formula (i-1-13) include the compounds represented by the following structural formulae (i-1-13.1) to (i-1-13.8):

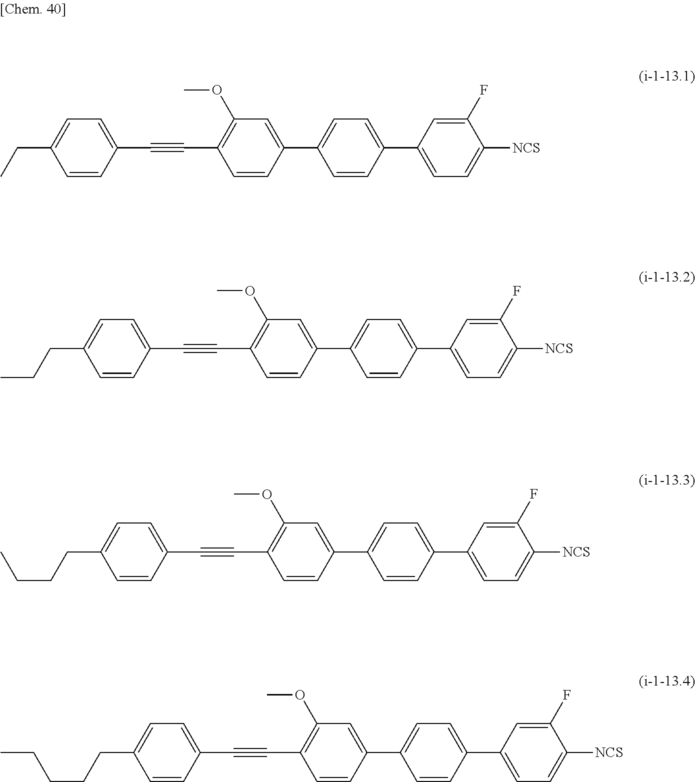

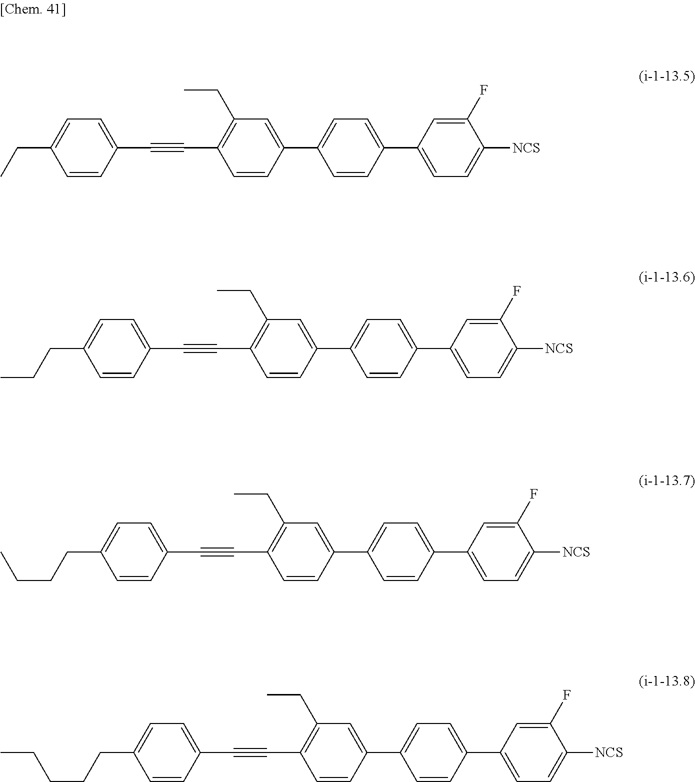

Specific examples of the compounds represented by the general formula (i-1-14) include the compounds represented by the following structural formulae (i-1-14.1) to (i-1-14.4):

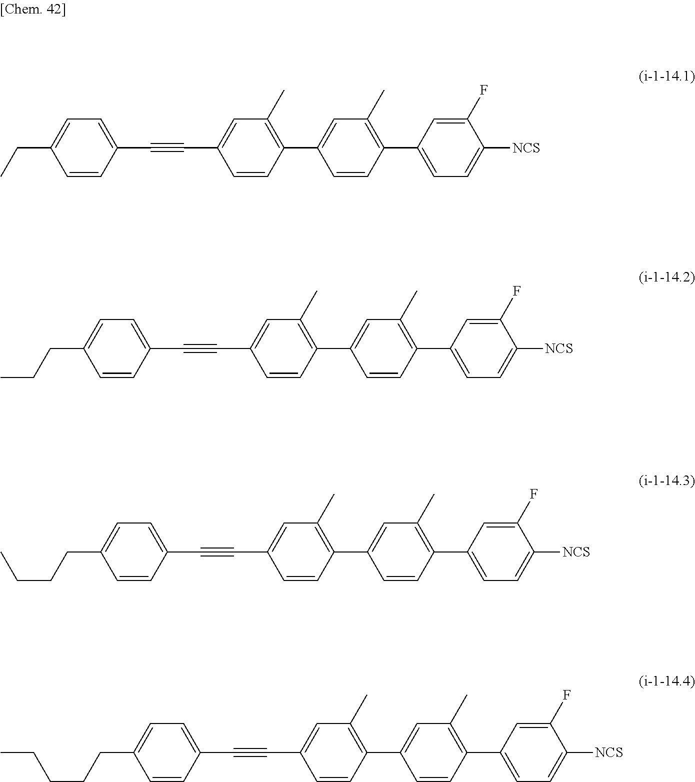

Specific examples of the compounds represented by the general formula (i-1-15) include the compounds represented by the following structural formulae (i-1-15.1) to (i-1-15.4):

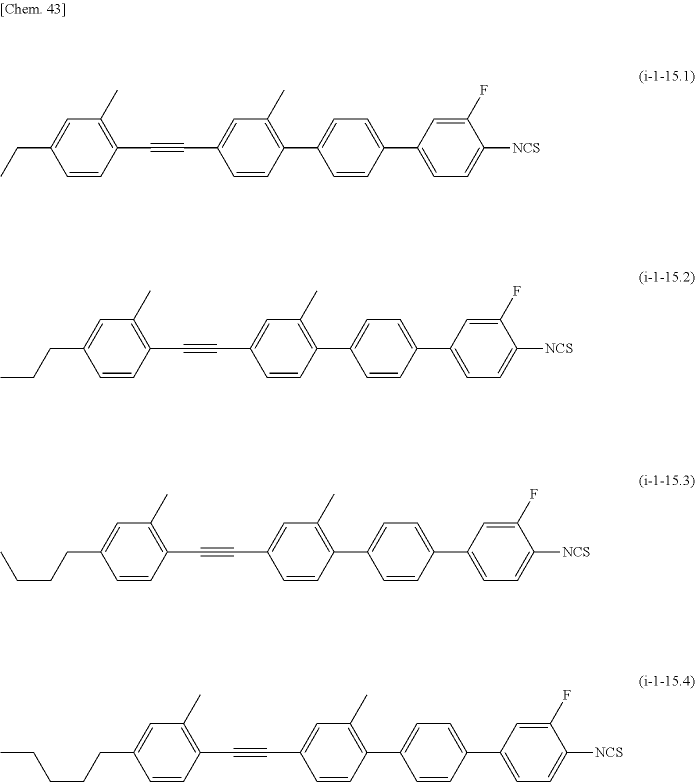

Specific examples of the compounds represented by the general formula (i-1-16) include the compounds represented by the following structural formulae (i-1-16.1) to (i-1-16.4):

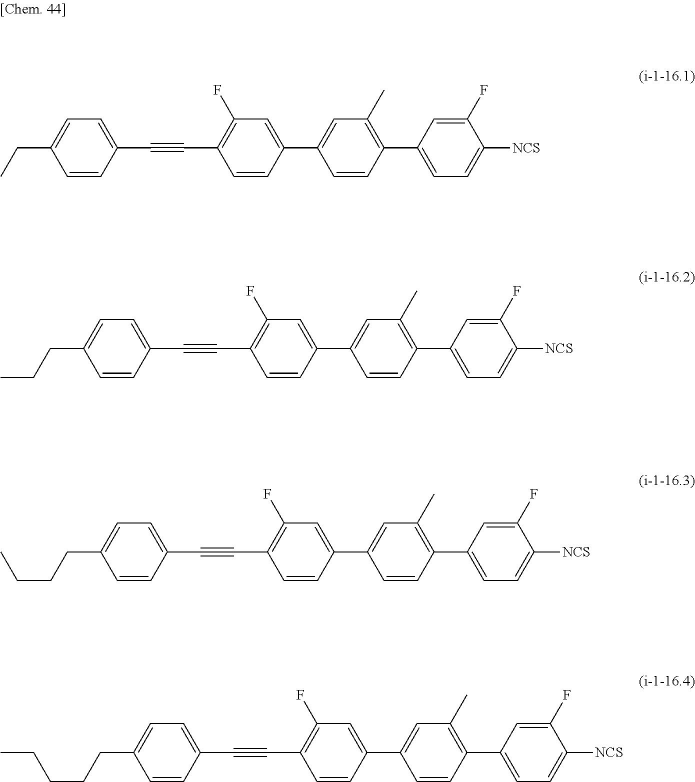

Specific examples of the compounds represented by the general formula (i-1-17) include the compounds represented by the following structural formulae (i-1-17.1) to (i-1-17.4):

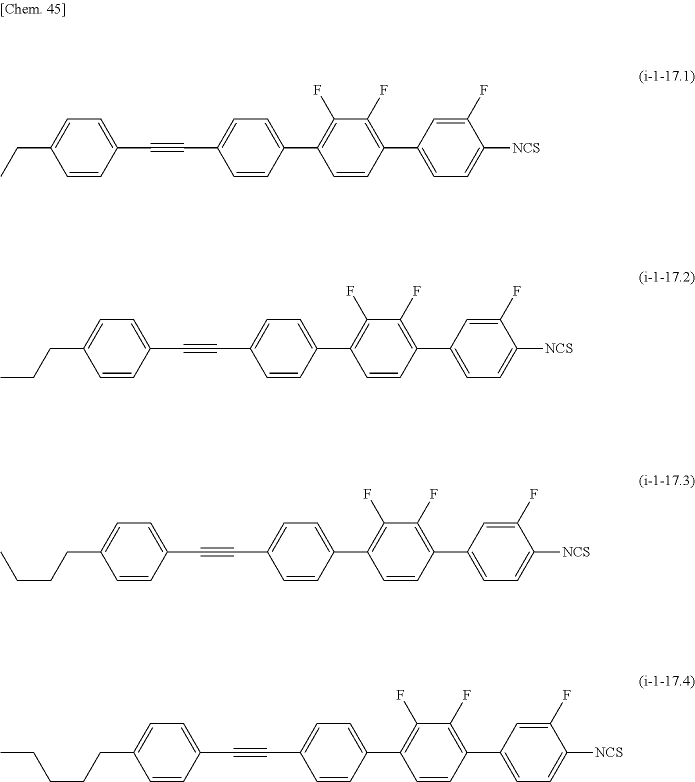

Specific examples of the compounds represented by the general formula (i-1-18) include the compounds represented by the following structural formulae (i-1-18.1) to (i-1-18.4):

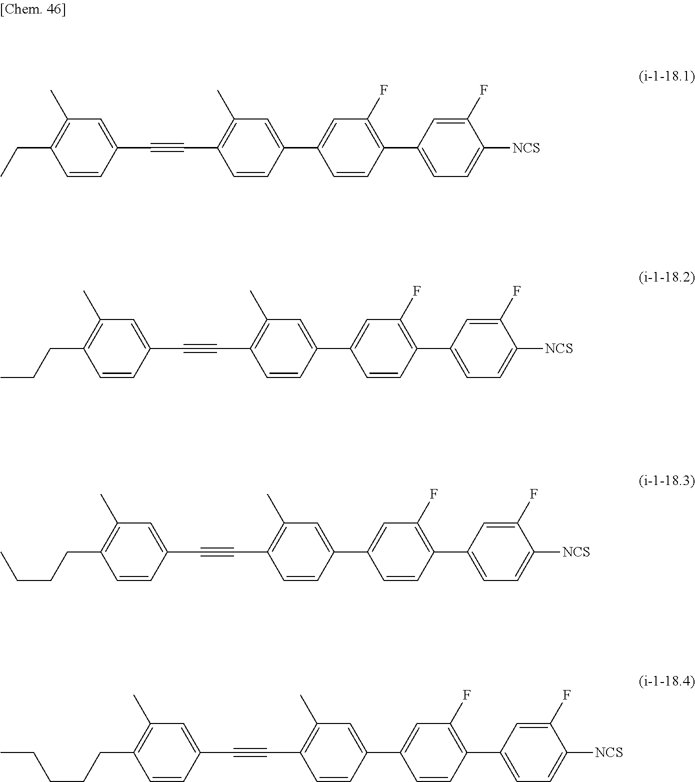

Specific examples of the compounds represented by the general formula (i-1-19) include the compounds represented by the following structural formulae (i-1-19.1) to (i-1-19.4):

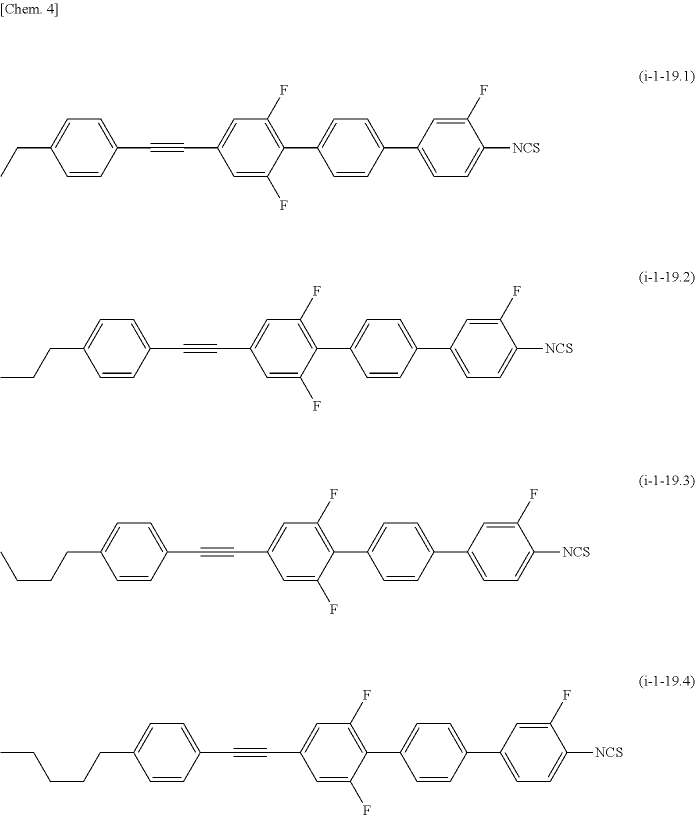

Specific examples of the compounds represented by the general formula (i-1-20) include the compounds represented by the following structural formulae (i-1-20.1) to (i-1-20.4):

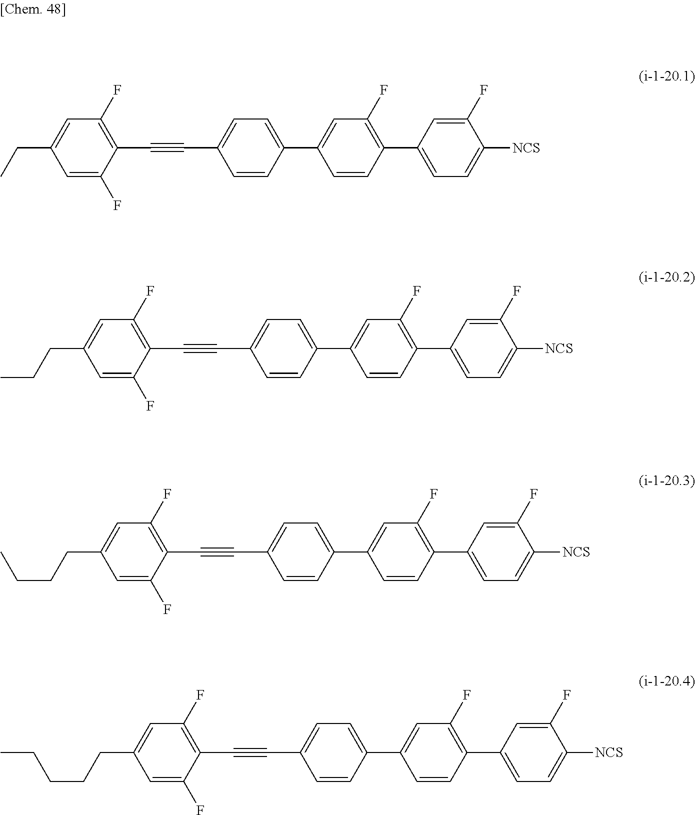

Compounds represented by the general formula (i-2) are preferably the compounds represented by the following general formulae (i-2-1) to (i-2-18):

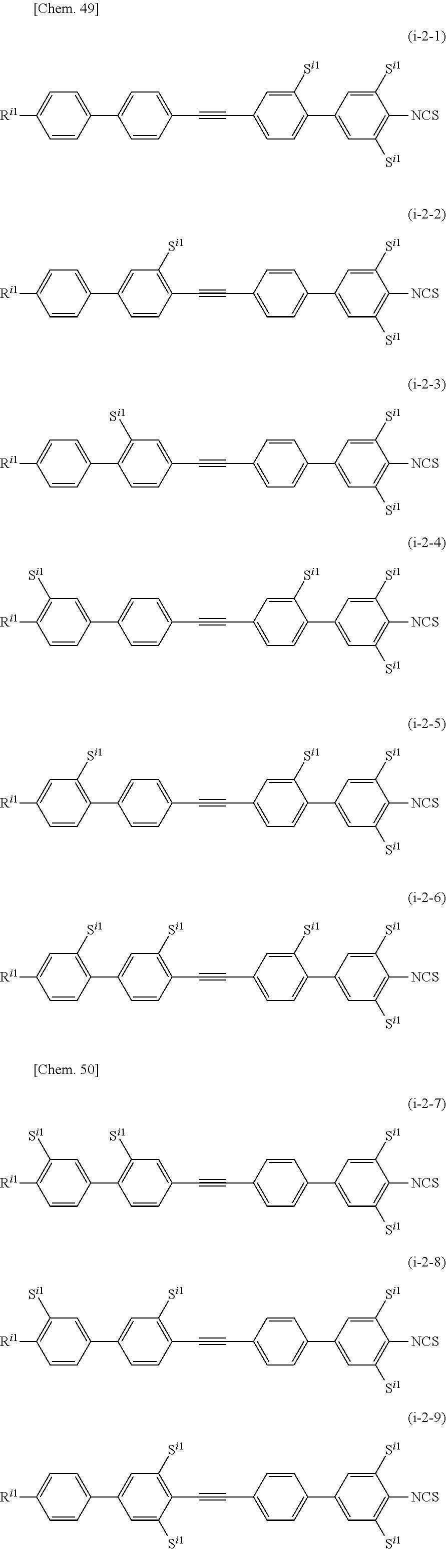

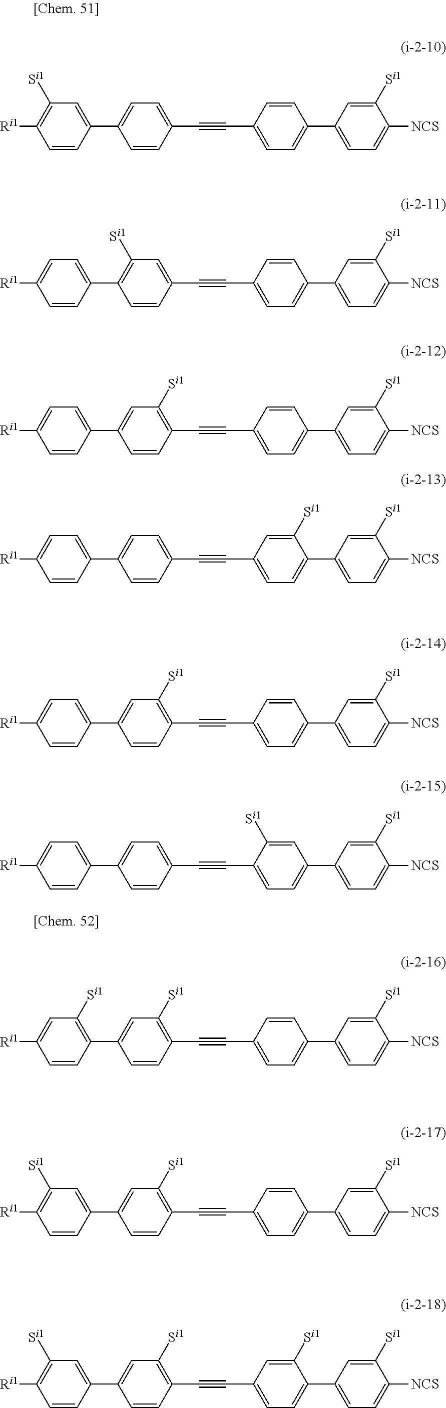

In the general formulae (i-2-1) to (i-2-18), Rⁱ¹ and Sⁱ¹ each independently denote the same as Rⁱ¹ and Sⁱ¹ in the general formula (i).

Specific examples of the compounds represented by the general formula (i-2-1) include the compounds represented by the following structural formulae (i-2-1.1) to (i-2-1.4):

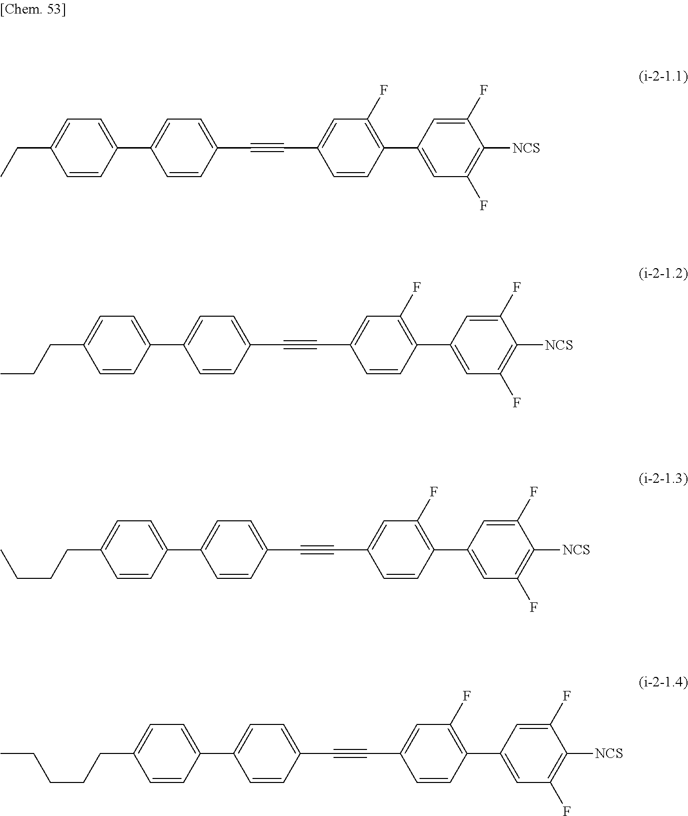

Specific examples of the compounds represented by the general formula (i-2-2) include the compounds represented by the following structural formulae (i-2-2.1) to (i-2-2.8):

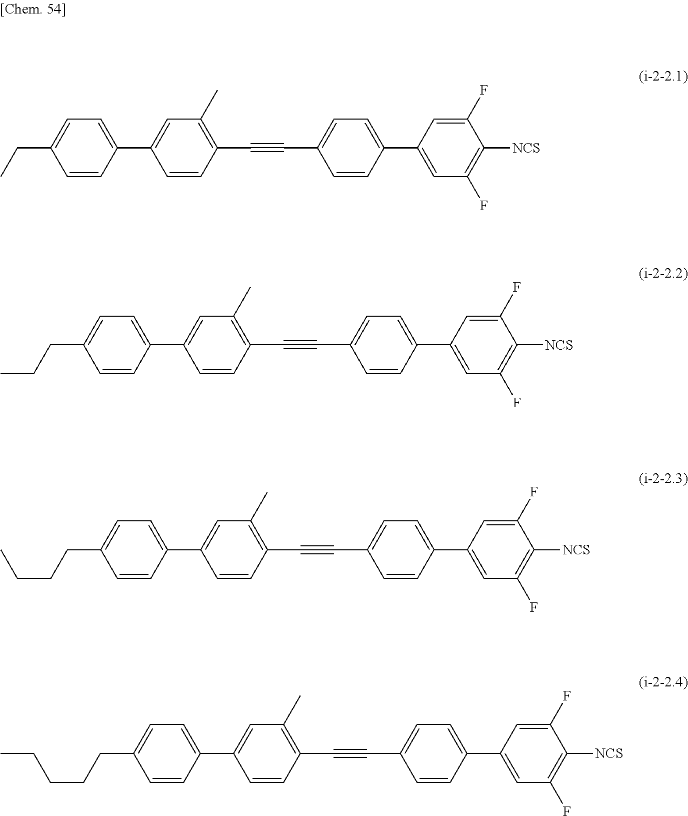

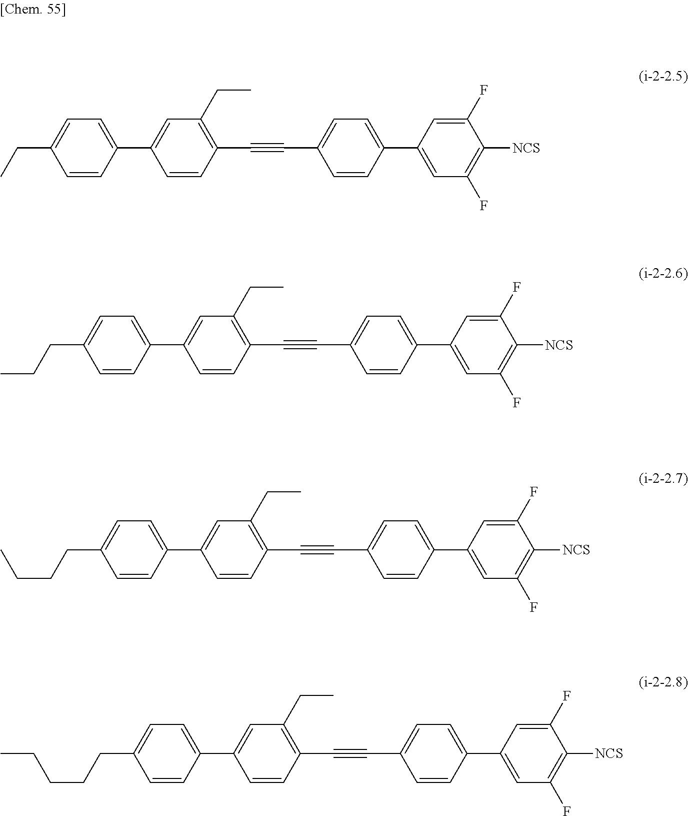

Specific examples of the compounds represented by the general formula (i-2-3) include the compounds represented by the following structural formulae (i-2-3.1) to (i-2-3.8):

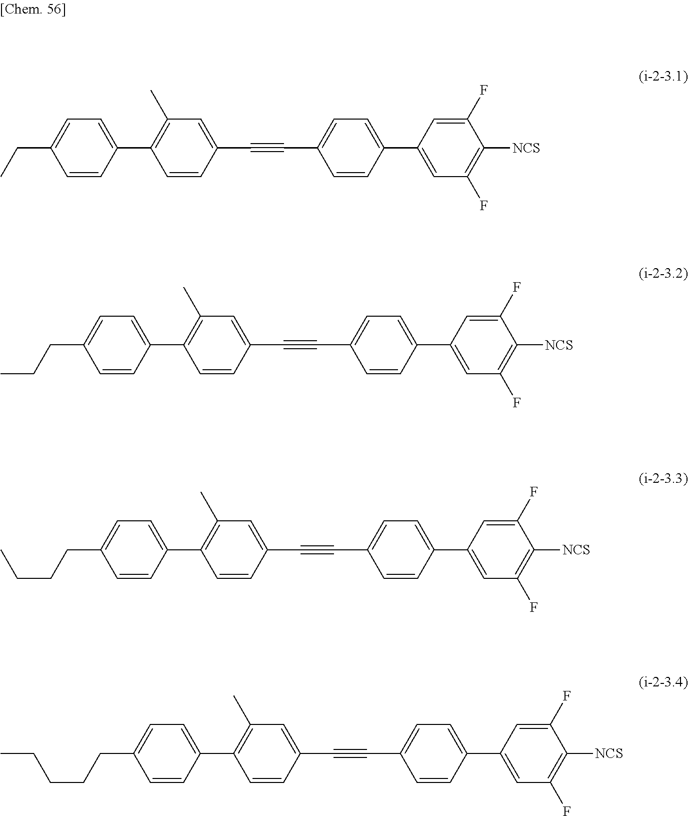

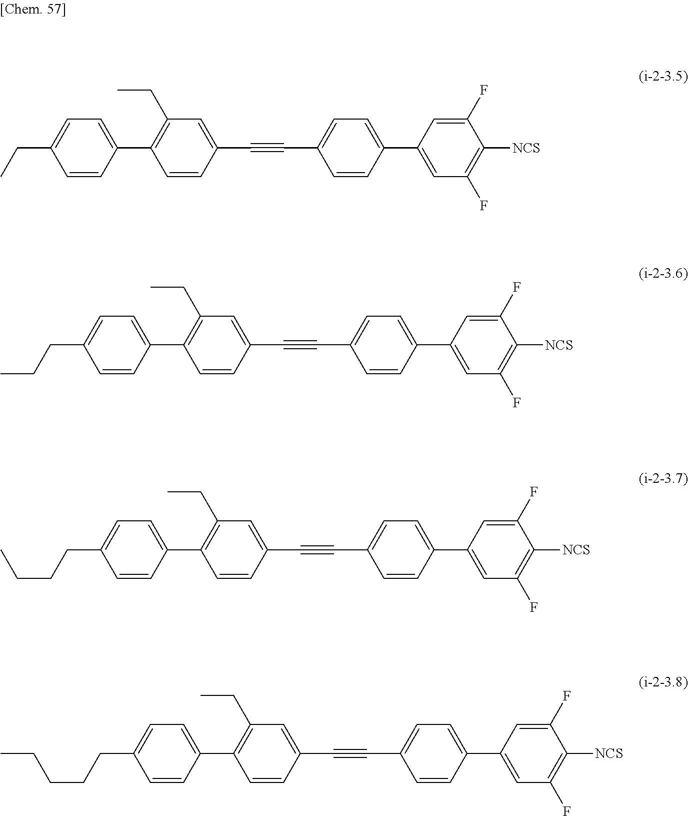

Specific examples of the compounds represented by the general formula (i-2-4) include the compounds represented by the following structural formulae (i-2-4.1) to (i-2-4.4):

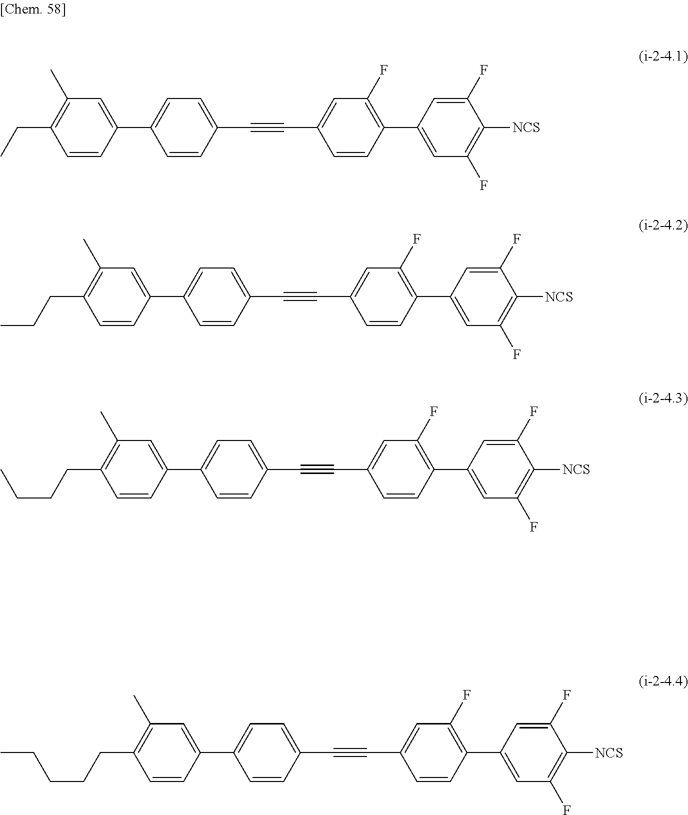

Specific examples of the compounds represented by the general formula (i-2-5) include the compounds represented by the following structural formulae (i-2-5.1) to (i-2-5.4):

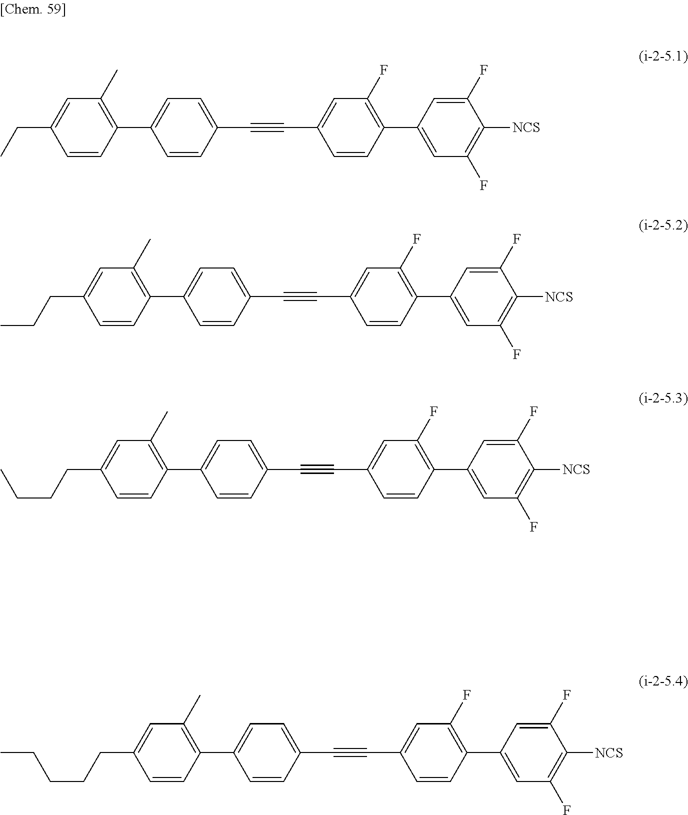

Specific examples of the compounds represented by the general formula (i-2-6) include the compounds represented by the following structural formulae (i-2-6.1) to (i-2-6.4):

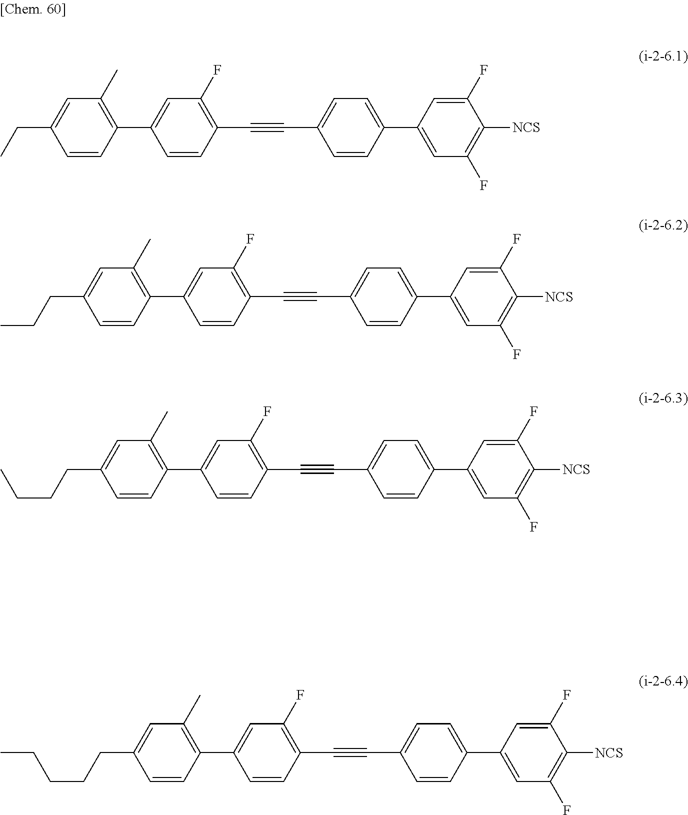

Specific examples of the compounds represented by the general formula (i-2-7) include the compounds represented by the following structural formulae (i-2-7.1) to (i-2-7.15):

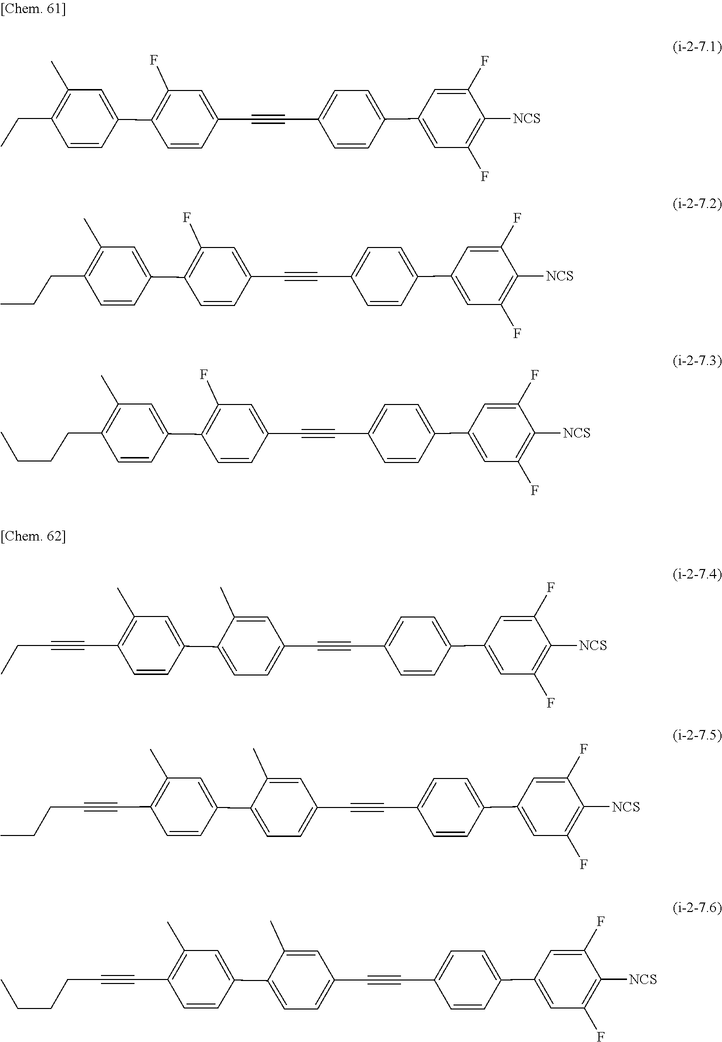

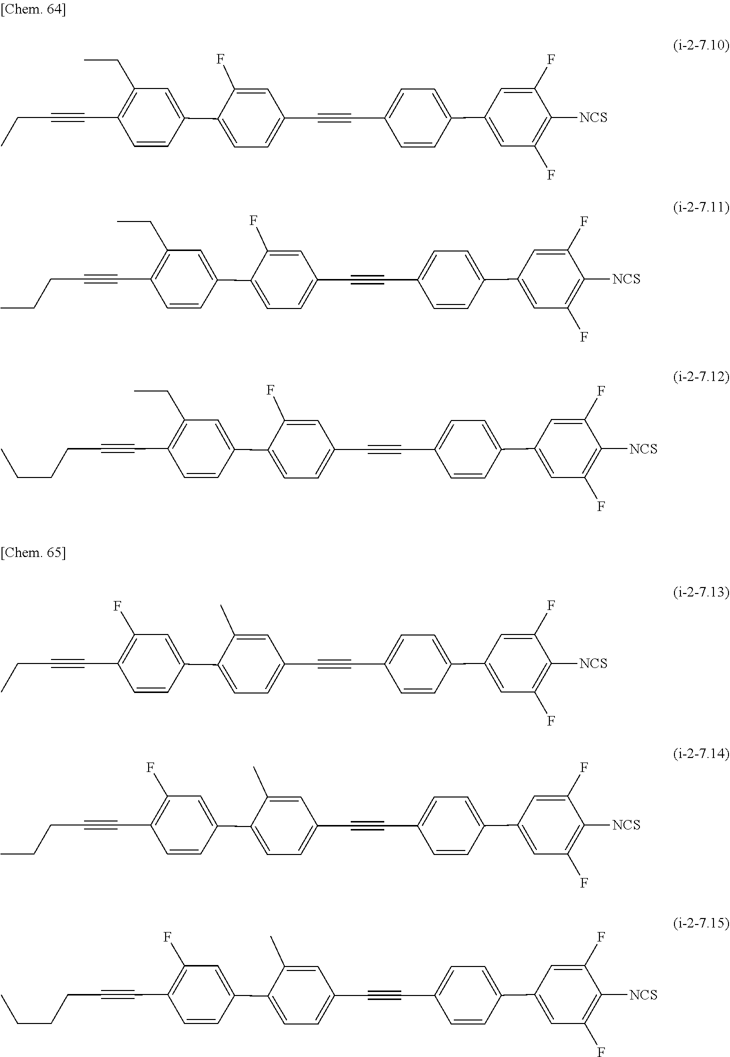

Specific examples of the compounds represented by the general formula (i-2-8) include the compounds represented by the following structural formulae (i-2-8.1) to (i-2-8.8):

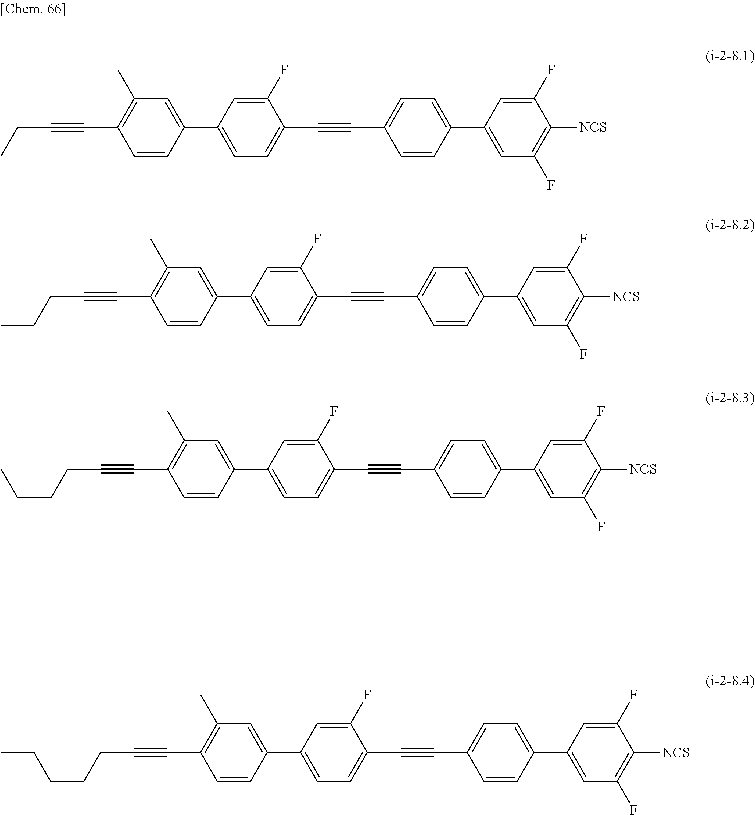

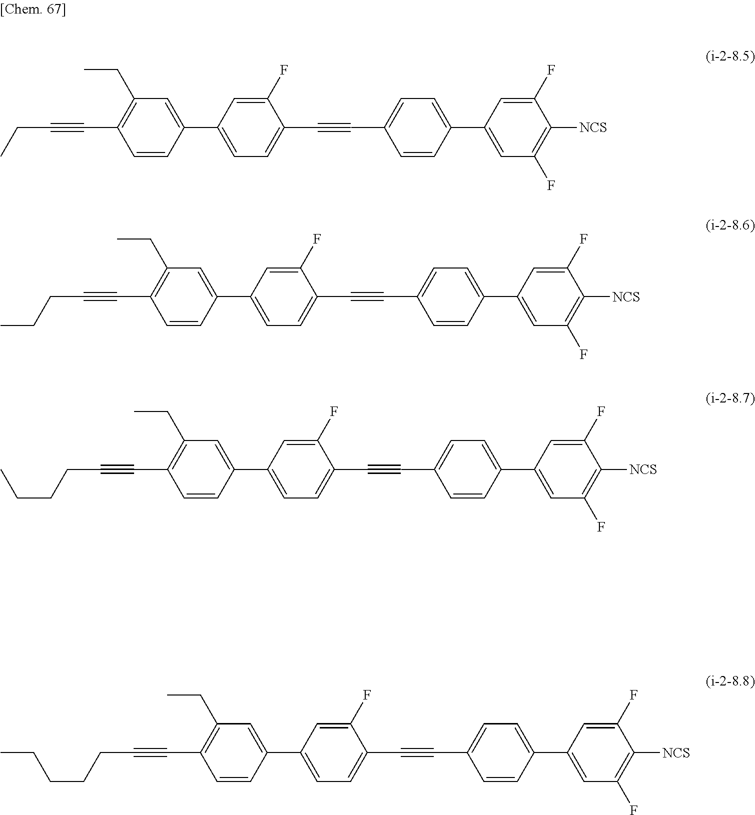

Specific examples of the compounds represented by the general formula (i-2-9) include the compounds represented by the following structural formulae (i-2-9.1) to (i-2-9.4):

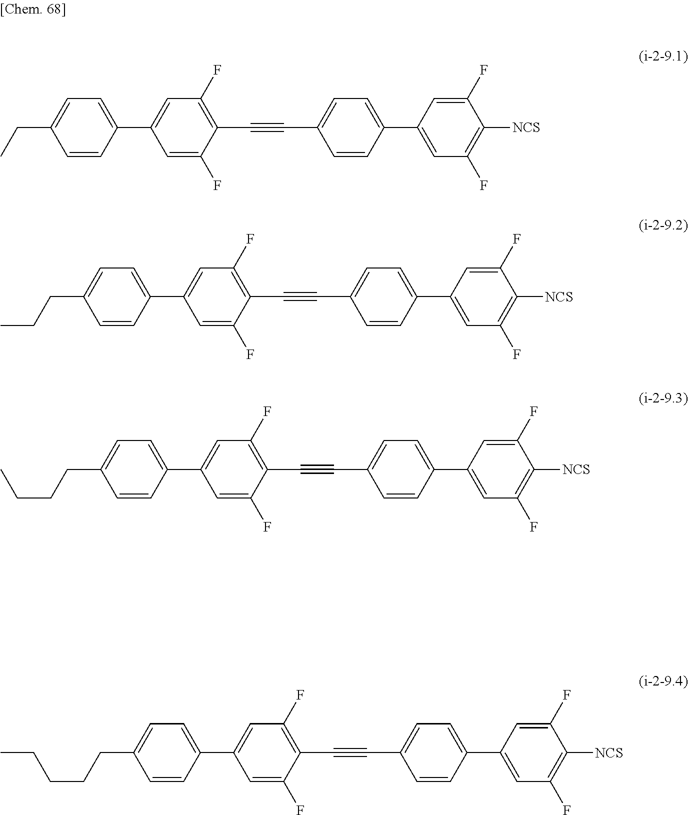

Specific examples of the compounds represented by the general formula (i-2-10) include the compounds represented by the following structural formulae (i-2-10.1) to (i-2-10.4):

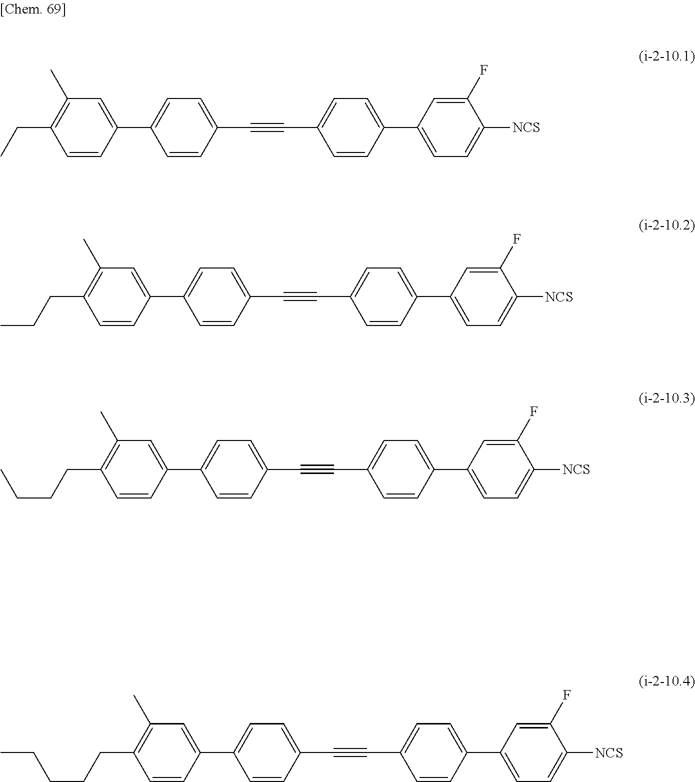

Specific examples of the compounds represented by the general formula (i-2-11) include the compounds represented by the following structural formulae (i-2-11.1) to (i-2-11.8):

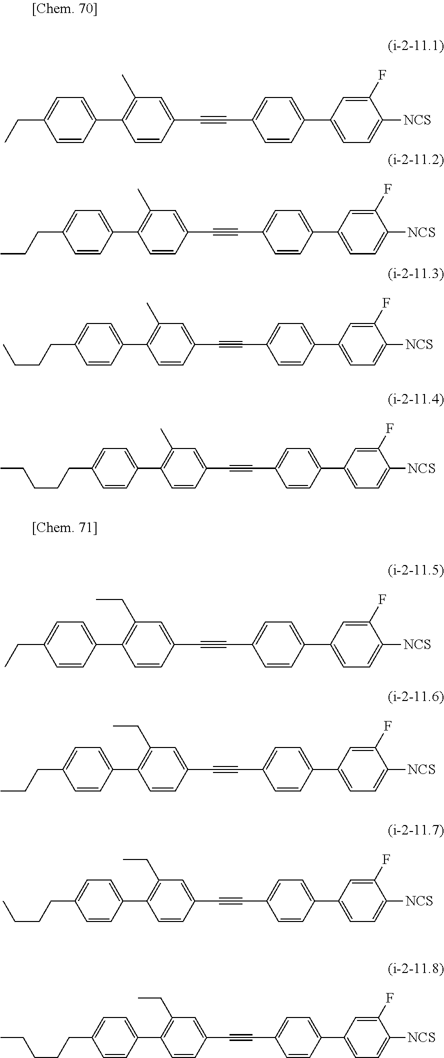

Specific examples of the compounds represented by the general formula (i-2-12) include the compounds represented by the following structural formulae (i-2-12.1) to (i-2-12.4):

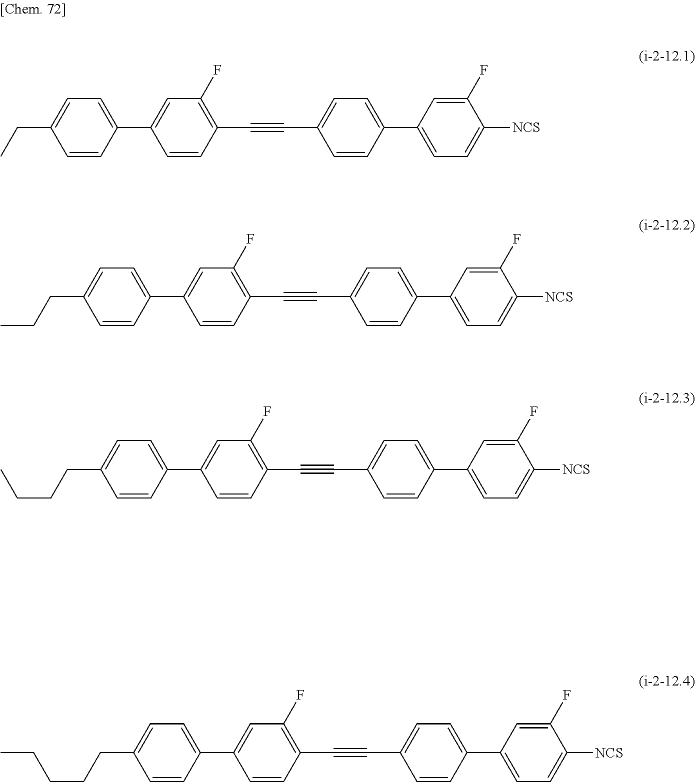

Specific examples of the compounds represented by the general formula (i-2-13) include the compounds represented by the following structural formulae (i-2-13.1) to (i-2-13.4):

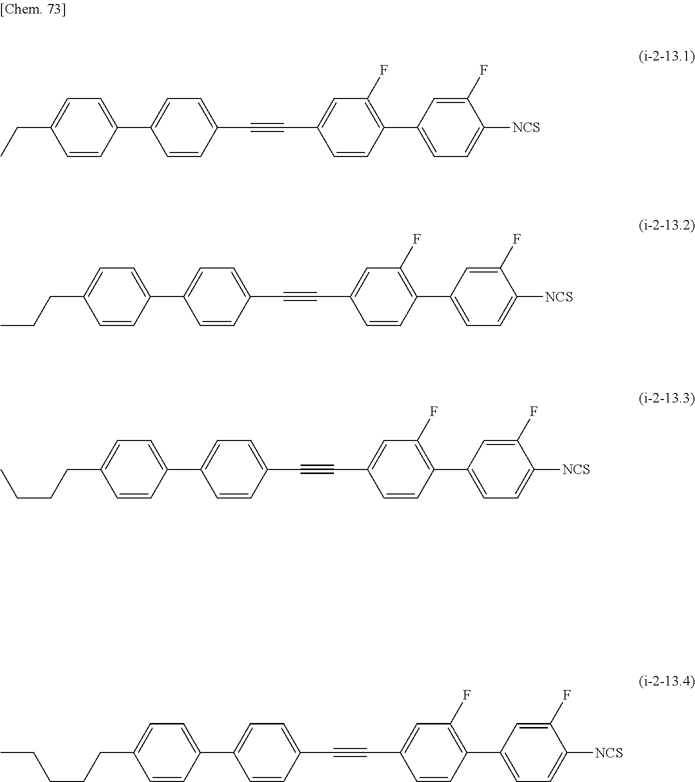

Specific examples of the compounds represented by the general formula (i-2-14) include the compounds represented by the following structural formulae (i-2-14.1) to (i-2-14.4):

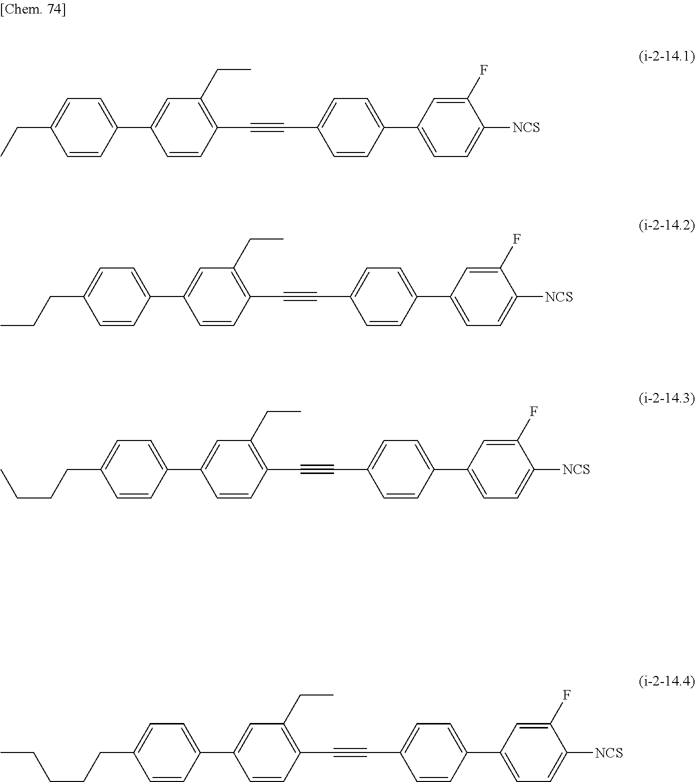

Specific examples of the compounds represented by the general formula (i-2-15) include the compounds represented by the following structural formulae (i-2-15.1) to (i-2-15.4):

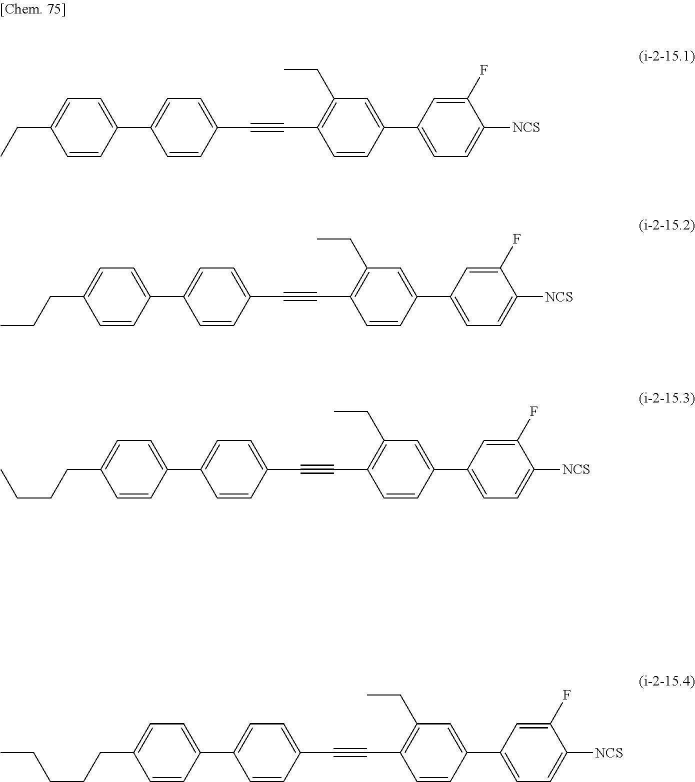

Specific examples of the compounds represented by the general formula (i-2-16) include the compounds represented by the following structural formulae (i-2-16.1) to (i-2-16.4):

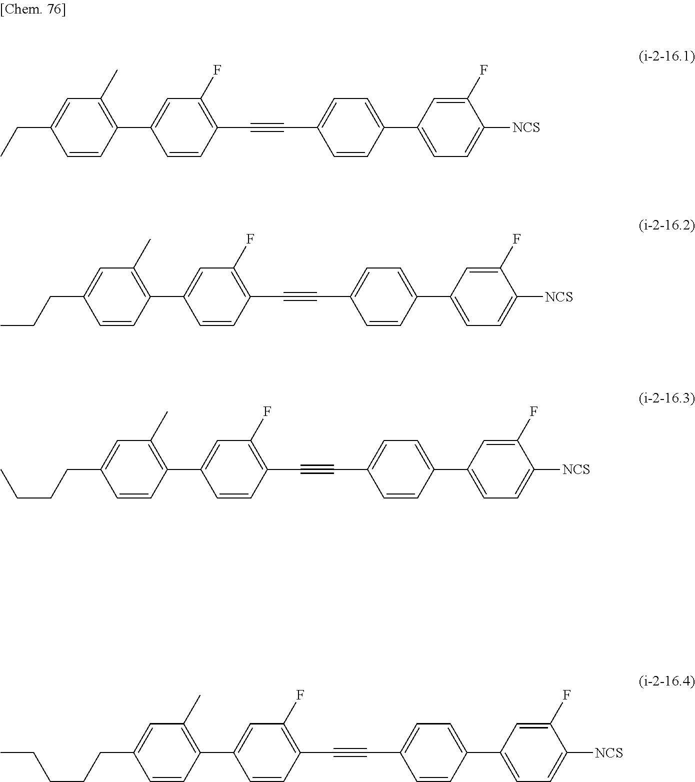

Specific examples of the compounds represented by the general formula (i-2-17) include the compounds represented by the following structural formulae (i-2-17.1) to (i-2-17.4):

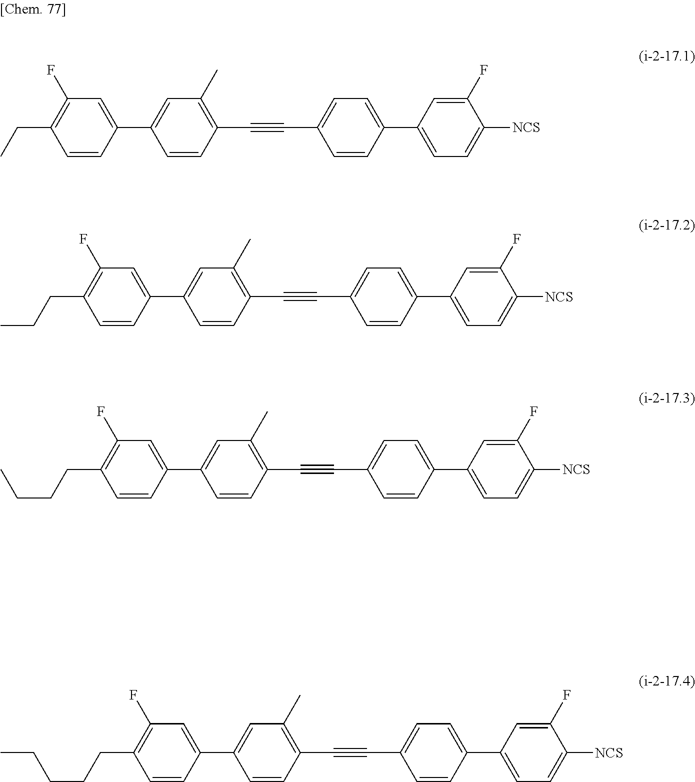

Specific examples of the compounds represented by the general formula (i-2-18) include the compounds represented by the following structural formulae (i-2-18.1) to (i-2-18.4):

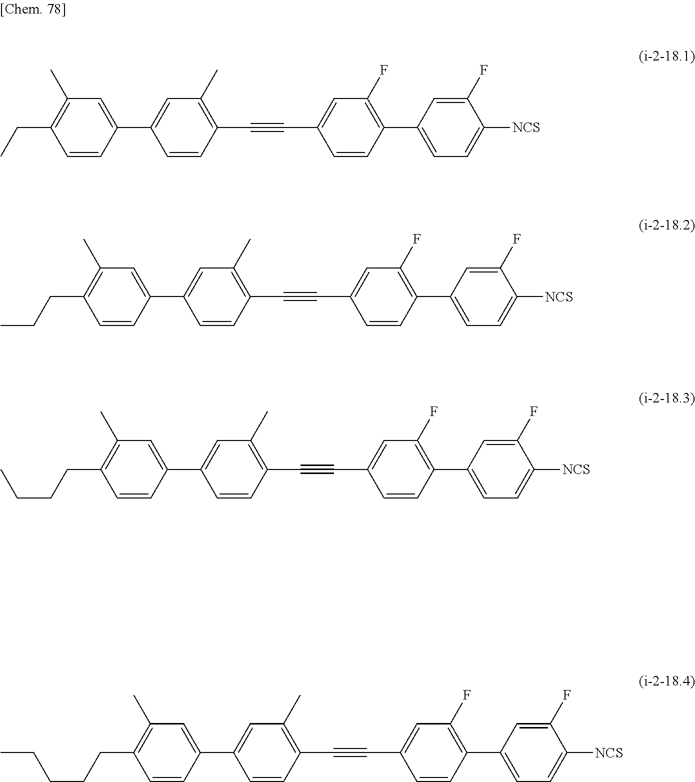

Compounds represented by the general formula (i-3) are preferably the compounds represented by the following general formulae (1-3-1) to (Jan. 3, 2022):

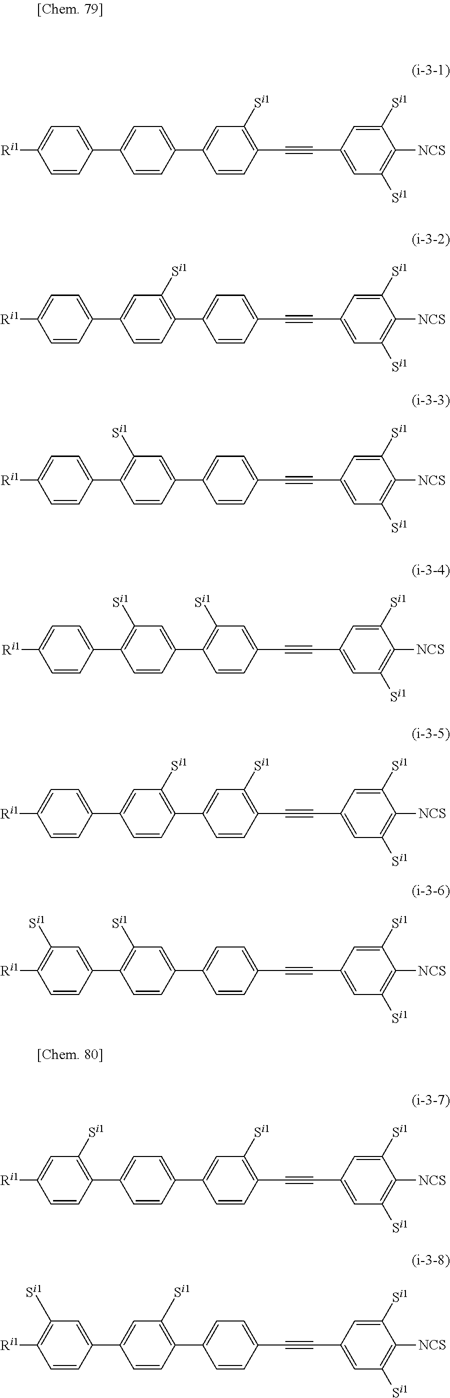

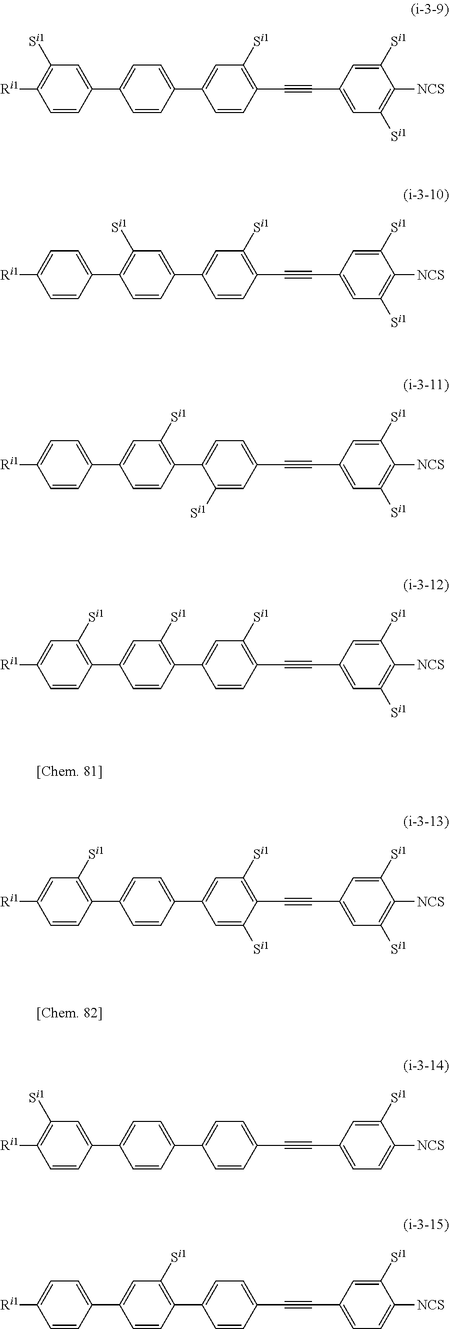

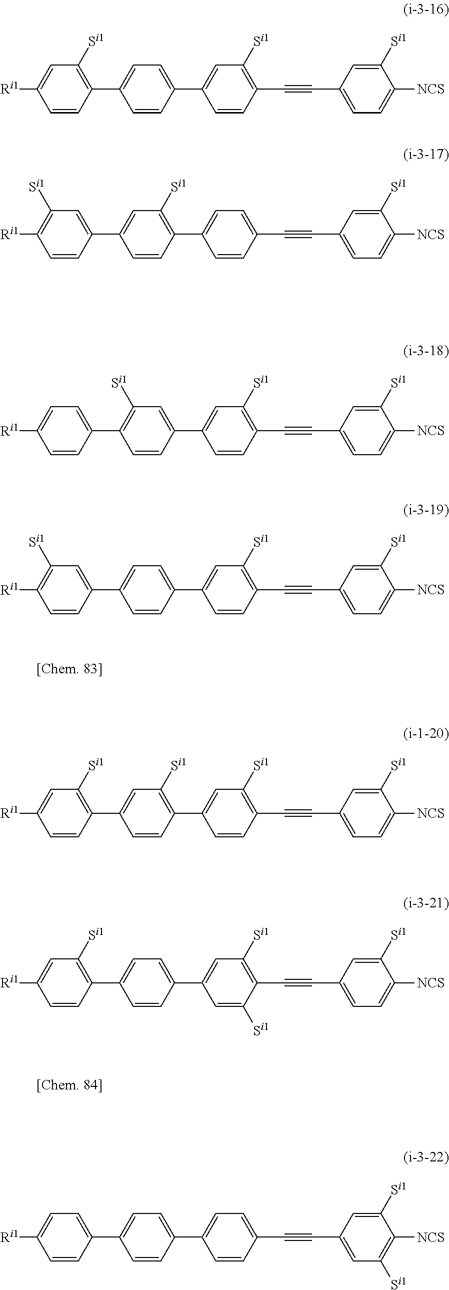

In the general formulae (i-3-1) to (i-3-22), Rⁱ¹ and Sⁱ¹ each independently denote the same as Rⁱ¹ and Sⁱ¹ in the general formula (i).

Specific examples of the compounds represented by the general formula (i-3-1) include the compounds represented by the following structural formulae (i-3-1.1) to (i-3-1.4):

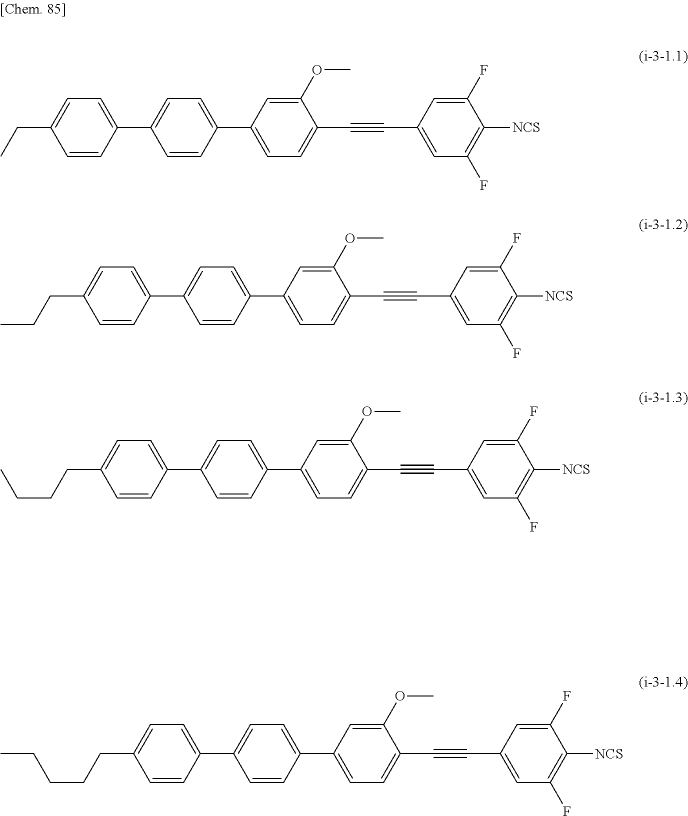

Specific examples of the compounds represented by the general formula (i-3-2) include the compounds represented by the following structural formulae (i-3-2.1) to (i-3-2.8):

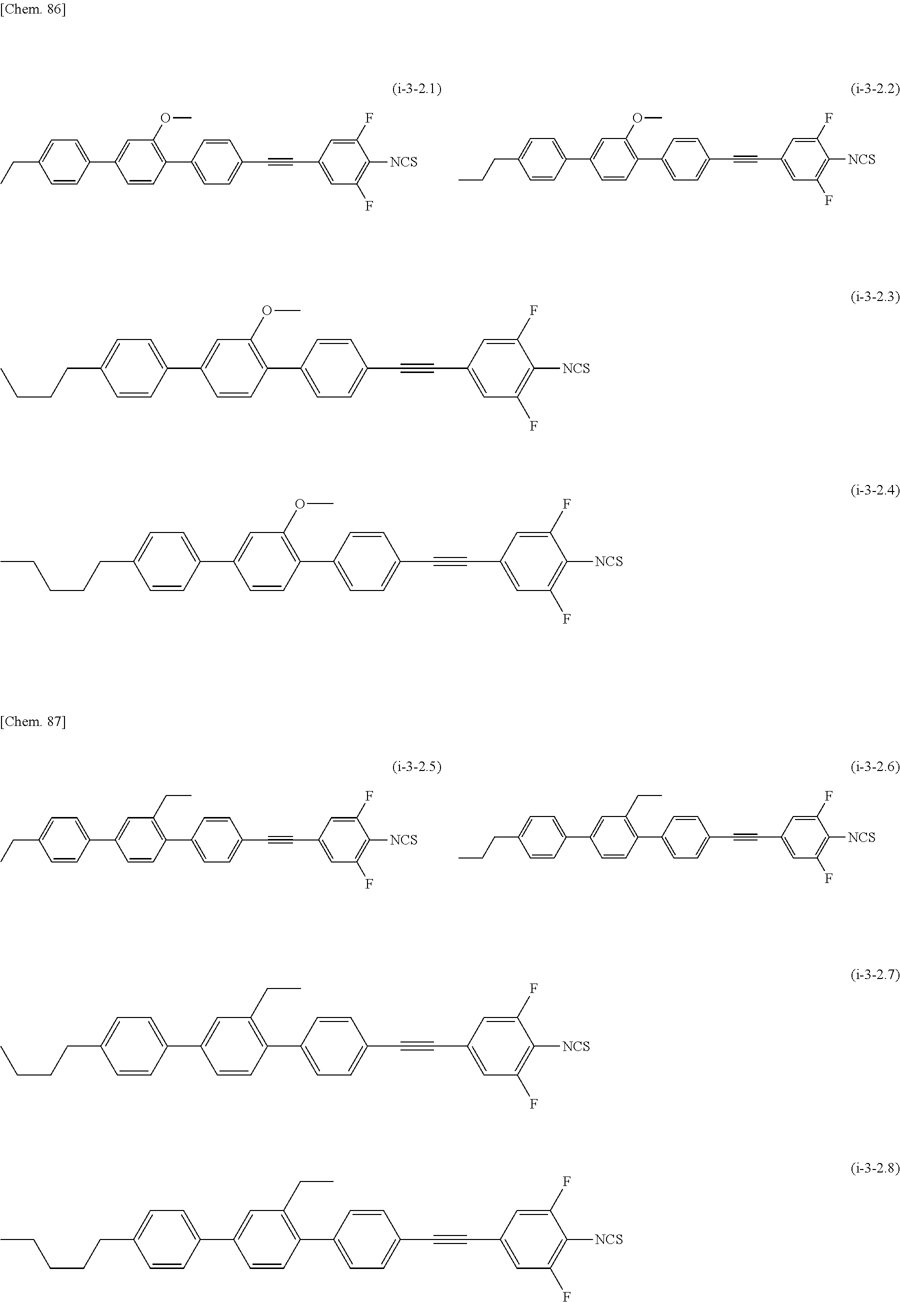

Specific examples of the compounds represented by the general formula (i-3-3) include the compounds represented by the following structural formulae (i-3-3.1) to (i-3-3.4):

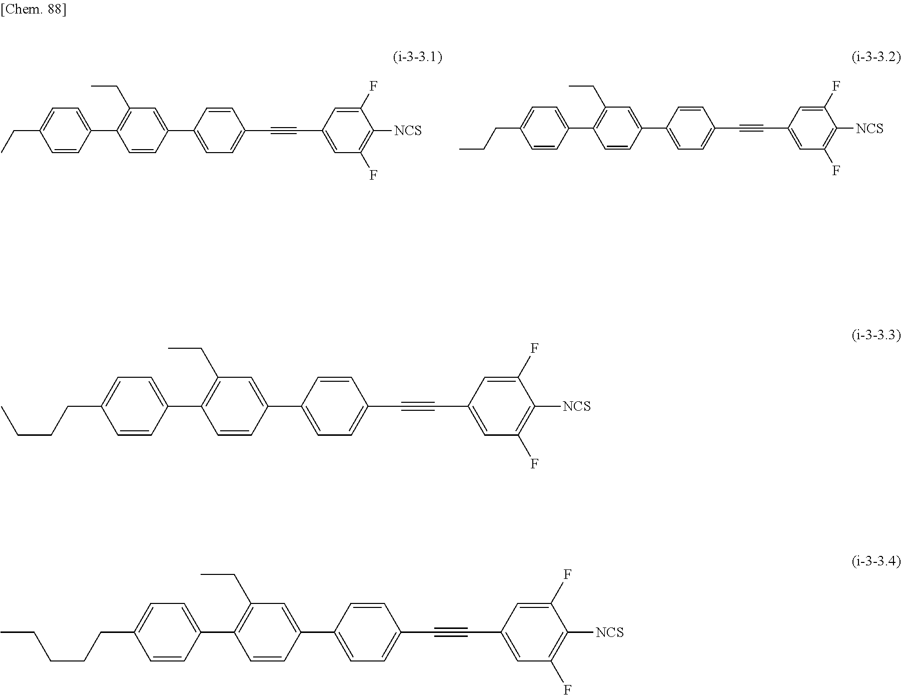

Specific examples of the compounds represented by the general formula (i-3-4) include the compounds represented by the following structural formulae (i-3-4.1) to (i-3-4.8):

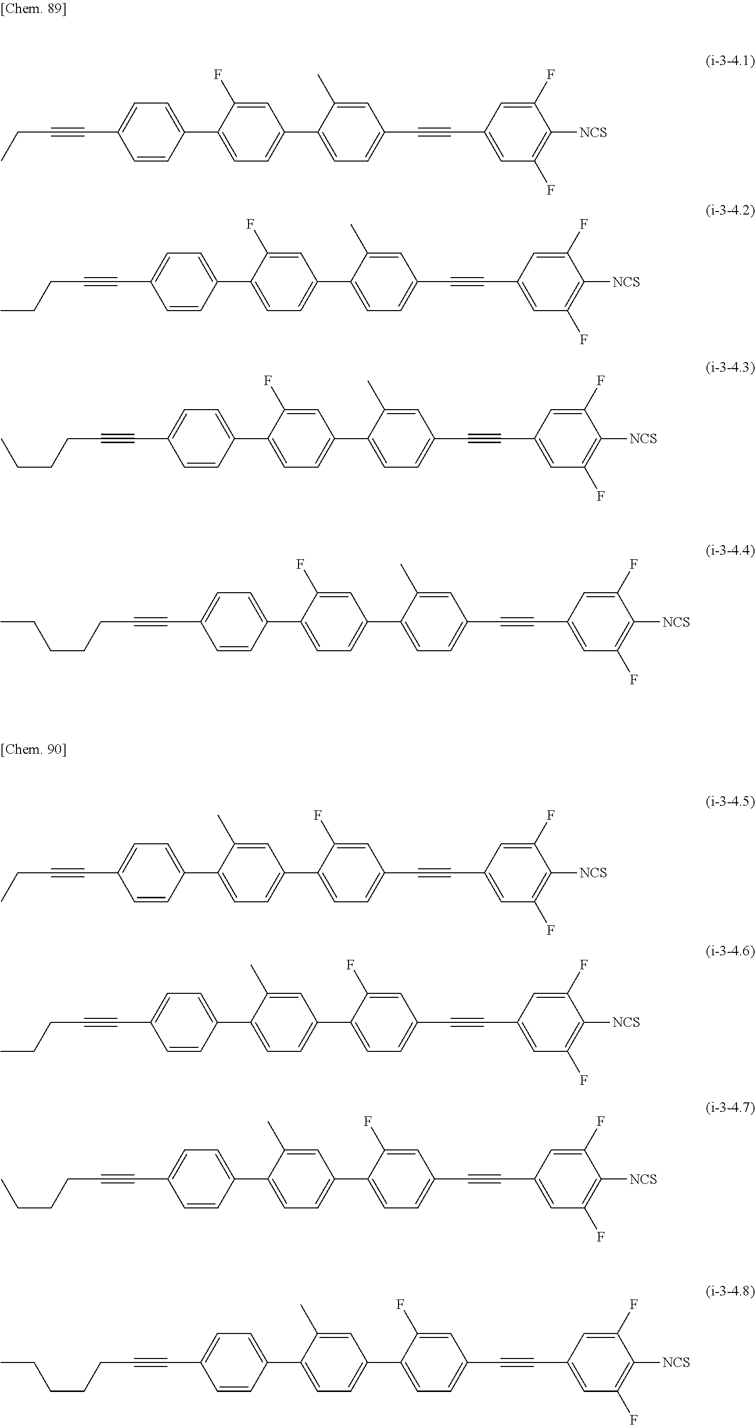

Specific examples of the compounds represented by the general formula (i-3-5) include the compounds represented by the following structural formulae (1-3-5.1) to (1-3-5.4):

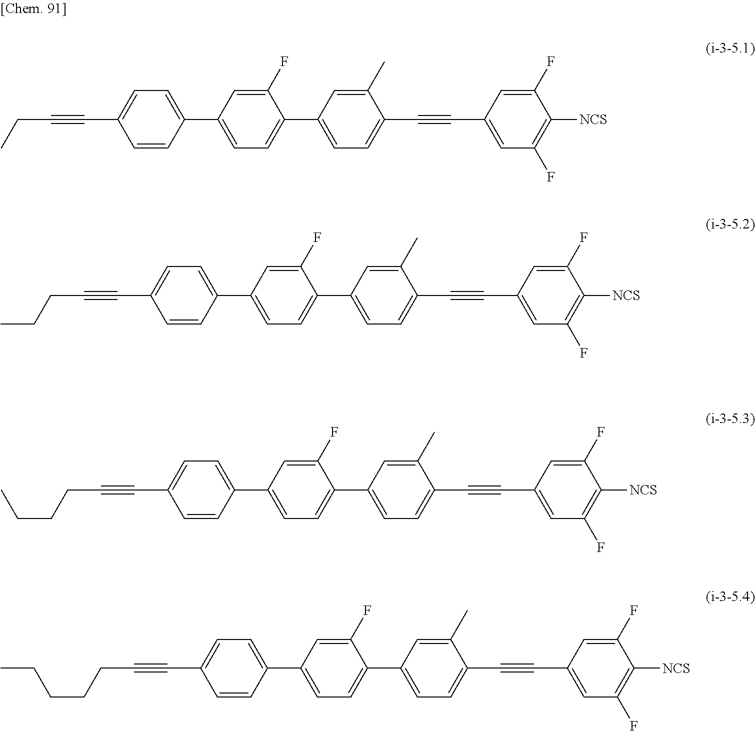

Specific examples of the compounds represented by the general formula (i-3-6) include the compounds represented by the following structural formulae (i-3-6.1) to (i-3-6.24):

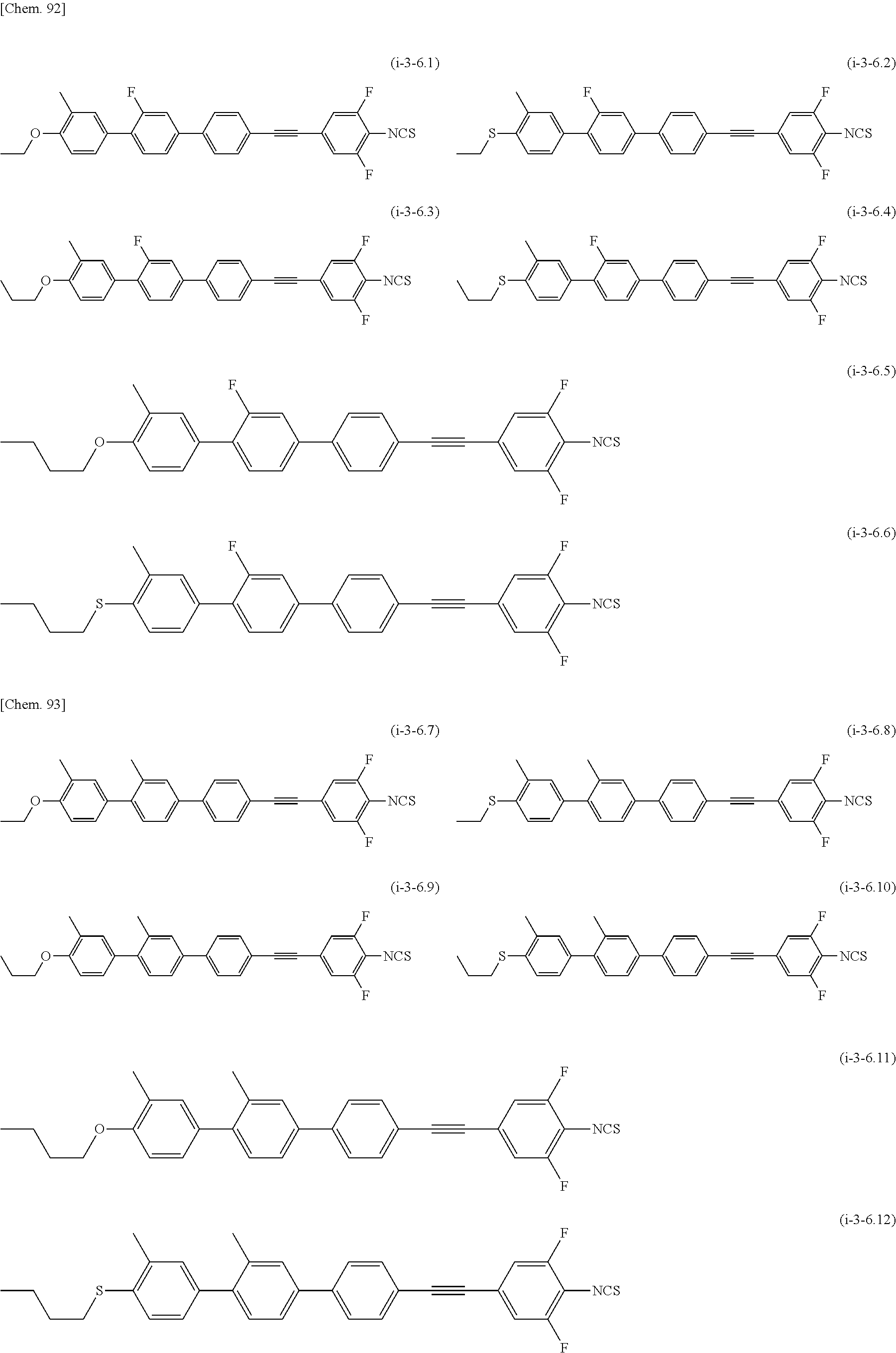

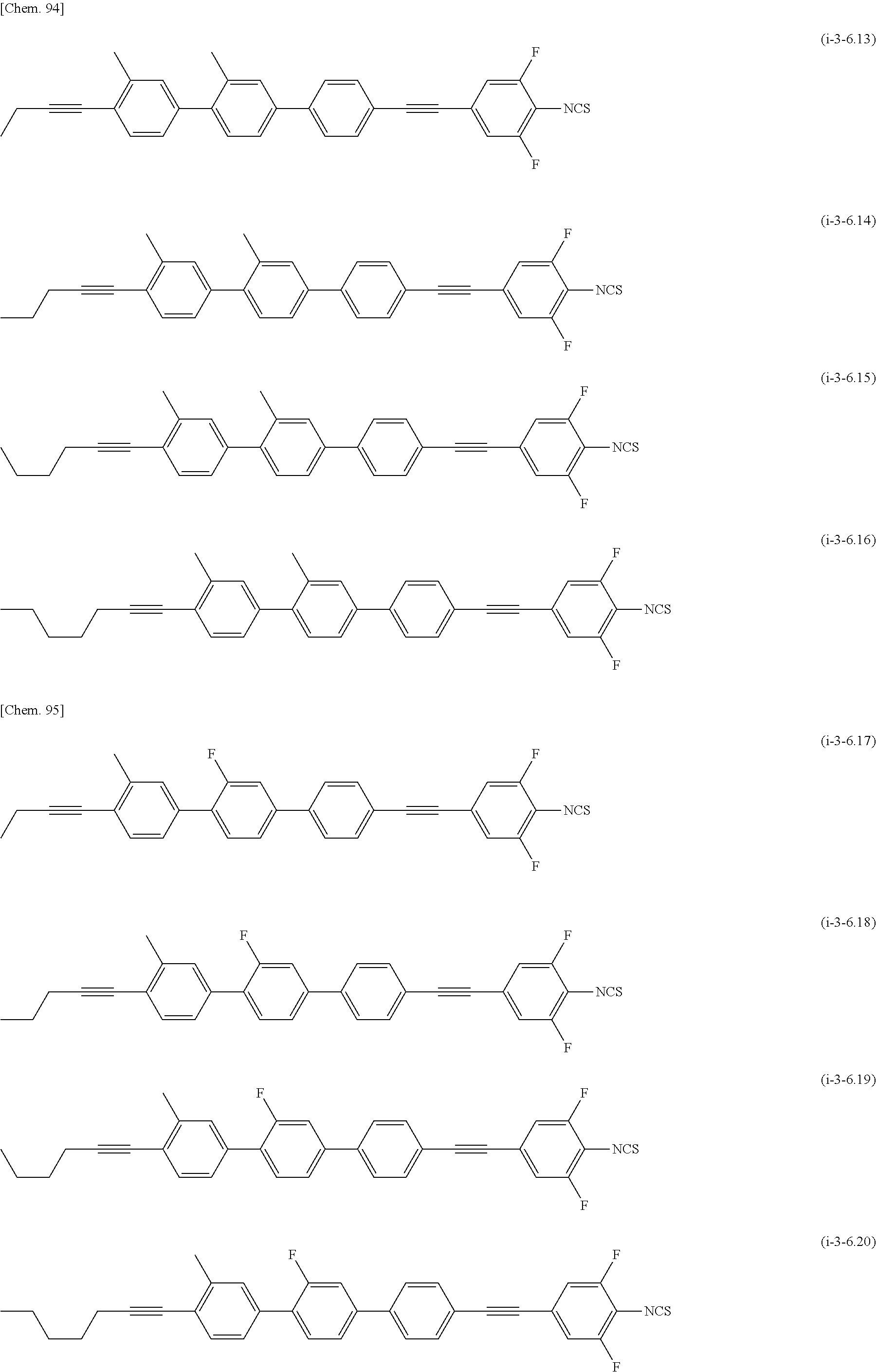

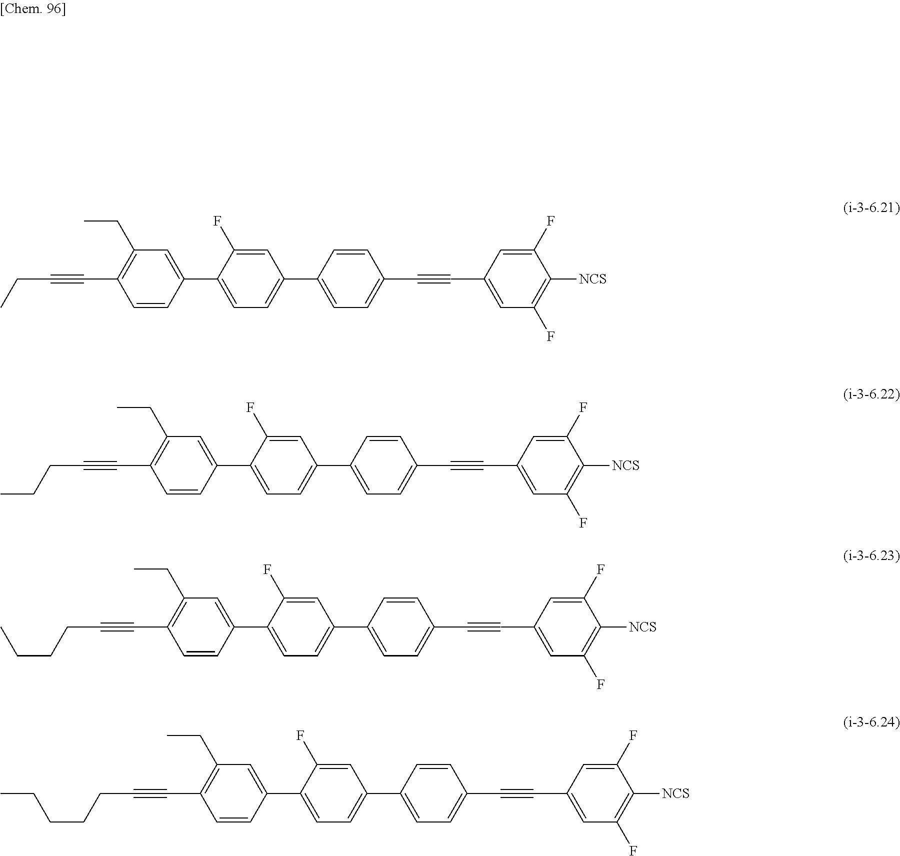

Specific examples of the compounds represented by the general formula (i-3-7) include the compounds represented by the following structural formulae (i-3-7.1) to (i-3-7.4):

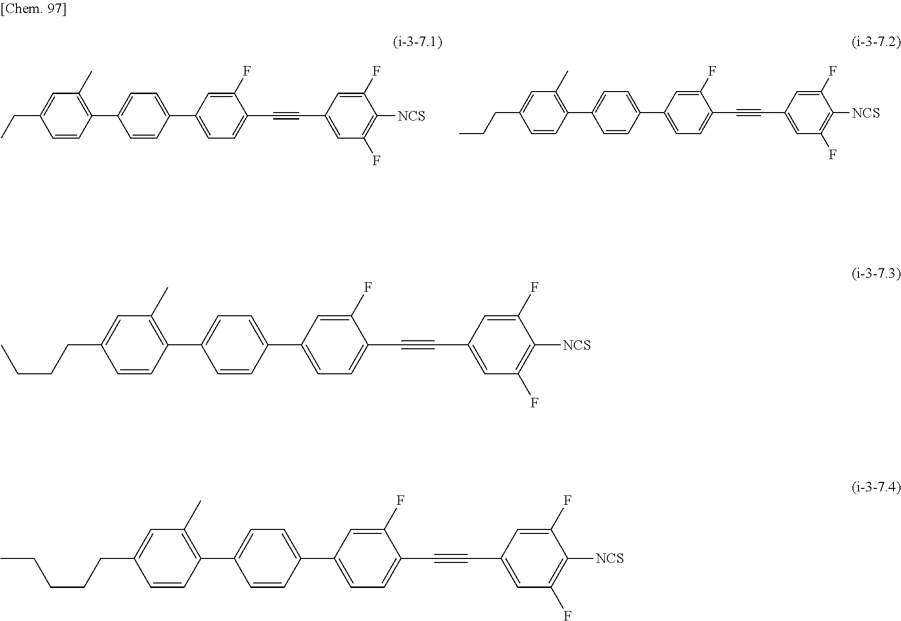

Specific examples of the compounds represented by the general formula (i-3-8) include the compounds represented by the following structural formulae (i-3-8.1) to (i-3-8.23):

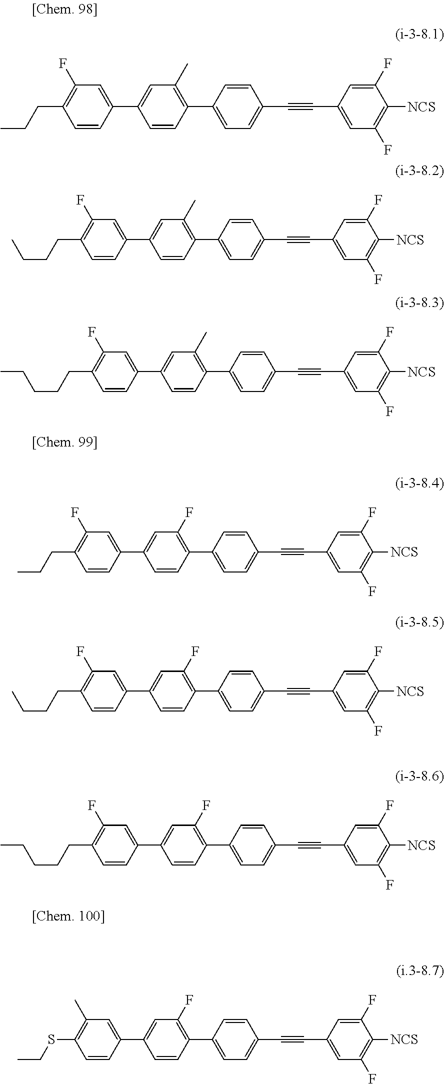

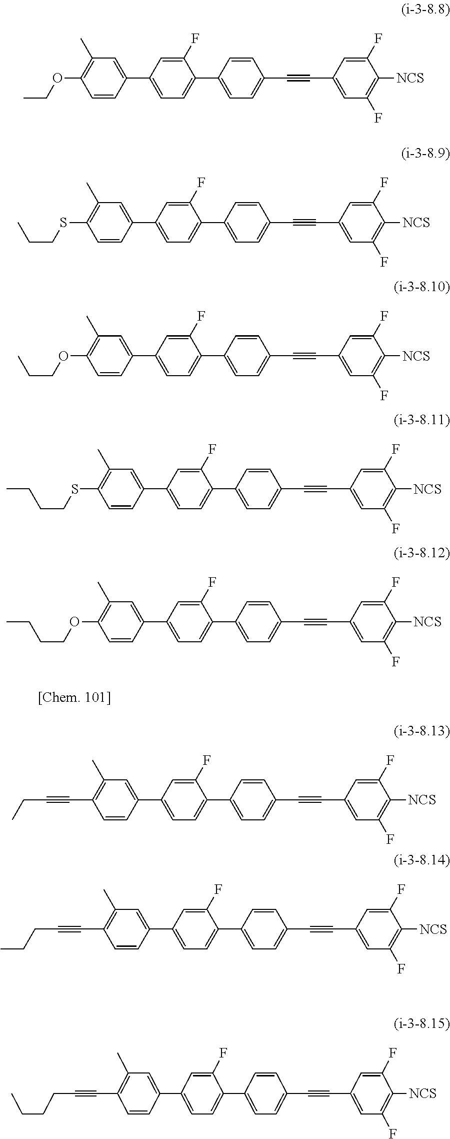

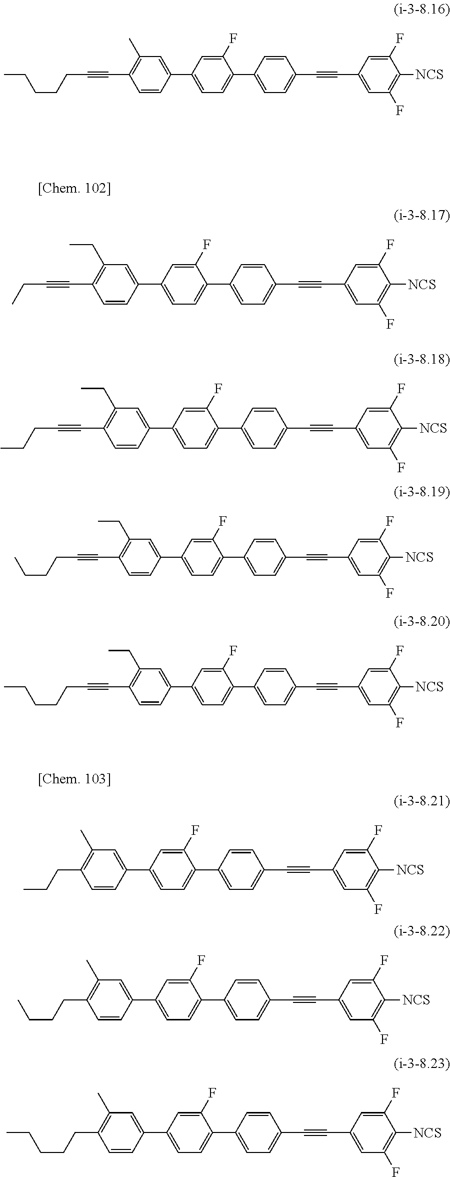

Specific examples of the compounds represented by the general formula (i-3-9) include the compounds represented by the following structural formulae (i-3-9.1) to (i-3-9.4):

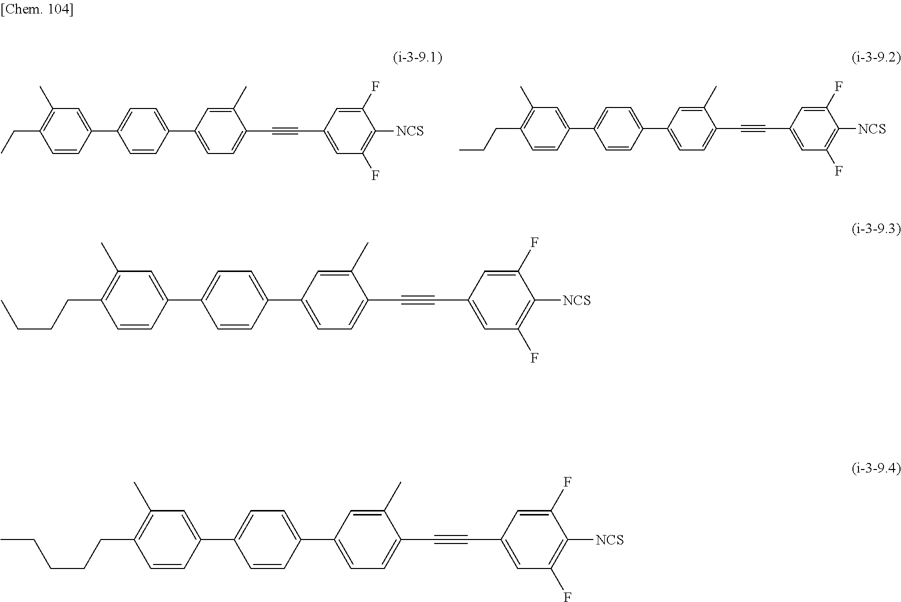

Specific examples of the compounds represented by the general formula (i-3-10) include the compounds represented by the following structural formulae (i-3-10.1) to (i-3-10.8):

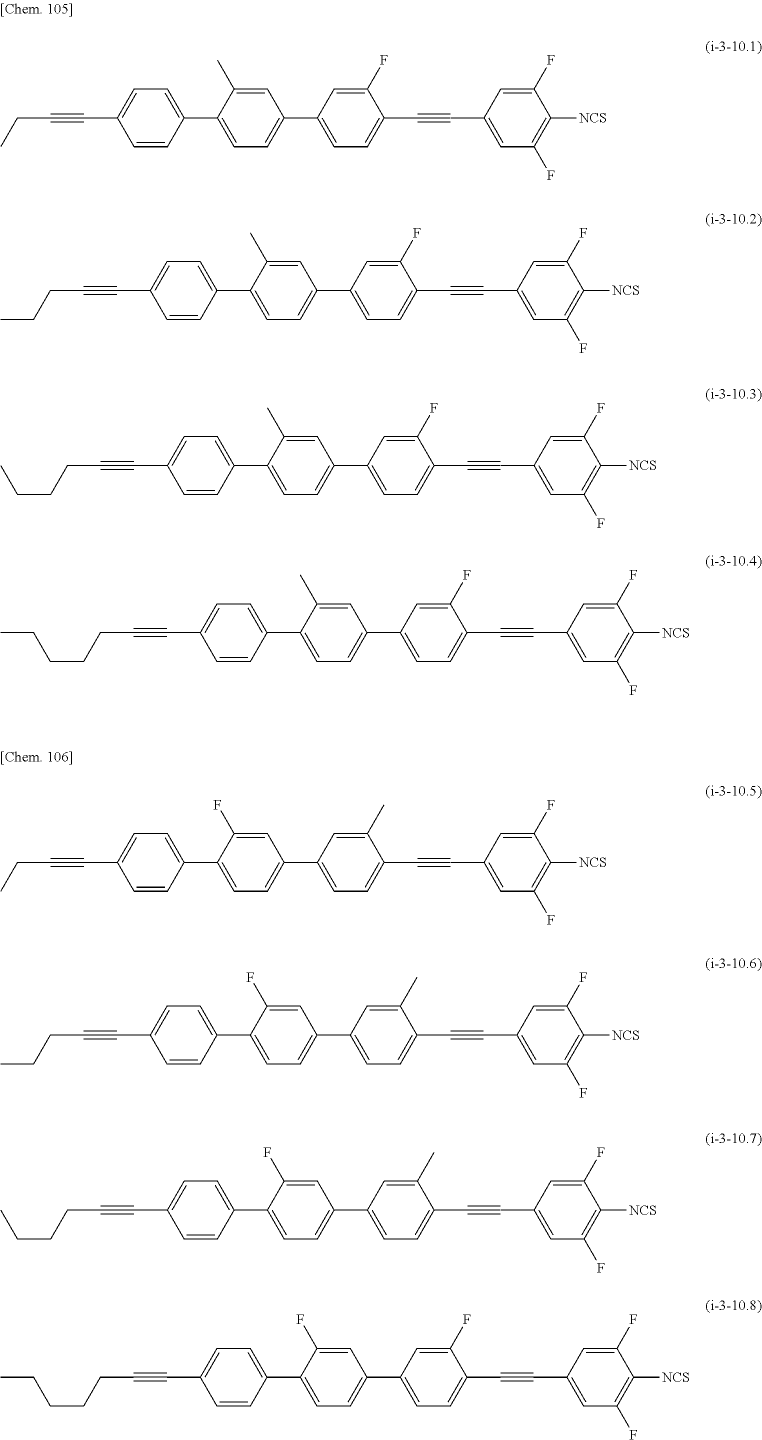

Specific examples of the compounds represented by the general formula (i-3-11) include the compounds represented by the following structural formulae (i-3-11.1) to (i-3-11.4):

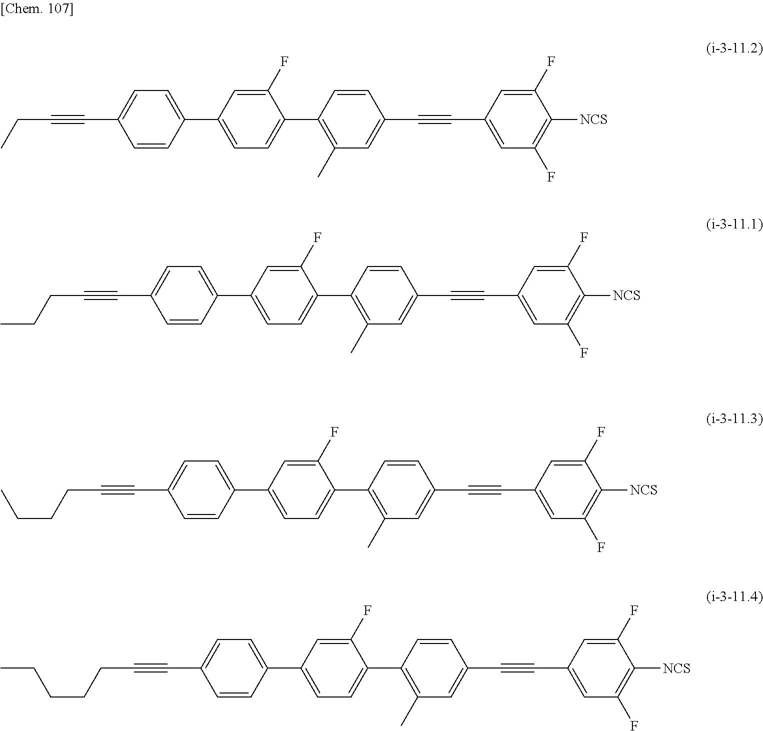

Specific examples of the compounds represented by the general formula (i-3-12) include the compounds represented by the following structural formulae (i-3-12.1) to (i-3-12.4):

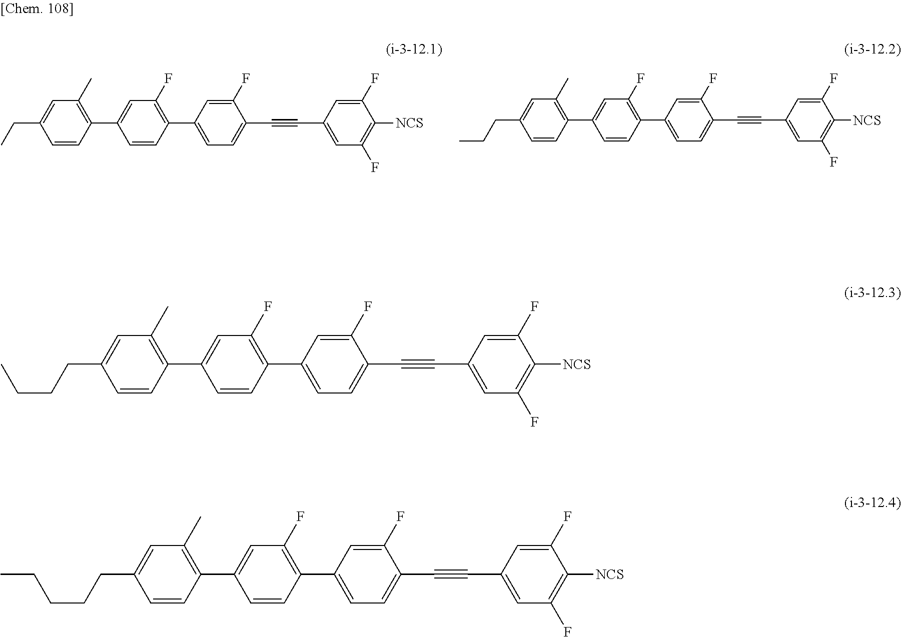

Specific examples of the compounds represented by the general formula (i-3-13) include the compounds represented by the following structural formulae (i-3-13.1) to (i-3-13.4):

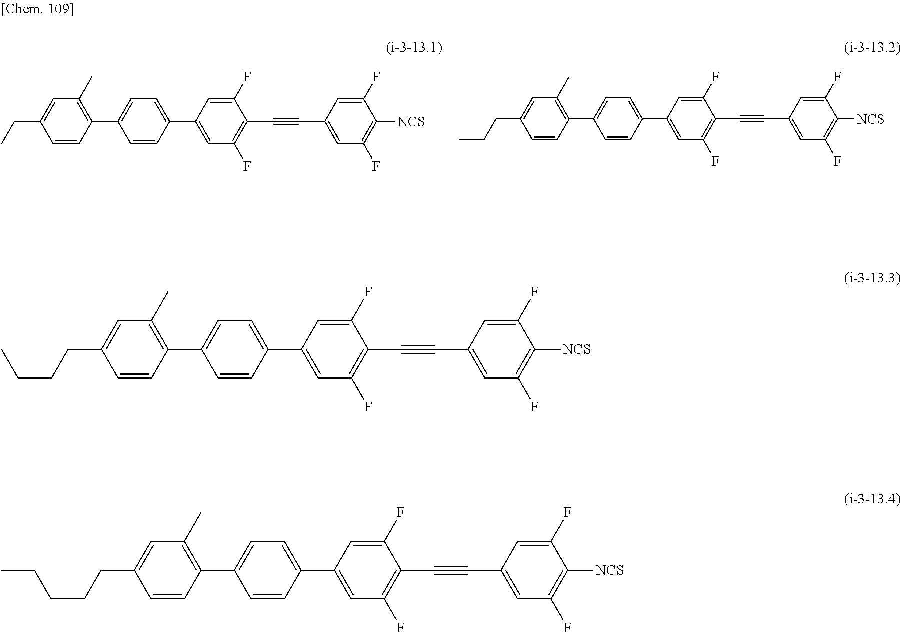

Specific examples of the compounds represented by the general formula (i-3-14) include the compounds represented by the following structural formulae (i-3-14.1) to (i-3-14.4):

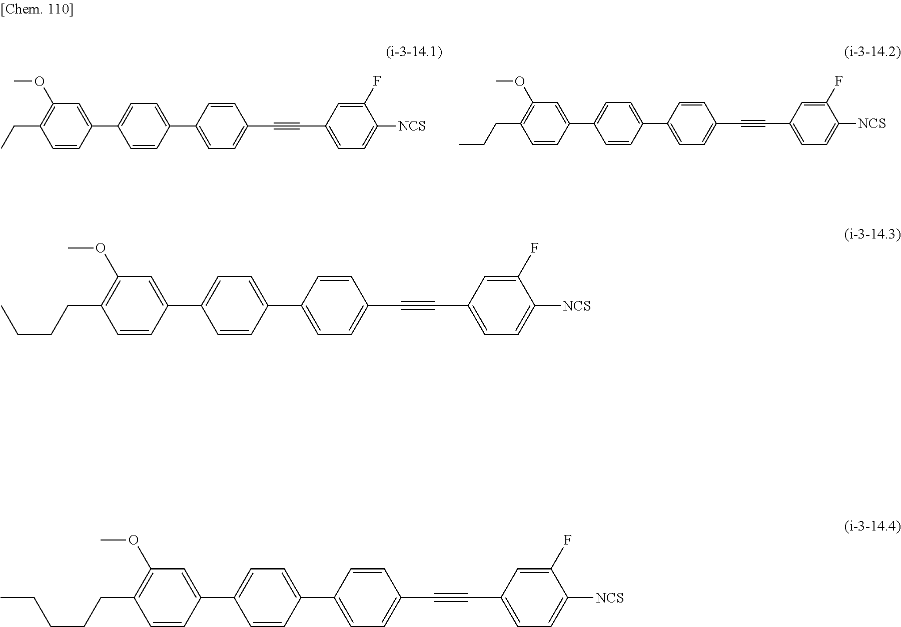

Specific examples of the compounds represented by the general formula (i-3-15) include the compounds represented by the following structural formulae (i-3-15.1) to (i-3-15.8):

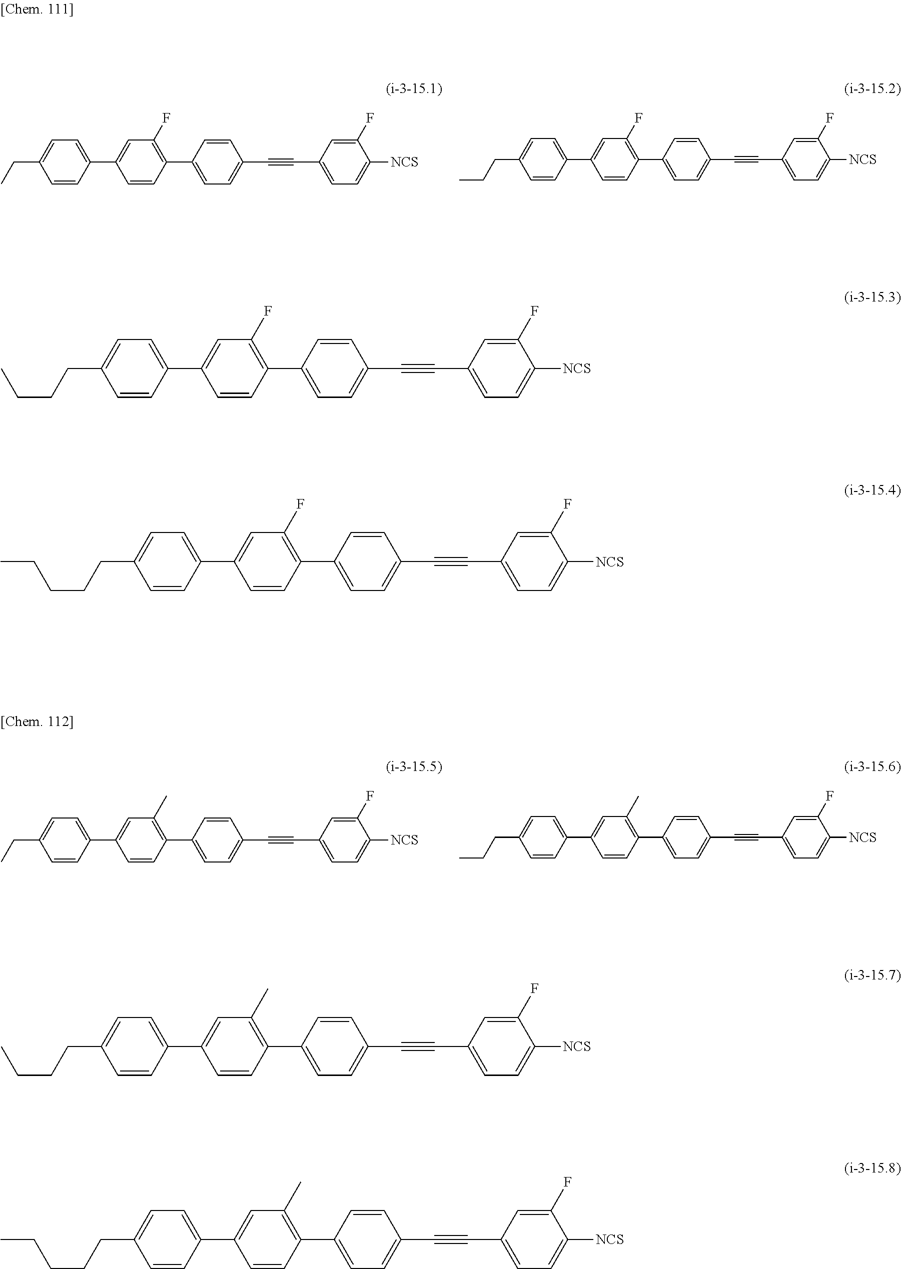

Specific examples of the compounds represented by the general formula (i-3-16) include the compounds represented by the following structural formulae (i-3-16.1) to (i-3-16.4):

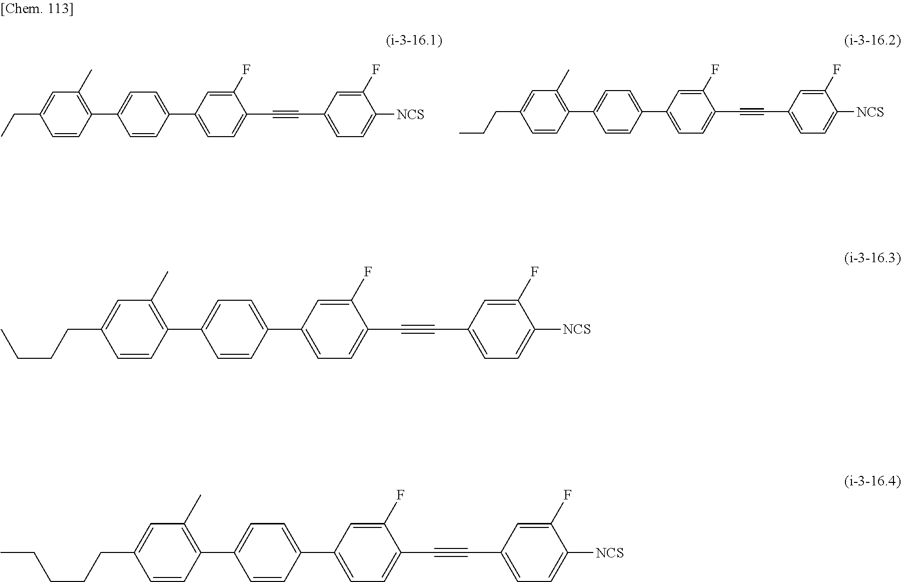

Specific examples of the compounds represented by the general formula (i-3-17) include the compounds represented by the following structural formulae (i-3-17.1) to (i-3-17.4):

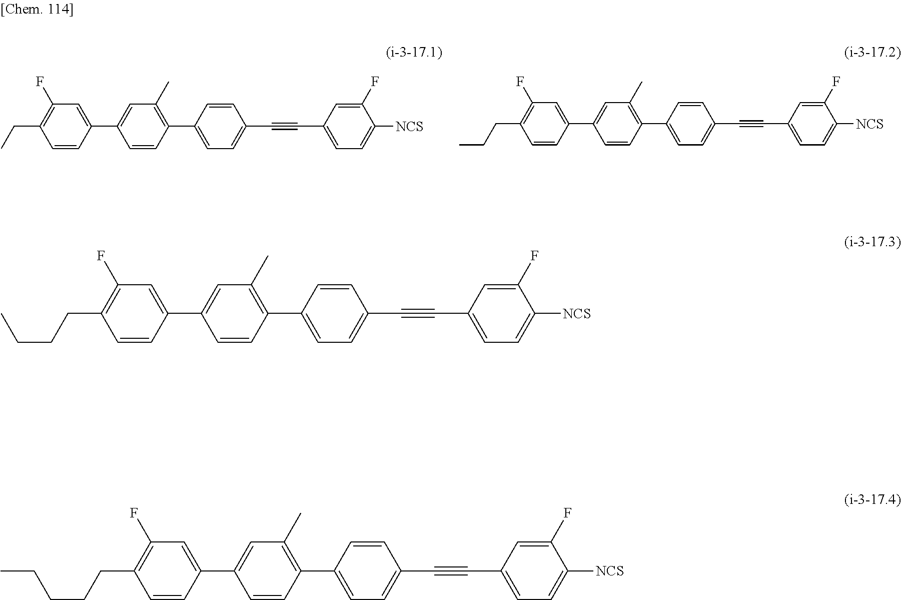

Specific examples of the compounds represented by the general formula (i-3-18) include the compounds represented by the following structural formulae (i-3-18.1) to (i-3-18.4):

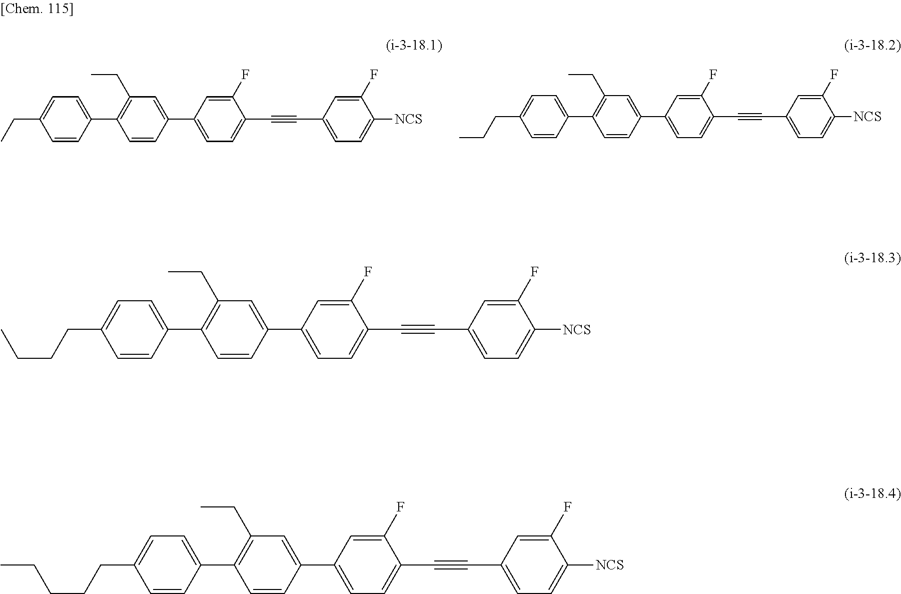

Specific examples of the compounds represented by the general formula (i-3-19) include the compounds represented by the following structural formulae (i-3-19.1) to (i-3-19.8):

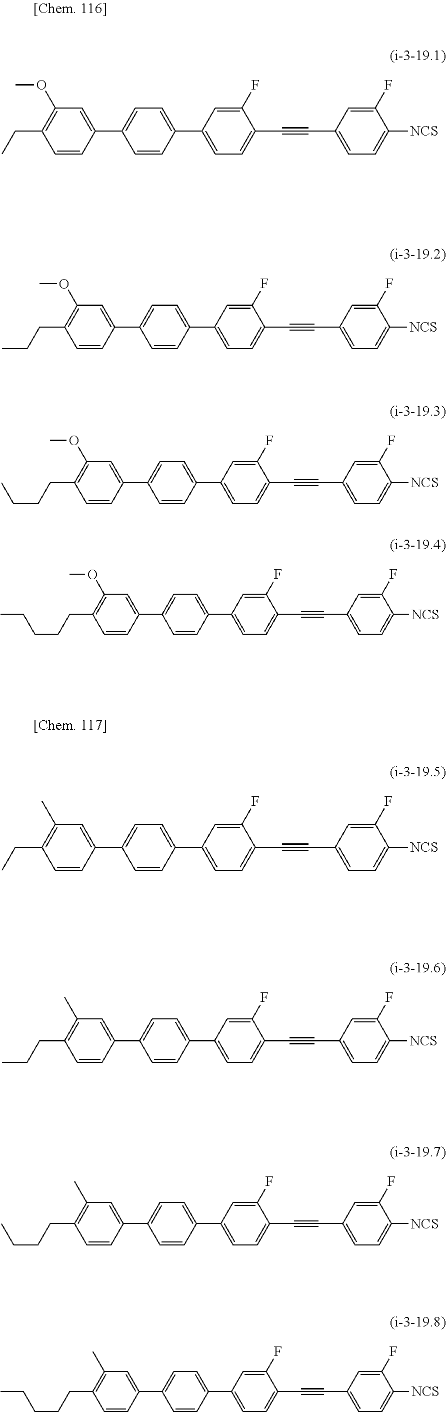

Specific examples of the compounds represented by the general formula (i-3-20) include the compounds represented by the following structural formulae (Jan. 3, 2020.1) to (Jan. 3, 2020.4):

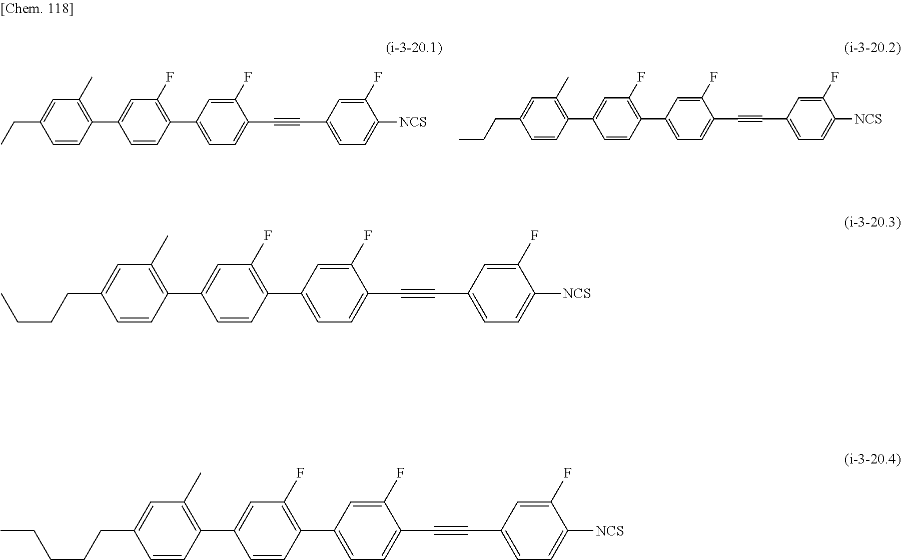

Specific examples of the compounds represented by the general formula (i-3-21) include the compounds represented by the following structural formulae (i-3-21.1) to (i-3-21.4):

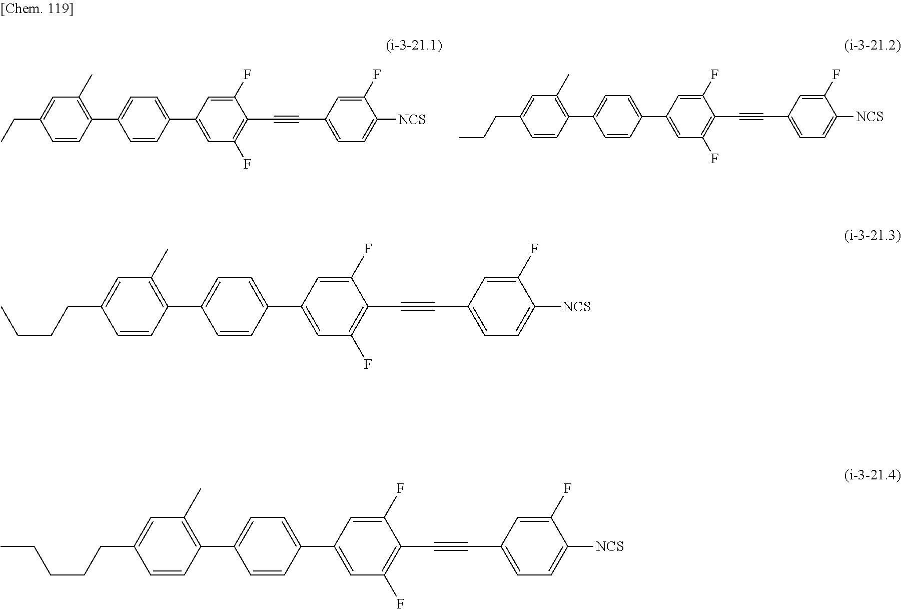

Specific examples of the compounds represented by the general formula (i-3-22) include the compound represented by the structural formula (i-3-22.1):

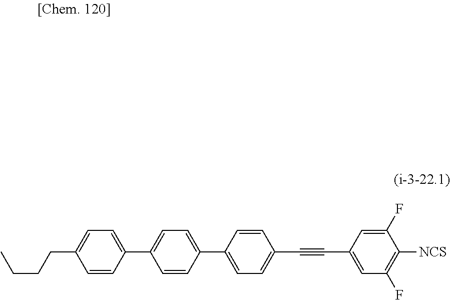

Compounds represented by the general formula (i-4) are preferably the compounds represented by the following general formulae (i-4-1):

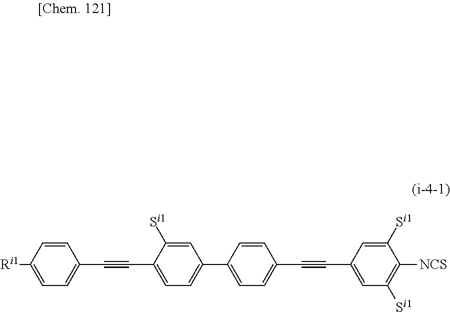

In the general formula (i-4-1), Rⁱ¹ and Sⁱ¹ each independently denote the same as Rⁱ¹ and Sⁱ¹ in the general formula (i).

Specific examples of the compounds represented by the general formula (i-4-1) include the compounds represented by the following structural formulae (i-4-1.1) to (i-4-1.3):

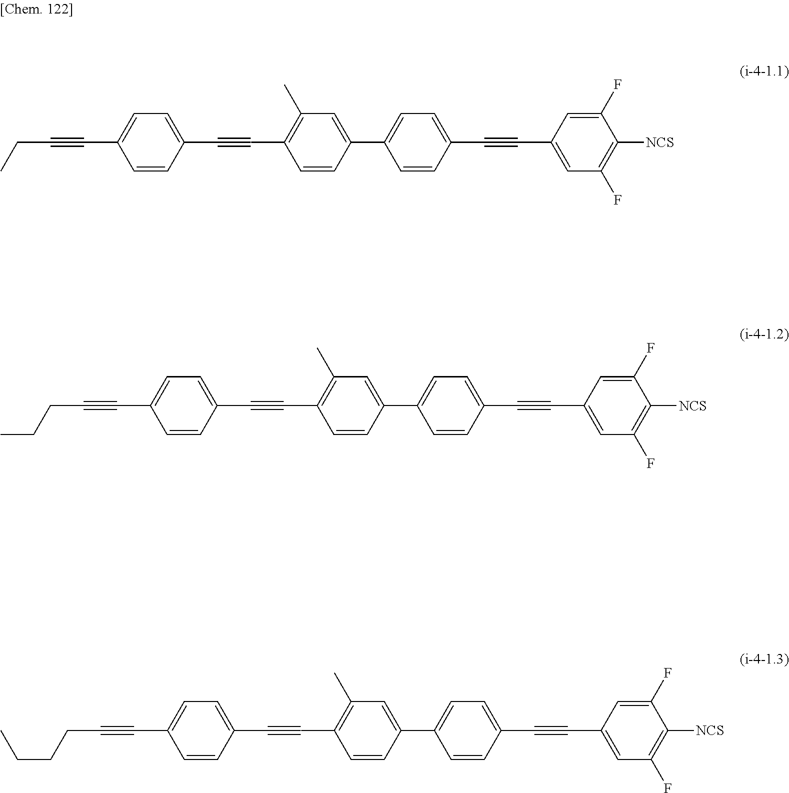

The number of types of compounds represented by the following formulae used in liquid crystal compositions is one or two or more, preferably one to ten, preferably one to five, or preferably one to three: the general formula (i), the general formulae (i-1) to (i-4), the general formulae (i-1-1) to (i-1-20), the general formulae (i-2-1) to (i-2-18), the general formulae (i-3-1) to (i-3-22), the general formula (i-4-1), the structural formulae (i-1-1.1) to (i-1-1.8), the structural formulae (i-1-2.1) to (i-1-2.4), the structural formulae (i-1-3.1) to (i-1-3.4), the structural formulae (i-1-4.1) to (i-1-4.4), the structural formulae (i-1-5.1) to (i-1-5.4), the structural formulae (i-1-6.1) to (i-1-6.4), the structural formulae (i-1-7.1) to (i-1-7.8), the structural formulae (i-1-8.1) to (i-1-8.8), the structural formulae (i-1-9.1) to (i-1-9.8), the structural formulae (i-1-10.1) to (i-1-10.4), the structural formulae (i-1-11.1) to (i-1-11.4), the structural formulae (i-1-12.1) to (i-1-12.8), the structural formulae (i-1-13.1) to (i-1-13.8), the structural formulae (i-1-14.1) to (i-1-14.4), the structural formulae (i-1-15.1) to (i-1-15.4), the structural formulae (i-1-16.1) to (i-1-16.4), the structural formulae (i-1-17.1) to (i-1-17.4), the structural formulae (i-1-18.1) to (i-1-18.4), the structural formulae (i-1-19.1) to (i-1-19.4), the structural formulae (i-1-20.1) to (i-1-20.4), the structural formulae (i-2-1.1) to (i-2-1.4), the structural formulae (i-2-2.1) to (i-2-2.8), the structural formulae (i-2-3.1) to (i-2-3.8), the structural formulae (i-2-4.1) to (i-2-4.4), the structural formulae (i-2-5.1) to (i-2-5.4), the structural formulae (i-2-6.1) to (i-2-6.4), the structural formulae (i-2-7.1) to (i-2-7.15), the structural formulae (i-2-8.1) to (i-2-8.8), the structural formulae (i-2-9.1) to (i-2-9.4), the structural formulae (i-2-10.1) to (i-2-10.4), the structural formulae (i-2-11.1) to (i-2-11.8), the structural formulae (i-2-12.1) to (i-2-12.4), the structural formulae (i-2-13.1) to (i-2-13.4), the structural formulae (i-2-14.1) to (i-2-14.4), the structural formulae (i-2-15.1) to (i-2-15.4), the structural formulae (i-2-16.1) to (i-2-16.4), the structural formulae (i-2-17.1) to (i-2-17.4), the structural formulae (i-2-18.1) to (i-2-18.4), the structural formulae (i-3-1.1) to (i-3-1.4), the structural formulae (i-3-2.1) to (i-3-2.8), the structural formulae (i-3-3.1) to (i-3-3.4), the structural formulae (i-3-4.1) to (i-3-4.8), the structural formulae (i-3-5.1) to (i-3-5.4), the structural formulae (i-3-6.1) to (i-3-6.24), the structural formulae (i-3-7.1) to (i-3-7.4), the structural formulae (i-3-8.1) to (i-3-8.23), the structural formulae (i-3-9.1) to (i-3-9.4), the structural formulae (i-3-10.1) to (i-3-10.8), the structural formulae (i-3-11.1) to (i-3-11.4), the structural formulae (i-3-12.1) to (i-3-12.4), the structural formulae (i-3-13.1) to (i-3-13.4), the structural formulae (i-3-14.1) to (i-3-14.4), the structural formulae (i-3-15.1) to (i-3-15.8), the structural formulae (i-3-16.1) to (i-3-16.4), the structural formulae (i-3-17.1) to (i-3-17.4), the structural formulae (i-3-18.1) to (i-3-18.4), the structural formulae (i-3-19.1) to (i-3-19.8), the structural formulae (i-3-20.1) to (i-3-20.4), the structural formulae (i-3-21.1) to (i-3-21.4), the structural formula (i-3-22.1), or the structural formulae (i-4-1.1) to (i-4-1.3).

The lower limit of the total amount of the compounds represented by the following formulae per 100% by mass of the liquid crystal composition is preferably 18 by mass or more, preferably 3% by mass or more, preferably 5% by mass or more, preferably 10% by mass or more, or preferably 15% by mass or more: the general formula (i), the general formulae (i-1) to (i-4), the general formulae (i-1-1) to (i-1-20), the general formulae (1-2-1) to (Jan. 2, 2018), the general formulae (1-3-1) to (i-3-22), the general formula (i-4-1), the structural formulae (i-1-1.1) to (i-1-1.8), the structural formulae (i-1-2.1) to (i-1-2.4), the structural formulae (i-1-3.1) to (i-1-3.4), the structural formulae (i-1-4.1) to (i-1-4.4), the structural formulae (i-1-5.1) to (i-1-5.4), the structural formulae (i-1-6.1) to (i-1-6.4), the structural formulae (i-1-7.1) to (i-1-7.8), the structural formulae (i-1-8.1) to (i-1-8.8), the structural formulae (i-1-9.1) to (i-1-9.8), the structural formulae (i-1-10.1) to (i-1-10.4), the structural formulae (i-1-11.1) to (i-1-11.4), the structural formulae (i-1-12.1) to (i-1-12.8), the structural formulae (i-1-13.1) to (i-1-13.8), the structural formulae (i-1-14.1) to (i-1-14.4), the structural formulae (i-1-15.1) to (i-1-15.4), the structural formulae (i-1-16.1) to (i-1-16.4), the structural formulae (i-1-17.1) to (i-1-17.4), the structural formulae (i-1-18.1) to (i-1-18.4), the structural formulae (i-1-19.1) to (i-1-19.4), the structural formulae (i-1-20.1) to (i-1-20.4), the structural formulae (i-2-1.1) to (i-2-1.4), the structural formulae (i-2-2.1) to (i-2-2.8), the structural formulae (i-2-3.1) to (i-2-3.8), the structural formulae (i-2-4.1) to (i-2-4.4), the structural formulae (i-2-5.1) to (i-2-5.4), the structural formulae (i-2-6.1) to (i-2-6.4), the structural formulae (i-2-7.1) to (i-2-7.15), the structural formulae (i-2-8.1) to (i-2-8.8), the structural formulae (i-2-9.1) to (i-2-9.4), the structural formulae (i-2-10.1) to (i-2-10.4), the structural formulae (i-2-11.1) to (i-2-11.8), the structural formulae (i-2-12.1) to (i-2-12.4), the structural formulae (i-2-13.1) to (i-2-13.4), the structural formulae (i-2-14.1) to (i-2-14.4), the structural formulae (i-2-15.1) to (i-2-15.4), the structural formulae (i-2-16.1) to (i-2-16.4), the structural formulae (i-2-17.1) to (i-2-17.4), the structural formulae (i-2-18.1) to (i-2-18.4), the structural formulae (i-3-1.1) to (i-3-1.4), the structural formulae (i-3-2.1) to (i-3-2.8), the structural formulae (i-3-3.1) to (i-3-3.4), the structural formulae (i-3-4.1) to (i-3-4.8), the structural formulae (i-3-5.1) to (i-3-5.4), the structural formulae (i-3-6.1) to (i-3-6.24), the structural formulae (i-3-7.1) to (i-3-7.4), the structural formulae (i-3-8.1) to (i-3-8.23), the structural formulae (i-3-9.1) to (i-3-9.4), the structural formulae (i-3-10.1) to (i-3-10.8), the structural formulae (i-3-11.1) to (i-3-11.4), the structural formulae (i-3-12.1) to (i-3-12.4), the structural formulae (i-3-13.1) to (i-3-13.4), the structural formulae (i-3-14.1) to (i-3-14.4), the structural formulae (i-3-15.1) to (i-3-15.8), the structural formulae (i-3-16.1) to (i-3-16.4), the structural formulae (i-3-17.1) to (i-3-17.4), the structural formulae (i-3-18.1) to (i-3-18.4), the structural formulae (i-3-19.1) to (i-3-19.8), the structural formulae (i-3-20.1) to (i-3-20.4), the structural formulae (i-3-21.1) to (i-3-21.4), the structural formula (i-3-22.1), or the structural formulae (i-4-1.1) to (i-4-1.3).

The upper limit of the total amount of the compounds represented by the following formulae per 100% by mass of the liquid crystal composition is preferably 35% by mass or less, preferably 30% by mass or less, preferably 25% by mass or less, preferably 20% by mass or less, preferably 15% by mass or less, preferably 10% by mass or less, or preferably 5% by mass or less: the general formula (i), the general formulae (i-1) to (i-4), the general formulae (i-1-1) to (i-1-20), the general formulae (i-2-1) to (i-2-18), the general formulae (i-3-1) to (i-3-22), the general formula (i-4-1), the structural formulae (i-1-1.1) to (i-1-1.8), the structural formulae (i-1-2.1) to (i-1-2.4), the structural formulae (i-1-3.1) to (i-1-3.4), the structural formulae (i-1-4.1) to (i-1-4.4), the structural formulae (i-1-5.1) to (i-1-5.4), the structural formulae (i-1-6.1) to (i-1-6.4), the structural formulae (i-1-7.1) to (i-1-7.8), the structural formulae (i-1-8.1) to (i-1-8.8), the structural formulae (i-1-9.1) to (i-1-9.8), the structural formulae (i-1-10.1) to (i-1-10.4), the structural formulae (i-1-11.1) to (i-1-11.4), the structural formulae (i-1-12.1) to (i-1-12.8), the structural formulae (i-1-13.1) to (i-1-13.8), the structural formulae (i-1-14.1) to (i-1-14.4), the structural formulae (i-1-15.1) to (i-1-15.4), the structural formulae (i-1-16.1) to (i-1-16.4), the structural formulae (i-1-17.1) to (i-1-17.4), the structural formulae (i-1-18.1) to (i-1-18.4), the structural formulae (i-1-19.1) to (i-1-19.4), the structural formulae (i-1-20.1) to (i-1-20.4), the structural formulae (i-2-1.1) to (i-2-1.4), the structural formulae (i-2-2.1) to (i-2-2.8), the structural formulae (i-2-3.1) to (i-2-3.8), the structural formulae (i-2-4.1) to (i-2-4.4), the structural formulae (i-2-5.1) to (i-2-5.4), the structural formulae (i-2-6.1) to (i-2-6.4), the structural formulae (i-2-7.1) to (i-2-7.15), the structural formulae (i-2-8.1) to (i-2-8.8), the structural formulae (i-2-9.1) to (i-2-9.4), the structural formulae (i-2-10.1) to (i-2-10.4), the structural formulae (i-2-11.1) to (i-2-11.8), the structural formulae (i-2-12.1) to (i-2-12.4), the structural formulae (i-2-13.1) to (i-2-13.4), the structural formulae (i-2-14.1) to (i-2-14.4), the structural formulae (i-2-15.1) to (i-2-15.4), the structural formulae (i-2-16.1) to (i-2-16.4), the structural formulae (i-2-17.1) to (i-2-17.4), the structural formulae (i-2-18.1) to (i-2-18.4), the structural formulae (i-3-1.1) to (i-3-1.4), the structural formulae (i-3-2.1) to (i-3-2.8), the structural formulae (i-3-3.1) to (i-3-3.4), the structural formulae (i-3-4.1) to (i-3-4.8), the structural formulae (i-3-5.1) to (i-3-5.4), the structural formulae (i-3-6.1) to (i-3-6.24), the structural formulae (i-3-7.1) to (i-3-7.4), the structural formulae (i-3-8.1) to (i-3-8.23), the structural formulae (i-3-9.1) to (i-3-9.4), the structural formulae (i-3-10.1) to (i-3-10.8), the structural formulae (i-3-11.1) to (i-3-11.4), the structural formulae (i-3-12.1) to (i-3-12.4), the structural formulae (i-3-13.1) to (i-3-13.4), the structural formulae (i-3-14.1) to (i-3-14.4), the structural formulae (i-3-15.1) to (i-3-15.8), the structural formulae (i-3-16.1) to (i-3-16.4), the structural formulae (i-3-17.1) to (i-3-17.4), the structural formulae (i-3-18.1) to (i-3-18.4), the structural formulae (i-3-19.1) to (i-3-19.8), the structural formulae (i-3-20.1) to (i-3-20.4), the structural formulae (i-3-21.1) to (i-3-21.4), the structural formula (i-3-22.1), or the structural formulae (i-4-1.1) to (i-4-1.3).

The total content of the compounds represented by the following formulae per 100% by mass of the liquid crystal composition preferably ranges from 1% to 35% by mass, preferably 1% to 30% by mass, or preferably 3% to 25% by mass, from the perspective of solubility, Δn, and/or Δεᵣ: the general formula (i), the general formulae (i-1) to (i-4), the general formulae (i-1-1) to (i-1-20), the general formulae (i-2-1) to (i-2-18), the general formulae (i-3-1) to (i-3-22), the general formula (i-4-1), the structural formulae (i-1-1.1) to (i-1-1.8), the structural formulae (i-1-2.1) to (i-1-2.4), the structural formulae (i-1-3.1) to (i-1-3.4), the structural formulae (i-1-4.1) to (i-1-4.4), the structural formulae (i-1-5.1) to (i-1-5.4), the structural formulae (i-1-6.1) to (i-1-6.4), the structural formulae (i-1-7.1) to (i-1-7.8), the structural formulae (i-1-8.1) to (i-1-8.8), the structural formulae (i-1-9.1) to (i-1-9.8), the structural formulae (i-1-10.1) to (i-1-10.4), the structural formulae (i-1-11.1) to (i-1-11.4), the structural formulae (i-1-12.1) to (i-1-12.8), the structural formulae (i-1-13.1) to (i-1-13.8), the structural formulae (i-1-14.1) to (i-1-14.4), the structural formulae (i-1-15.1) to (i-1-15.4), the structural formulae (i-1-16.1) to (i-1-16.4), the structural formulae (i-1-17.1) to (i-1-17.4), the structural formulae (i-1-18.1) to (i-1-18.4), the structural formulae (i-1-19.1) to (i-1-19.4), the structural formulae (i-1-20.1) to (i-1-20.4), the structural formulae (i-2-1.1) to (i-2-1.4), the structural formulae (i-2-2.1) to (i-2-2.8), the structural formulae (i-2-3.1) to (i-2-3.8), the structural formulae (i-2-4.1) to (i-2-4.4), the structural formulae (i-2-5.1) to (i-2-5.4), the structural formulae (i-2-6.1) to (i-2-6.4), the structural formulae (i-2-7.1) to (i-2-7.15), the structural formulae (i-2-8.1) to (i-2-8.8), the structural formulae (i-2-9.1) to (i-2-9.4), the structural formulae (i-2-10.1) to (i-2-10.4), the structural formulae (i-2-11.1) to (i-2-11.8), the structural formulae (i-2-12.1) to (i-2-12.4), the structural formulae (i-2-13.1) to (i-2-13.4), the structural formulae (i-2-14.1) to (i-2-14.4), the structural formulae (i-2-15.1) to (i-2-15.4), the structural formulae (i-2-16.1) to (i-2-16.4), the structural formulae (i-2-17.1) to (i-2-17.4), the structural formulae (i-2-18.1) to (i-2-18.4), the structural formulae (i-3-1.1) to (i-3-1.4), the structural formulae (i-3-2.1) to (i-3-2.8), the structural formulae (i-3-3.1) to (i-3-3.4), the structural formulae (i-3-4.1) to (i-3-4.8), the structural formulae (i-3-5.1) to (i-3-5.4), the structural formulae (i-3-6.1) to (i-3-6.24), the structural formulae (i-3-7.1) to (i-3-7.4), the structural formulae (i-3-8.1) to (i-3-8.23), the structural formulae (i-3-9.1) to (i-3-9.4), the structural formulae (i-3-10.1) to (i-3-10.8), the structural formulae (i-3-11.1) to (i-3-11.4), the structural formulae (i-3-12.1) to (i-3-12.4), the structural formulae (i-3-13.1) to (i-3-13.4), the structural formulae (i-3-14.1) to (i-3-14.4), the structural formulae (i-3-15.1) to (i-3-15.8), the structural formulae (i-3-16.1) to (i-3-16.4), the structural formulae (i-3-17.1) to (i-3-17.4), the structural formulae (i-3-18.1) to (i-3-18.4), the structural formulae (i-3-19.1) to (i-3-19.8), the structural formulae (i-3-20.1) to (i-3-20.4), the structural formulae (i-3-21.1) to (i-3-21.4), the structural formula (i-3-22.1), or the structural formulae (i-4-1.1) to (i-4-1.3).

The compounds represented by the general formula (i) (including subordinate concepts) can be synthesized by a known synthesis method, and some examples are described below.

### (Production Method 1) Production of Compound Represented by Formula (S-7):

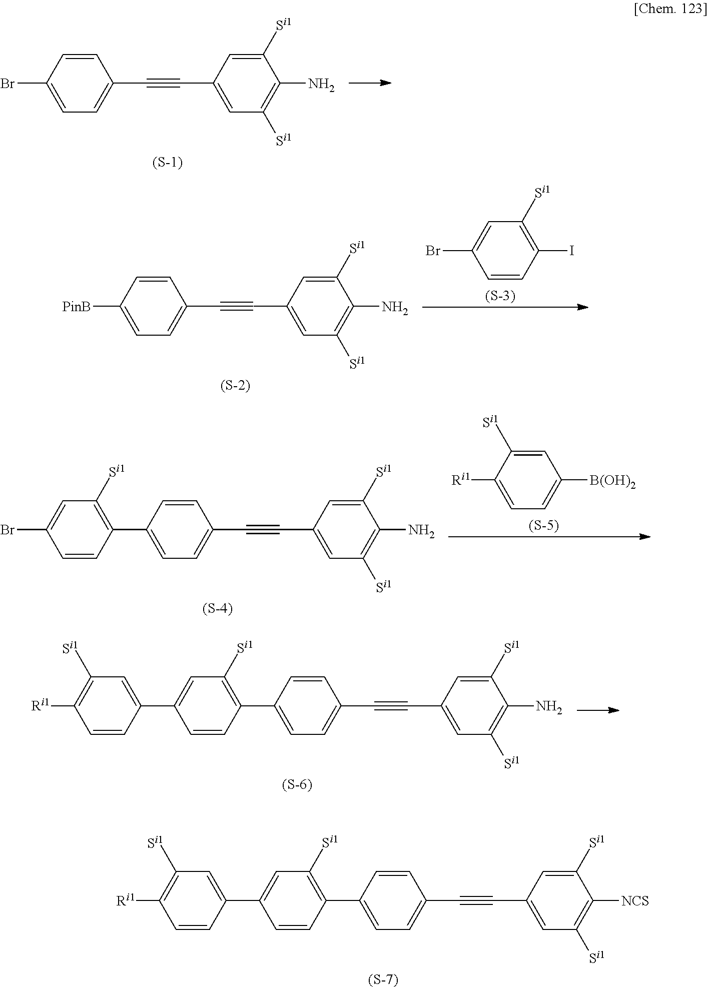

(In the general formulae (S-1) to (S-7), Rⁱ¹ and Sⁱ¹ denote the same as Rⁱ¹ and Sⁱ¹ in the general formula (i).)

First, a compound represented by the general formula (S-1) can be reacted with bis(pinacolato)diboron to produce a compound represented by the general formula (S-2).

The reaction method is, for example, a method of performing Suzuki coupling in the presence of a metal catalyst and a base.

Specific examples of the metal catalyst include [1,1′-bis(diphenylphosphino)ferrocene]palladium (II) dichloride, palladium (II) acetate, dichlorobis[di-tert-butyl(p-dimethylaminophenyl)phosphino] palladium (II), dichlorobis(triphenylphosphine) palladium (II), and tetrakis(triphenylphosphine) palladium (0).

When palladium (II) acetate is used as the palladium catalyst, a ligand, such as triphenylphosphine or 2-dicyclohexylphosphino-2′,4′,6′-triisopropylbiphenyl, may be added.

Specific examples of the base include potassium acetate, potassium carbonate, potassium phosphate, and cesium carbonate.

Next, a compound represented by the general formula (S-2) can be reacted with a compound represented by the general formula (S-3) to produce a compound represented by the general formula (S-4).

The reaction method is, for example, a method of performing Suzuki coupling in the presence of a metal catalyst and a base.

Specific examples of the metal catalyst include [1,1′-bis(diphenylphosphino)ferrocene]palladium (II) dichloride, palladium (II) acetate, dichlorobis[di-tert-butyl(p-dimethylaminophenyl)phosphino] palladium (II), dichlorobis(triphenylphosphine) palladium (II), and tetrakis(triphenylphosphine) palladium (0).

When palladium (II) acetate is used as the palladium catalyst, a ligand, such as triphenylphosphine or 2-dicyclohexylphosphino-2′,4′,6′-triisopropylbiphenyl, may be added.

Specific examples of the base include potassium carbonate, potassium phosphate, and cesium carbonate.

Furthermore, a compound represented by the general formula (S-4) can be reacted with a compound represented by the general formula (S-5) to produce a compound represented by the general formula (S-6).

The reaction method is, for example, a method of performing Suzuki coupling in the presence of a metal catalyst and a base.

Specific examples of the metal catalyst include [1,1′-bis(diphenylphosphino)ferrocene]palladium (II) dichloride, palladium (II) acetate, dichlorobis[di-tert-butyl(p-dimethylaminophenyl)phosphino] palladium (II), dichlorobis(triphenylphosphine) palladium (II), and tetrakis(triphenylphosphine) palladium (0).

When palladium (II) acetate is used as the palladium catalyst, a ligand, such as triphenylphosphine or 2-dicyclohexylphosphino-2′,4′,6′-triisopropylbiphenyl, may be added.

Specific examples of the base include potassium carbonate, potassium phosphate, and cesium carbonate.

Finally, an amino group of the compound represented by the general formula (S-6) can be reacted with 1,1-thiocarbonyldiimidazole, 1,1-thiocarbonyl di-2 (1H)-pyridone, thiophosgene, or the like to produce a compound represented by the general formula (S-7) as a target product.

### (Production Method 2) Production of Compound Represented by Formula (S-13):

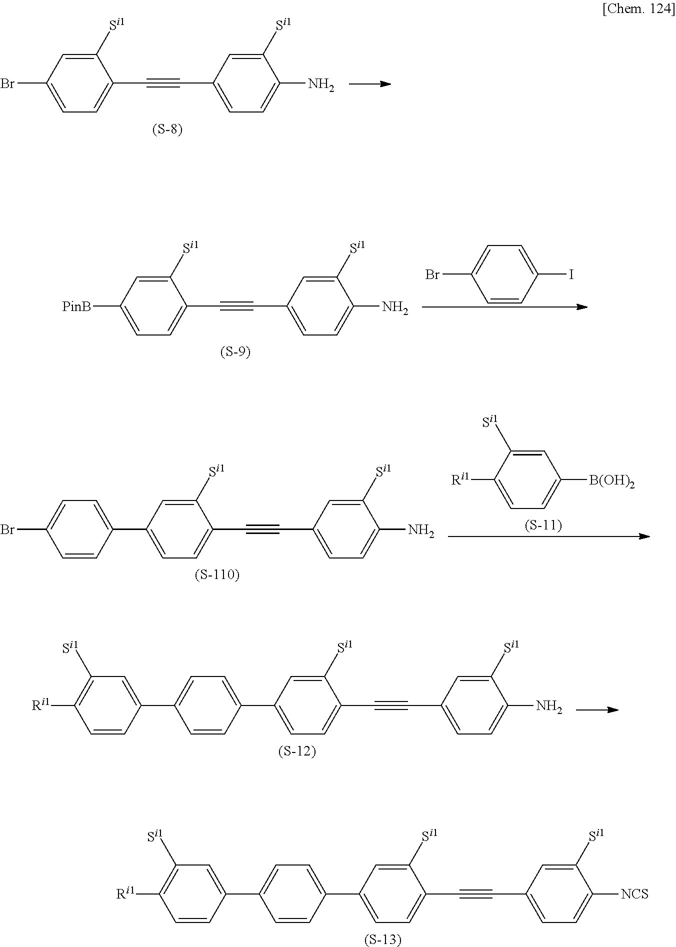

(In the general formulae (S-8) to (S-13), Rⁱ¹ and Sⁱ¹ denote the same as Rⁱ¹ and Sⁱ¹ in the general formula (i).)

First, a compound represented by the general formula (S-8) can be reacted with bis(pinacolato)diboron to produce a compound represented by the general formula (S-9).

The reaction method is, for example, a method of performing Suzuki coupling in the presence of a metal catalyst and a base.

Specific examples of the metal catalyst include [1,1′-bis(diphenylphosphino)ferrocene]palladium (II) dichloride, palladium (II) acetate, dichlorobis[di-tert-butyl(p-dimethylaminophenyl)phosphino] palladium (II), dichlorobis(triphenylphosphine) palladium (II), and tetrakis(triphenylphosphine) palladium (0).

When palladium (II) acetate is used as the palladium catalyst, a ligand, such as triphenylphosphine or 2-dicyclohexylphosphino-2′,4′,6′-triisopropylbiphenyl, may be added.

Specific examples of the base include potassium acetate, potassium carbonate, potassium phosphate, and cesium carbonate.

Next, a compound represented by the general formula (S-9) can be reacted with 1-bromo-4-iodobenzene to produce a compound represented by the general formula (S-10).

The reaction method is, for example, a method of performing Suzuki coupling in the presence of a metal catalyst and a base.

Specific examples of the metal catalyst include [1,1′-bis(diphenylphosphino)ferrocene]palladium (II) dichloride, palladium (II) acetate, dichlorobis[di-tert-butyl(p-dimethylaminophenyl)phosphino] palladium (II), dichlorobis(triphenylphosphine) palladium (II), and tetrakis(triphenylphosphine) palladium (0).

When palladium (II) acetate is used as the palladium catalyst, a ligand, such as triphenylphosphine or 2-dicyclohexylphosphino-2′,4′,6′-triisopropylbiphenyl, may be added.

Specific examples of the base include potassium carbonate, potassium phosphate, and cesium carbonate.

Furthermore, a compound represented by the general formula (S-10) can be reacted with a compound represented by the general formula (S-11) to produce a compound represented by the general formula (S-12).

The reaction method is, for example, a method of performing Suzuki coupling in the presence of a metal catalyst and a base.

Specific examples of the metal catalyst include [1,1′-bis(diphenylphosphino)ferrocene]palladium (II) dichloride, palladium (II) acetate, dichlorobis[di-tert-butyl(p-dimethylaminophenyl)phosphino] palladium (II), dichlorobis(triphenylphosphine) palladium (II), and tetrakis(triphenylphosphine) palladium (0).

When palladium (II) acetate is used as the palladium catalyst, a ligand, such as triphenylphosphine or 2-dicyclohexylphosphino-2′,4′,6′-triisopropylbiphenyl, may be added.

Specific examples of the base include potassium carbonate, potassium phosphate, and cesium carbonate.

Finally, an amino group of the compound represented by the general formula (S-12) can be reacted with 1,1-thiocarbonyldiimidazole, 1,1-thiocarbonyl di-2 (1H)-pyridone, thiophosgene, or the like to produce a compound represented by the general formula (S-13) as a target product.

### (Production Method 3) Production of Compound Represented by Formula (S-19):

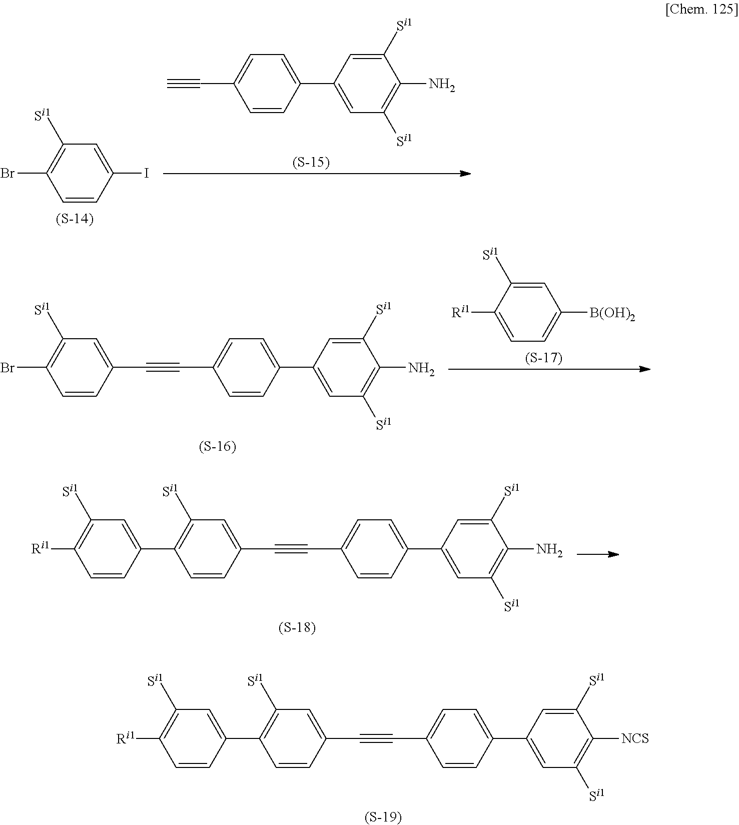

(In the general formulae (S-14) to (S-19), Rⁱ¹ and Sⁱ¹ denote the same as Rⁱ¹ and Sⁱ¹ in the general formula (i).)

First, a compound represented by the general formula (S-14) can be reacted with a compound represented by the general formula (S-15) to produce a compound represented by the general formula (S-16).

The reaction method is, for example, a Sonogashira coupling reaction using a palladium catalyst, a copper catalyst, and a base.

Specific examples of the palladium catalyst include [1,1′-bis(diphenylphosphino)ferrocene]palladium (II) dichloride, palladium (II) acetate, dichlorobis[di-tert-butyl(p-dimethylaminophenyl)phosphino] palladium (II), dichlorobis(triphenylphosphine) palladium (II), and tetrakis(triphenylphosphine) palladium (0).

When palladium (II) acetate is used as the palladium catalyst, a ligand, such as triphenylphosphine or 2-dicyclohexylphosphino-2′,4′,6′-triisopropylbiphenyl, may be added.

Specific examples of the copper catalyst include copper (I) iodide.

Specific examples of the base include triethylamine and diisopropylamine.

Next, a compound represented by the general formula (S-16) can be reacted with a compound represented by the general formula (S-17) to produce a compound represented by the general formula (S-18).

The reaction method is, for example, a method of performing Suzuki coupling in the presence of a metal catalyst and a base.

Specific examples of the metal catalyst include [1,1′-bis(diphenylphosphino)ferrocene]palladium (II) dichloride, palladium (II) acetate, dichlorobis[di-tert-butyl(p-dimethylaminophenyl)phosphino] palladium (II), dichlorobis(triphenylphosphine) palladium (II), and tetrakis(triphenylphosphine) palladium (0).

When palladium (II) acetate is used as the palladium catalyst, a ligand, such as triphenylphosphine or 2-dicyclohexylphosphino-2′,4′,6′-triisopropylbiphenyl, may be added.

Specific examples of the base include potassium carbonate, potassium phosphate, and cesium carbonate.

Finally, an amino group of the compound represented by the general formula (S-18) can be reacted with 1,1-thiocarbonyldiimidazole, 1,1-thiocarbonyl di-2 (1H)-pyridone, thiophosgene, or the like to produce a compound represented by the general formula (S-19) as a target product.

(Compound Represented by General Formula (ii))

From the perspective of solubility, Δn, and/or Δεᵣ, a liquid crystal composition according to the present invention preferably contains one or two or more compounds represented by the following general formula (ii) with an isothiocyanate group (—NCS).

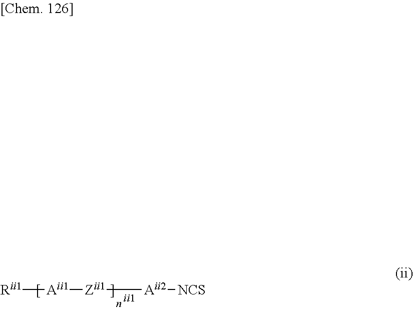

In the general formula (ii), Rⁱⁱ¹ denotes an alkyl group with 1 to 20 carbon atoms.

The alkyl group is a linear, branched, or cyclic alkyl group, preferably a linear alkyl group.

The number of carbon atoms in the alkyl group preferably ranges from 2 to 10 or preferably 2 to 6.

One or two or more —CH₂— groups in the alkyl group are each independently optionally substituted with —O—, —S—, —CO—, and/or —CS—.

One or two or more —CH₂—CH₂— groups in the alkyl group are optionally substituted with —CH═CH—, —CO—O—, —O—CO—, —CO—S—, —S—CO—, —CO—NH—, —NH—CO—, —CH═CH—, —CF═CF—, and/or —C≡C—.

One or two or more hydrogen atoms in the alkyl group are each independently optionally substituted with a halogen atom.

The halogen atom may be a fluorine atom, a chlorine atom, a bromine atom, or an iodine atom.

When the alkyl group is substituted with a predetermined group, oxygen atoms are not directly bonded to each other.

From the perspective of the stability of the compound, sulfur atoms are preferably not directly bonded to each other, and/or an oxygen atom and a sulfur atom are preferably not directly bonded to each other.

For example, Rⁱⁱ¹ can denote an alkoxy group with 1 to 19 carbon atoms by substituting one —CH₂— in the alkyl group with —O—.

The alkoxy group is a linear, branched, or cyclic alkoxy group, preferably a linear alkoxy group.

The number of carbon atoms in the alkoxy group preferably ranges from 2 to 10 or preferably 2 to 6.

Rⁱⁱ¹ can denote an alkylsulfanyl group (alkylthio group) with 1 to 19 carbon atoms by substituting one —CH₂— in the alkyl group with —S—.

The alkylsulfanyl group is a linear, branched, or cyclic alkylsulfanyl group, preferably a linear alkylsulfanyl group.

The number of carbon atoms in the alkylsulfanyl group preferably ranges from 1 to 10 or preferably 1 to 6.

Rⁱⁱ¹ can denote an alkenyl group with 2 to 20 carbon atoms by substituting one or two or more —CH₂—CH₂— groups in the alkyl group with —CH═CH—.

The alkenyl group is a linear, branched, or cyclic alkenyl group, preferably a linear alkenyl group.

The number of carbon atoms in the alkenyl group preferably ranges from 2 to 10 or preferably 2 to 6.

Rⁱⁱ¹ can denote an alkynyl group with 2 to 20 carbon atoms by substituting one or two or more —CH₂—CH₂— groups in the alkyl group with —C≡C—.

The alkynyl group is a linear, branched, or cyclic alkynyl group, preferably a linear alkynyl group.

The number of carbon atoms in the alkynyl group preferably ranges from 2 to 10 or preferably 2 to 6.

The alkynyl group is preferably an alkynyl group represented by the following formula (Rⁱⁱ¹-A) from the perspective of the ease of synthesis and elongation of a conjugated system.

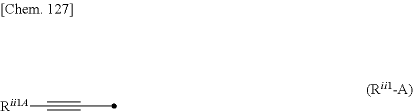

In the formula (Rⁱⁱ¹-A), R^{ii1A} denotes an alkyl group with 1 to 18 carbon atoms.

The alkyl group with 1 to 18 carbon atoms is a linear, branched, or cyclic alkyl group, preferably a linear alkyl group.

The number of carbon atoms in the alkyl group with 1 to 18 carbon atoms preferably ranges from 1 to 8.

One or two or more —CH₂— groups in the alkyl group are each independently optionally substituted with —O—, —S—, —CO—, and/or —CS—.

One or two or more —CH₂—CH₂— groups in the alkyl group are each independently optionally substituted with —CO—O—, —O—CO—, —CO—S—, —S—CO—, —CO—NH—, —NH—CO—, —CH═CH—, —CF═CF—, and/or —C≡C—.

One or two or more hydrogen atoms in the alkyl group are each independently optionally substituted with a halogen atom.

The halogen atom may be a fluorine atom, a chlorine atom, a bromine atom, or an iodine atom.

When the alkyl group is substituted with a predetermined group, oxygen atoms are not directly bonded to each other.

From the perspective of the stability of the compound, sulfur atoms are preferably not directly bonded to each other, and/or an oxygen atom and a sulfur atom are preferably not directly bonded to each other.

In the formula (Rⁱⁱ¹-A), a black dot represents a bonding arm to Aⁱⁱ¹.

Rⁱⁱ¹ can denote an alkenyloxy group with 2 to 19 carbon atoms by substituting one —CH₂— in the alkyl group with —O— and substituting one or two or more —CH₂—CH₂— groups with —CH═CH—.

The alkenyloxy group is a linear, branched, or cyclic alkenyloxy group, preferably a linear alkenyloxy group.

The number of carbon atoms in the alkenyloxy group preferably ranges from 2 to 10 or preferably 2 to 6.

Rⁱⁱ¹ can denote an alkyl halide group with 1 to 20 carbon atoms by substituting one or two or more hydrogen atoms in the alkyl group with a halogen atom.

The alkyl halide group is a linear, branched, or cyclic alkyl halide group, preferably a linear alkyl halide group.

The number of carbon atoms in the alkyl halide group preferably ranges from 2 to 10 or preferably 2 to 6.

Rⁱⁱ¹ can denote an alkoxy halide group with 1 to 19 carbon atoms by substituting one —CH₂— in the alkyl group with —O— and substituting one or two or more hydrogen atoms in the alkyl group with a halogen atom.

The alkoxy halide group is a linear, branched, or cyclic alkoxy halide group, preferably a linear alkoxy halide group.

The number of carbon atoms in the alkoxy halide group preferably ranges from 2 to 10 or preferably 2 to 6.

Specific examples of the alkyl group with 1 to 20 carbon atoms (including substituted ones) in Rⁱⁱ¹ include the groups represented by the formulae (Rⁱⁱ¹-1) to (Rⁱⁱ¹-56):

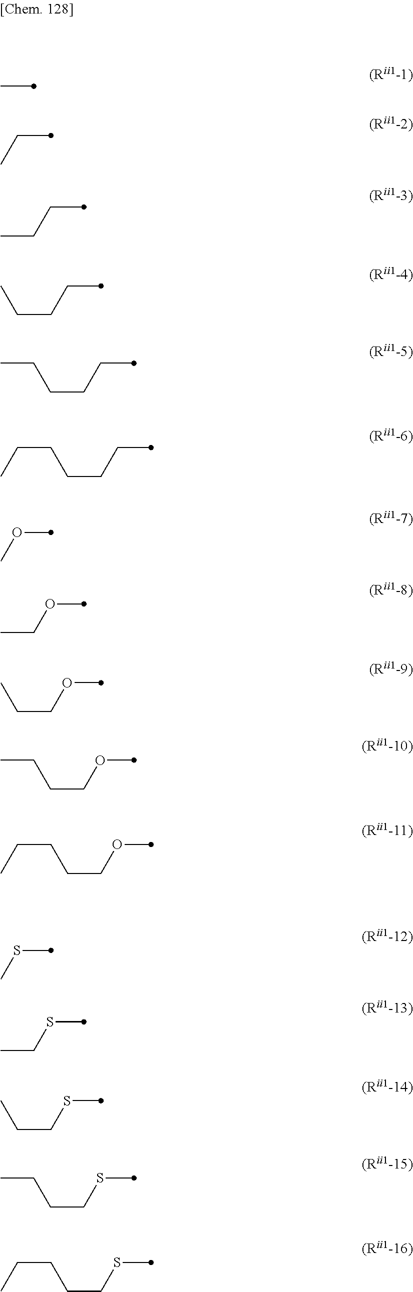

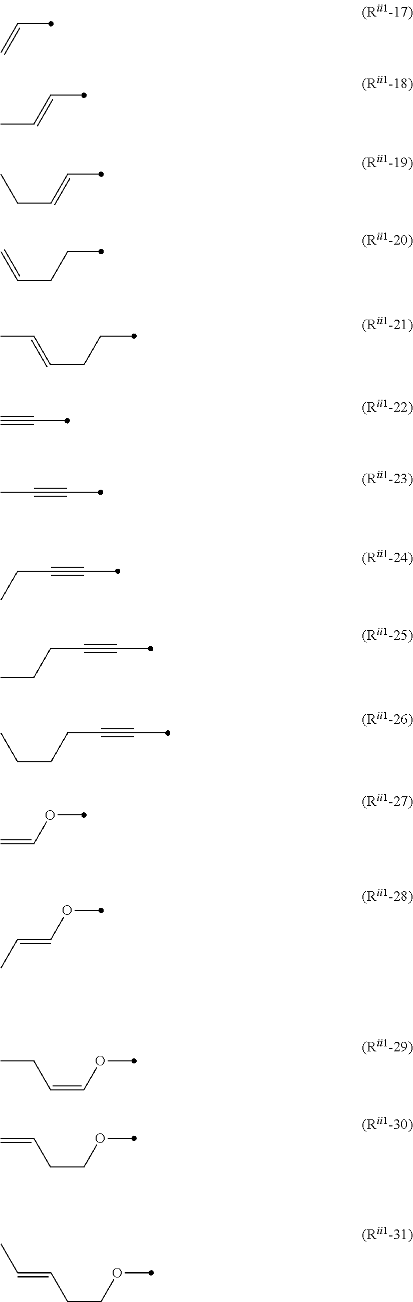

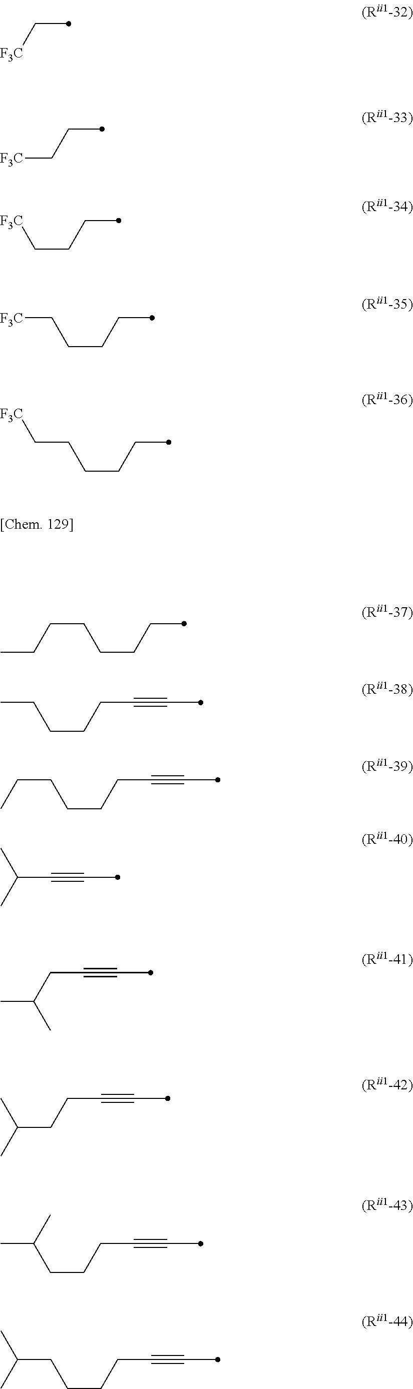

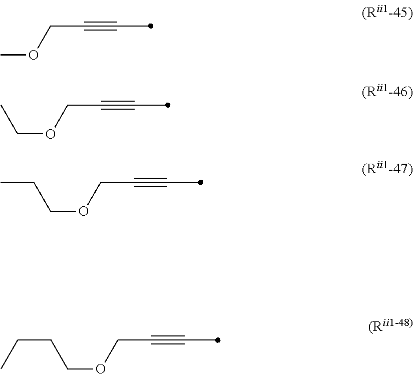

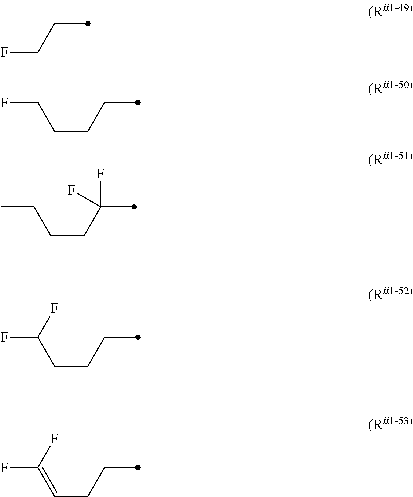

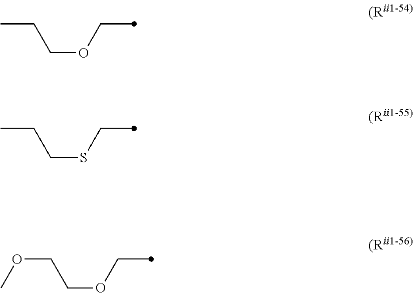

In the formulae (Rⁱⁱ¹-1) to (Rⁱⁱ¹-56), a black dot represents a bonding arm to Aⁱⁱ¹.

When the ring structure to which Rⁱⁱ¹ is bonded is a phenyl group (aromatic), a linear alkyl group with 1 to 5 carbon atoms, a linear alkoxy group with 1 to 4 carbon atoms, or an alkenyl group with 4 or 5 carbon atoms is preferred. When the ring structure to which Rⁱⁱ¹ is bonded is a saturated ring structure, such as cyclohexane, pyran, or dioxane, a linear alkyl group with 1 to 5 carbon atoms, a linear alkoxy group with 1 to 4 carbon atoms, or a linear alkenyl group with 2 to 5 carbon atoms is preferred.

To stabilize a nematic phase, in Rⁱⁱ¹, the total number of carbon atoms and, if present, oxygen atoms is preferably 5 or less, and a straight chain is preferred.

From the perspective of solubility, Rⁱⁱ¹ preferably denotes a linear or branched alkyl group with 2 to 8 carbon atoms, a linear alkoxy group with 2 to 8 carbon atoms, a linear alkoxy halide group with 1 to 8 carbon atoms, a linear alkynyl group with 2 to 8 carbon atoms, or a linear alkylsulfanyl group with 1 to 6 carbon atoms.

In the general formula (ii), Aⁱⁱ¹ and Aⁱⁱ² each independently denote a group selected from the group consisting of the groups (a), (b), (c), and (d):
(a) a 1,4-cyclohexylene group (one —CH₂— or two or more nonadjacent —CH₂— groups in this group are optionally substituted with —O— and/or —S—),
(b) a 1,4-phenylene group (one —CH═ or two or more —CH═ groups in this group are optionally substituted with —N═),
(c) a 1,4-cyclohexenylene group, a bicyclo[2.2.2]octane-1,4-diyl group, a naphthalene-2,6-diyl group, a naphthalene-1,4-diyl group, a 1,2,3,4-tetrahydronaphthalene-2,6-diyl group, a 5,6,7,8-tetrahydronaphthalene-1,4-diyl group, a decahydronaphthalene-2,6-diyl group, an anthracene-2,6-diyl group, an anthracene-1,4-diyl group, an anthracene-9,10-diyl group, a phenanthrene-2,7-diyl group, a 2,3-dihydroindene-2,5-diyl group, a 2,3-dihydroindene-1,5-diyl group, and a 2,3-dihydroindene-3,5-diyl group (one —CH═ or two or more —CH═ groups in the naphthalene-2,6-diyl group, the naphthalene-1,4-diyl group, the 1,2,3,4-tetrahydronaphthalene-2,6-diyl group, the 5,6,7,8-tetrahydronaphthalene-1,4-diyl group, the anthracene-2,6-diyl group, the anthracene-1,4-diyl group, the anthracene-9,10-diyl group, or the phenanthrene-2,7-diyl group are optionally substituted with —N═), and
(d) a thiophene-2,5-diyl group, a benzothiophene-2,5-diyl group, a benzothiophene-2,6-diyl group, a dibenzothiophene-3,7-diyl group, a dibenzothiophene-2,6-diyl group, a thieno[3,2-b]thiophene-2,5-diyl group, and a benzo[1,2-b: 4,5-b′]dithiophene-2,6-diyl group (one —CH═ or two or more —CH═ groups in this group are optionally substituted with —N═).

One or two or more hydrogen atoms in Aⁱⁱ¹ and Aⁱⁱ² are each independently optionally substituted with a substituent Sⁱⁱ¹.

The substituent Sⁱⁱ¹ denotes any one of a halogen atom, a pentafluorosulfanyl group, a nitro group, a cyano group, an isocyano group, an amino group, a hydroxy group, a mercapto group, a methylamino group, a dimethylamino group, a diethylamino group, a diisopropylamino group, a trimethylsilyl group, a dimethylsilyl group, a thioisocyano group, and an alkyl group with 1 to 20 carbon atoms.

The halogen atom may be a fluorine atom, a chlorine atom, a bromine atom, or an iodine atom.

The alkyl group with 1 to 20 carbon atoms is a linear, branched, or cyclic alkyl group, preferably a linear alkyl group.

The number of carbon atoms in the alkyl group with 1 to 20 carbon atoms preferably ranges from 2 to 10 or preferably 2 to 6.

One or two or more —CH₂— groups in the alkyl group are each independently optionally substituted with —O—, —S—, —CO—, and/or —CS—.

One or two or more —CH₂—CH₂— groups in the alkyl group are each independently optionally substituted with —CO—O—, —O—CO—, —CO—S—, —S—CO—, —CO—NH—, —NH—CO—, —CH═CH—, —CF═CF—, and/or —C≡C—.

One or two or more hydrogen atoms in the alkyl group are each independently optionally substituted with a halogen atom.

The halogen atom may be a fluorine atom, a chlorine atom, a bromine atom, or an iodine atom.

When the alkyl group is substituted with a predetermined group, oxygen atoms are not directly bonded to each other.

From the perspective of the stability of the compound, sulfur atoms are preferably not directly bonded to each other, and/or an oxygen atom and a sulfur atom are preferably not directly bonded to each other.

For example, the substituent Sⁱⁱ¹ can denote an alkoxy group with 1 to 19 carbon atoms by substituting one —CH₂— in the alkyl group with —O—.

The alkoxy group is a linear, branched, or cyclic alkoxy group, preferably a linear alkoxy group.

The number of carbon atoms in the alkoxy group preferably ranges from 2 to 10 or preferably 2 to 6.

The substituent Sⁱⁱ¹ can denote an alkylsulfanyl group (alkylthio group) with 1 to 19 carbon atoms by substituting one —CH₂— in the alkyl group with —S—.

The alkylsulfanyl group is a linear, branched, or cyclic alkylsulfanyl group, preferably a linear alkylsulfanyl group.

The number of carbon atoms in the alkylsulfanyl group preferably ranges from 2 to 10 or preferably 2 to 6.

The substituent Sⁱⁱ¹ can denote an alkenyl group with 2 to 20 carbon atoms by substituting one or two or more —CH₂—CH₂— groups in the alkyl group with —CH═CH—.

The alkenyl group is a linear, branched, or cyclic alkenyl group, preferably a linear alkenyl group.

The number of carbon atoms in the alkenyl group preferably ranges from 2 to 10 or preferably 2 to 6.

The substituent Sⁱⁱ¹ can denote an alkynyl group with 2 to 20 carbon atoms by substituting one or two or more —CH₂—CH₂— groups in the alkyl group with —C≡C—.

The alkynyl group is a linear, branched, or cyclic alkynyl group, preferably a linear alkynyl group.

The number of carbon atoms in the alkynyl group preferably ranges from 2 to 10 or preferably 2 to 6.

The substituent Sⁱⁱ¹ can denote an alkenyloxy group with 2 to 19 carbon atoms by substituting one —CH₂— in the alkyl group with —O— and substituting one or two or more —CH₂—CH₂— groups with —CH═CH—.

The alkenyloxy group is a linear, branched, or cyclic alkenyloxy group, preferably a linear alkenyloxy group.

The number of carbon atoms in the alkenyloxy group preferably ranges from 2 to 10 or preferably 2 to 6.

The substituent Sⁱⁱ¹ can denote an alkyl halide group with 1 to 20 carbon atoms by substituting one or two or more hydrogen atoms in the alkyl group with a halogen atom.

The alkyl halide group is a linear, branched, or cyclic alkyl halide group, preferably a linear alkyl halide group.

The number of carbon atoms in the alkyl halide group preferably ranges from 2 to 10 or preferably 2 to 6.

The substituent Sⁱⁱ¹ can denote an alkoxy halide group with 1 to 19 carbon atoms by substituting one —CH₂— in the alkyl group with —O— and substituting one or two or more hydrogen atoms in the alkyl group with a halogen atom.

The alkoxy halide group is a linear, branched, or cyclic alkoxy halide group, preferably a linear alkoxy halide group.

The number of carbon atoms in the alkoxy halide group preferably ranges from 2 to 10 or preferably 2 to 6.

Specific examples of the alkyl group with 1 to 20 carbon atoms (including substituted ones) in the substituent Sⁱⁱ¹ include the groups represented by the formulae (S^{ii1R}-1) to (S^{ii1R}-36):

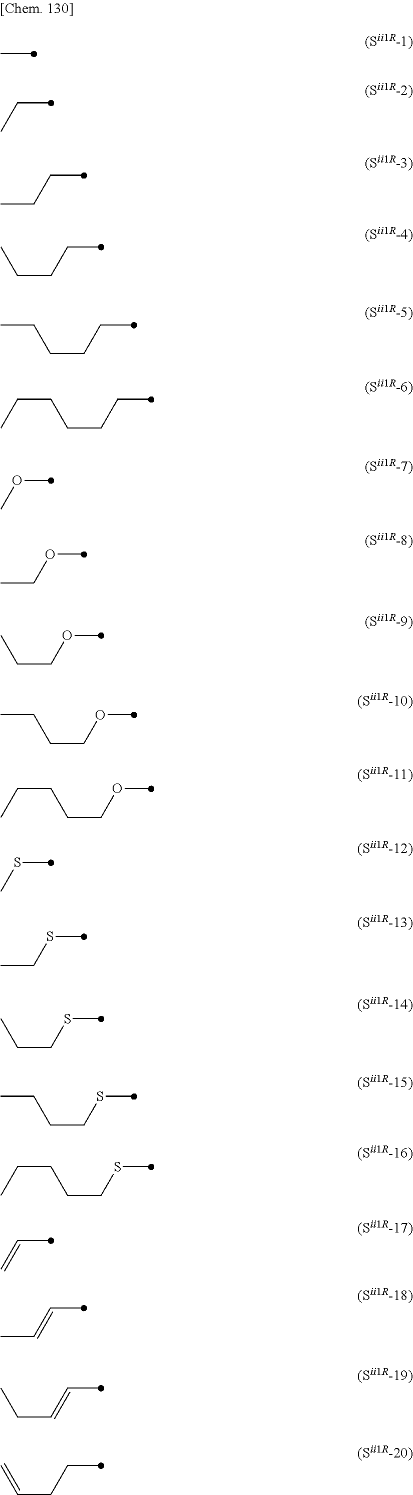

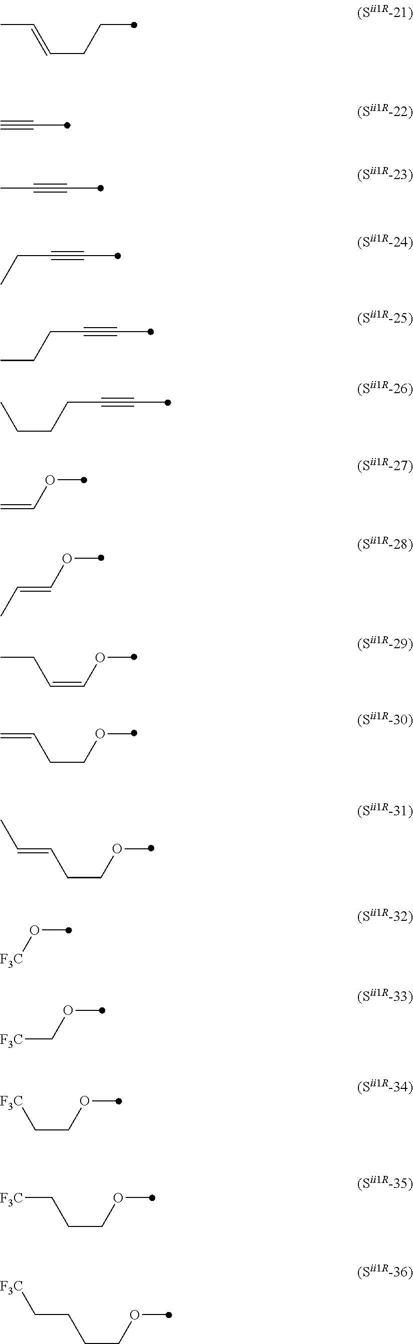

In the formulae (S^{ii1R}-1) to (S^{ii1R}-36), a black dot represents a bonding arm to Aⁱⁱ¹ or Aⁱⁱ².

The substituent Sⁱⁱ¹ is preferably a linear alkyl group with 1 to 6 carbon atoms, a fluorine atom, or a chlorine atom.

At least one Aⁱⁱ¹ or Aⁱⁱ² is preferably substituted with at least one substituent Sⁱⁱ¹, preferably with a halogen atom, or preferably with a fluorine atom.

A plurality of substituents Sⁱⁱ¹, if present, may be the same or different.

The substitution position of the substituent Sⁱⁱ¹ in Aⁱⁱ¹ is preferably any one of the following formulae (Aⁱⁱ¹-SP-1) to (Aⁱⁱ¹-SP-12).

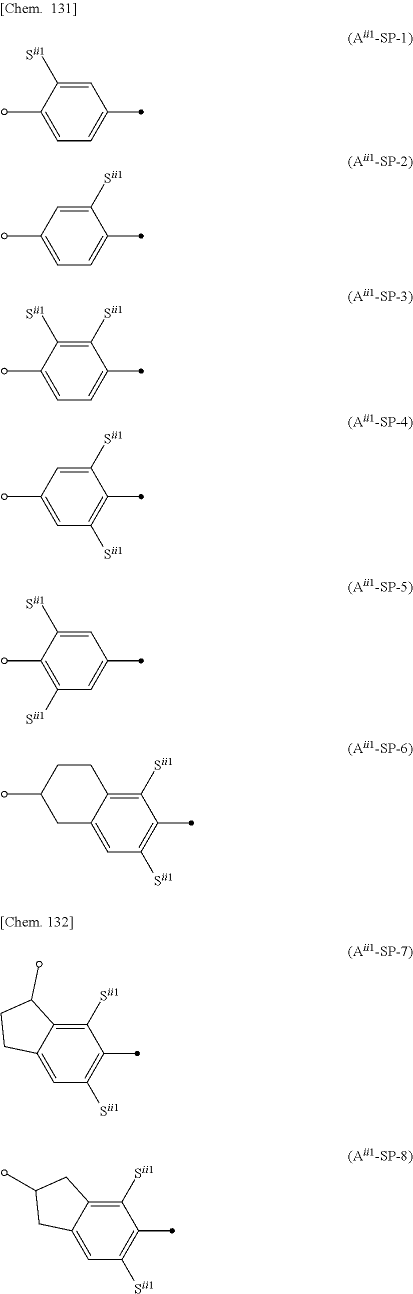

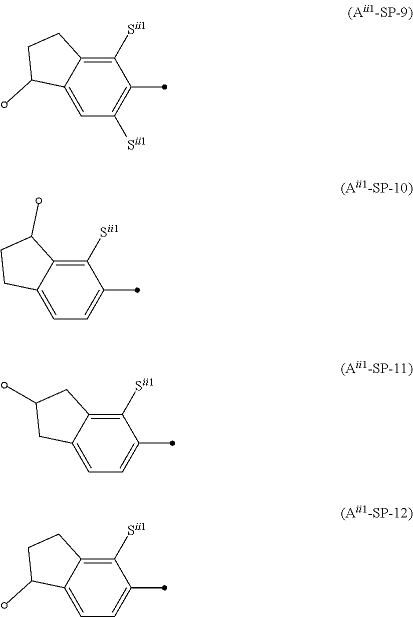

In the formulae (Aⁱⁱ¹-SP-1) to (Aⁱⁱ¹-SP-12), a white dot represents a bonding arm to Rⁱⁱ¹ or Zⁱⁱ¹, and a black dot represents a bonding arm to Zⁱⁱ¹.

The substitution position of the substituent Sⁱⁱ¹ in Aⁱⁱ² is preferably any one of the following formulae (Aⁱⁱ²-SP-1) to (Aⁱⁱ²—SP-8):

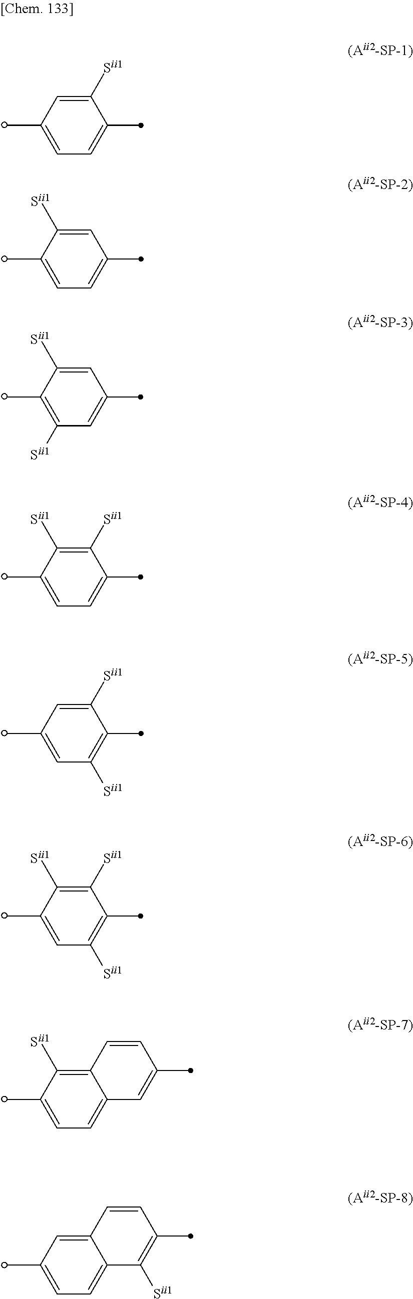

In the formulae (Aⁱⁱ²-SP-1) to (Aⁱⁱ²-SP-8), a white dot represents a bonding arm to Zⁱⁱ¹, and a black dot represents a bonding arm to an isothiocyanate group (—NCS).

More specifically, Aⁱⁱ¹ preferably denotes any one of the following formulae (Aⁱⁱ¹-1) to (Aⁱⁱ¹-34):

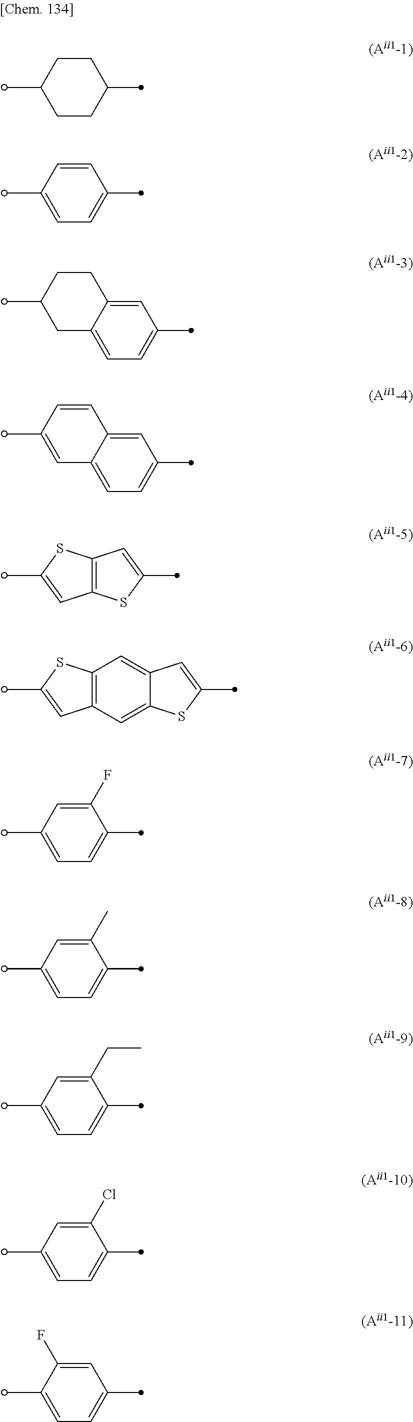

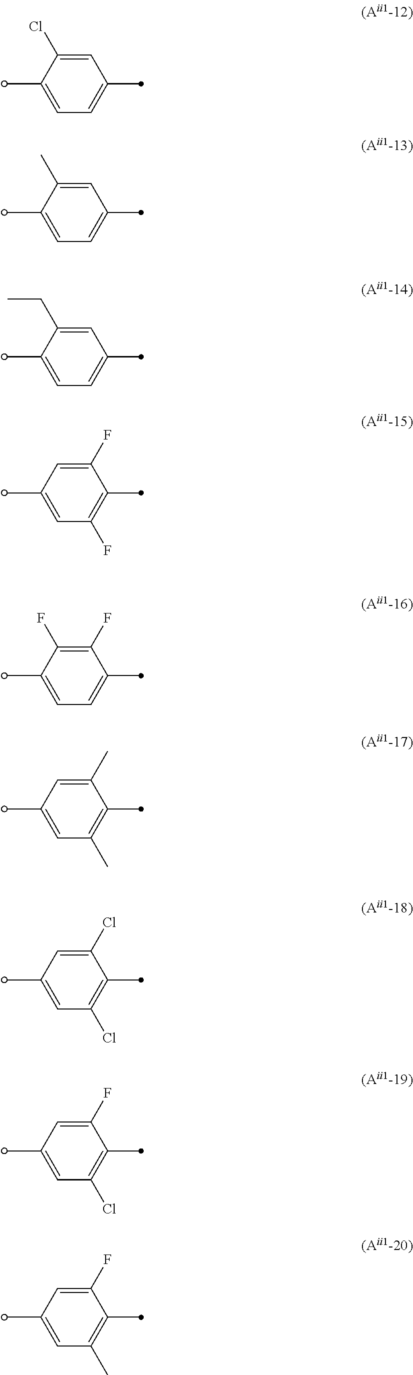

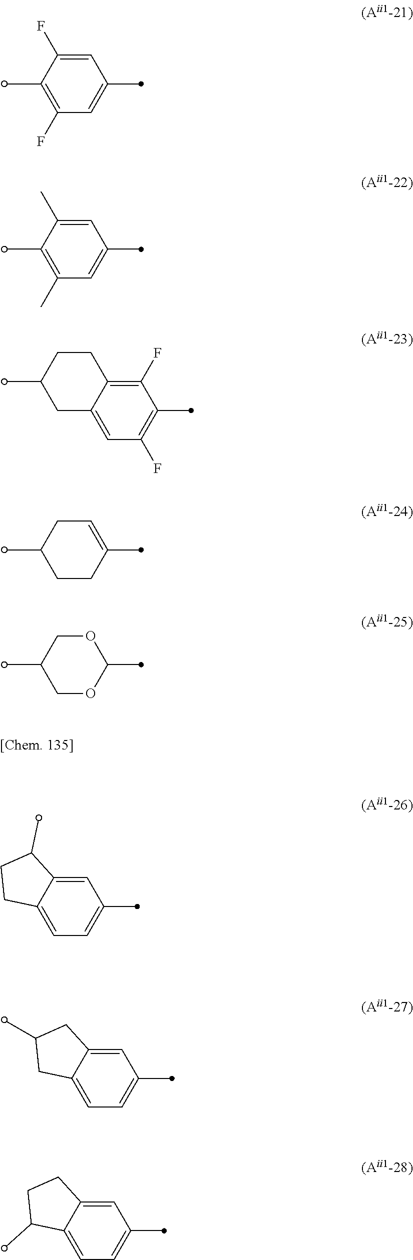

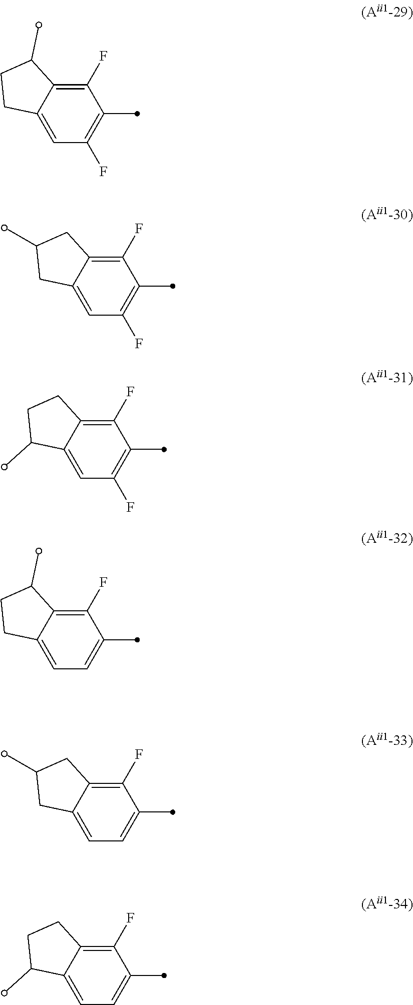

In the formulae (Aⁱⁱ¹-1) to (Aⁱⁱ¹-34), a white dot represents a bonding arm to Rⁱⁱ¹ or Zⁱⁱ¹, and a black dot represents a bonding arm to Zⁱⁱ¹.

More specifically, Aⁱⁱ² preferably denotes any one of the following formulae (Aⁱⁱ²-1) to (Aⁱⁱ²-10):

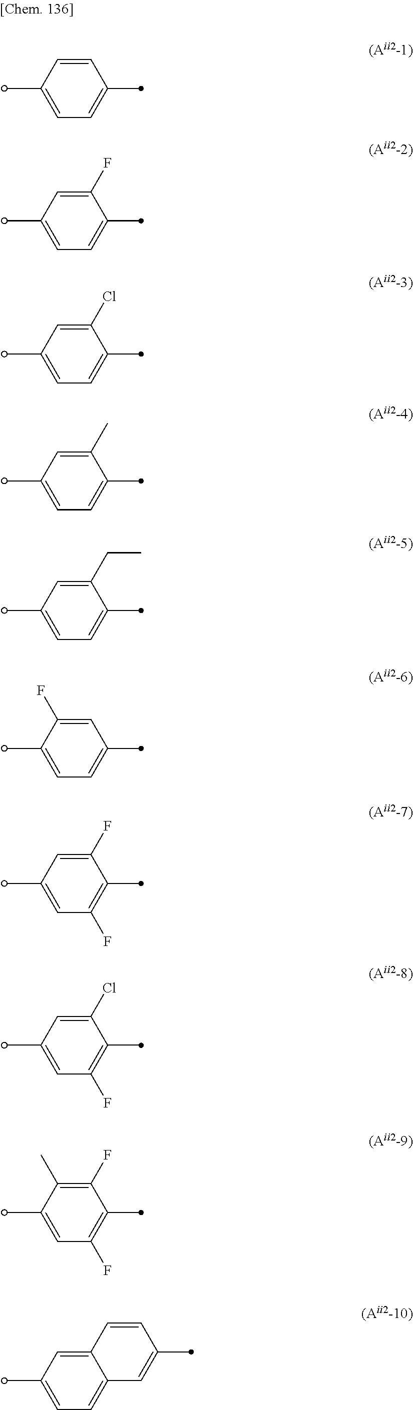

In the formulae (Aⁱⁱ²-1) to (Aⁱⁱ²-10), a white dot represents a bonding arm to Zⁱⁱ¹, and a black dot represents a bonding arm to an isothiocyanate group (—NCS).

In the general formula (ii), Zⁱⁱ¹ denotes any one of a single bond and an alkylene group with 1 to 20 carbon atoms.

One or two or more —CH₂— groups in the alkylene group are each independently optionally substituted with —O—, —CF₂—, and/or —CO—.

One or two or more —CH₂—CH₂— groups in the alkylene group are each independently optionally substituted with —CH₂—CH(CH₃)—, —CH(CH₃)—CH₂—, —CH═CH—, —CF═CF—, —CH═C(CH₃)—, —C(CH₃)═CH—, —CH═N—, —N═CH—, —N═N—, —C≡C—, —CO—O—, and/or —O—CO—.

When the alkylene group with 1 to 20 carbon atoms is substituted with a predetermined group, oxygen atoms are not directly bonded to each other.

From the perspective of the stability of the compound, sulfur atoms are preferably not directly bonded to each other, and/or an oxygen atom and a sulfur atom are preferably not directly bonded to each other.

Specific examples of the alkylene group with 1 to 20 carbon atoms (including substituted ones) include the groups represented by the formulae (Zⁱⁱ¹-1) to (Zⁱⁱ¹-24):

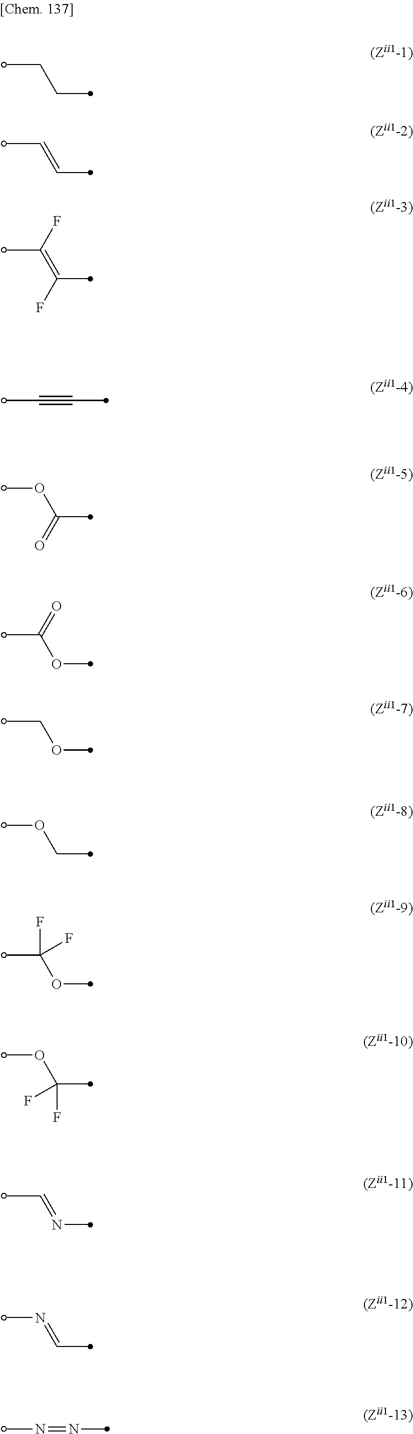

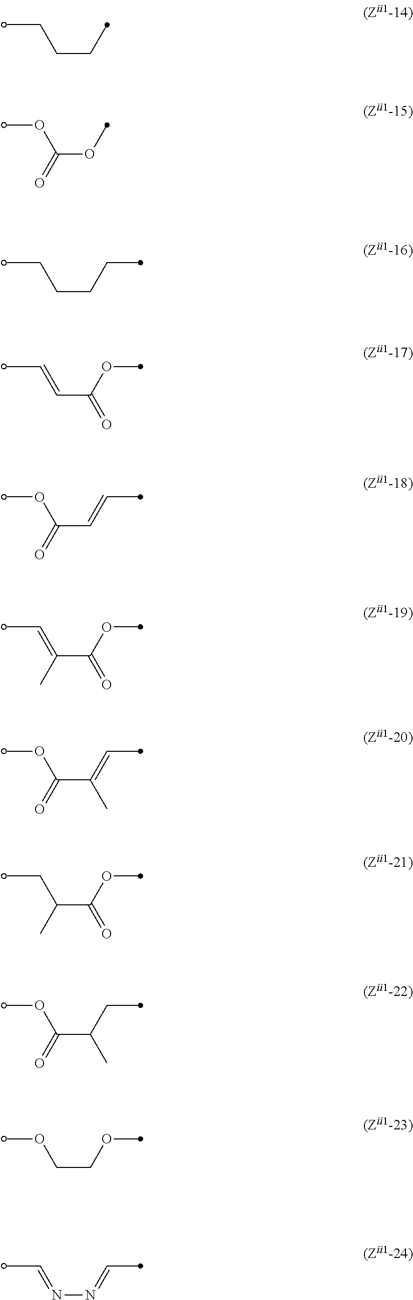

In the formulae (Zⁱⁱ¹-1) to (Zⁱⁱ¹-24), a white dot represents a bonding arm to Aⁱⁱ¹, and a black dot represents a bonding arm to Aⁱⁱ¹ or Aⁱⁱ².

In the general formula (ii), nⁱⁱ¹ denotes an integer in the range of 1 to 4, preferably 1 or 2.

When nⁱⁱ¹ is 1, from the perspective of Δn and/or Δεᵣ, Zⁱⁱ¹ preferably denotes a single bond or —C≡C—.

When nⁱⁱ¹ is 2, from the perspective of Δn and/or Δεᵣ, Zⁱⁱ¹ preferably denotes a single bond or —C≡C—.

In the general formula (ii), a plurality of Aⁱⁱ¹, if present, may be the same or different, and a plurality of Zⁱⁱ¹, if present, may be the same or different.

Among the compounds represented by the general formula (ii), however, the compounds represented by the general formula (i) (including subordinate concepts) are excluded.

Compounds represented by the general formula (ii) are preferably compounds represented by the general formulae (ii-1) to (ii-8):

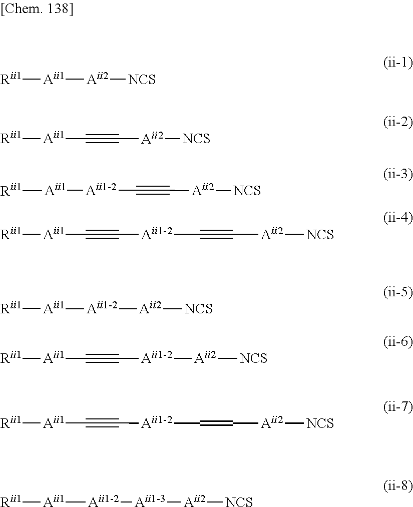

In the general formulae (ii-1) to (ii-8), Rⁱⁱ¹, Aⁱⁱ¹, and Aⁱⁱ² denote the same as Rⁱⁱ¹, Aⁱⁱ¹, and Aⁱⁱ², respectively, in the general formula (ii).

Aⁱⁱ¹-2 and Aⁱⁱ¹-3 in the general formulae (ii-3) to (ii-8) are each independently defined in the same way as Aⁱⁱ¹ in the general formula (ii).

Compounds represented by the general formula (ii-1) are preferably the compounds represented by the following general formulae (ii-1-1) to (ii-1-4):

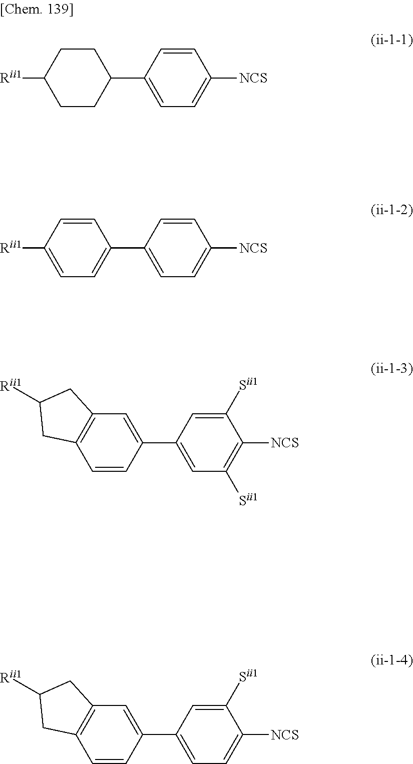

In the general formulae (ii-1-1) to (ii-1-4), Rⁱⁱ¹ and Sⁱⁱ¹ each independently denote the same as Rⁱⁱ¹ and Sⁱⁱ¹ in the general formula (ii).

Specific examples of the compounds represented by the general formula (ii-1-1) include the compounds represented by the following structural formulae (ii-1-1.1) to (ii-1-1.4):

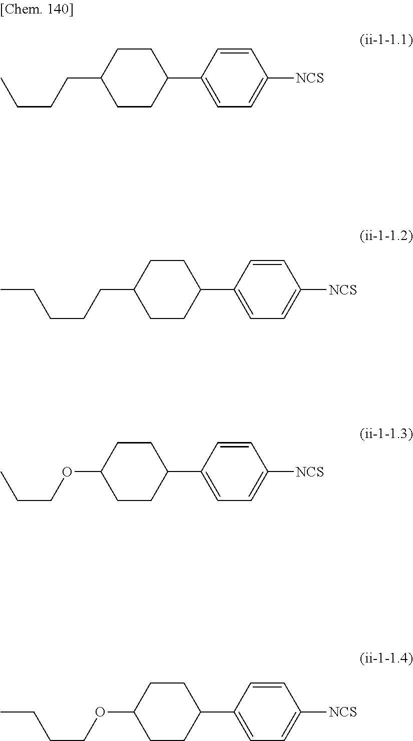

Specific examples of the compounds represented by the general formula (ii-1-2) include the compounds represented by the following structural formulae (ii-1-2.1) to (ii-1-2.6):

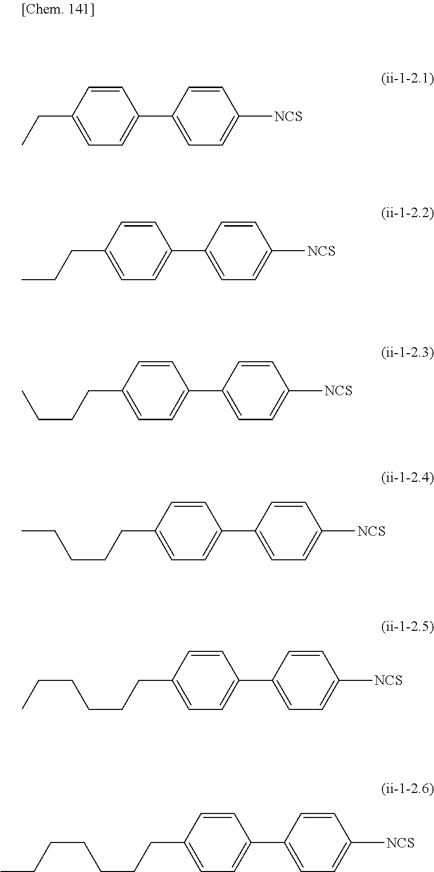

Specific examples of the compounds represented by the general formula (ii-1-3) include the compounds represented by the following structural formulae (ii-1-3.1) to (ii-1-3.3):

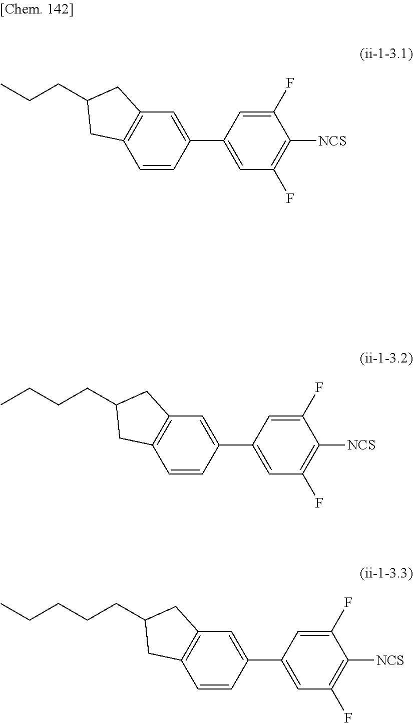

Specific examples of the compounds represented by the general formula (ii-1-4) include the compounds represented by the following structural formulae (ii-1-4.1) to (ii-1-4.3):

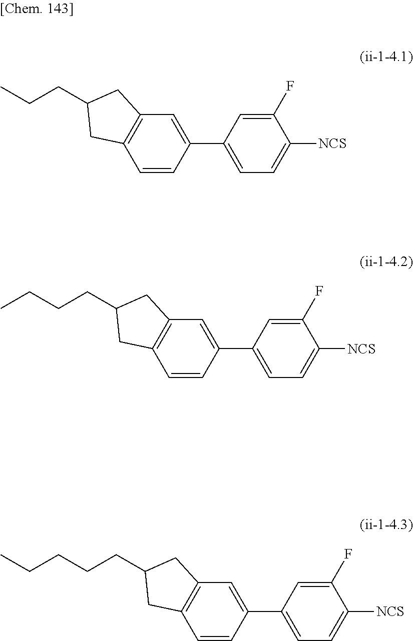

Compounds represented by the general formula (ii-2) are preferably the compounds represented by the following general formulae (ii-2-1) to (ii-2-14):

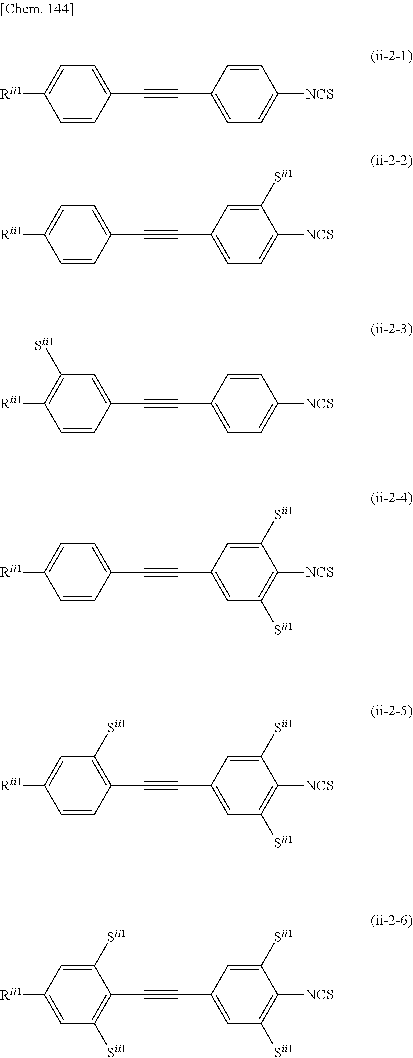

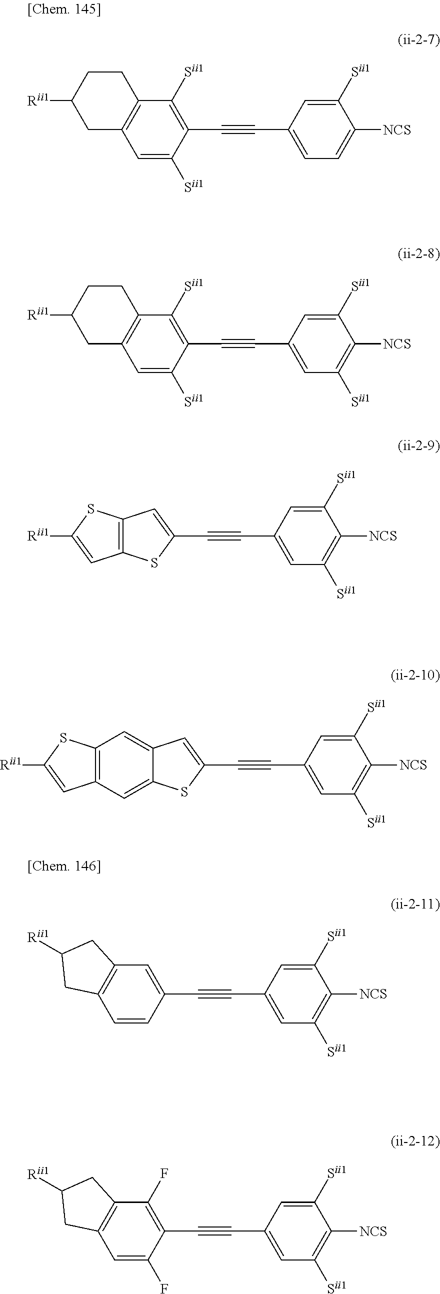

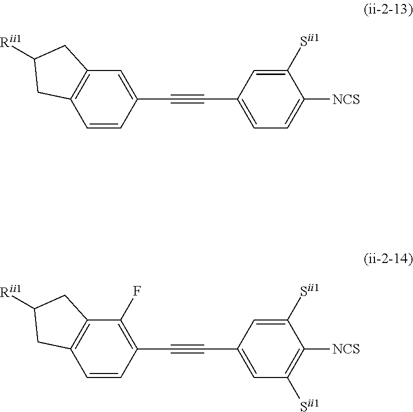

In the general formulae (ii-2-1) to (ii-2-14), Rⁱⁱ¹ and Sⁱⁱ¹ each independently denote the same as Rⁱⁱ¹ and Sⁱⁱ¹ in the general formula (ii).

Specific examples of the compounds represented by the general formula (ii-2-1) include the compounds represented by the following structural formulae (ii-2-1.1) to (ii-2-1.4):

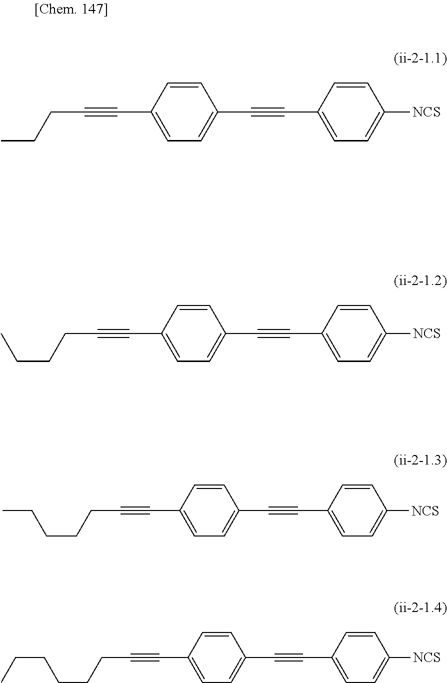

Specific examples of the compounds represented by the general formula (ii-2-2) include the compounds represented by the following structural formulae (ii-2-2.1) to (ii-2-2.10):

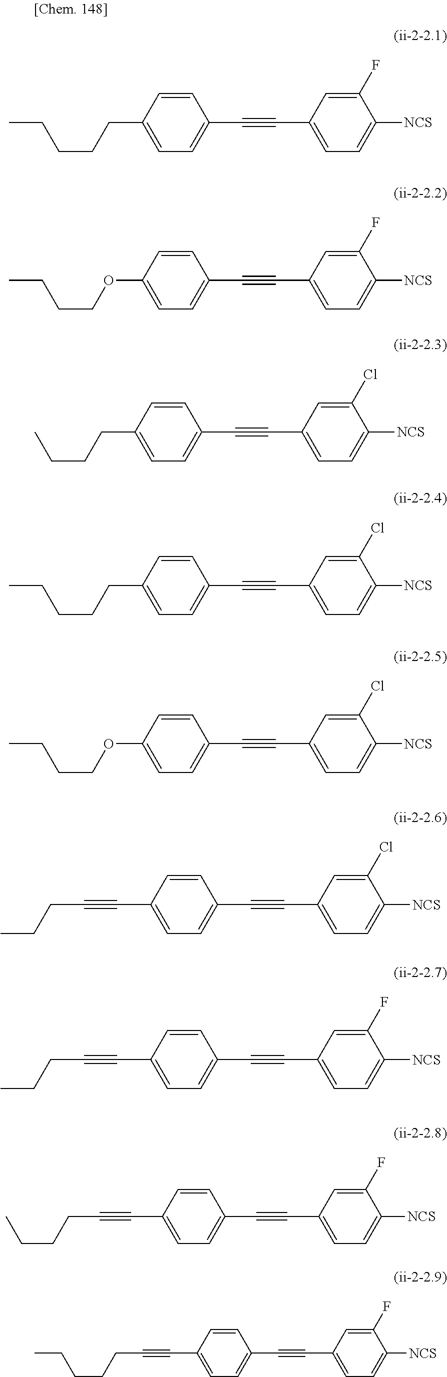

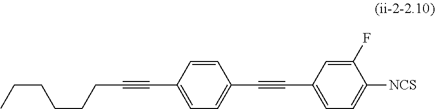

Specific examples of the compounds represented by the general formula (ii-2-3) include the compounds represented by the following structural formulae (ii-2-3.1) to (ii-2-3.3):

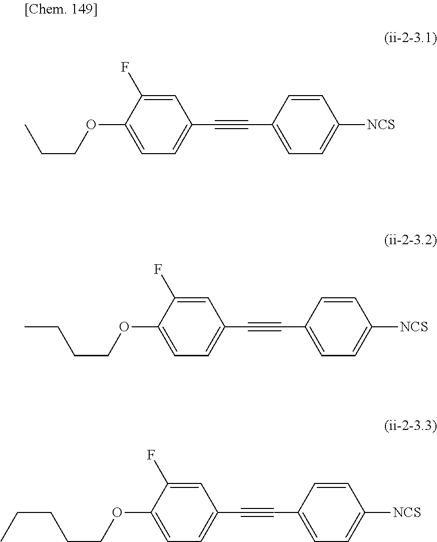

Specific examples of the compounds represented by the general formula (ii-2-4) include the compounds represented by the following structural formulae (ii-2-4.1) to (ii-2-4.8):

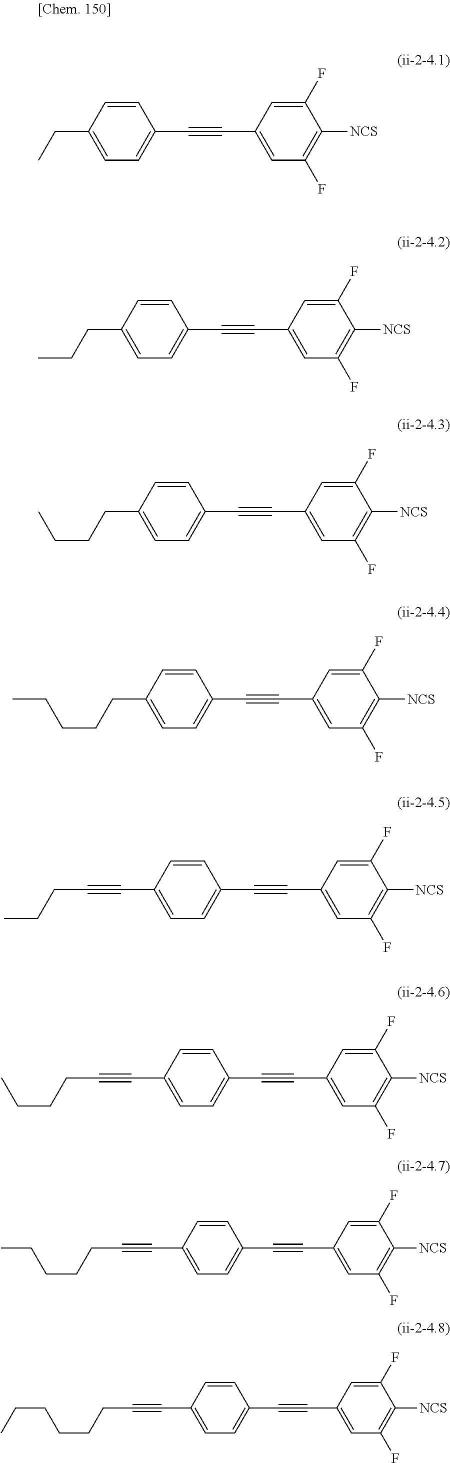

Specific examples of the compounds represented by the general formula (ii-2-5) include the compounds represented by the following structural formulae (ii-2-5.1) to (ii-2-5.4):

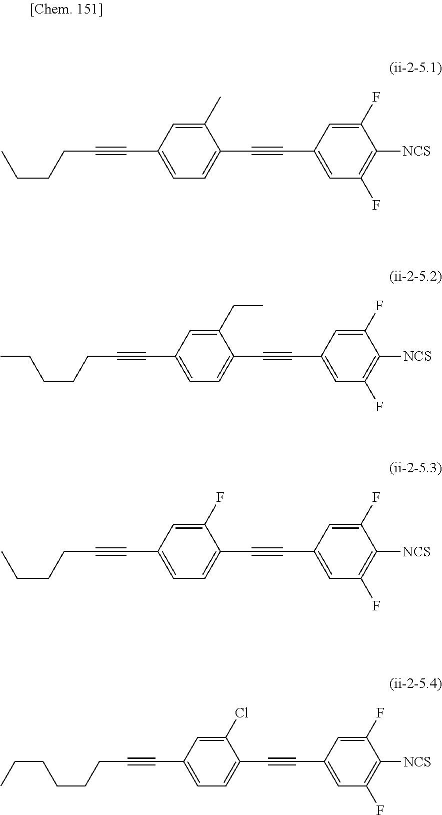

Specific examples of the compounds represented by the general formula (ii-2-6) include the compounds represented by the following structural formulae (ii-2-6.1) to (ii-2-6.4):

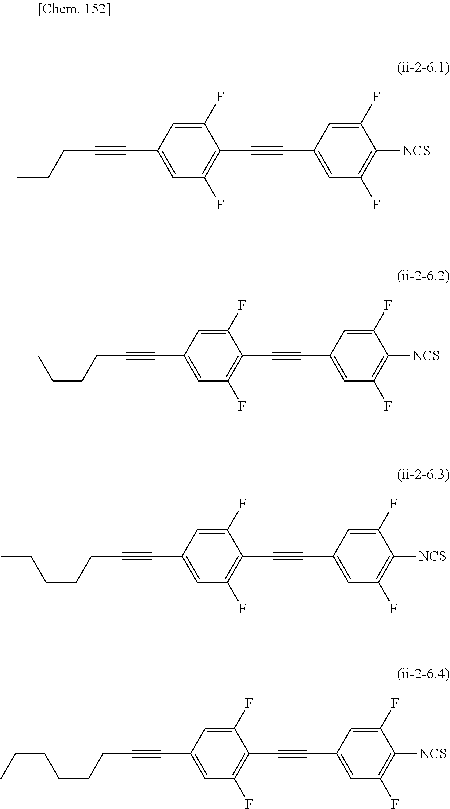

Specific examples of the compounds represented by the general formula (ii-2-7) include the compounds represented by the following structural formulae (ii-2-7.1) to (ii-2-7.3):

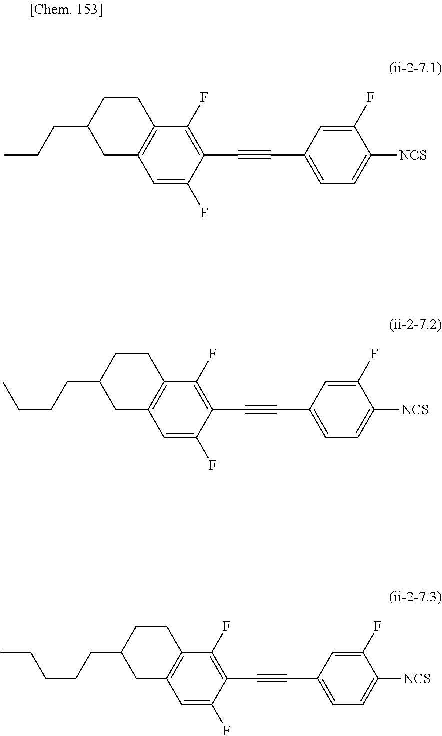

Specific examples of the compounds represented by the general formula (ii-2-8) include the compounds represented by the following structural formulae (ii-2-8.1) to (ii-2-8.3):

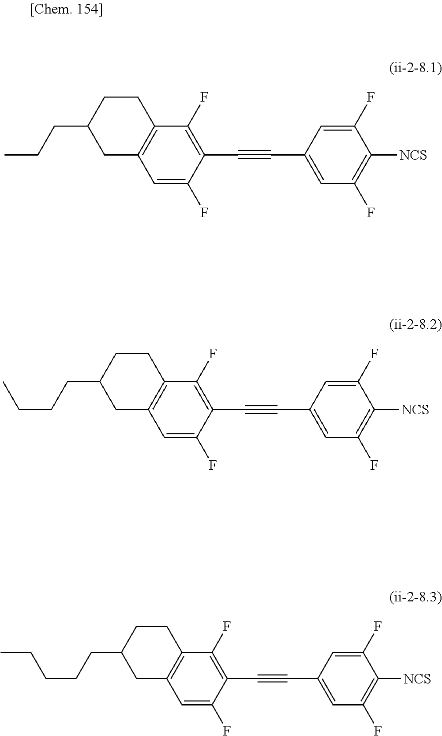

Specific examples of the compounds represented by the general formula (ii-2-9) include the compounds represented by the following structural formulae (ii-2-9.1) to (ii-2-9.4):

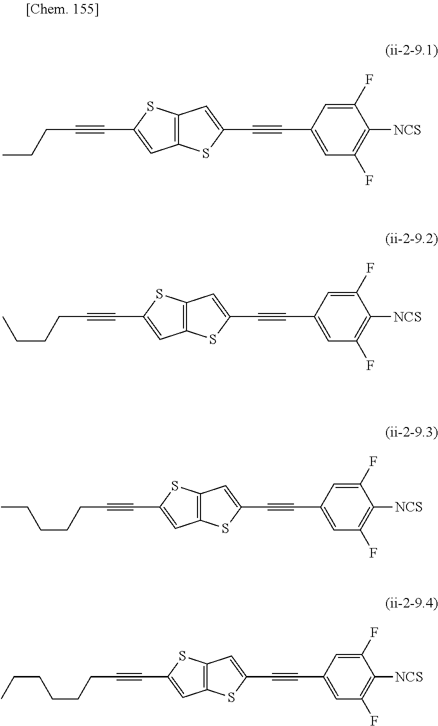

Specific examples of the compounds represented by the general formula (ii-2-10) include the compounds represented by the following structural formulae (ii-2-10.1) to (ii-2-10.4):

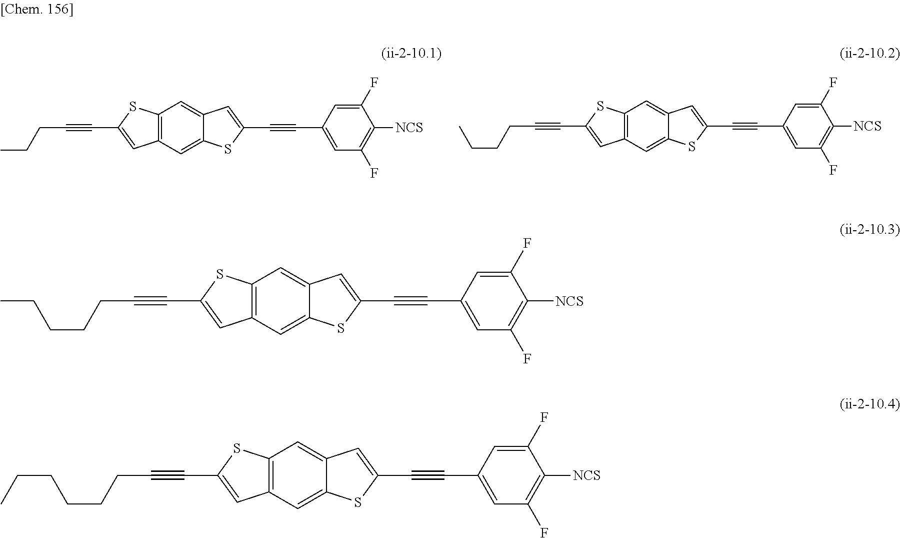

Specific examples of the compounds represented by the general formula (ii-2-11) include the compounds represented by the following structural formulae (ii-2-11.1) to (ii-2-11.3):

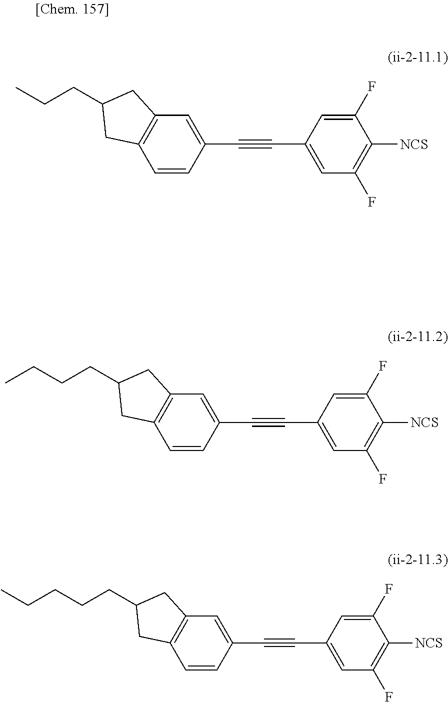

Specific examples of the compounds represented by the general formula (ii-2-12) include the compounds represented by the following structural formulae (ii-2-12.1) to (ii-2-12.3):

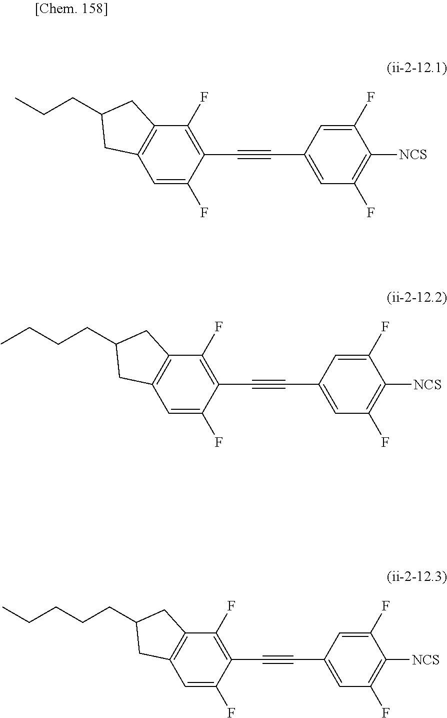

Specific examples of the compounds represented by the general formula (ii-2-13) include the compounds represented by the following structural formulae (ii-2-13.1) to (ii-2-13.3):

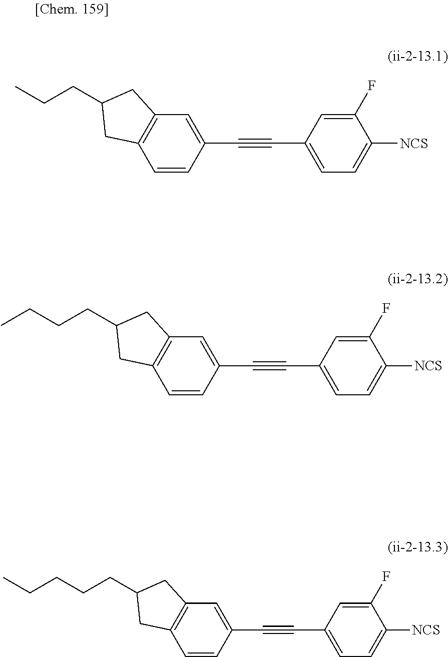

Specific examples of the compounds represented by the general formula (ii-2-14) include the compound represented by the following structural formula (ii-2-14.1):

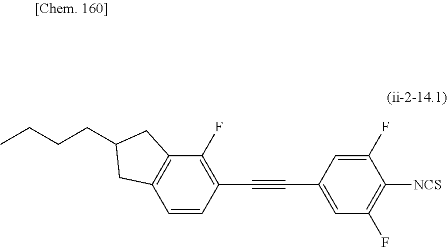

Compounds represented by the general formula (ii-3) are preferably the compounds represented by the following general formulae (ii-3-1) to (ii-3-22):

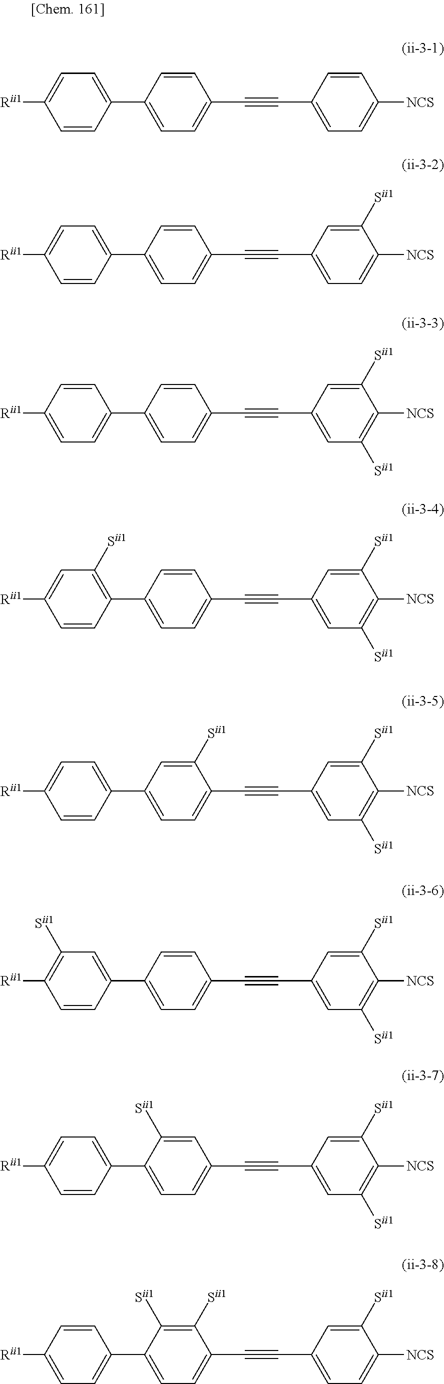

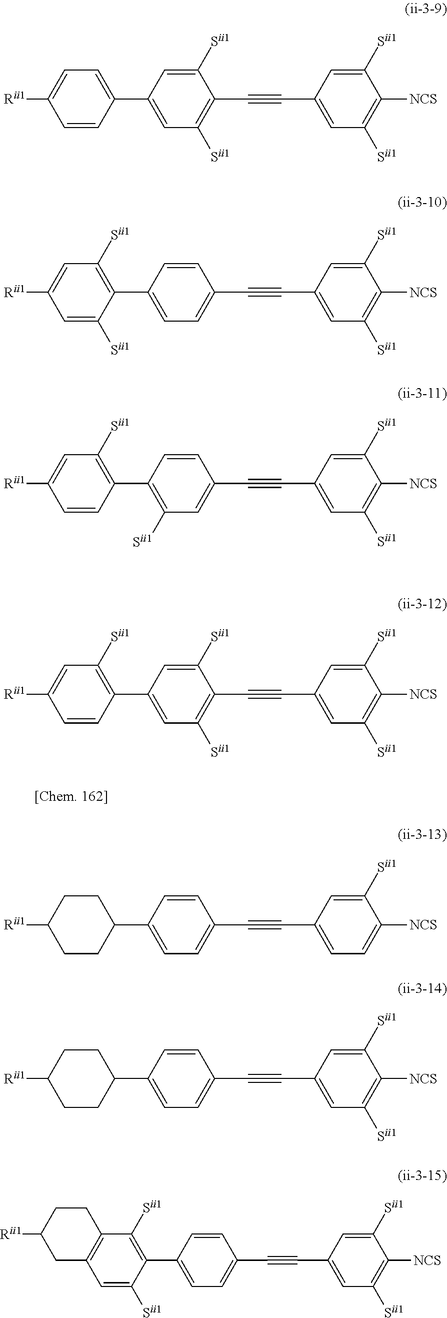

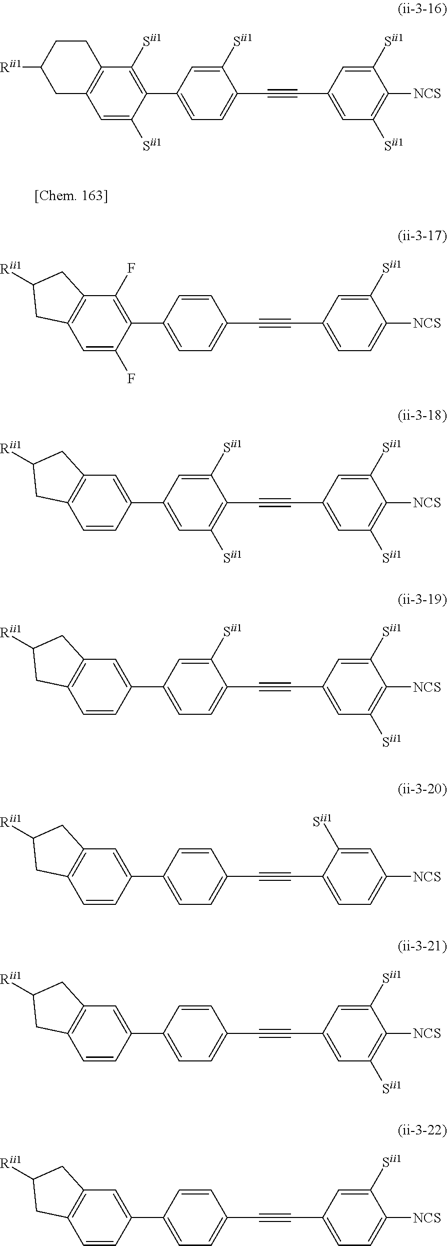

In the general formulae (ii-3-1) to (ii-3-22), Rⁱⁱ¹ and Sⁱⁱ¹ each independently denote the same as Rⁱⁱ¹ and Sⁱⁱ¹ in the general formula (ii).

Specific examples of the compounds represented by the general formula (ii-3-1) include the compounds represented by the following structural formulae (ii-3-1.1) to (ii-3-1.4):

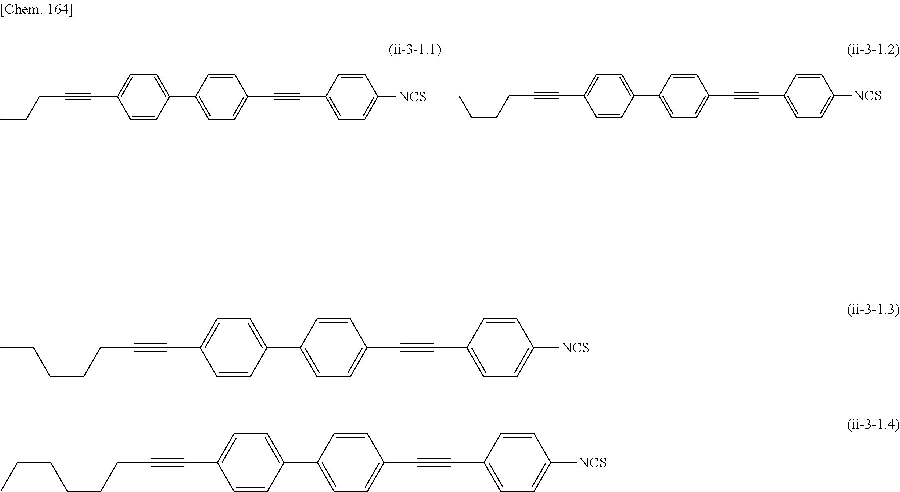

Specific examples of the compounds represented by the general formula (ii-3-2) include the compounds represented by the following structural formulae (ii-3-2.1) to (ii-3-2.4):

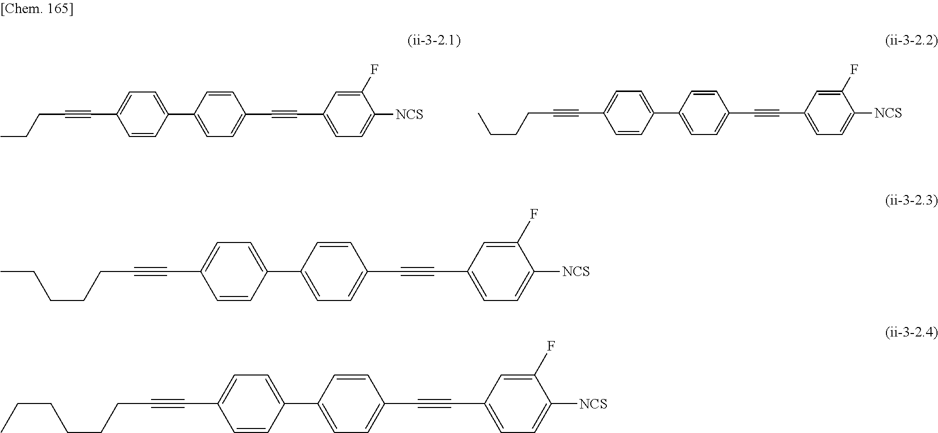

Specific examples of the compounds represented by the general formula (ii-3-3) include the compounds represented by the following structural formulae (ii-3-3.1) to (ii-3-3.7):

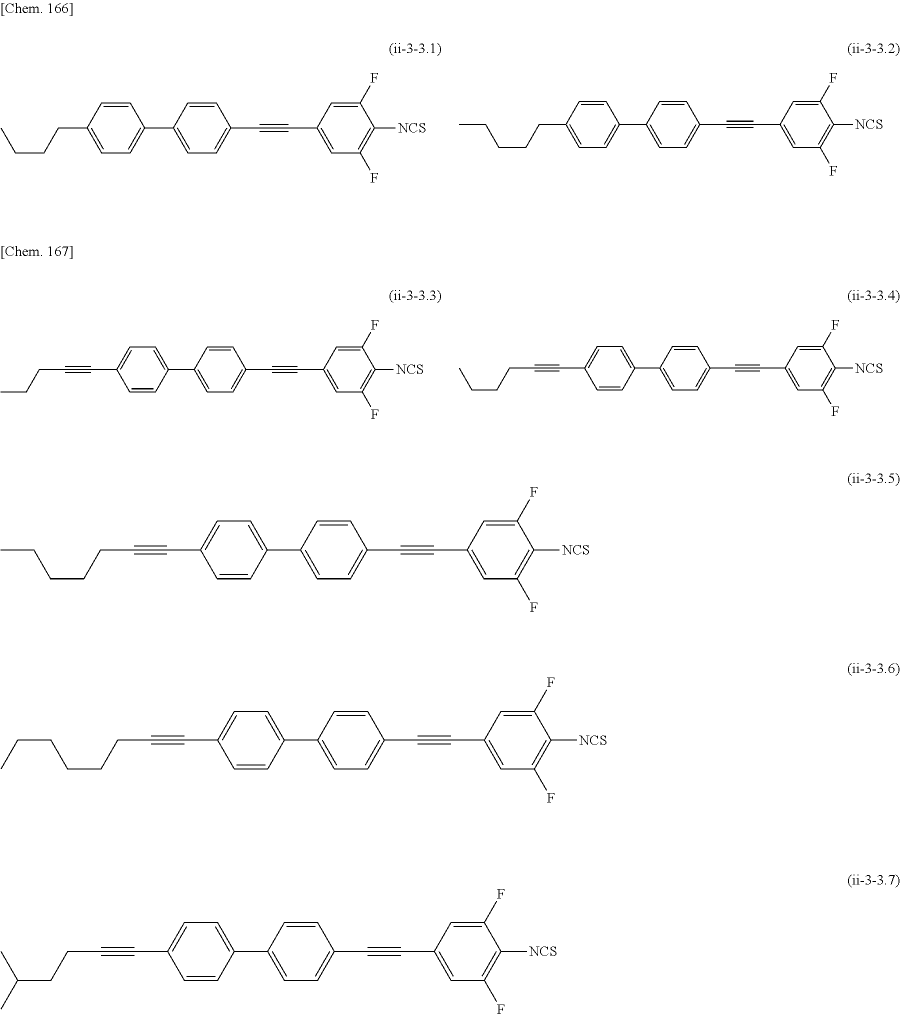

Specific examples of the compounds represented by the general formula (ii-3-4) include the compounds represented by the following structural formulae (ii-3-4.1) to (ii-3-4.5):

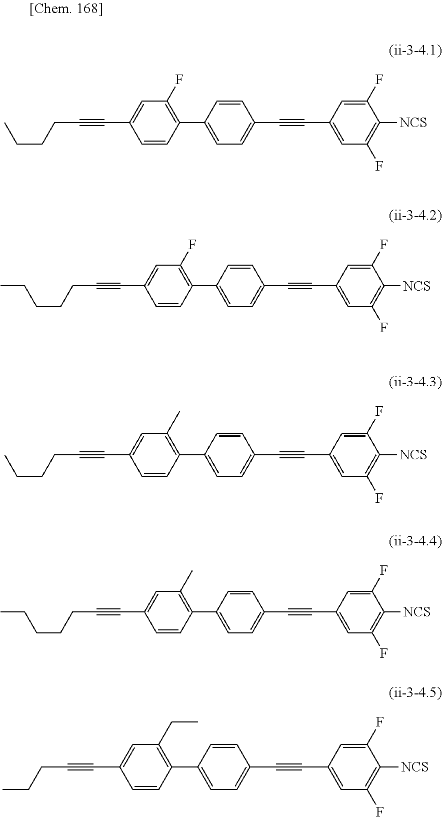

Specific examples of the compounds represented by the general formula (ii-3-5) include the compounds represented by the following structural formulae (ii-3-5.1) to (ii-3-5.7):

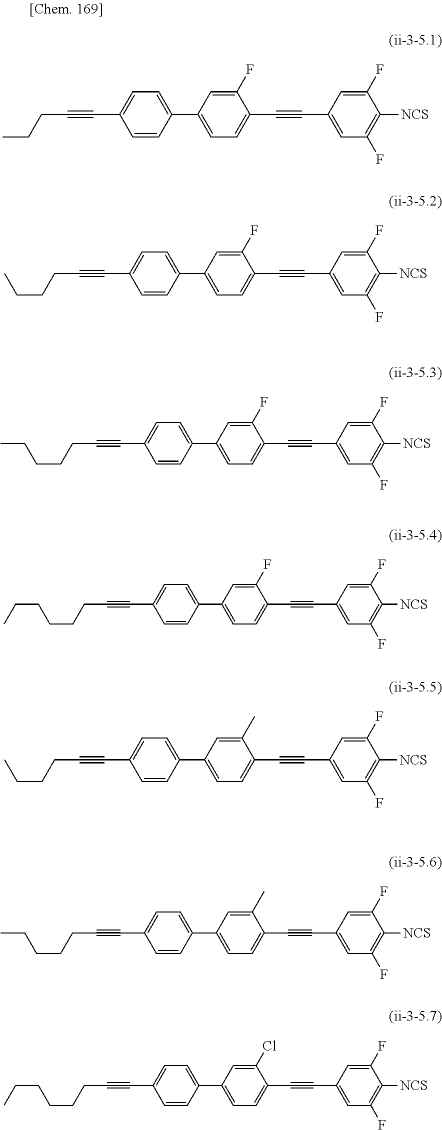

Specific examples of the compounds represented by the general formula (ii-3-6) include the compounds represented by the following structural formulae (ii-3-6.1) to (ii-3-6.3):

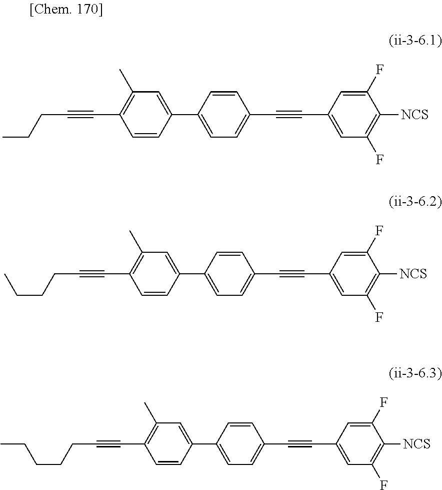

Specific examples of the compounds represented by the general formula (ii-3-7) include the compounds represented by the following structural formulae (ii-3-7.1) to (ii-3-7.6):

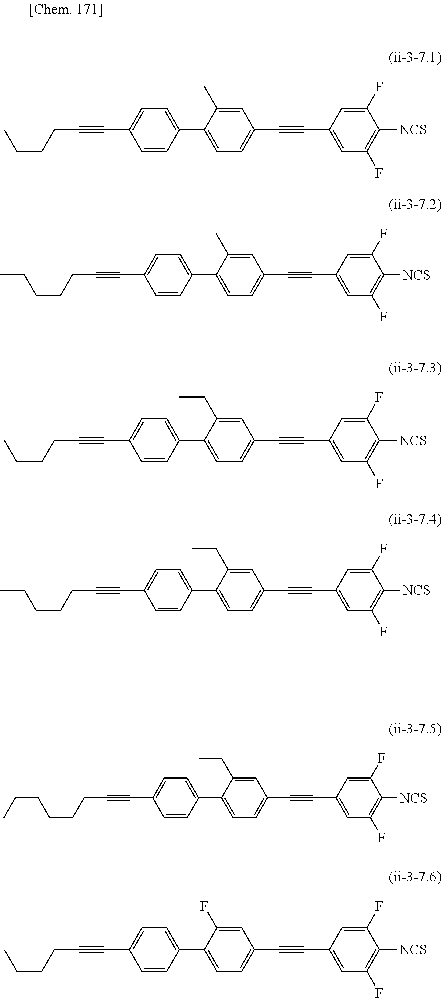

Specific examples of the compounds represented by the general formula (ii-3-8) include the compounds represented by the following structural formulae (ii-3-8.1) to (ii-3-8.3):

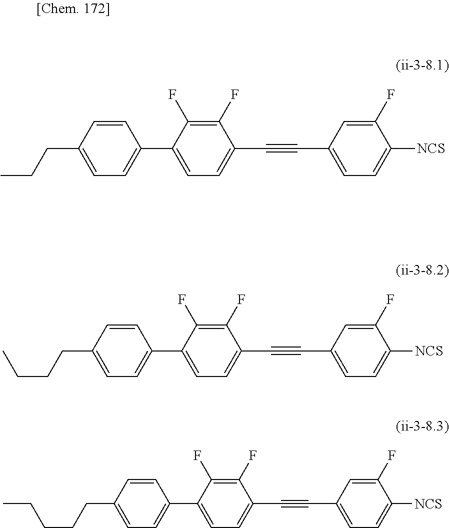

Specific examples of the compounds represented by the general formula (ii-3-9) include the compounds represented by the following structural formulae (ii-3-9.1) to (ii-3-9.4):

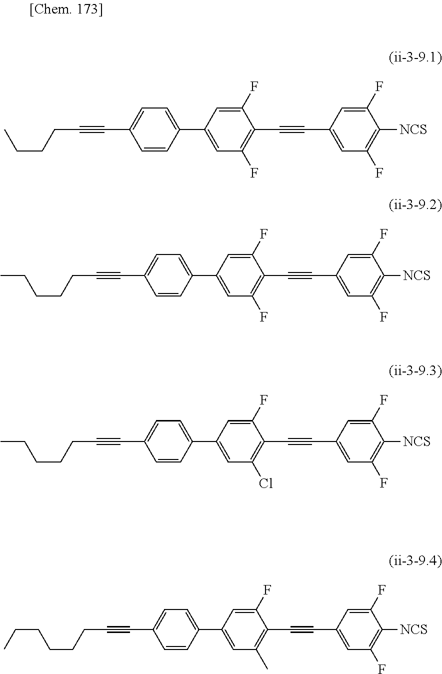

Specific examples of the compounds represented by the general formula (ii-3-10) include the compounds represented by the following structural formulae (ii-3-10.1) to (ii-3-10.3):

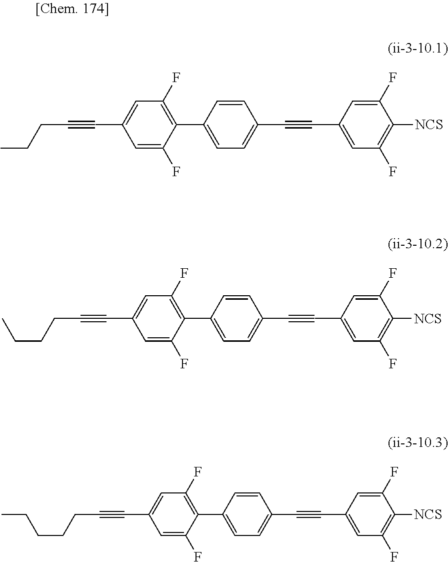

Specific examples of the compounds represented by the general formula (ii-3-11) include the compounds represented by the following structural formulae (ii-3-11.1) to (ii-3-11.3):

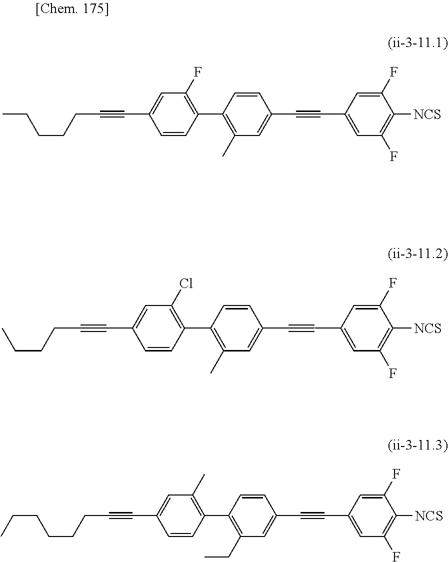

Specific examples of the compounds represented by the general formula (ii-3-12) include the compounds represented by the following structural formulae (ii-3-12.1) to (ii-3-12.3):

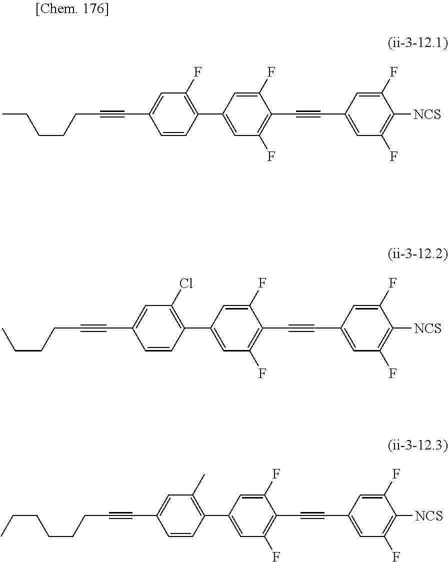

Specific examples of the compounds represented by the general formula (ii-3-13) include the compounds represented by the following structural formulae (ii-3-13.1) to (ii-3-13.4):

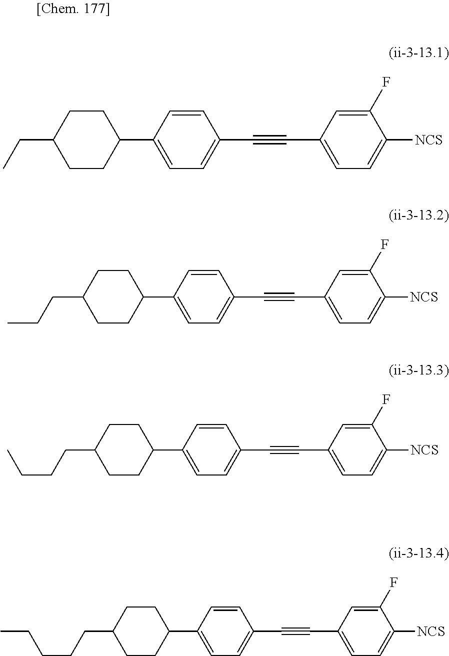

Specific examples of the compounds represented by the general formula (ii-3-14) include the compounds represented by the following structural formulae (ii-3-14.1) to (ii-3-14.3):

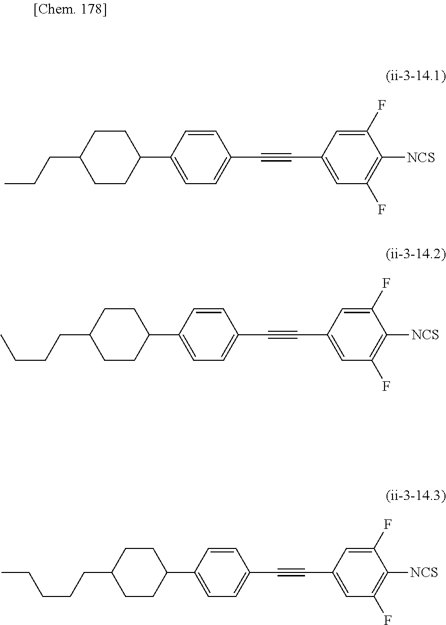

Specific examples of the compounds represented by the general formula (ii-3-15) include the compounds represented by the following structural formulae (ii-3-15.1) to (ii-3-15.3):

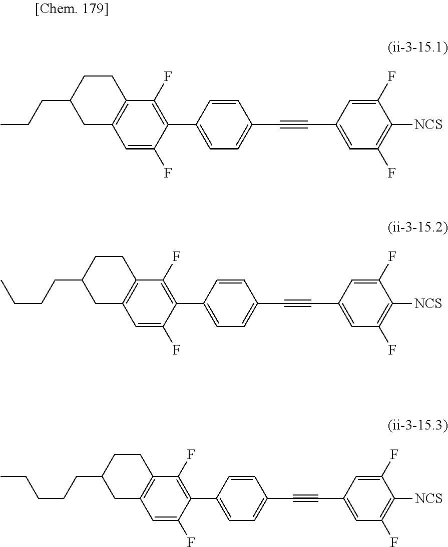

Specific examples of the compounds represented by the general formula (ii-3-16) include the compounds represented by the following structural formulae (ii-3-16.1) to (ii-3-16.3):

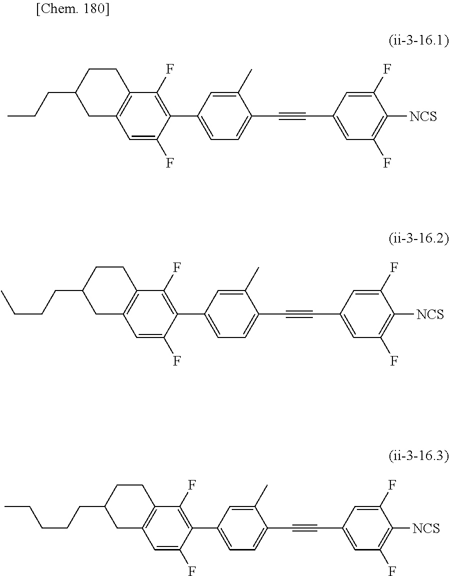

Specific examples of the compounds represented by the general formula (ii-3-17) include the compounds represented by the following structural formulae (ii-3-17.1) to (ii-3-17.3):

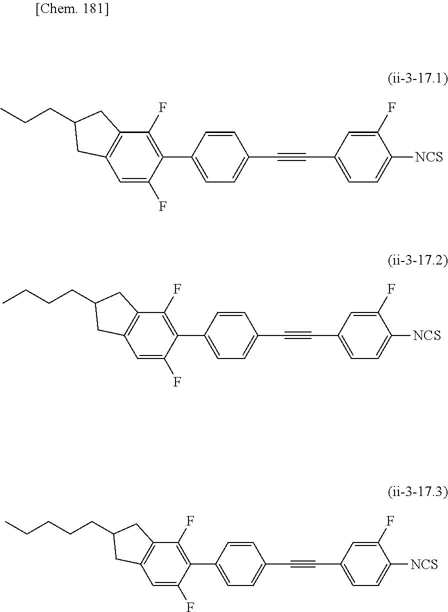

Specific examples of the compounds represented by the general formula (ii-3-18) include the compounds represented by the following structural formulae (ii-3-18.1) to (ii-3-18.3):

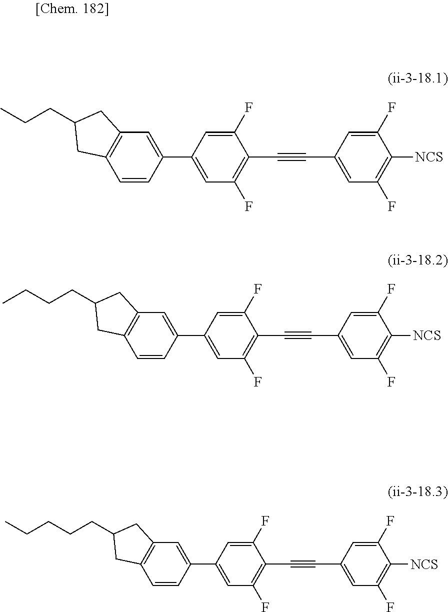

Specific examples of the compounds represented by the general formula (ii-3-19) include the compounds represented by the following structural formulae (ii-3-19.1) to (ii-3-19.3):

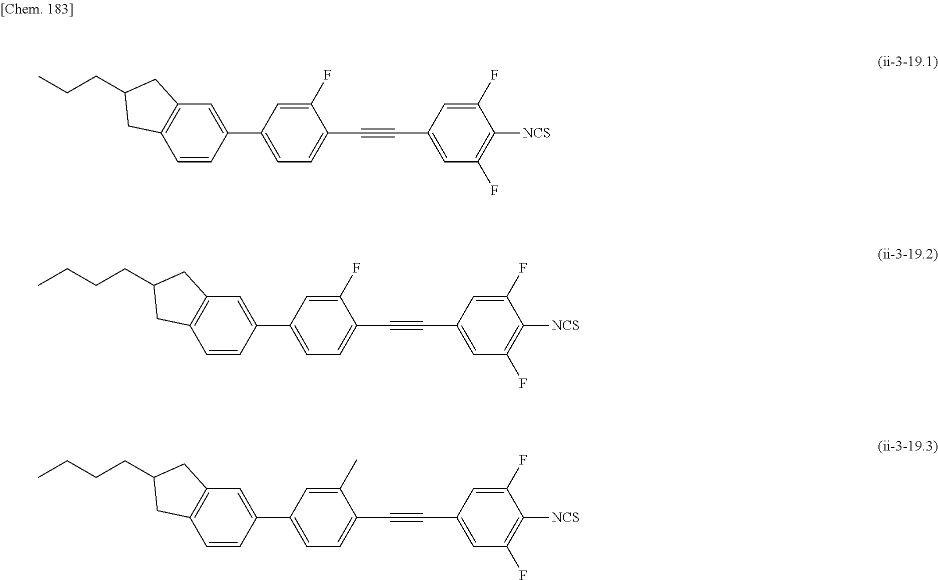

Specific examples of the compounds represented by the general formula (ii-3-20) include the compounds represented by the following structural formulae (ii-3-20.1) to (ii-3-20.3):

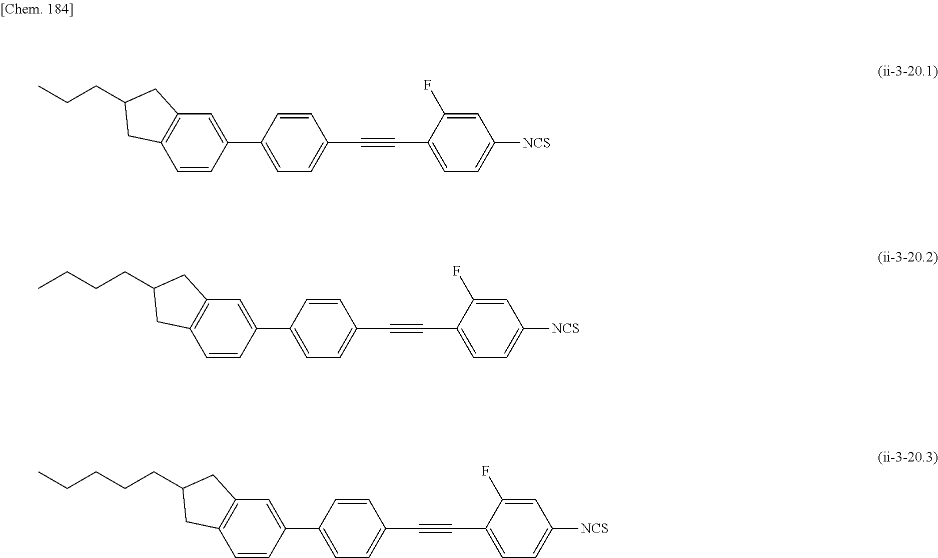

Specific examples of the compounds represented by the general formula (ii-3-21) include the compounds represented by the following structural formulae (ii-3-21.1) to (ii-3-21.4):

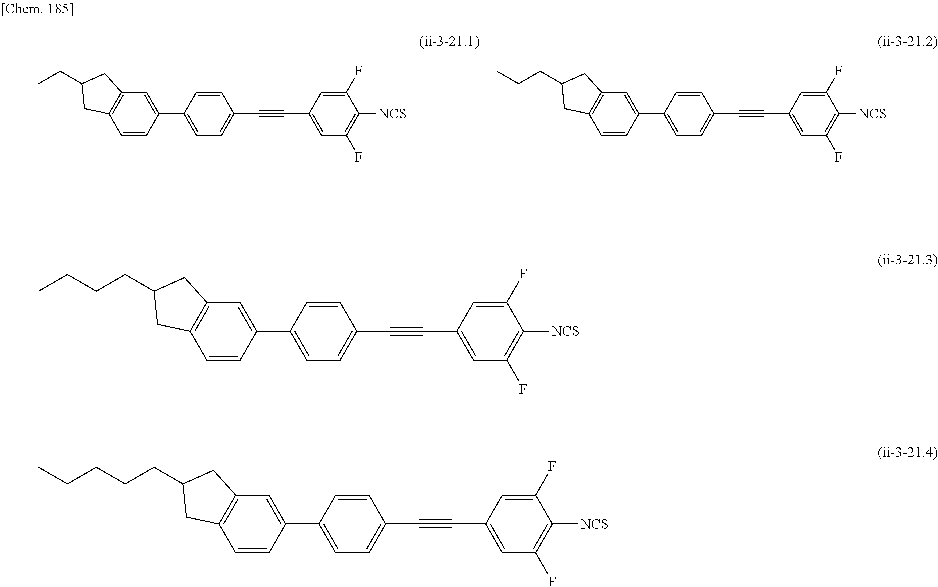

Specific examples of the compounds represented by the general formula (ii-3-22) include the compounds represented by the following structural formulae (ii-3-22.1) to (ii-3-22.3):

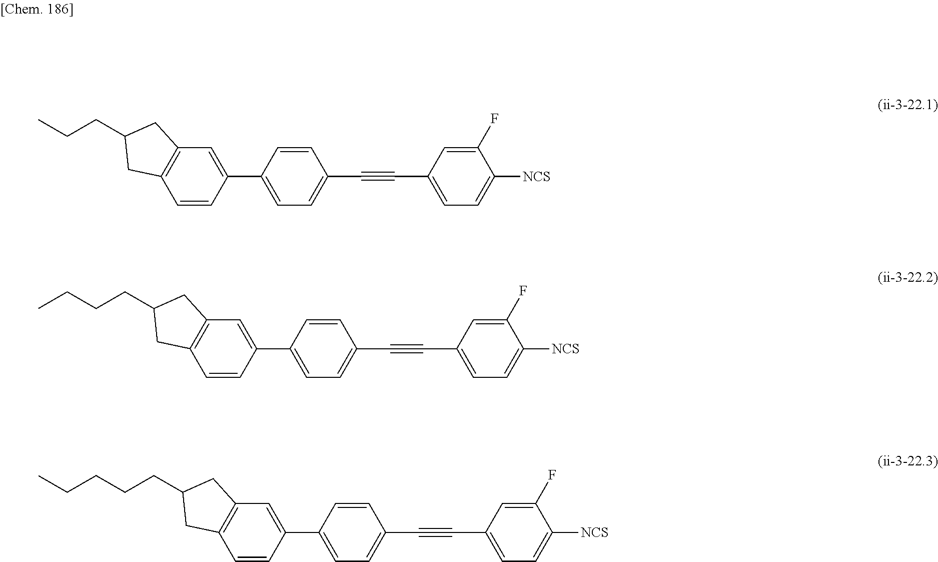

Compounds represented by the general formula (ii-4) are preferably the compounds represented by the following general formulae (ii-4-1) to (ii-4-30):

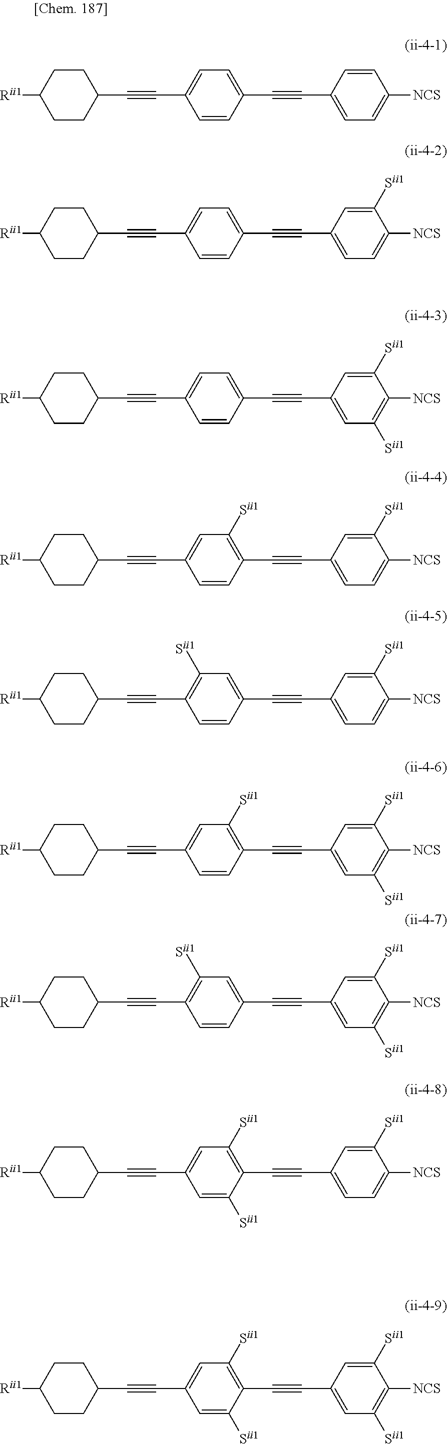

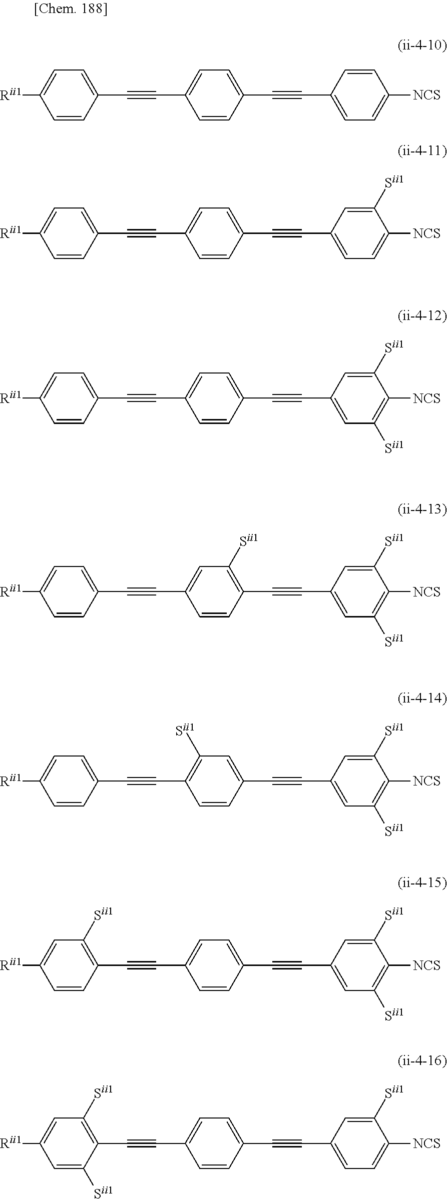

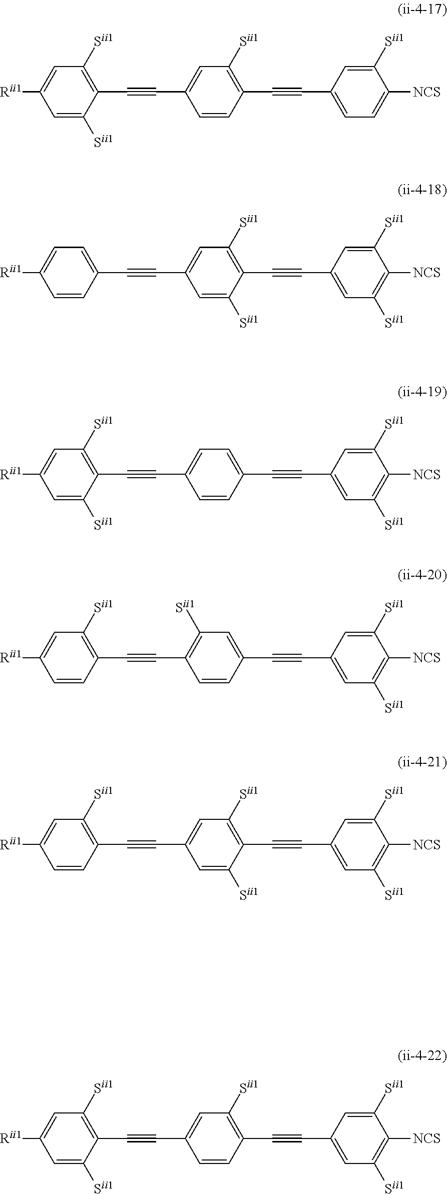

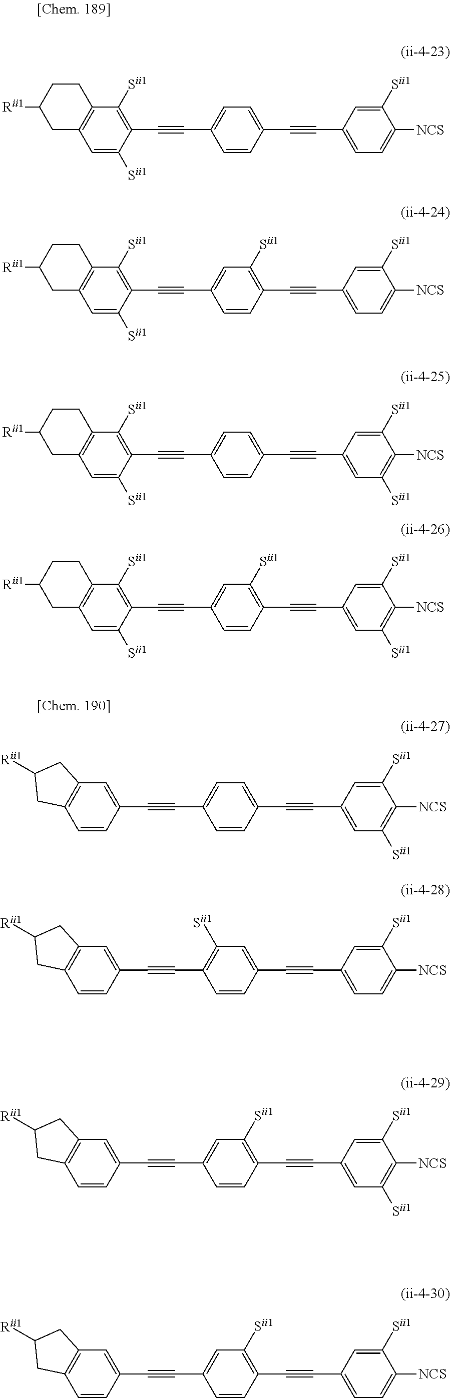

In the general formulae (ii-4-1) to (ii-4-30), Rⁱⁱ¹ and Sⁱⁱ¹ each independently denote the same as Rⁱⁱ¹ and Sⁱⁱ¹ in the general formula (ii).

Specific examples of the compounds represented by the general formula (ii-4-1) include the compounds represented by the following structural formulae (ii-4-1.1) to (ii-4-1.4):

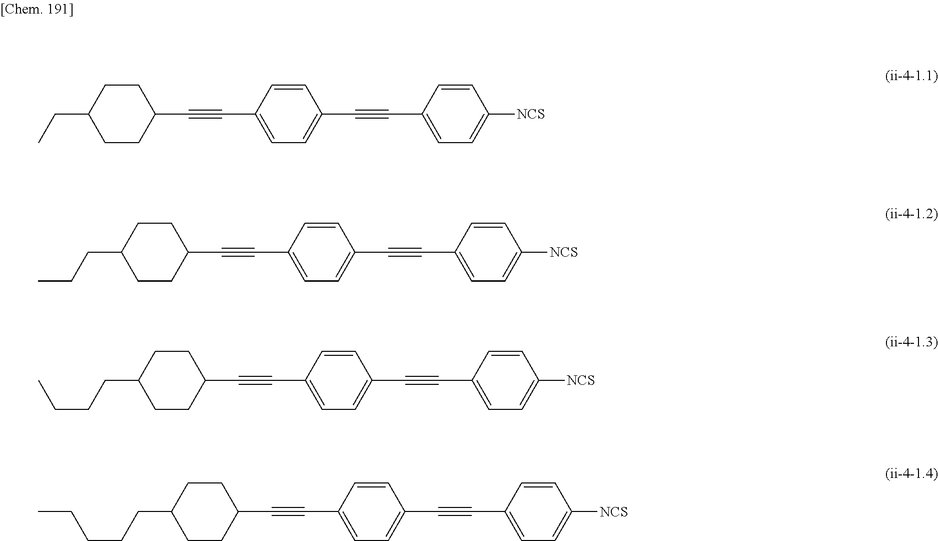

Specific examples of the compounds represented by the general formula (ii-4-2) include the compounds represented by the following structural formulae (ii-4-2.1) to (ii-4-2.4):

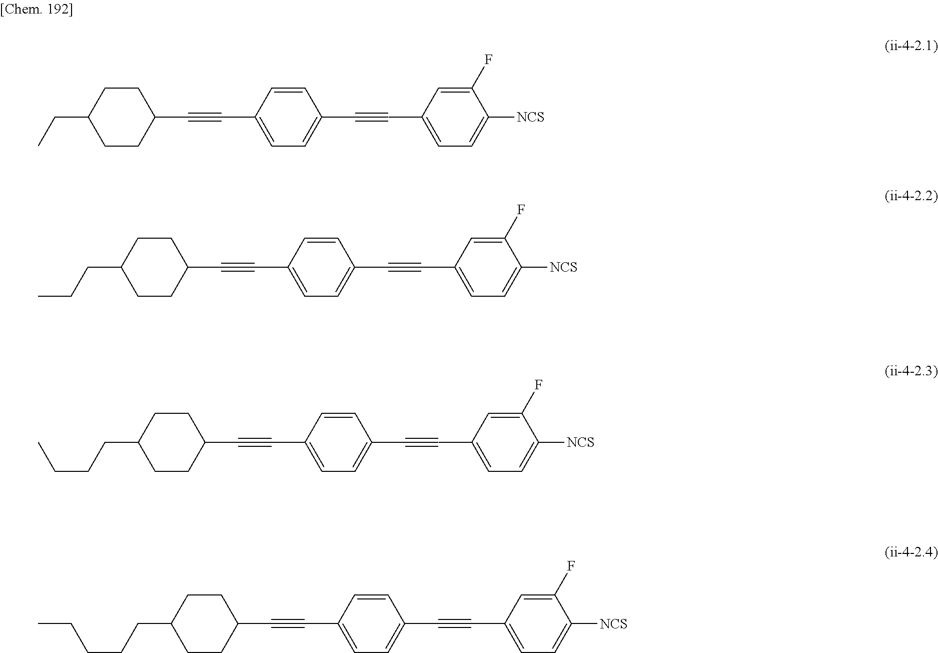

Specific examples of the compounds represented by the general formula (ii-4-3) include the compounds represented by the following structural formulae (ii-4-3.1) to (ii-4-3.8):

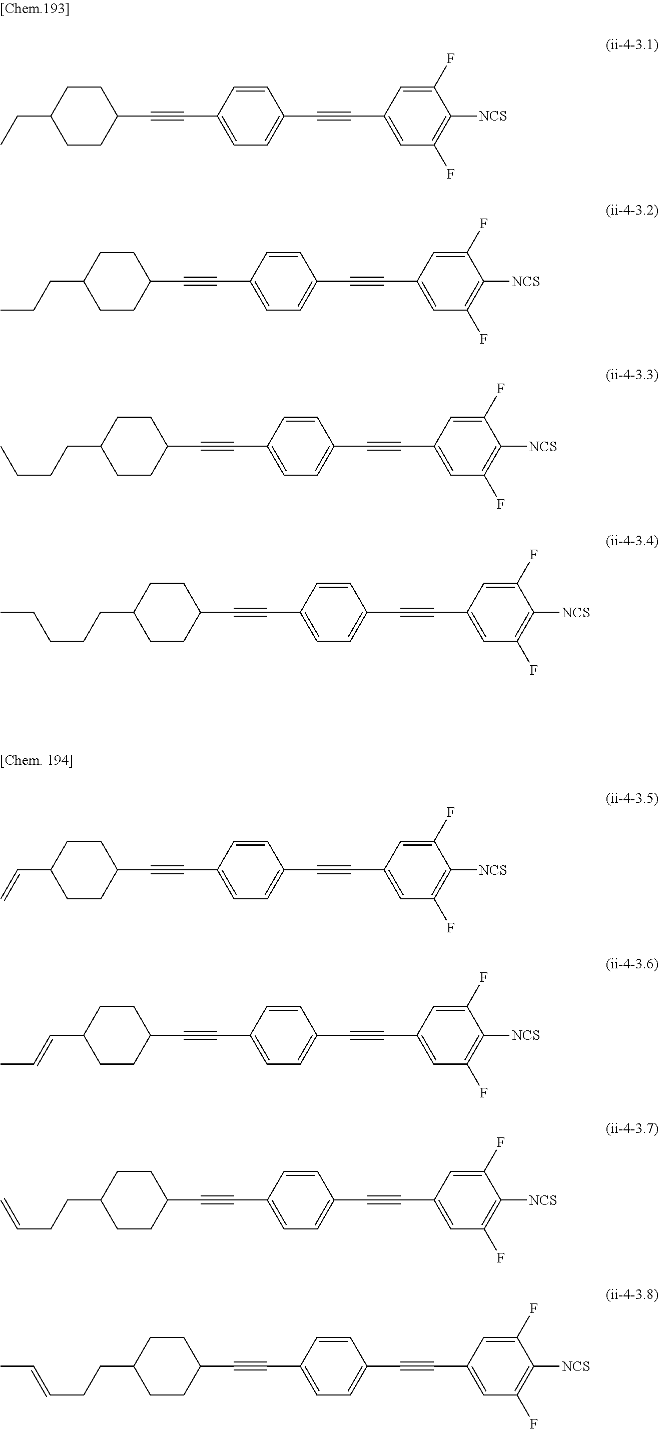

Specific examples of the compounds represented by the general formula (ii-4-4) include the compounds represented by the following structural formulae (ii-4-4.1) to (ii-4-4.5):

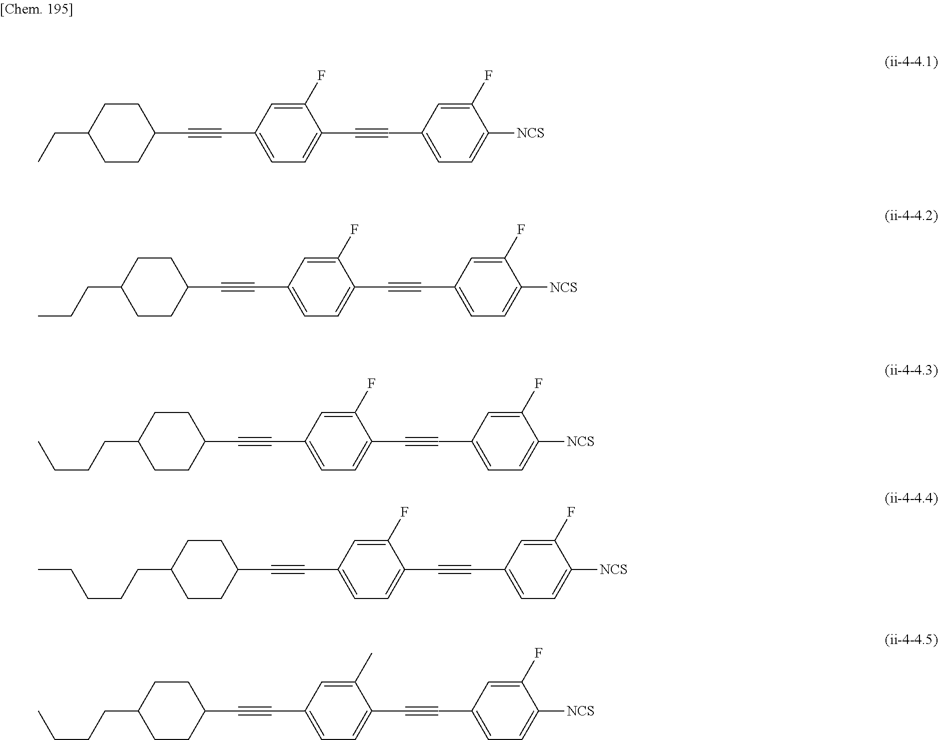

Specific examples of the compounds represented by the general formula (ii-4-5) include the compound represented by the following structural formula (ii-4-5.1):

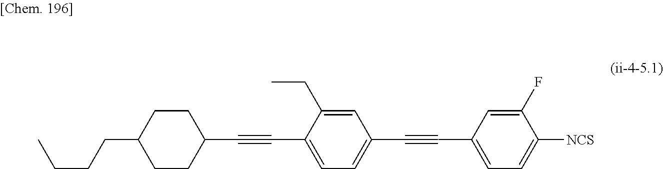

Specific examples of the compounds represented by the general formula (ii-4-6) include the compounds represented by the following structural formulae (ii-4-6.1) to (ii-4-6.5):

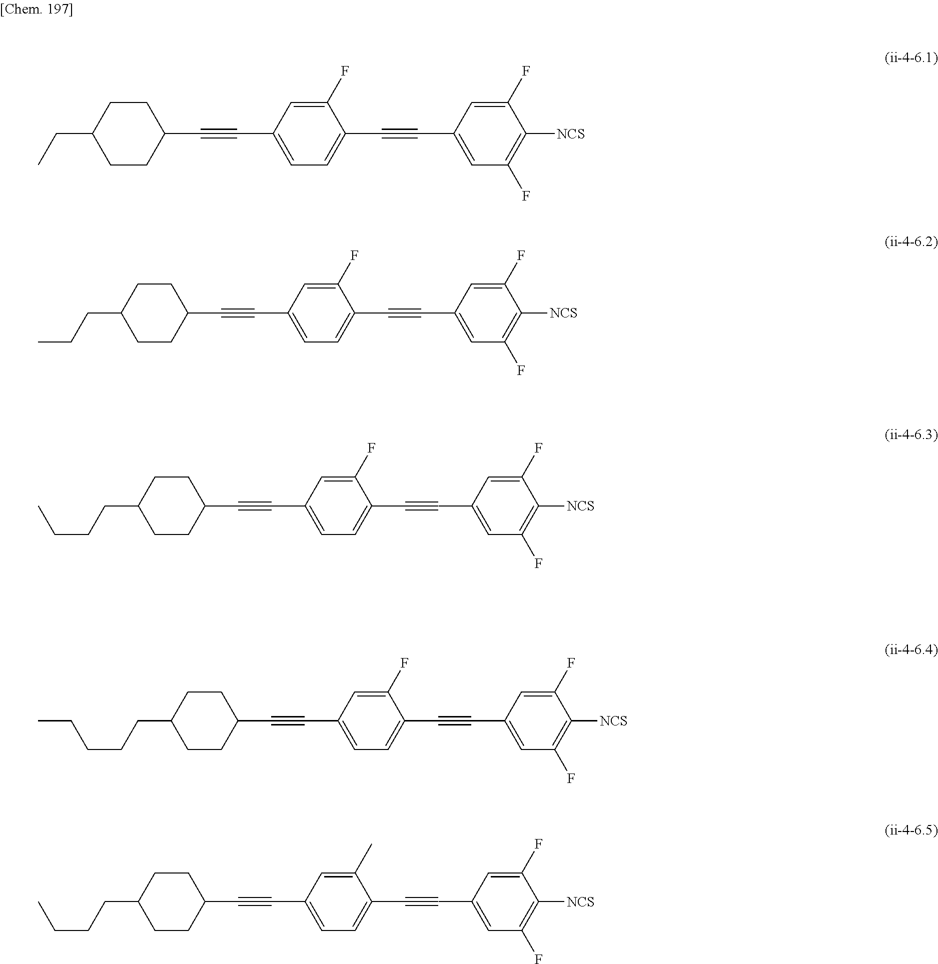

Specific examples of the compounds represented by the general formula (ii-4-7) include the compound represented by the following structural formula (ii-4-7.1):

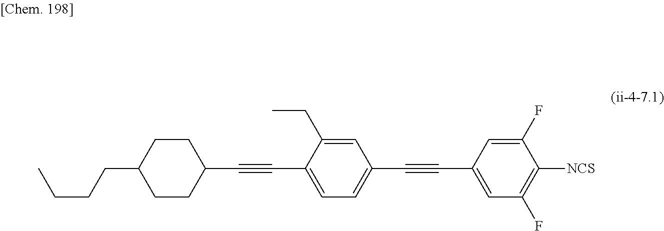

Specific examples of the compounds represented by the general formula (ii-4-8) include the compounds represented by the following structural formulae (ii-4-8.1) to (ii-4-8.4):

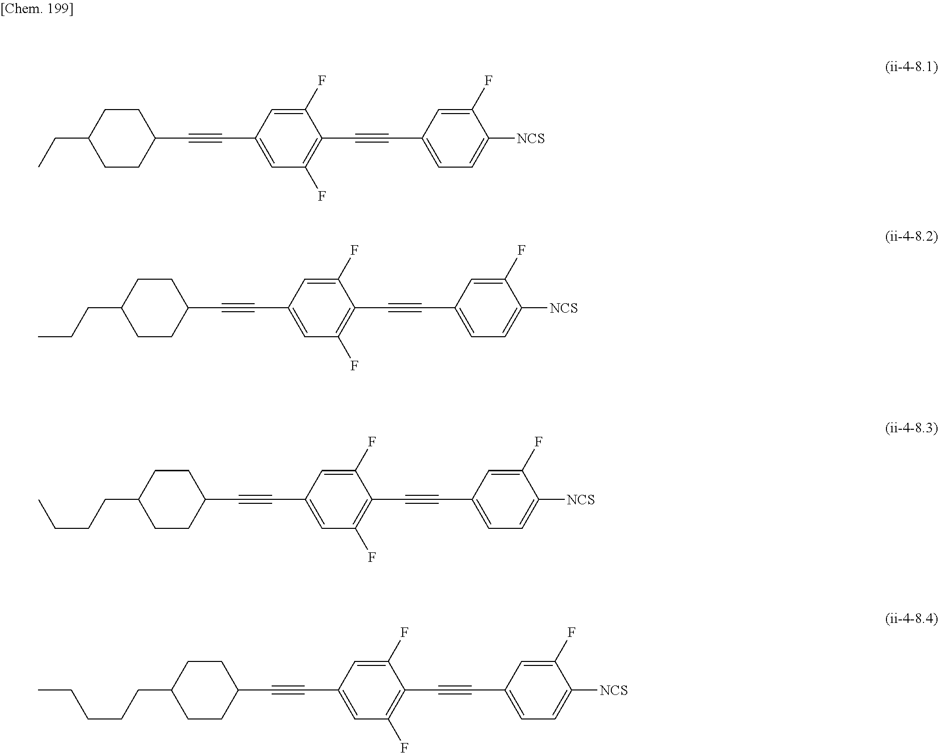

Specific examples of the compounds represented by the general formula (ii-4-9) include the compounds represented by the following structural formulae (ii-4-9.1) to (ii-4-9.4):

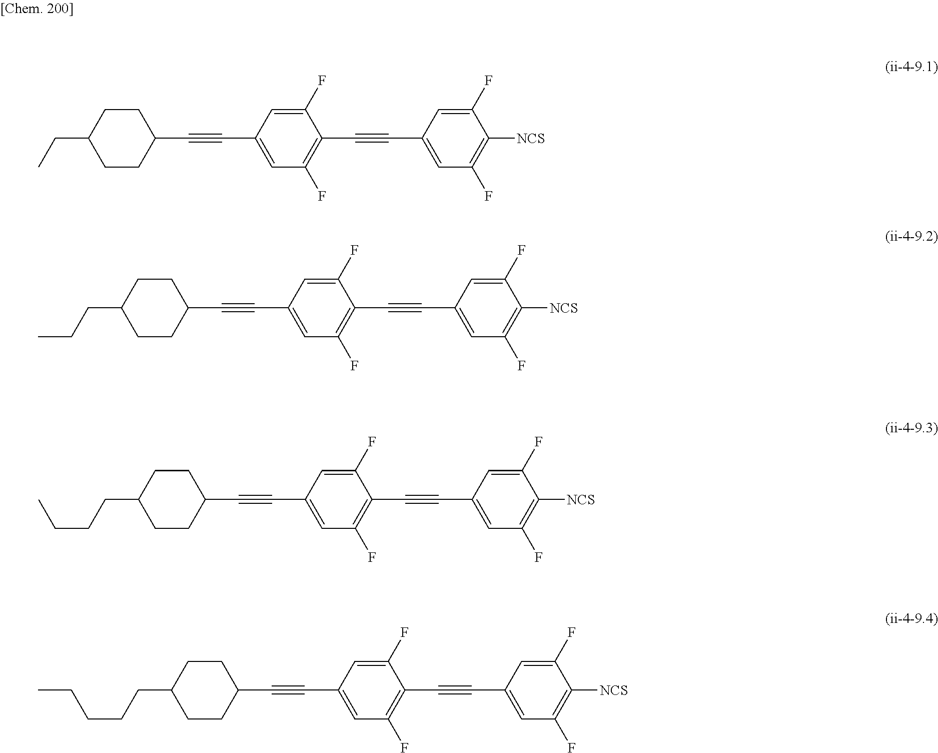

Specific examples of the compounds represented by the general formula (ii-4-10) include the compounds represented by the following structural formulae (ii-4-10.1) to (ii-4-10.4):

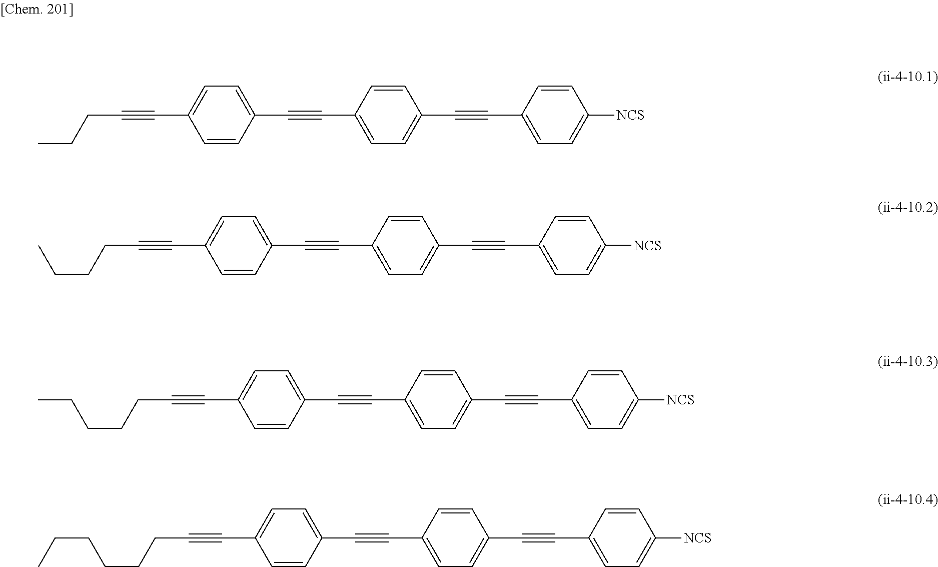

Specific examples of the compounds represented by the general formula (ii-4-11) include the compounds represented by the following structural formulae (ii-4-11.1) to (ii-4-11.5):

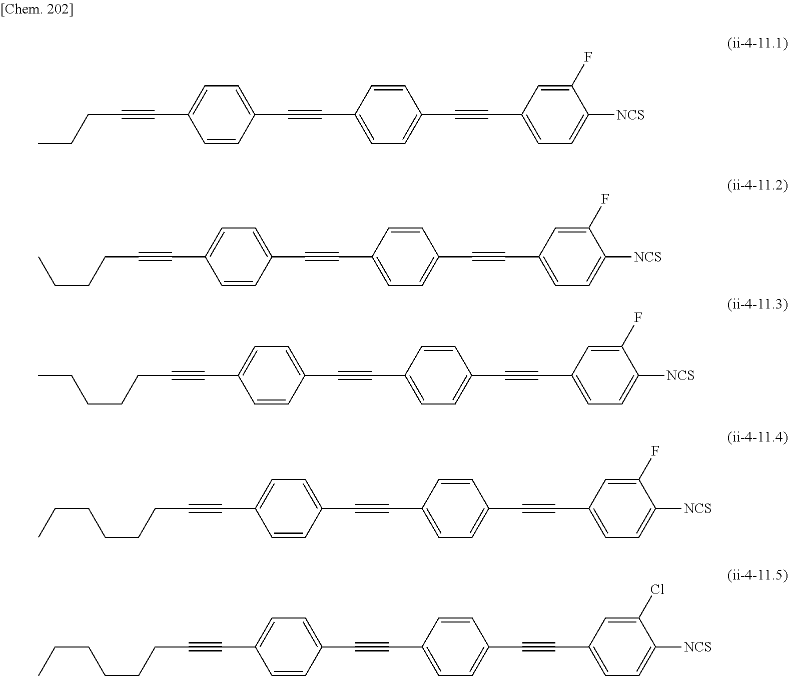

Specific examples of the compounds represented by the general formula (ii-4-12) include the compounds represented by the following structural formulae (ii-4-12.1) to (ii-4-12.5):

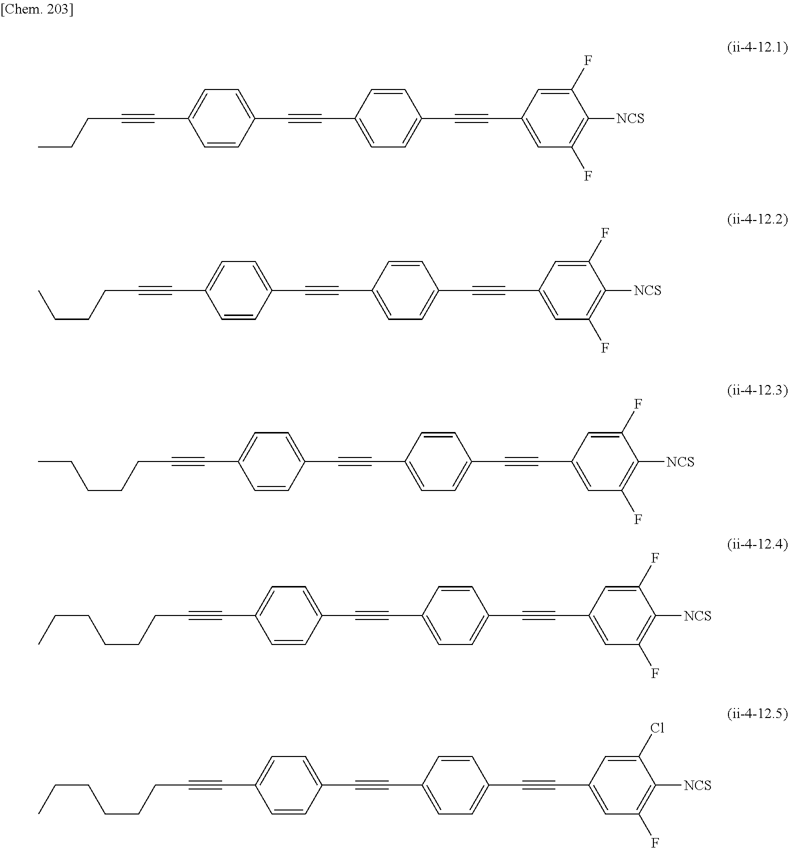

Specific examples of the compounds represented by the general formula (ii-4-13) include the compounds represented by the following structural formulae (ii-4-13.1) to (ii-4-13.4):

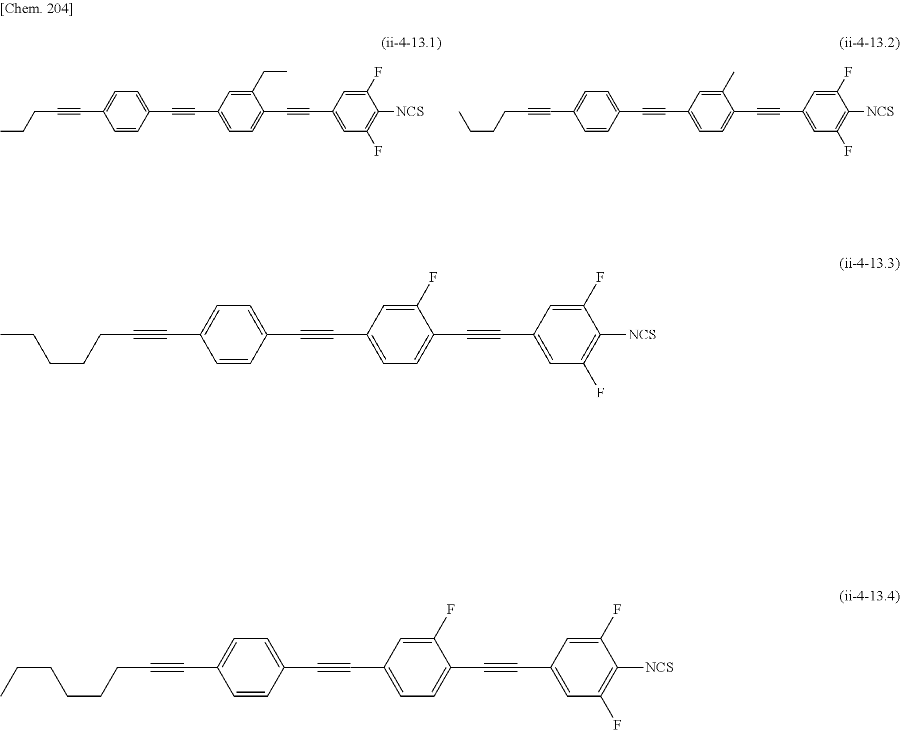

Specific examples of the compounds represented by the general formula (ii-4-14) include the compounds represented by the following structural formulae (ii-4-14.1) to (ii-4-14.4):

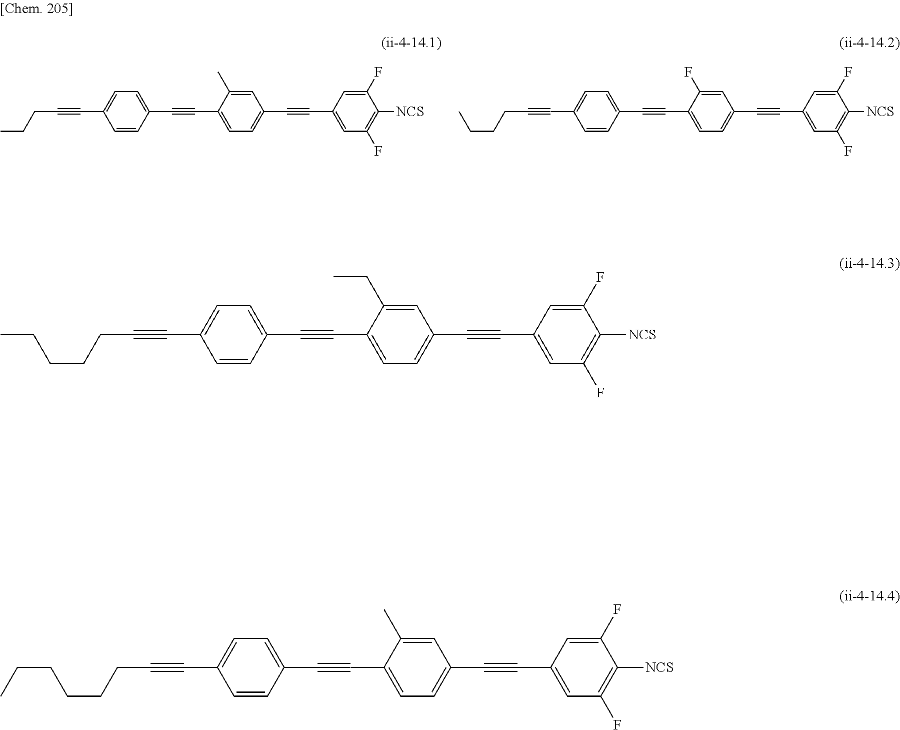

Specific examples of the compounds represented by the general formula (ii-4-15) include the compounds represented by the following structural formulae (ii-4-15.1) to (ii-4-15.6):

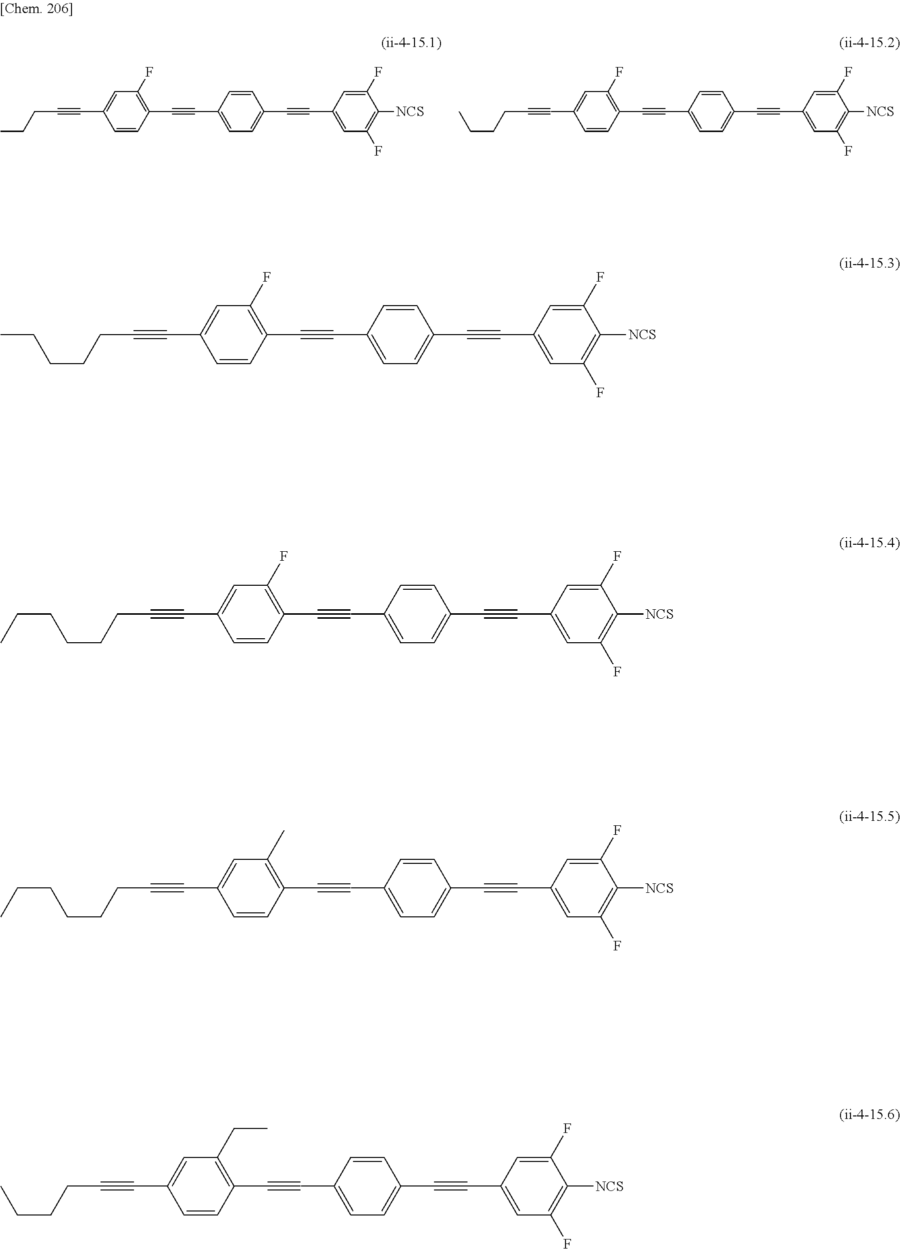

Specific examples of the compounds represented by the general formula (ii-4-16) include the compounds represented by the following structural formulae (ii-4-16.1) to (ii-4-16.3):

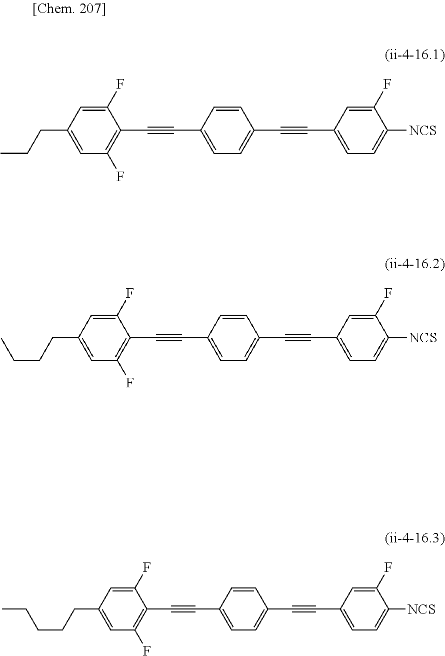

Specific examples of the compounds represented by the general formula (ii-4-17) include the compounds represented by the following structural formulae (ii-4-17.1) to (ii-4-17.3):

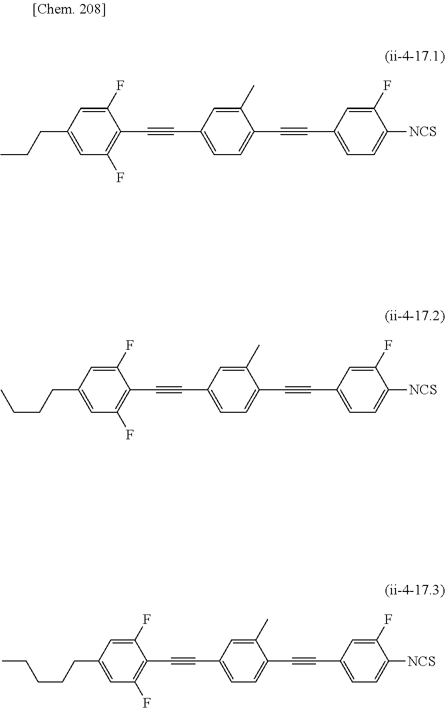

Specific examples of the compounds represented by the general formula (ii-4-18) include the compounds represented by the following structural formulae (ii-4-18.1) to (ii-4-18.4):

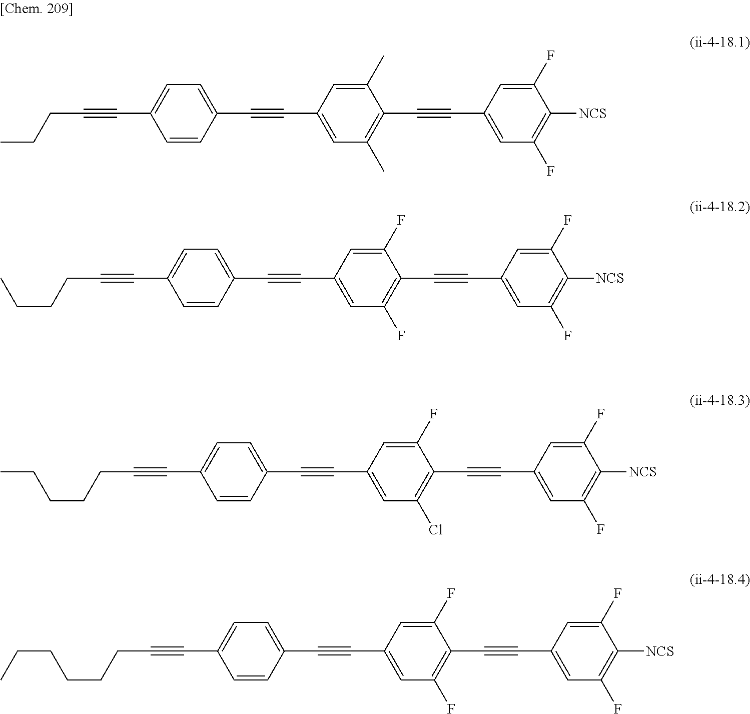

Specific examples of the compounds represented by the general formula (ii-4-19) include the compounds represented by the following structural formulae (ii-4-19.1) to (ii-4-19.8):

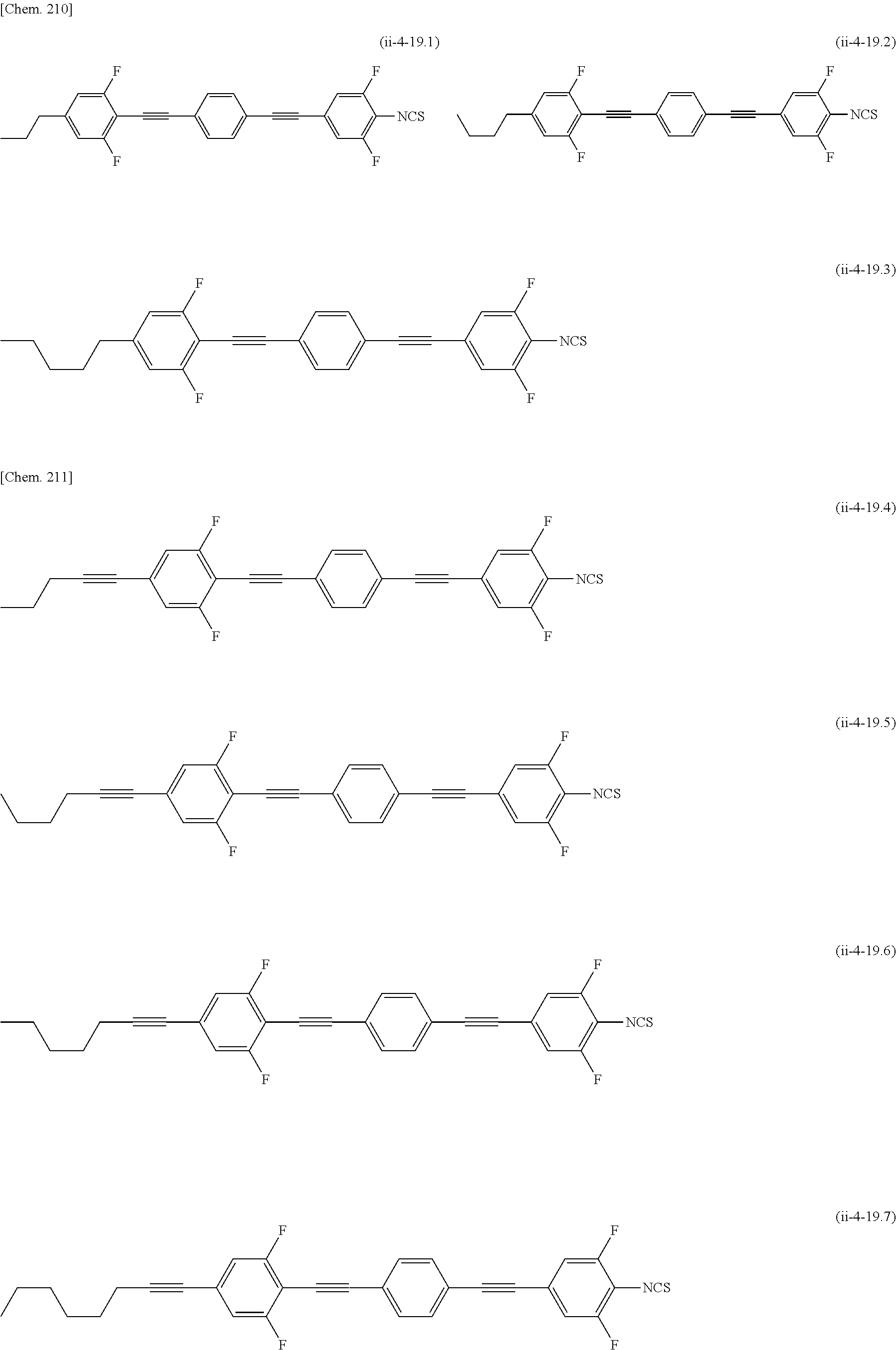

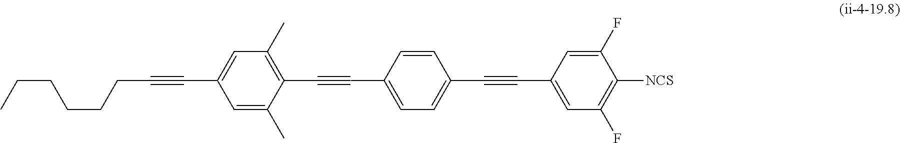

Specific examples of the compounds represented by the general formula (ii-4-20) include the compounds represented by the following structural formulae (ii-4-20.1) to (ii-4-20.4):

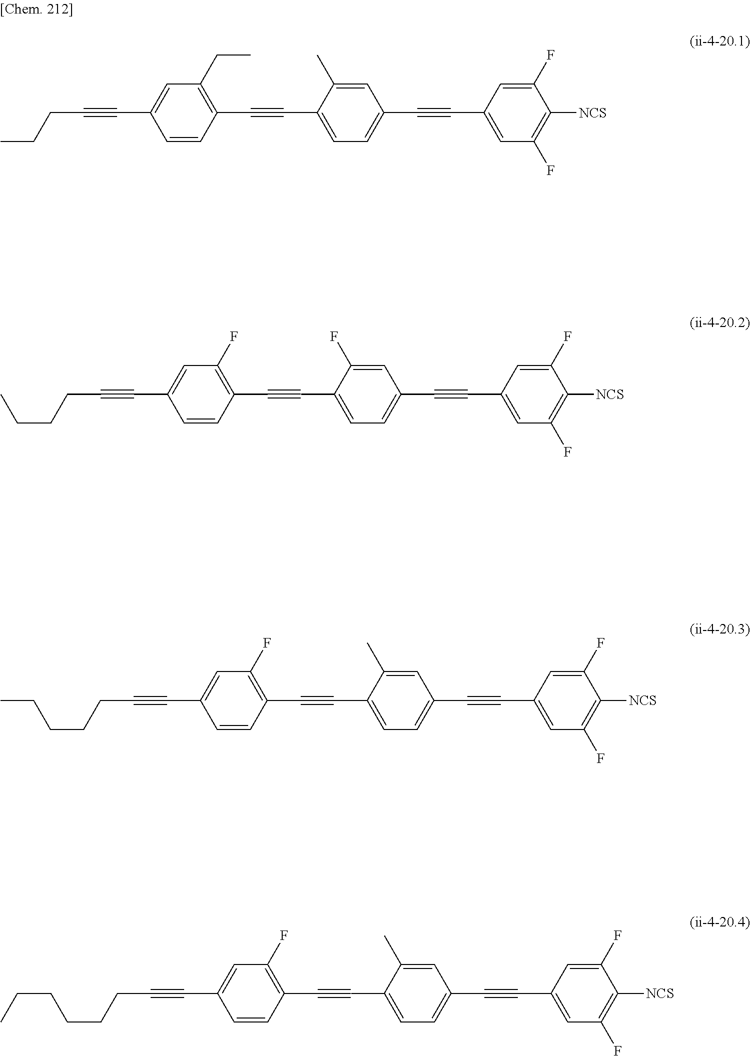

Specific examples of the compounds represented by the general formula (ii-4-21) include the compounds represented by the following structural formulae (ii-4-21.1) to (ii-4-21.4):

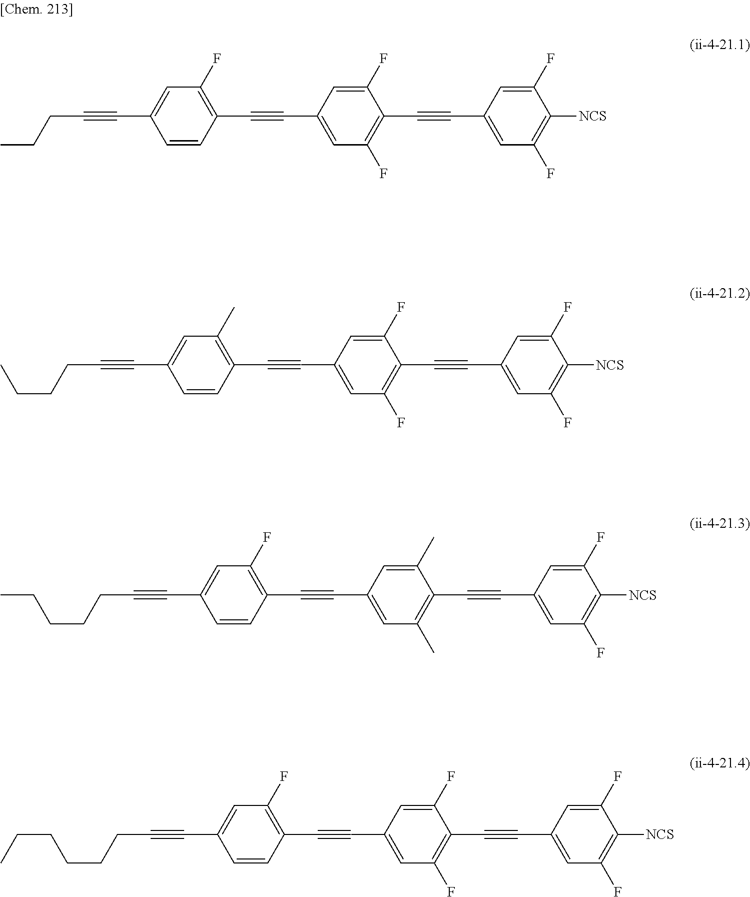

Specific examples of the compounds represented by the general formula (ii-4-22) include the compounds represented by the following structural formulae (ii-4-22.1) to (ii-4-22.3):

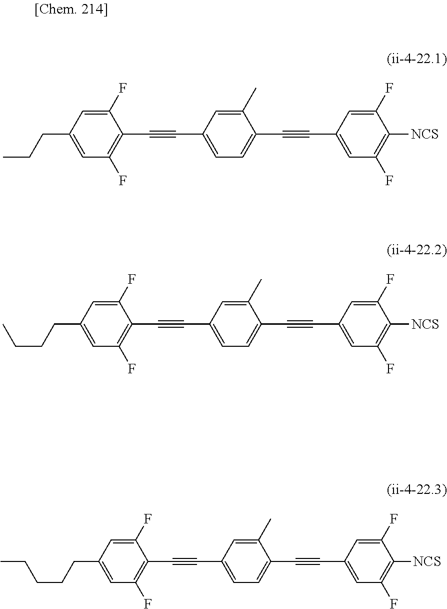

Specific examples of the compounds represented by the general formula (ii-4-23) include the compounds represented by the following structural formulae (ii-4-23.1) to (ii-4-23.3):

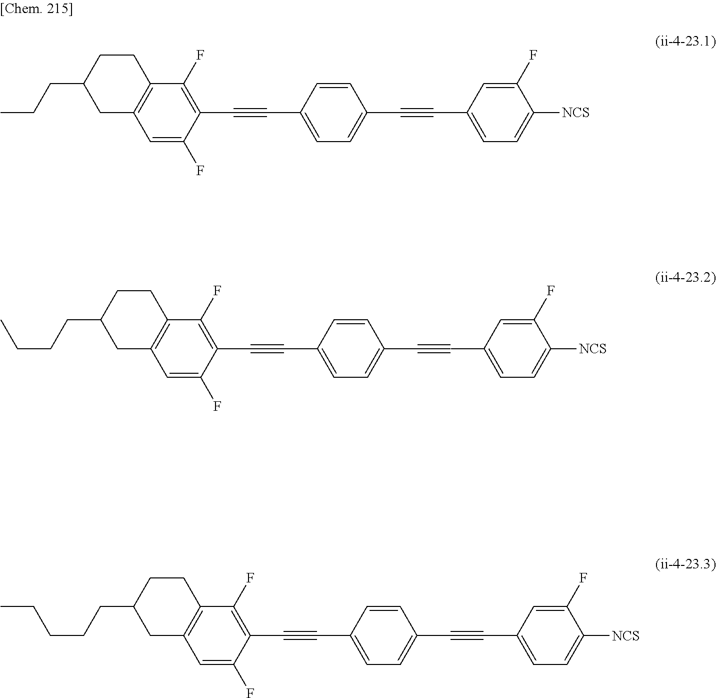

Specific examples of the compounds represented by the general formula (ii-4-24) include the compounds represented by the following structural formulae (ii-4-24.1) to (ii-4-24.3):

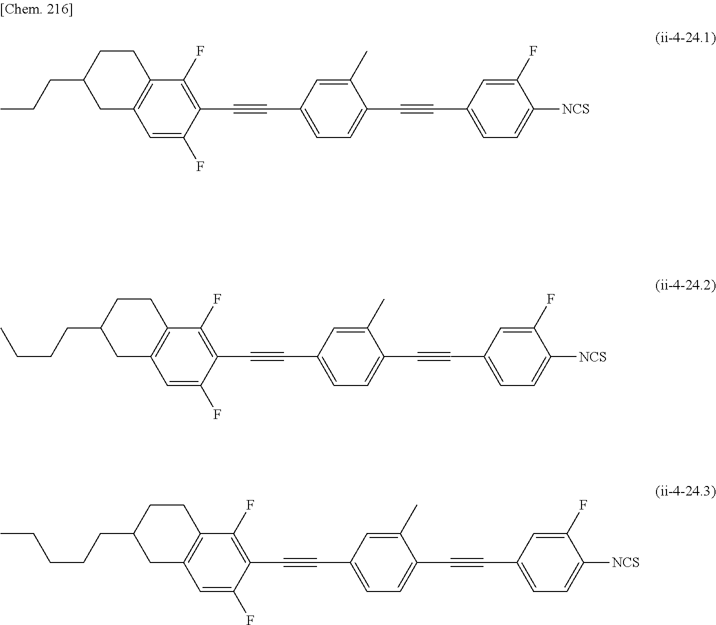

Specific examples of the compounds represented by the general formula (ii-4-25) include the compounds represented by the following structural formulae (ii-4-25.1) to (ii-4-25.3):

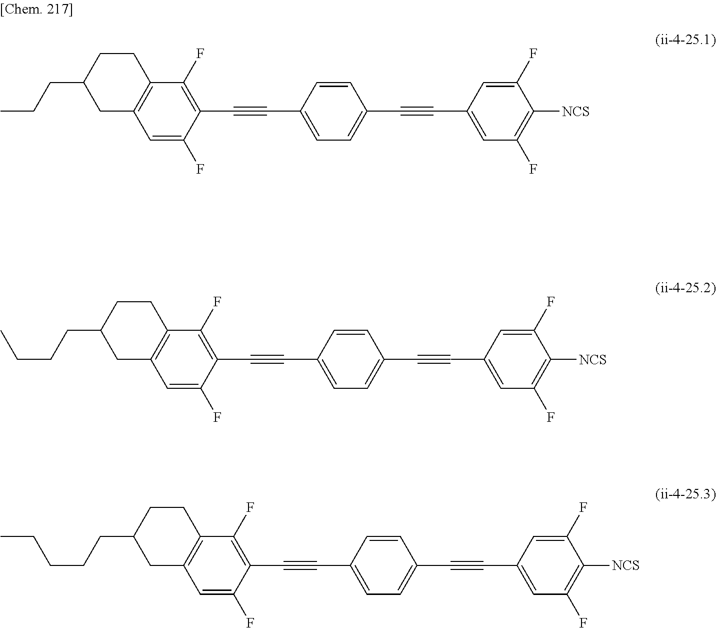

Specific examples of the compounds represented by the general formula (ii-4-26) include the compounds represented by the following structural formulae (ii-4-26.1) to (ii-4-26.3):

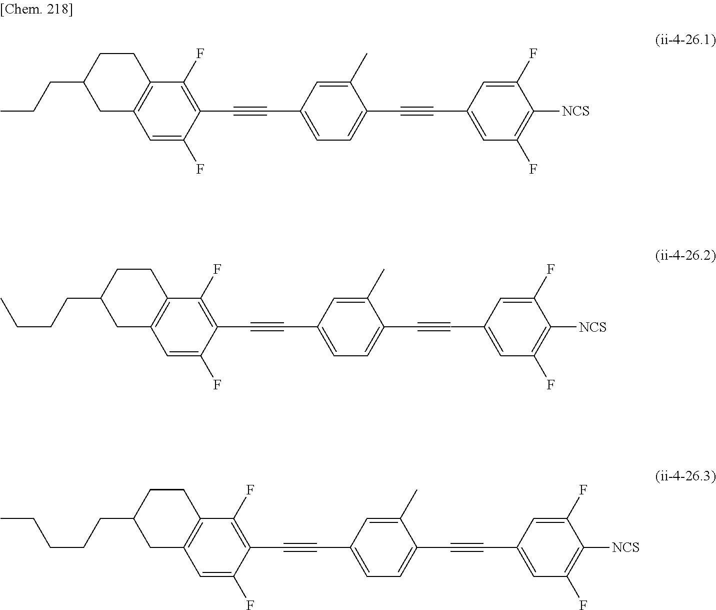

Specific examples of the compounds represented by the general formula (ii-4-27) include the compounds represented by the following structural formulae (ii-4-27.1) and (ii-4-27.2):

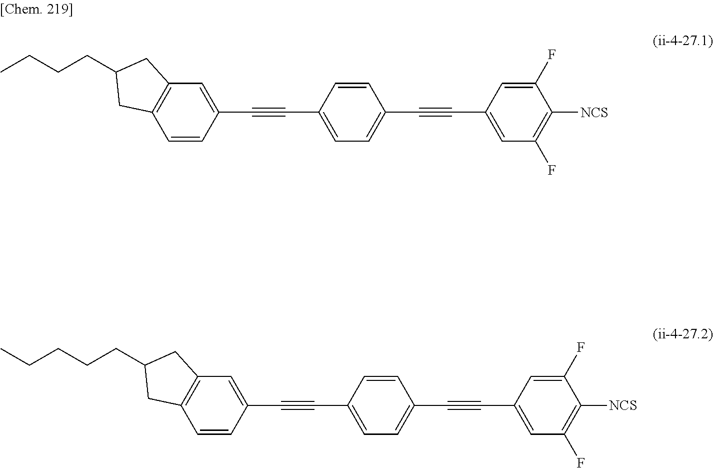

Specific examples of the compounds represented by the general formula (ii-4-28) include the compounds represented by the following structural formulae (ii-4-28.1) and (ii-4-28.2):

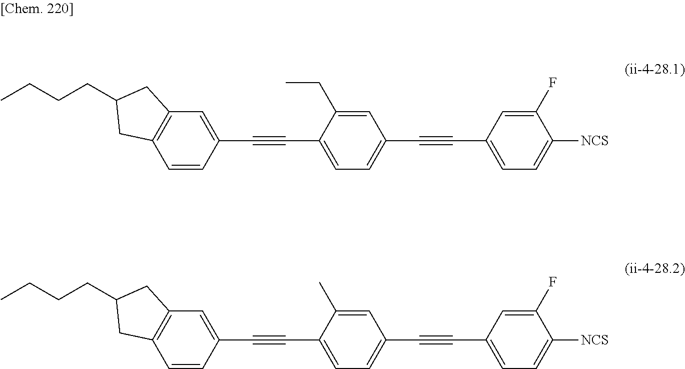

Specific examples of the compounds represented by the general formula (ii-4-29) include the compounds represented by the following structural formulae (ii-4-29.1) to (ii-4-29.4):

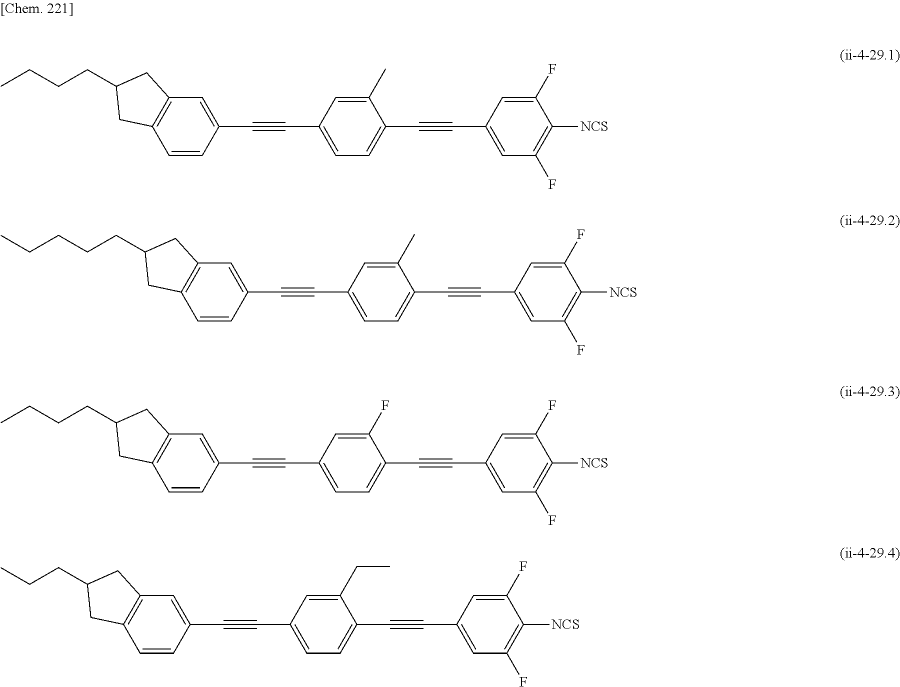

Specific examples of the compounds represented by the general formula (ii-4-30) include the compounds represented by the following structural formulae (ii-4-30.1) and (ii-4-30.2):

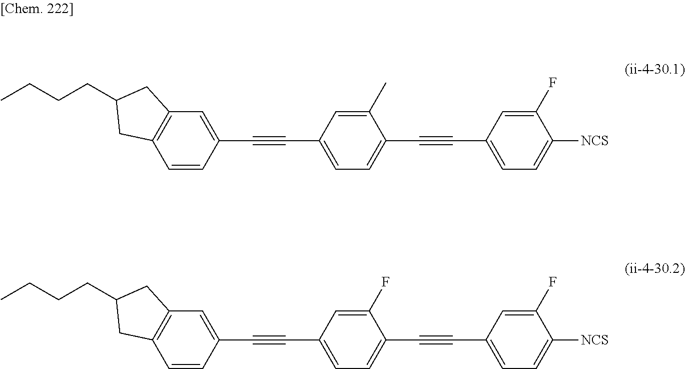

Compounds represented by the general formula (ii-5) are preferably the compounds represented by the following general formulae (ii-5-1) to (ii-5-8):

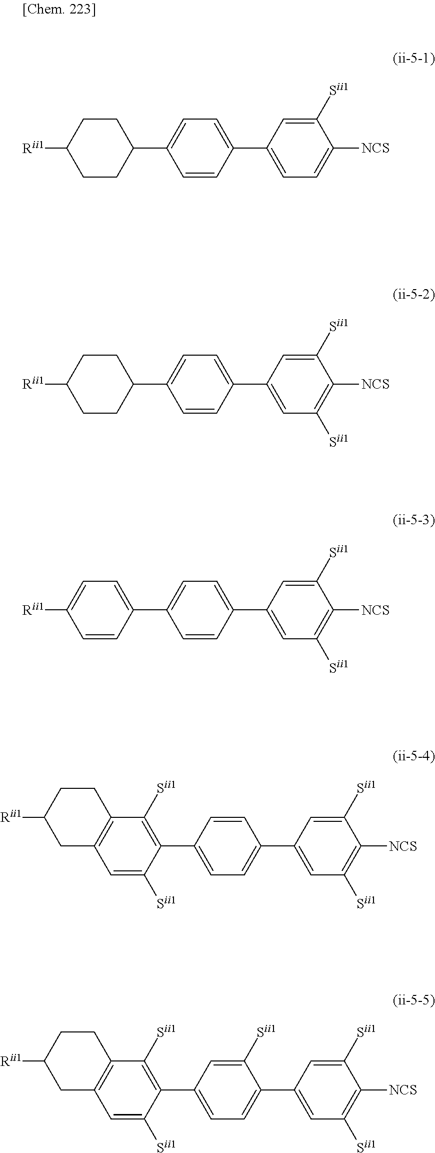

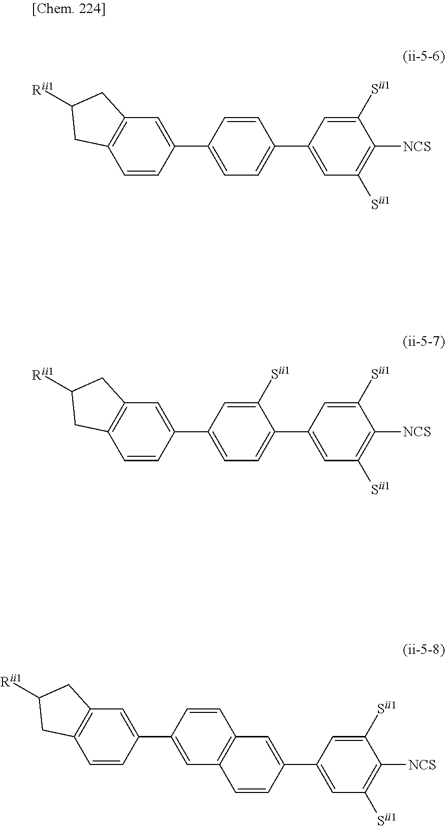

In the general formulae (ii-5-1) to (ii-5-8), Rⁱⁱ¹ and Sⁱⁱ¹ each independently denote the same as Rⁱⁱ¹ and Sⁱⁱ¹ in the general formula (ii).

Specific examples of the compounds represented by the general formula (ii-5-1) include the compounds represented by the following structural formulae (ii-5-1.1) to (ii-5-1.4):

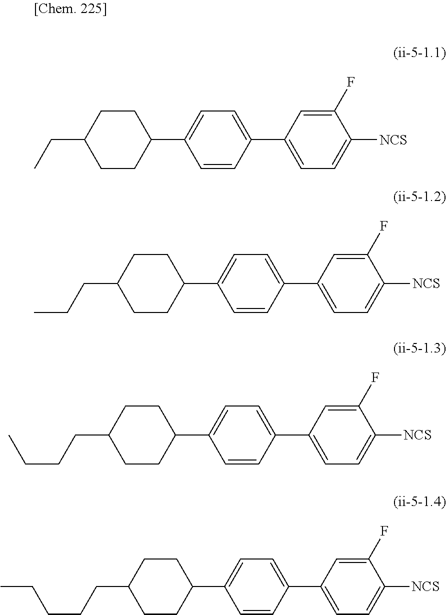

Specific examples of the compounds represented by the general formula (ii-5-2) include the compounds represented by the following structural formulae (ii-5-2.1) to (ii-5-2.4):

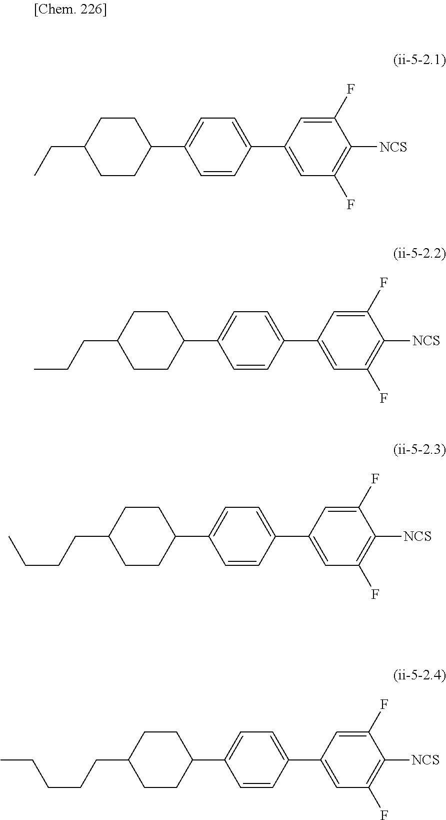

Specific examples of the compounds represented by the general formula (ii-5-3) include the compound represented by the following structural formula (ii-5-3.1):

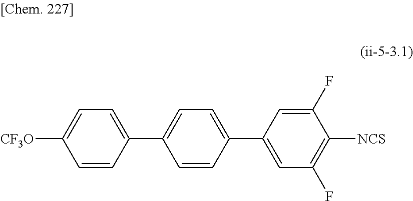

Specific examples of the compounds represented by the general formula (ii-5-4) include the compounds represented by the following structural formulae (ii-5-4.1) to (ii-5-4.3):

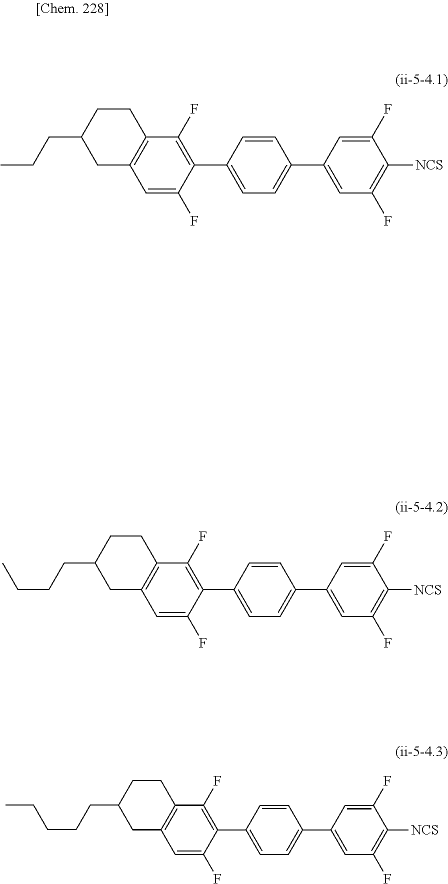

Specific examples of the compounds represented by the general formula (ii-5-5) include the compounds represented by the following structural formulae (ii-5-5.1) to (ii-5-5.3):

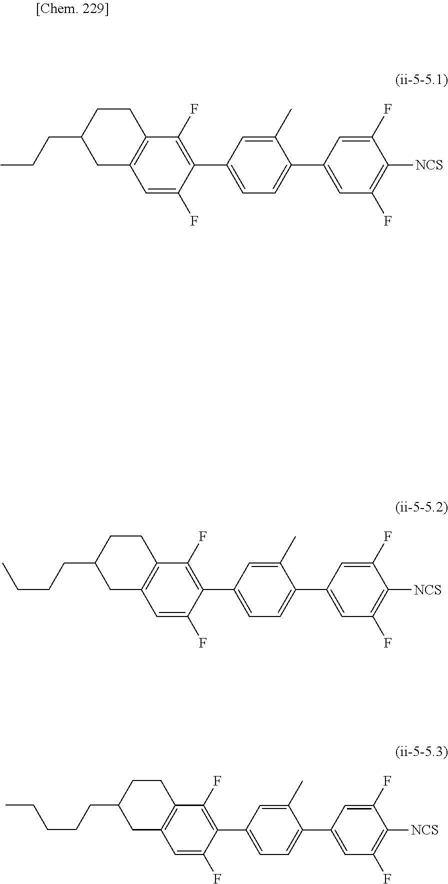

Specific examples of the compounds represented by the general formula (ii-5-6) include the compounds represented by the following structural formulae (ii-5-6.1) to (ii-5-6.3):

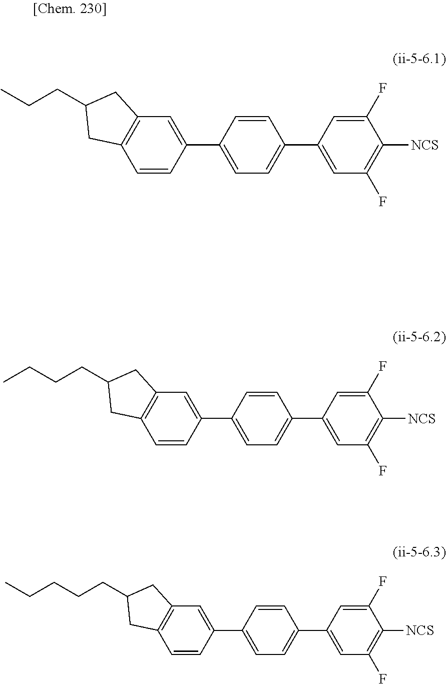

Specific examples of the compounds represented by the general formula (ii-5-7) include the compounds represented by the following structural formulae (ii-5-7.1) to (ii-5-7.3):

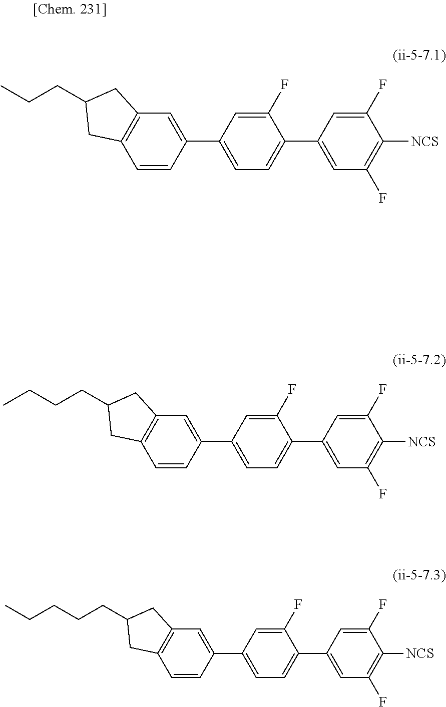

Specific examples of the compounds represented by the general formula (ii-5-8) include the compounds represented by the following structural formulae (ii-5-8.1) to (ii-5-8.3):

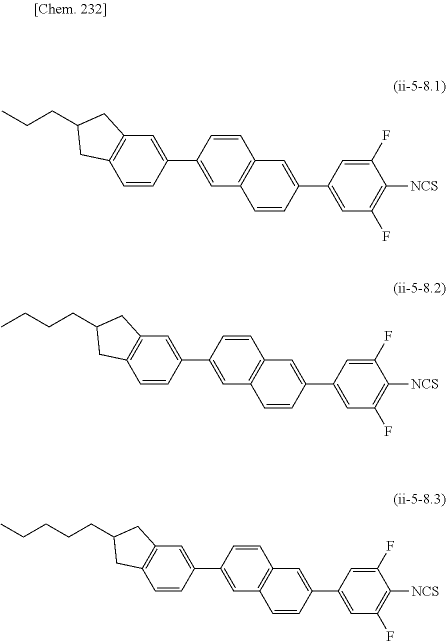

Compounds represented by the general formula (ii-6) are preferably the compounds represented by the following general formulae (ii-6-1) to (ii-6-36):

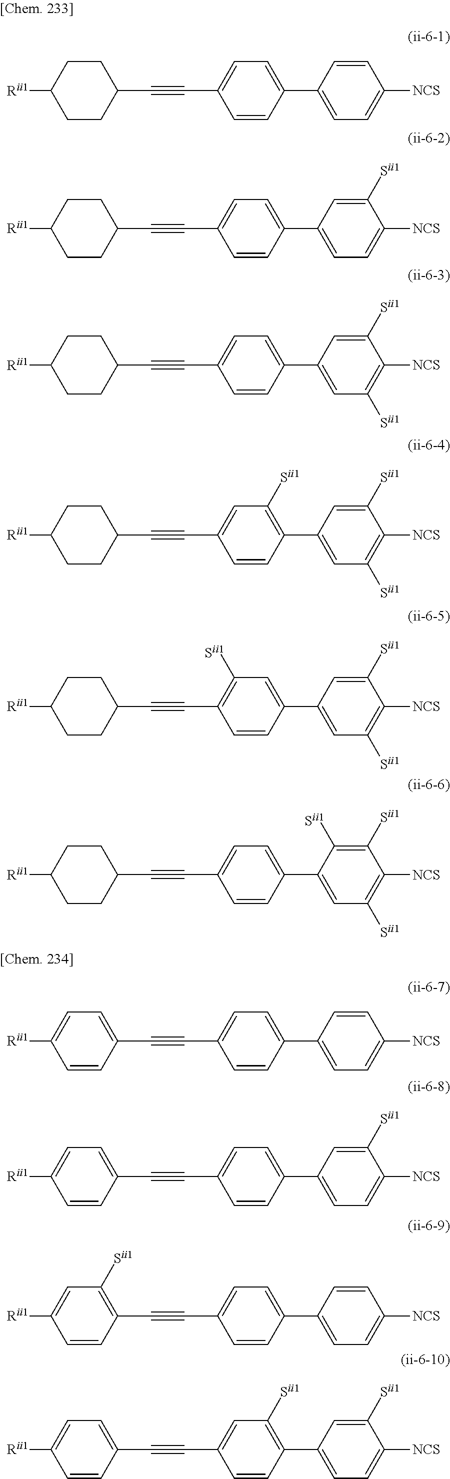

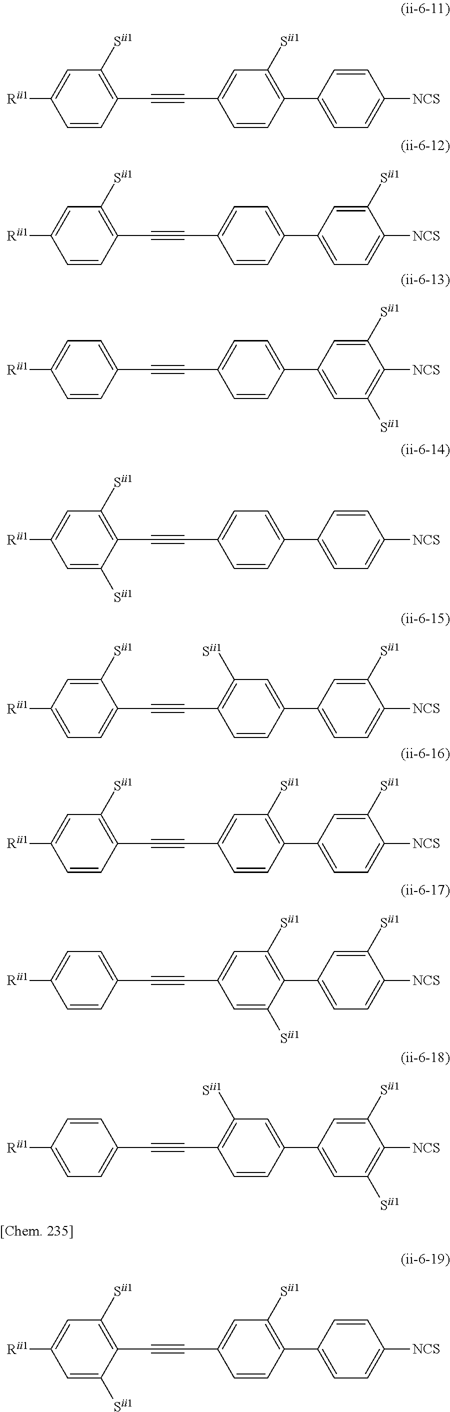

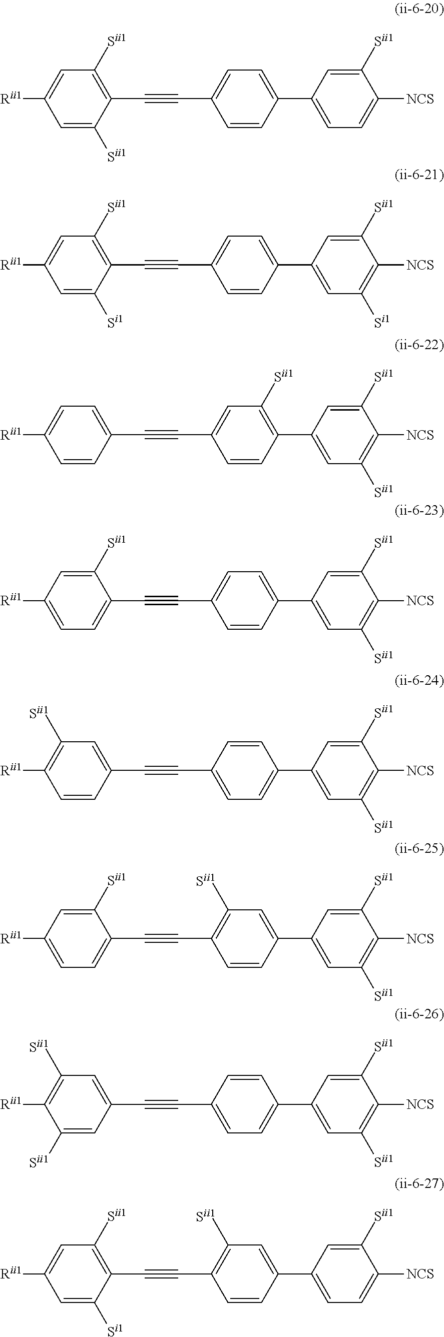

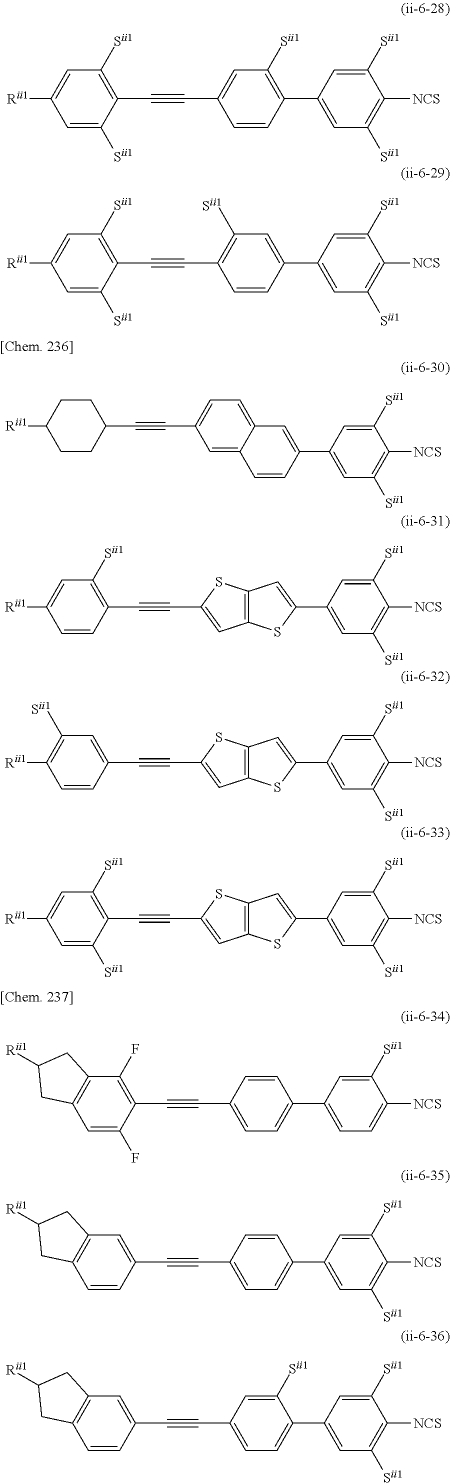

In the general formulae (ii-6-1) to (ii-6-36), Rⁱⁱ¹ and Sⁱⁱ¹ each independently denote the same as Rⁱⁱ¹ and Sⁱⁱ¹ in the general formula (ii).

Specific examples of the compounds represented by the general formula (ii-6-1) include the compounds represented by the following structural formulae (ii-6-1.1) to (ii-6-1.4):

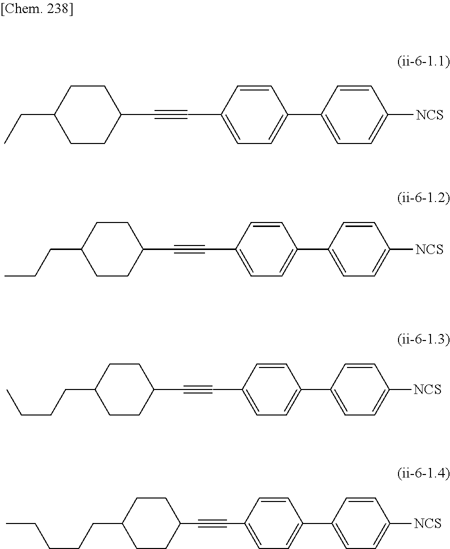

Specific examples of the compounds represented by the general formula (ii-6-2) include the compounds represented by the following structural formulae (ii-6-2.1) to (ii-6-2.4):

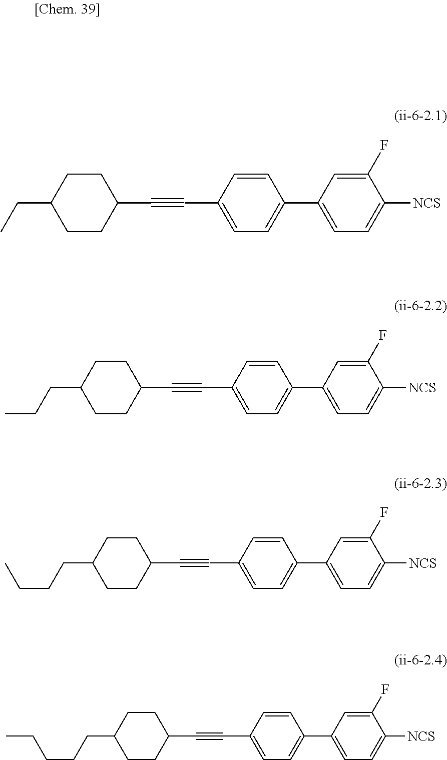

Specific examples of the compounds represented by the general formula (ii-6-3) include the compounds represented by the following structural formulae (ii-6-3.1) to (ii-6-3.8):

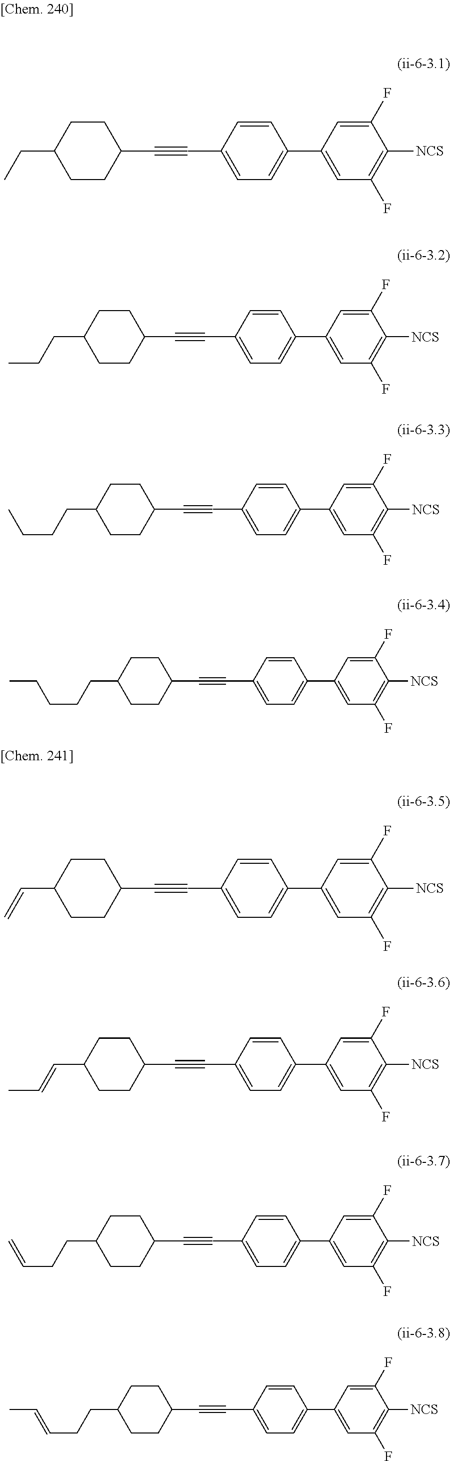

Specific examples of the compounds represented by the general formula (ii-6-4) include the compounds represented by the following structural formulae (ii-6-4.1) to (ii-6-4.4):

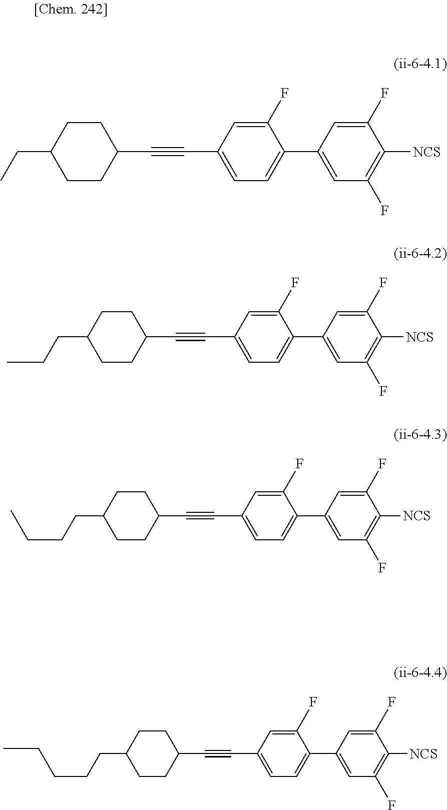

Specific examples of the compounds represented by the general formula (ii-6-5) include the compounds represented by the following structural formulae (ii-6-5.1) to (ii-6-5.4):

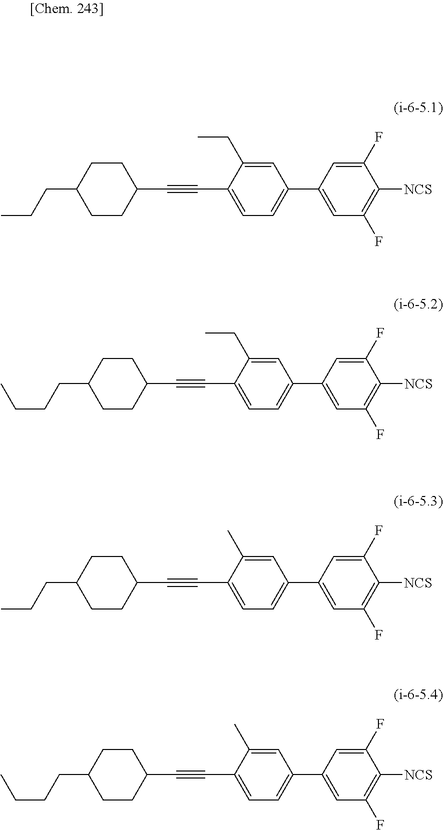

Specific examples of the compounds represented by the general formula (ii-6-6) include the compounds represented by the following structural formulae (ii-6-6.1) and (ii-6-6.2):

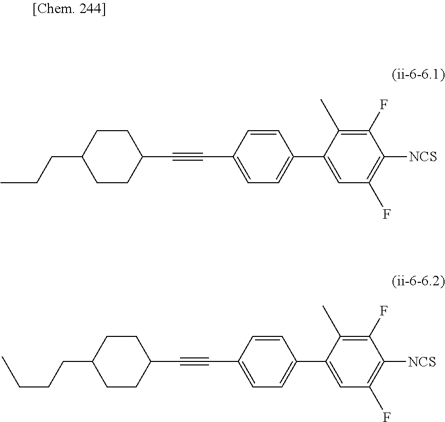

Specific examples of the compounds represented by the general formula (ii-6-7) include the compounds represented by the following structural formulae (ii-6-7.1) to (ii-6-7.8):

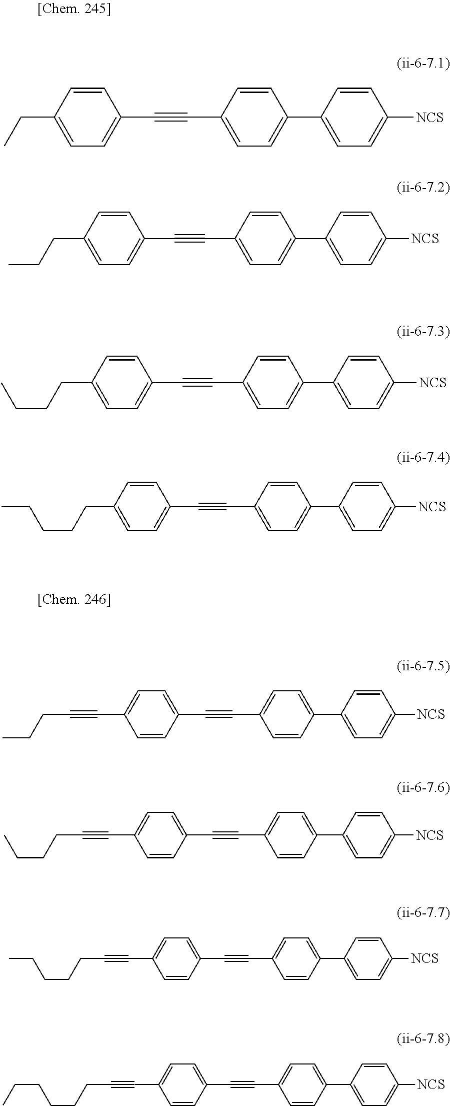

Specific examples of the compounds represented by the general formula (ii-6-8) include the compounds represented by the following structural formulae (ii-6-8.1) to (ii-6-8.9):

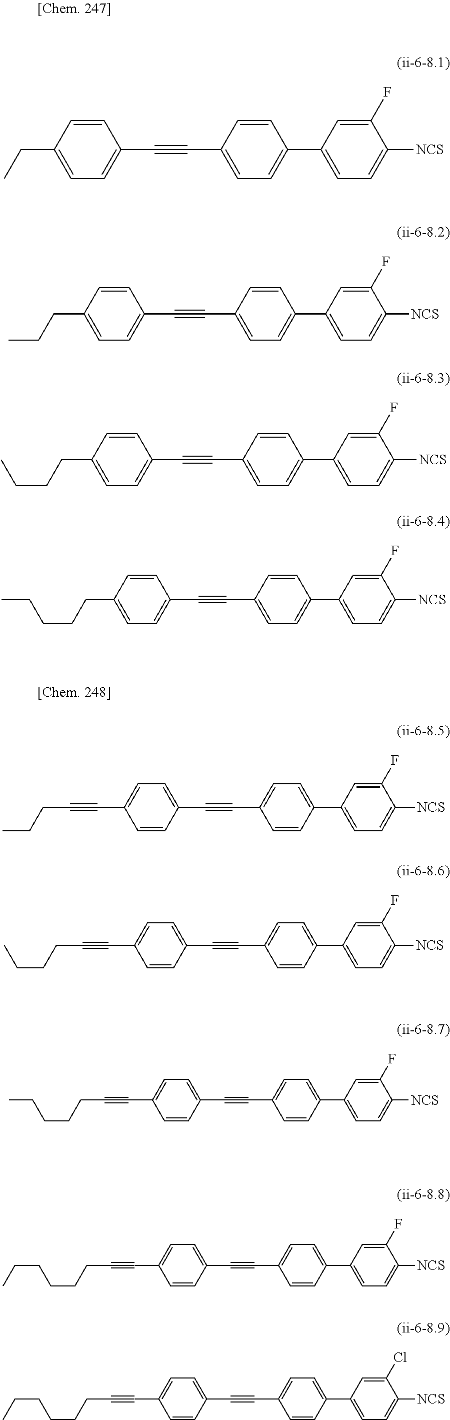

Specific examples of the compounds represented by the general formula (ii-6-9) include the compounds represented by the following structural formulae (ii-6-9.1) to (ii-6-9.4):

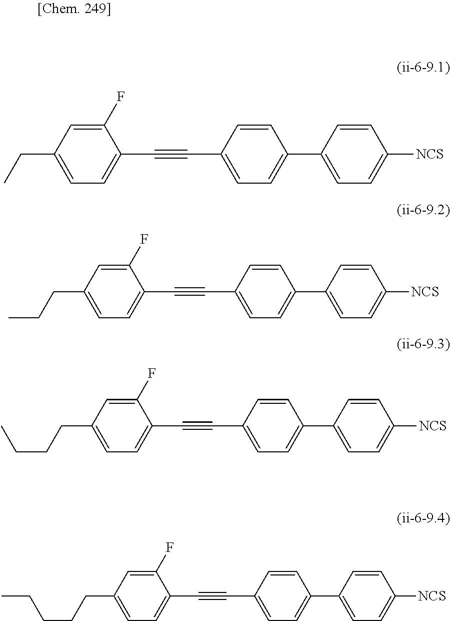

Specific examples of the compounds represented by the general formula (ii-6-10) include the compounds represented by the following structural formulae (ii-6-10.1) to (ii-6-10.4):

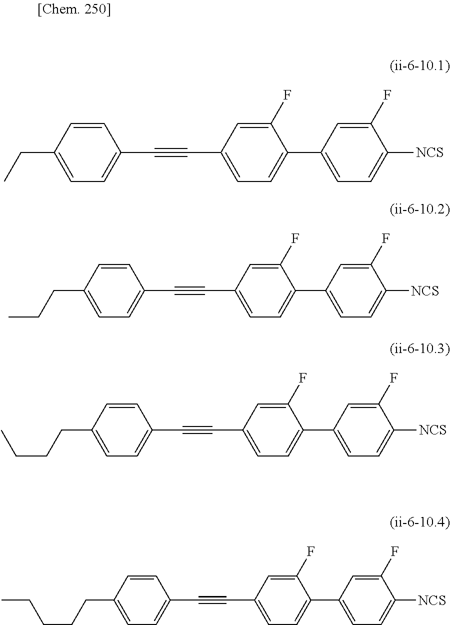

Specific examples of the compounds represented by the general formula (ii-6-11) include the compounds represented by the following structural formulae (ii-6-11.1) to (ii-6-11.4):

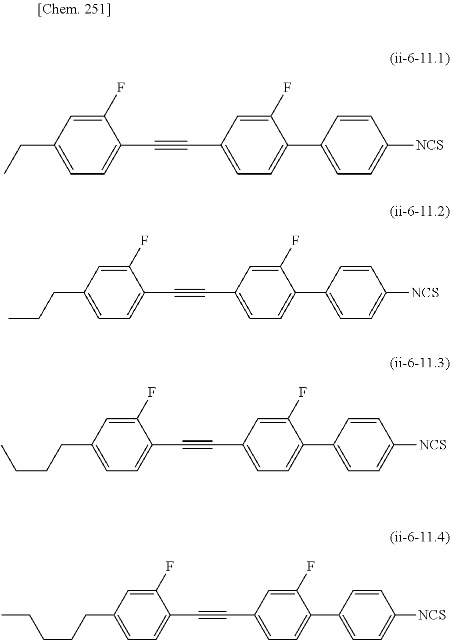

Specific examples of the compounds represented by the general formula (ii-6-12) include the compounds represented by the following structural formulae (ii-6-12.1) to (ii-6-12.4):

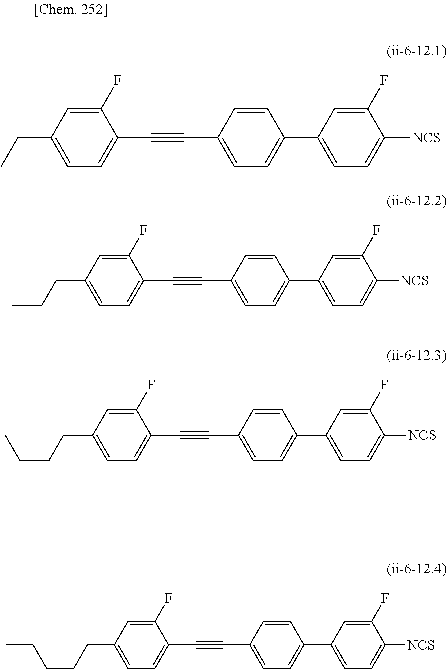

Specific examples of the compounds represented by the general formula (ii-6-13) include the compounds represented by the following structural formulae (ii-6-13.1) to (ii-6-13.20):

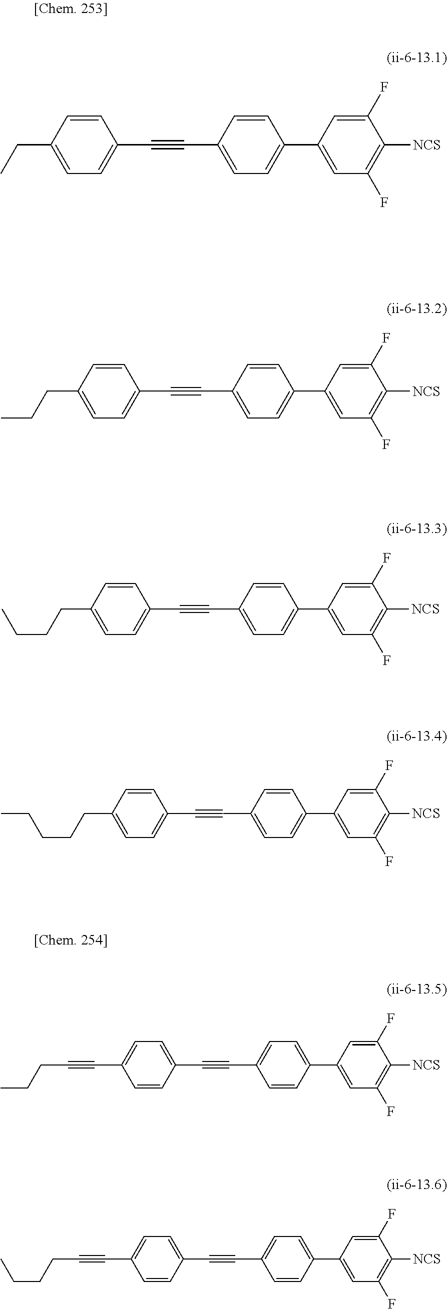

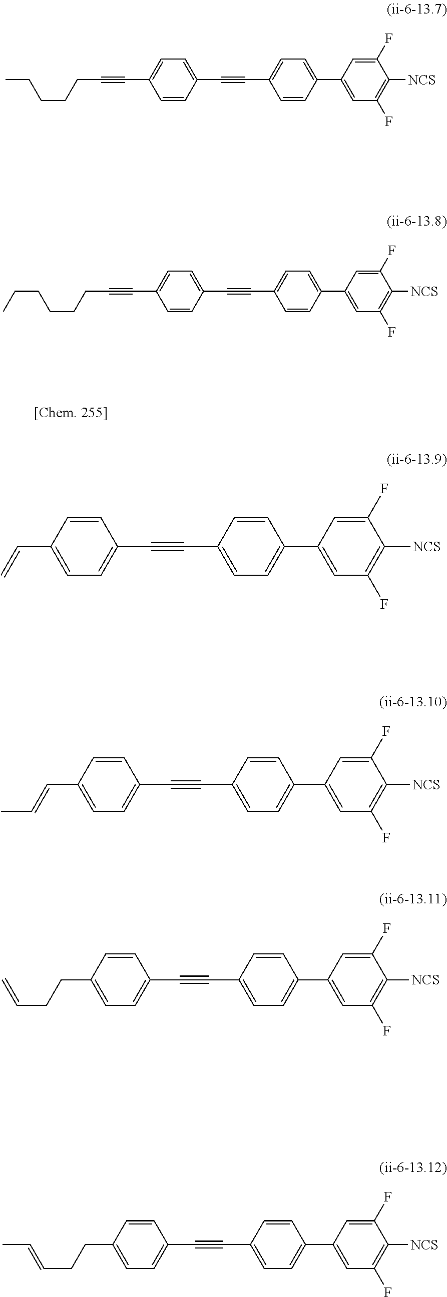

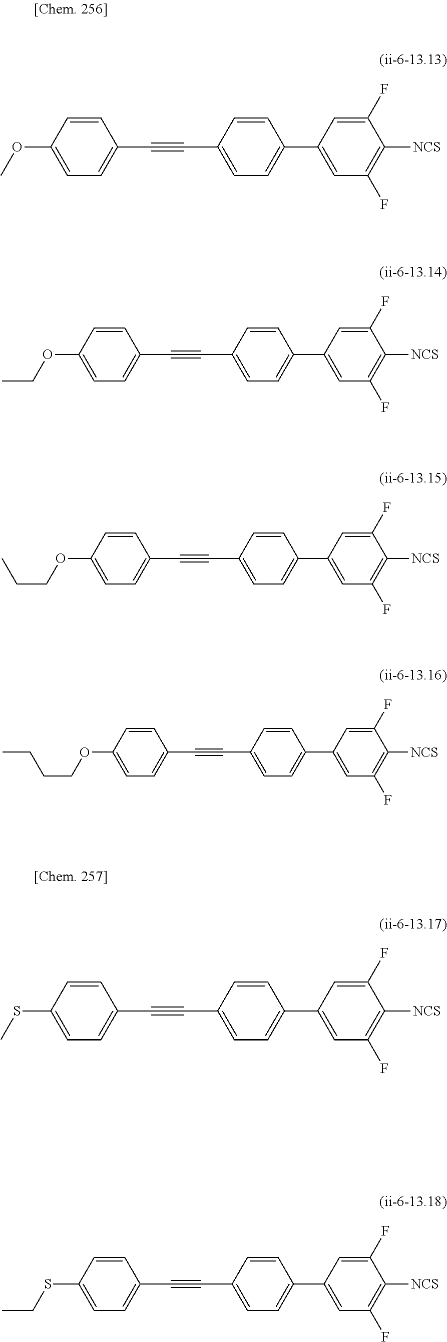

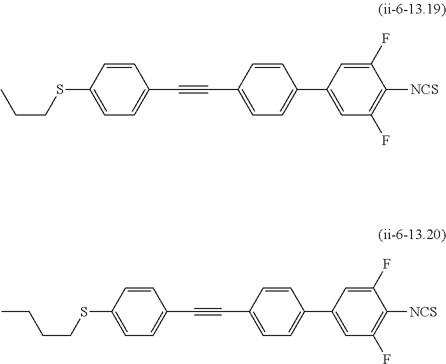

Specific examples of the compounds represented by the general formula (ii-6-14) include the compounds represented by the following structural formulae (ii-6-14.1) to (ii-6-14.4):

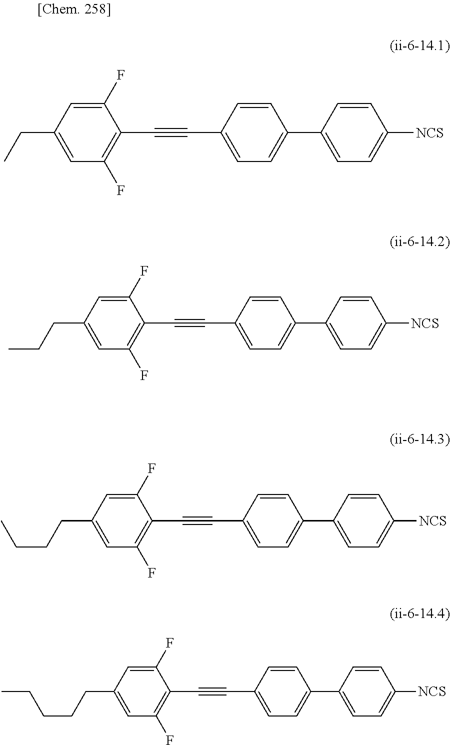

Specific examples of the compounds represented by the general formula (ii-6-15) include the compound represented by the following structural formula (ii-6-15.1):

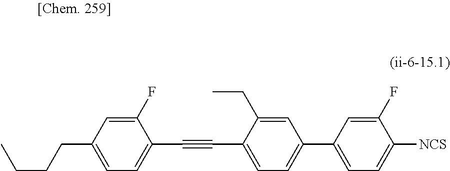

Specific examples of the compounds represented by the general formula (ii-6-16) include the compounds represented by the following structural formulae (ii-6-16.1) to (ii-6-16.5):

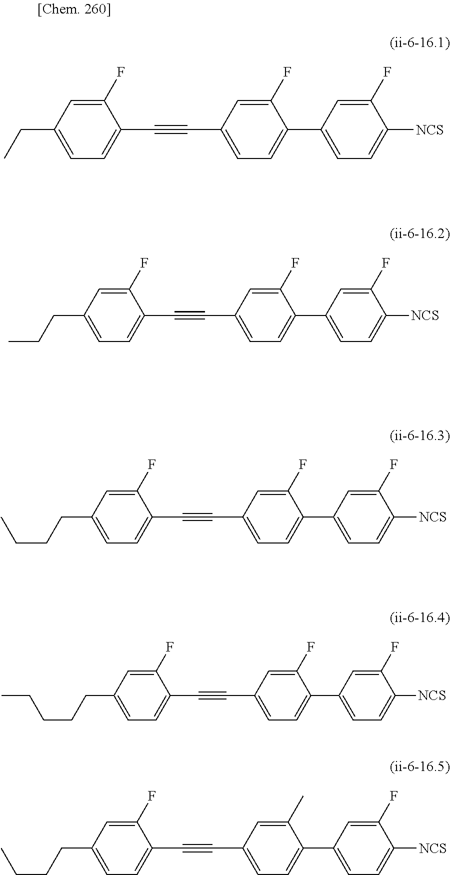

Specific examples of the compounds represented by the general formula (ii-6-17) include the compounds represented by the following structural formulae (ii-6-17.1) to (ii-6-17.4):

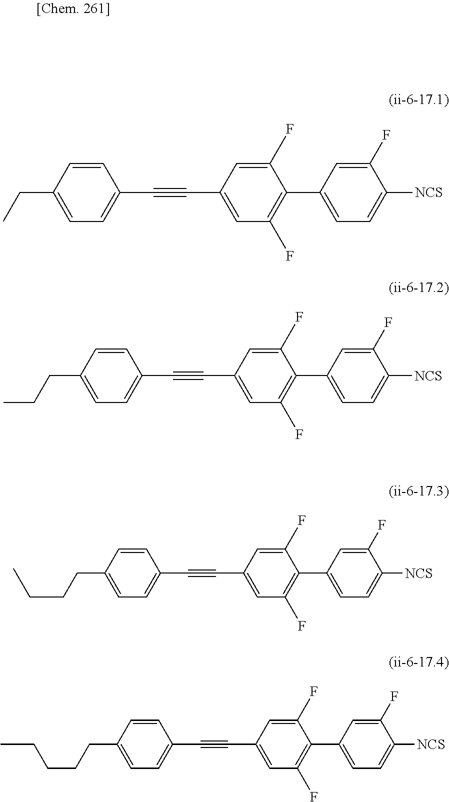

Specific examples of the compounds represented by the general formula (ii-6-18) include the compounds represented by the following structural formulae (ii-6-18.1) to (ii-6-18.8):

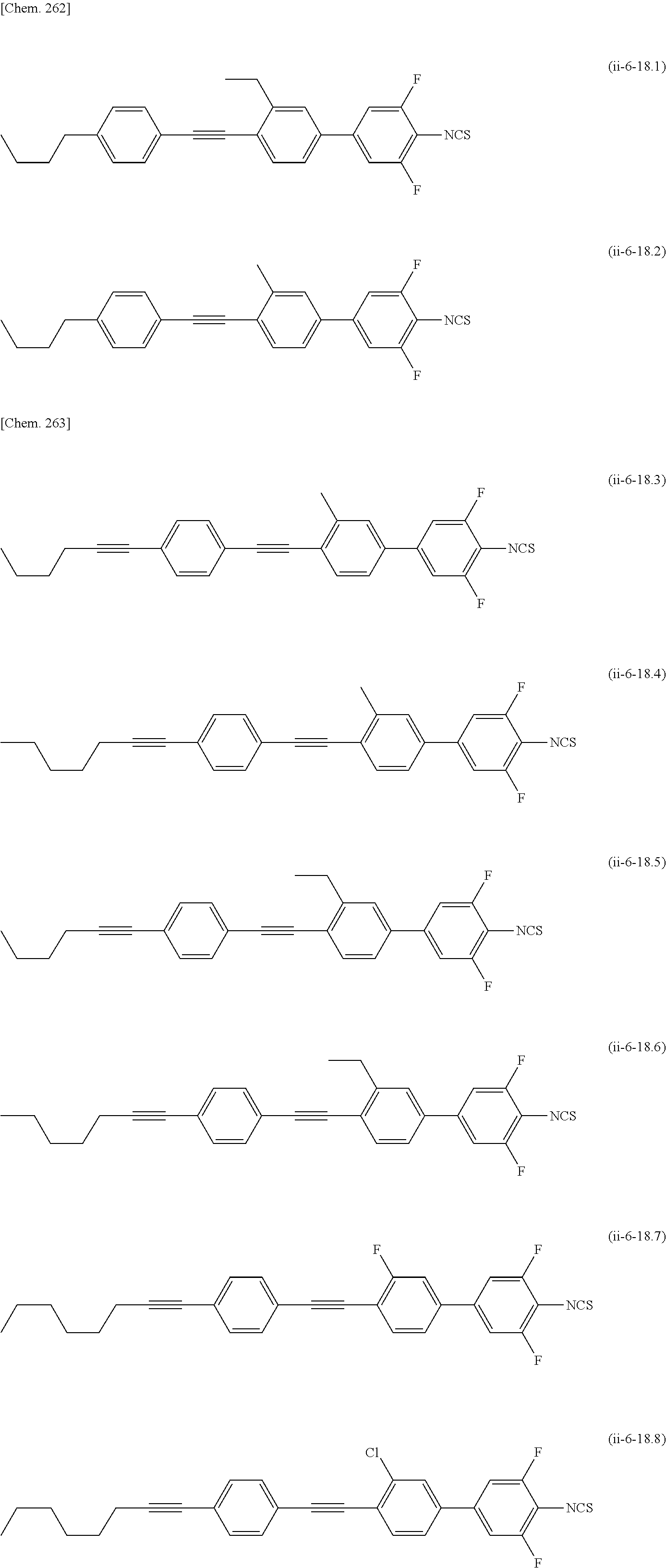

Specific examples of the compounds represented by the general formula (ii-6-19) include the compounds represented by the following structural formulae (ii-6-19.1) to (ii-6-19.4):

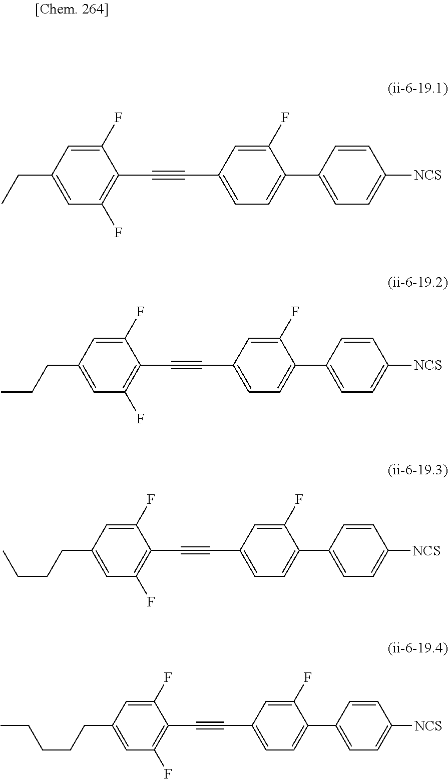

Specific examples of the compounds represented by the general formula (ii-6-20) include the compounds represented by the following structural formulae (ii-6-20.1) to (ii-6-20.4):

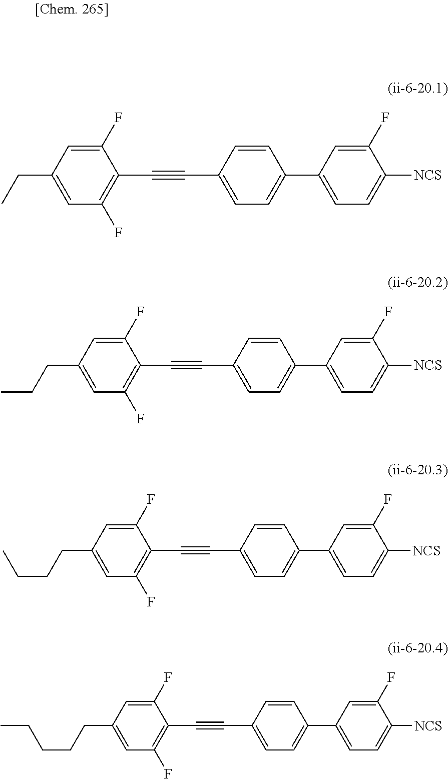

Specific examples of the compounds represented by the general formula (ii-6-21) include the compounds represented by the following structural formulae (ii-6-21.1) to (ii-6-21.4):

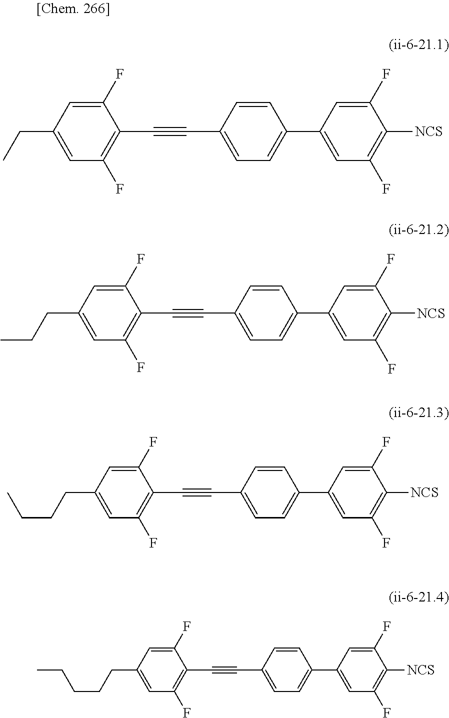

Specific examples of the compounds represented by the general formula (ii-6-22) include the compounds represented by the following structural formulae (ii-6-22.1) to (ii-6-22.14):

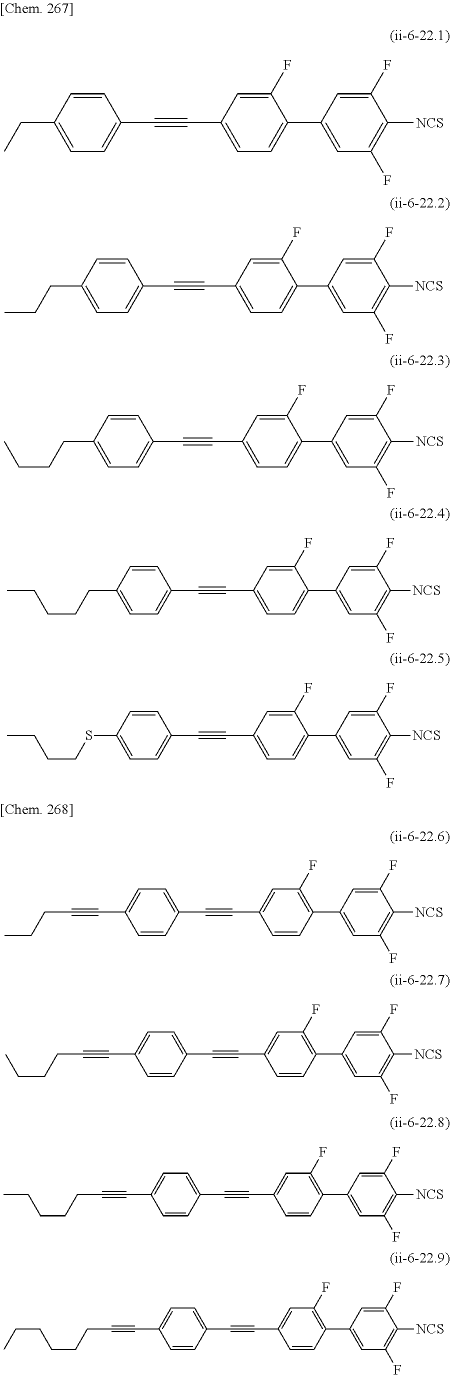

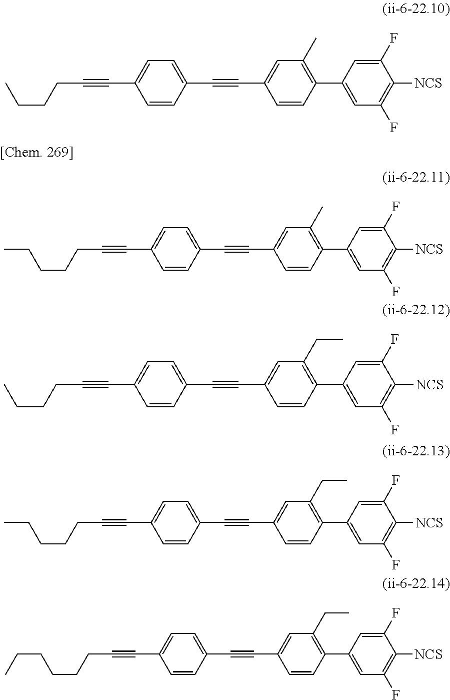

Specific examples of the compounds represented by the general formula (ii-6-23) include the compounds represented by the following structural formulae (ii-6-23.1) to (ii-6-23.10):

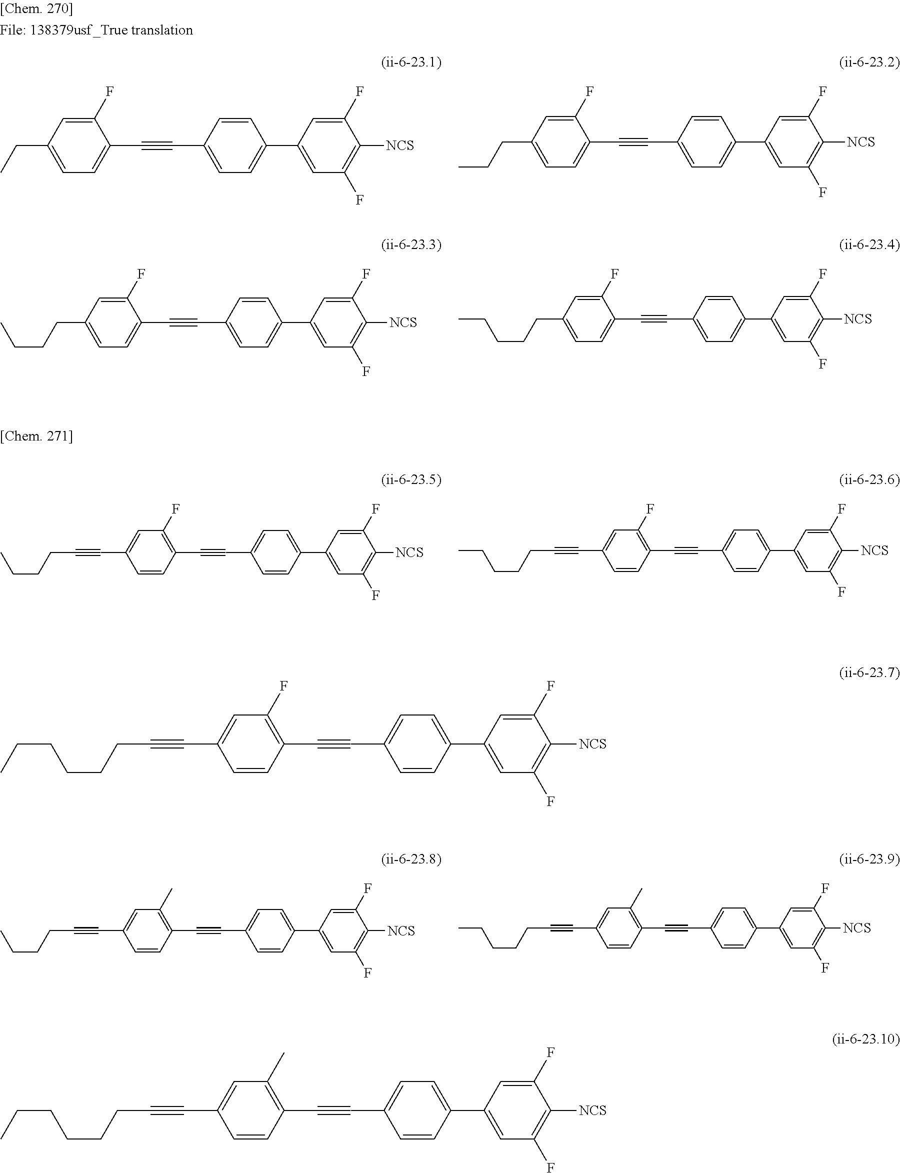

Specific examples of the compounds represented by the general formula (ii-6-24) include the compounds represented by the following structural formulae (ii-6-24.1) and (ii-6-24.2):

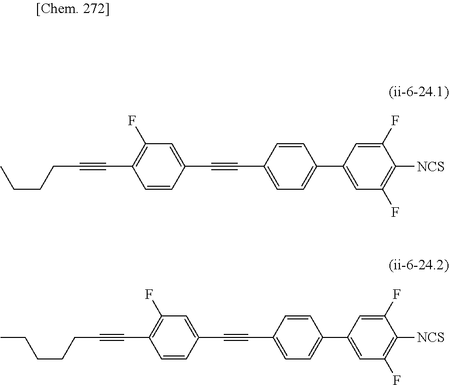

Specific examples of the compounds represented by the general formula (ii-6-25) include the compounds represented by the following structural formulae (ii-6-25.1) to (ii-6-25.5):

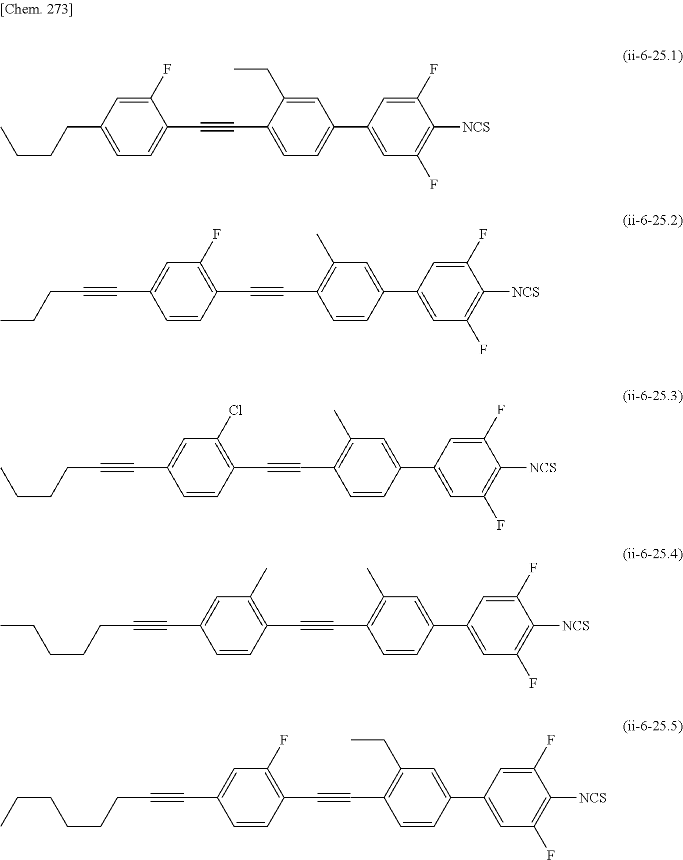

Specific examples of the compounds represented by the general formula (ii-6-26) include the compounds represented by the following structural formulae (ii-6-26.1) and (ii-6-26.2):

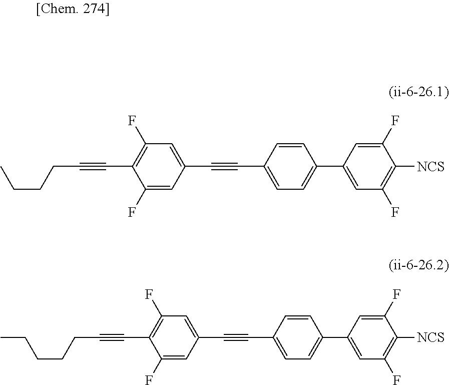

Specific examples of the compounds represented by the general formula (ii-6-27) include the compound represented by the following structural formula (ii-6-27.1):

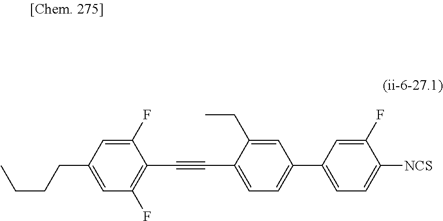

Specific examples of the compounds represented by the general formula (ii-6-28) include the compounds represented by the following structural formulae (ii-6-28.1) to (ii-6-28.5):

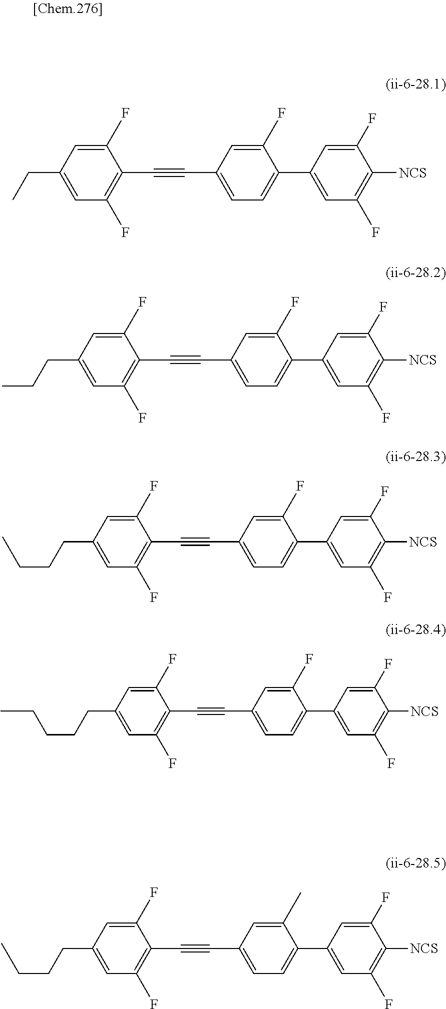

Specific examples of the compounds represented by the general formula (ii-6-29) include the compound represented by the following structural formula (ii-6-29.1):

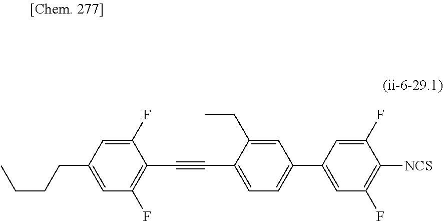

Specific examples of the compounds represented by the general formula (ii-6-30) include the compound represented by the following structural formula (ii-6-30.1):

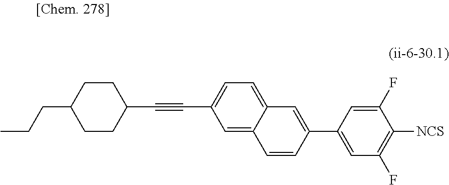

Specific examples of the compounds represented by the general formula (ii-6-31) include the compounds represented by the following structural formulae (ii-6-31.1) to (ii-6-31.5):

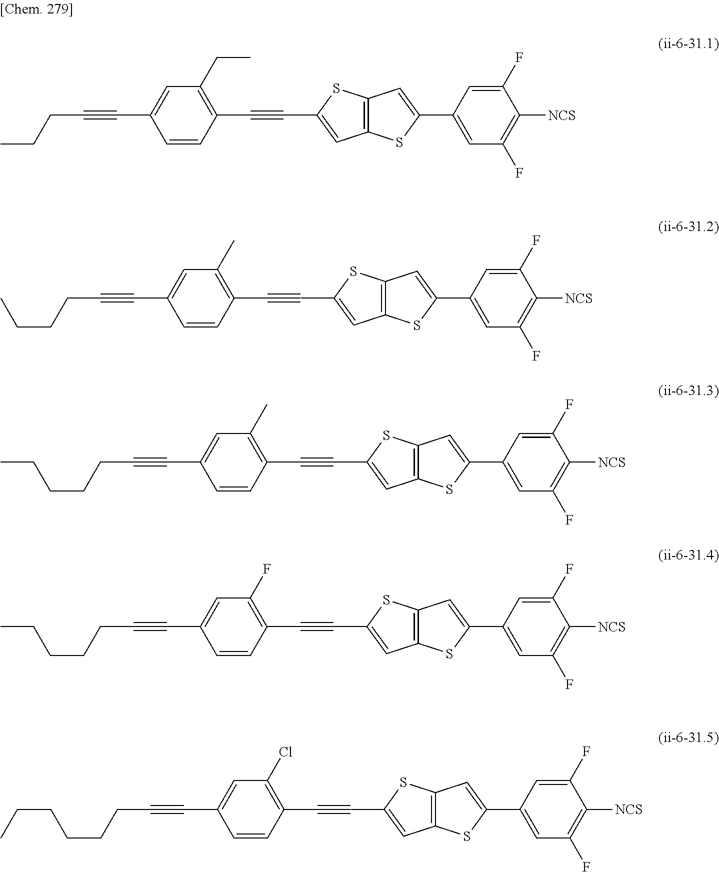

Specific examples of the compounds represented by the general formula (ii-6-32) include the compound represented by the following structural formula (ii-6-32.1):

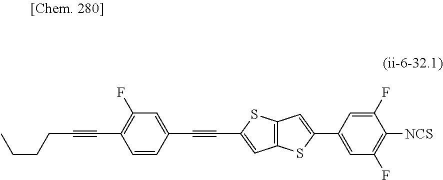

Specific examples of the compounds represented by the general formula (ii-6-33) include the compounds represented by the following structural formulae (ii-6-33.1) to (ii-6-33.4):

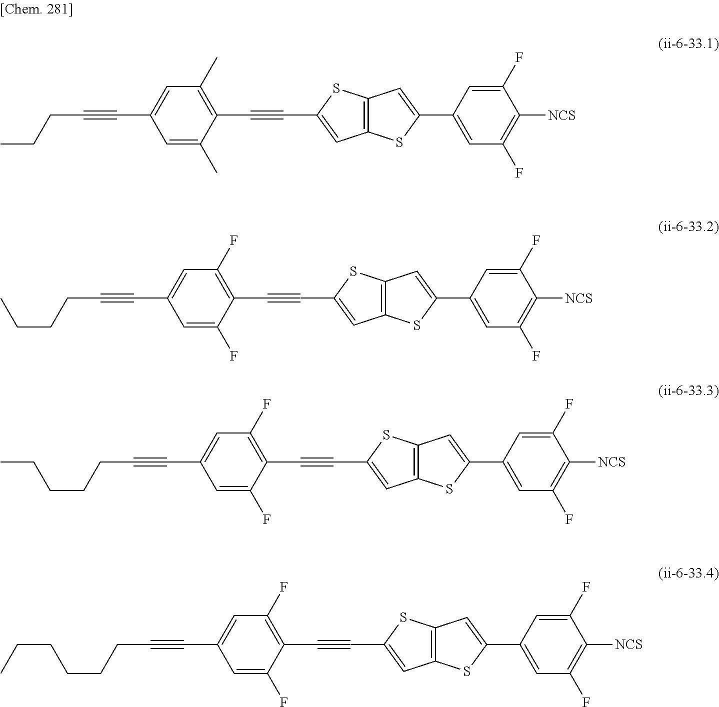

Specific examples of the compounds represented by the general formula (ii-6-34) include the compounds represented by the following structural formulae (ii-6-34.1) to (ii-6-34.3):

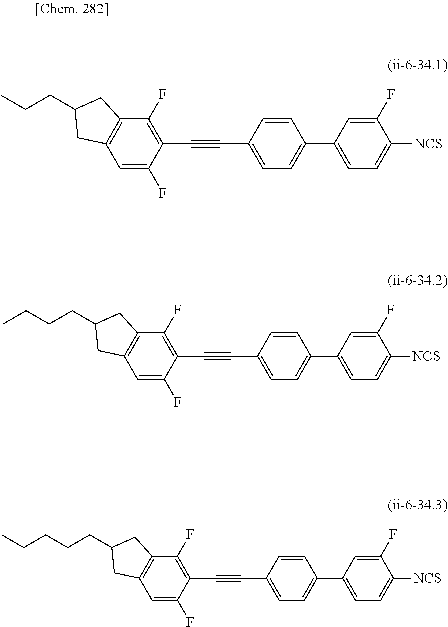

Specific examples of the compounds represented by the general formula (ii-6-35) include the compounds represented by the following structural formulae (ii-6-35.1) to (ii-6-35.4):

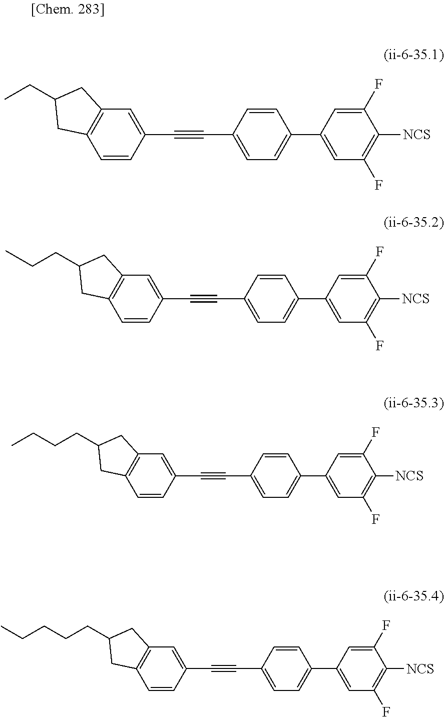

Specific examples of the compounds represented by the general formula (ii-6-36) include the compounds represented by the following structural formulae (ii-6-36.1) to (ii-6-36.3):

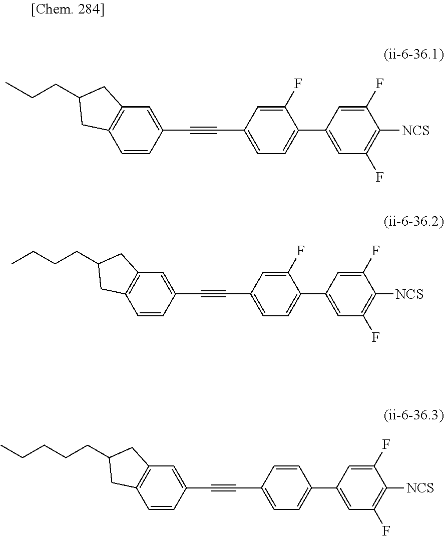

Compounds represented by the general formula (ii-7) are preferably the compounds represented by the following general formula (ii-7-1):

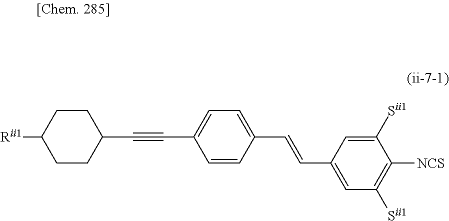

In the general formula (ii-7-1), Rⁱⁱ¹ and Sⁱⁱ¹ each independently denote the same as Rⁱⁱ¹ and Sⁱⁱ¹ in the general formula (ii).

Specific examples of the compounds represented by the general formula (ii-7-1) include the compound represented by the following structural formula (ii-7-1.1):

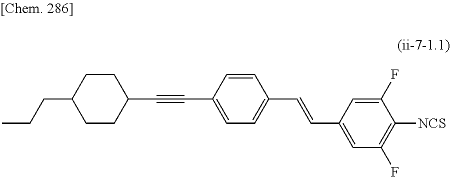

Compounds represented by the general formula (ii-8) are preferably the compounds represented by the following general formulae (ii-8-1) and (ii-8-2):

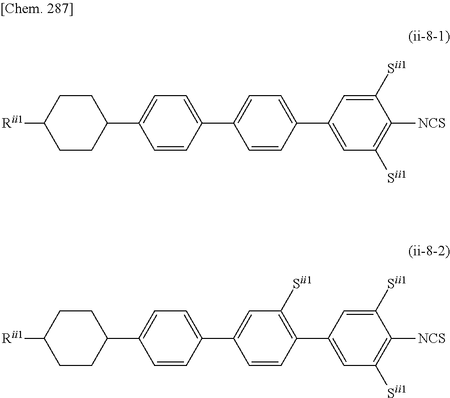

In the general formulae (ii-8-1) and (ii-8-2), Rⁱⁱ¹ and Sⁱⁱ¹ each independently denote the same as Rⁱⁱ¹ and Sⁱⁱ¹ in the general formula (ii).

Specific examples of the compounds represented by the general formula (ii-8-1) include the compounds represented by the following structural formulae (ii-8-1.1) to (ii-8-1.3):

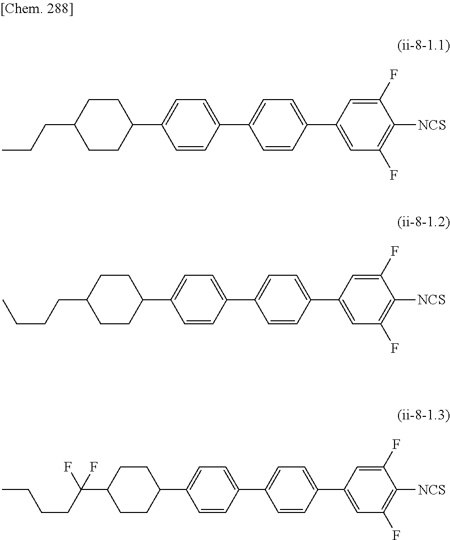

Specific examples of the compounds represented by the general formula (ii-8-2) include the compounds represented by the following structural formulae (ii-8-2.1) to (ii-8-2.6):

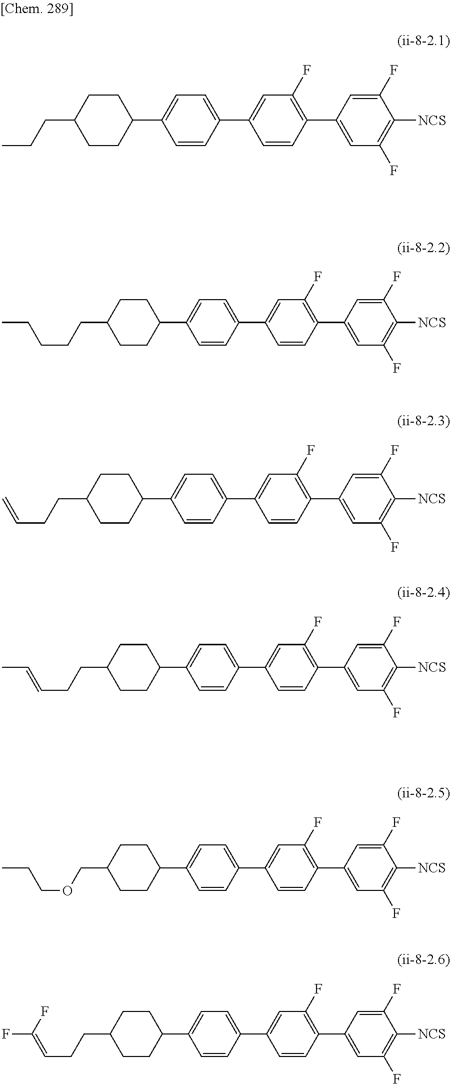

The number of types of compounds represented by the following formulae used in liquid crystal compositions is one or two or more, preferably 1 to 20, or preferably 2 to 15: the general formula (ii), the general formulae (ii-1) to (ii-8), the general formulae (ii-1-1) to (ii-1-4), the general formulae (ii-2-1) to (ii-2-14), the general formulae (ii-3-1) to (ii-3-22), the general formulae (ii-4-1) to (ii-4-30), the general formulae (ii-5-1) to (ii-5-8), the general formulae (ii-6-1) to (ii-6-36), the general formula (ii-7-1), the general formulae (ii-8-1) and (ii-8-2), the structural formulae (ii-1-1.1) to (ii-1-1.4), the structural formulae (ii-1-2.1) to (ii-1-2.6), the structural formulae (ii-1-3.1) to (ii-1-3.3), the structural formulae (ii-1-4.1) to (ii-1-4.3), the structural formulae (ii-2-1.1) to (ii-2-1.4), the structural formulae (ii-2-2.1) to (ii-2-2.10), the structural formulae (ii-2-3.1) to (ii-2-3.3), the structural formulae (ii-2-4.1) to (ii-2-4.8), the structural formulae (ii-2-5.1) to (ii-2-5.4), the structural formulae (ii-2-6.1) to (ii-2-6.4), the structural formulae (ii-2-7.1) to (ii-2-7.3), the structural formulae (ii-2-8.1) to (ii-2-8.3), the structural formulae (ii-2-9.1) to (ii-2-9.4), the structural formulae (ii-2-10.1) to (ii-2-10.4), the structural formulae (ii-2-11.1) to (ii-2-11.3), the structural formulae (ii-2-12.1) to (ii-2-12.3), the structural formulae (ii-2-13.1) to (ii-2-13.3), the structural formula (ii-2-14.1), the structural formulae (ii-3-1.1) to (ii-3-1.4), the structural formulae (ii-3-2.1) to (ii-3-2.4), the structural formulae (ii-3-3.1) to (ii-3-3.7), the structural formulae (ii-3-4.1) to (ii-3-4.5), the structural formulae (ii-3-5.1) to (ii-3-5.7), the structural formulae (ii-3-6.1) to (ii-3-6.3), the structural formulae (ii-3-7.1) to (ii-3-7.6), the structural formulae (ii-3-8.1) to (ii-3-8.3), the structural formulae (ii-3-9.1) to (ii-3-9.4), the structural formulae (ii-3-10.1) to (ii-3-10.3), the structural formulae (ii-3-11.1) to (ii-3-11.3), the structural formulae (ii-3-12.1) to (ii-3-12.3), the structural formulae (ii-3-13.1) to (ii-3-13.4), the structural formulae (ii-3-14.1) to (ii-3-14.3), the structural formulae (ii-3-15.1) to (ii-3-15.3), the structural formulae (ii-3-16.1) to (ii-3-16.3), the structural formulae (ii-3-17.1) to (ii-3-17.3), the structural formulae (ii-3-18.1) to (ii-3-18.3), the structural formulae (ii-3-19.1) to (ii-3-19.3), the structural formulae (ii-3-20.1) to (ii-3-20.3), the structural formulae (ii-3-21.1) to (ii-3-21.4), the structural formulae (ii-3-22.1) to (ii-3-22.3), the structural formulae (ii-4-1.1) to (ii-4-1.4), the structural formulae (ii-4-2.1) to (ii-4-2.4), the structural formulae (ii-4-3.1) to (ii-4-3.8), the structural formulae (ii-4-4.1) to (ii-4-4.5), the structural formula (ii-4-5.1), the structural formulae (ii-4-6.1) to (ii-4-6.5), the structural formula (ii-4-7.1), the structural formulae (ii-4-8.1) to (ii-4-8.4), the structural formulae (ii-4-9.1) to (ii-4-9.4), the structural formulae (ii-4-10.1) to (ii-4-10.4), the structural formulae (ii-4-11.1) to (ii-4-11.5), the structural formulae (ii-4-12.1) to (ii-4-12.5), the structural formulae (ii-4-13.1) to (ii-4-13.4), the structural formulae (ii-4-14.1) to (ii-4-14.4), the structural formulae (ii-4-15.1) to (ii-4-15.6), the structural formulae (ii-4-16.1) to (ii-4-16.3), the structural formulae (ii-4-17.1) to (ii-4-17.3), the structural formulae (ii-4-18.1) to (ii-4-18.4), the structural formulae (ii-4-19.1) to (ii-4-19.8), the structural formulae (ii-4-20.1) to (ii-4-20.4), the structural formulae (ii-4-21.1) to (ii-4-21.4), the structural formulae (ii-4-22.1) to (ii-4-22.3), the structural formulae (ii-4-23.1) to (ii-4-23.3), the structural formulae (ii-4-24.1) to (ii-4-24.3), the structural formulae (ii-4-25.1) to (ii-4-25.3), the structural formulae (ii-4-26.1) to (ii-4-26.3), the structural formulae (ii-4-27.1) and (ii-4-27.2), the structural formulae (ii-4-28.1) and (ii-4-28.2), the structural formulae (ii-4-29.1) to (ii-4-29.4), the structural formulae (ii-4-30.1) and (ii-4-30.2), the structural formulae (ii-5-1.1) to (ii-5-1.4), the structural formulae (ii-5-2.1) to (ii-5-2.4), the structural formula (ii-5-3.1), the structural formulae (ii-5-4.1) to (ii-5-4.3), the structural formulae (ii-5-5.1) to (ii-5-5.3), the structural formulae (ii-5-6.1) to (ii-5-6.3), the structural formulae (ii-5-7.1) to (ii-5-7.3), the structural formulae (ii-5-8.1) to (ii-5-8.3), the structural formulae (ii-6-1.1) to (ii-6-1.4), the structural formulae (ii-6-2.1) to (ii-6-2.4), the structural formulae (ii-6-3.1) to (ii-6-3.8), the structural formulae (ii-6-4.1) to (ii-6-4.4), the structural formulae (ii-6-5.1) to (ii-6-5.4), the structural formulae (ii-6-6.1) and (ii-6-6.2), the structural formulae (ii-6-7.1) to (ii-6-7.8), the structural formulae (ii-6-8.1) to (ii-6-8.9), the structural formulae (ii-6-9.1) to (ii-6-9.4), the structural formulae (ii-6-10.1) to (ii-6-10.4), the structural formulae (ii-6-11.1) to (ii-6-11.4), the structural formulae (ii-6-12.1) to (ii-6-12.4), the structural formulae (ii-6-13.1) to (ii-6-13.20), the structural formulae (ii-6-14.1) to (ii-6-14.4), the structural formula (ii-6-15.1), the structural formulae (ii-6-16.1) to (ii-6-16.5), the structural formulae (ii-6-17.1) to (ii-6-17.4), the structural formulae (ii-6-18.1) to (ii-6-18.8), the structural formulae (ii-6-19.1) to (ii-6-19.4), the structural formulae (ii-6-20.1) to (ii-6-20.4), the structural formulae (ii-6-21.1) to (ii-6-21.4), the structural formulae (ii-6-22.1) to (ii-6-22.14), the structural formulae (ii-6-23.1) to (ii-6-23.10), the structural formulae (ii-6-24.1) and (ii-6-24.2), the structural formulae (ii-6-25.1) to (ii-6-25.5), the structural formulae (ii-6-26.1) and (ii-6-26.2), the structural formula (ii-6-27.1), the structural formulae (ii-6-28.1) to (ii-6-28.5), the structural formula (ii-6-29.1), the structural formula (ii-6-30.1), the structural formulae (ii-6-31.1) to (ii-6-31.5), the structural formula (ii-6-32.1), the structural formulae (ii-6-33.1) to (ii-6-33.4), the structural formulae (ii-6-34.1) to (ii-6-34.3), the structural formulae (ii-6-35.1) to (ii-6-35.4), the structural formulae (ii-6-36.1) to (ii-6-36.3), the structural formula (ii-7-1.1), the structural formulae (ii-8-1.1) to (ii-8-1.3), or the structural formulae (ii-8-2.1) to (ii-8-2.6).

The lower limit of the total amount of the compounds represented by the following formulae per 100% by mass of the liquid crystal composition is preferably 18 by mass or more, preferably 5% by mass or more, preferably 10% by mass or more, preferably 15% by mass or more, preferably 20% by mass or more, preferably 25% by mass or more, preferably 308 by mass or more, preferably 35% by mass or more, preferably 40% by mass or more, preferably 45% by mass or more, preferably 75% by mass or more, preferably 80% by mass or more, or preferably 85% by mass or more: the general formula (ii), the general formulae (ii-1) to (ii-8), the general formulae (ii-1-1) to (ii-1-4), the general formulae (ii-2-1) to (ii-2-14), the general formulae (ii-3-1) to (ii-3-22), the general formulae (ii-4-1) to (ii-4-30), the general formulae (ii-5-1) to (ii-5-8), the general formulae (ii-6-1) to (ii-6-36), the general formula (ii-7-1), the general formulae (ii-8-1) and (ii-8-2), the structural formulae (ii-1-1.1) to (ii-1-1.4), the structural formulae (ii-1-2.1) to (ii-1-2.6), the structural formulae (ii-1-3.1) to (ii-1-3.3), the structural formulae (ii-1-4.1) to (ii-1-4.3), the structural formulae (ii-2-1.1) to (ii-2-1.4), the structural formulae (ii-2-2.1) to (ii-2-2.10), the structural formulae (ii-2-3.1) to (ii-2-3.3), the structural formulae (ii-2-4.1) to (ii-2-4.8), the structural formulae (ii-2-5.1) to (ii-2-5.4), the structural formulae (ii-2-6.1) to (ii-2-6.4), the structural formulae (ii-2-7.1) to (ii-2-7.3), the structural formulae (ii-2-8.1) to (ii-2-8.3), the structural formulae (ii-2-9.1) to (ii-2-9.4), the structural formulae (ii-2-10.1) to (ii-2-10.4), the structural formulae (ii-2-11.1) to (ii-2-11.3), the structural formulae (ii-2-12.1) to (ii-2-12.3), the structural formulae (ii-2-13.1) to (ii-2-13.3), the structural formula (ii-2-14.1), the structural formulae (ii-3-1.1) to (ii-3-1.4), the structural formulae (ii-3-2.1) to (ii-3-2.4), the structural formulae (ii-3-3.1) to (ii-3-3.7), the structural formulae (ii-3-4.1) to (ii-3-4.5), the structural formulae (ii-3-5.1) to (ii-3-5.7), the structural formulae (ii-3-6.1) to (ii-3-6.3), the structural formulae (ii-3-7.1) to (ii-3-7.6), the structural formulae (ii-3-8.1) to (ii-3-8.3), the structural formulae (ii-3-9.1) to (ii-3-9.4), the structural formulae (ii-3-10.1) to (ii-3-10.3), the structural formulae (ii-3-11.1) to (ii-3-11.3), the structural formulae (ii-3-12.1) to (ii-3-12.3), the structural formulae (ii-3-13.1) to (ii-3-13.4), the structural formulae (ii-3-14.1) to (ii-3-14.3), the structural formulae (ii-3-15.1) to (ii-3-15.3), the structural formulae (ii-3-16.1) to (ii-3-16.3), the structural formulae (ii-3-17.1) to (ii-3-17.3), the structural formulae (ii-3-18.1) to (ii-3-18.3), the structural formulae (ii-3-19.1) to (ii-3-19.3), the structural formulae (ii-3-20.1) to (ii-3-20.3), the structural formulae (ii-3-21.1) to (ii-3-21.4), the structural formulae (ii-3-22.1) to (ii-3-22.3), the structural formulae (ii-4-1.1) to (ii-4-1.4), the structural formulae (ii-4-2.1) to (ii-4-2.4), the structural formulae (ii-4-3.1) to (ii-4-3.8), the structural formulae (ii-4-4.1) to (ii-4-4.5), the structural formula (ii-4-5.1), the structural formulae (ii-4-6.1) to (ii-4-6.5), the structural formula (ii-4-7.1), the structural formulae (ii-4-8.1) to (ii-4-8.4), the structural formulae (ii-4-9.1) to (ii-4-9.4), the structural formulae (ii-4-10.1) to (ii-4-10.4), the structural formulae (ii-4-11.1) to (ii-4-11.5), the structural formulae (ii-4-12.1) to (ii-4-12.5), the structural formulae (ii-4-13.1) to (ii-4-13.4), the structural formulae (ii-4-14.1) to (ii-4-14.4), the structural formulae (ii-4-15.1) to (ii-4-15.6), the structural formulae (ii-4-16.1) to (ii-4-16.3), the structural formulae (ii-4-17.1) to (ii-4-17.3), the structural formulae (ii-4-18.1) to (ii-4-18.4), the structural formulae (ii-4-19.1) to (ii-4-19.8), the structural formulae (ii-4-20.1) to (ii-4-20.4), the structural formulae (ii-4-21.1) to (ii-4-21.4), the structural formulae (ii-4-22.1) to (ii-4-22.3), the structural formulae (ii-4-23.1) to (ii-4-23.3), the structural formulae (ii-4-24.1) to (ii-4-24.3), the structural formulae (ii-4-25.1) to (ii-4-25.3), the structural formulae (ii-4-26.1) to (ii-4-26.3), the structural formulae (ii-4-27.1) and (ii-4-27.2), the structural formulae (ii-4-28.1) and (ii-4-28.2), the structural formulae (ii-4-29.1) to (ii-4-29.4), the structural formulae (ii-4-30.1) and (ii-4-30.2), the structural formulae (ii-5-1.1) to (ii-5-1.4), the structural formulae (ii-5-2.1) to (ii-5-2.4), the structural formula (ii-5-3.1), the structural formulae (ii-5-4.1) to (ii-5-4.3), the structural formulae (ii-5-5.1) to (ii-5-5.3), the structural formulae (ii-5-6.1) to (ii-5-6.3), the structural formulae (ii-5-7.1) to (ii-5-7.3), the structural formulae (ii-5-8.1) to (ii-5-8.3), the structural formulae (ii-6-1.1) to (ii-6-1.4), the structural formulae (ii-6-2.1) to (ii-6-2.4), the structural formulae (ii-6-3.1) to (ii-6-3.8), the structural formulae (ii-6-4.1) to (ii-6-4.4), the structural formulae (ii-6-5.1) to (ii-6-5.4), the structural formulae (ii-6-6.1) and (ii-6-6.2), the structural formulae (ii-6-7.1) to (ii-6-7.8), the structural formulae (ii-6-8.1) to (ii-6-8.9), the structural formulae (ii-6-9.1) to (ii-6-9.4), the structural formulae (ii-6-10.1) to (ii-6-10.4), the structural formulae (ii-6-11.1) to (ii-6-11.4), the structural formulae (ii-6-12.1) to (ii-6-12.4), the structural formulae (ii-6-13.1) to (ii-6-13.20), the structural formulae (ii-6-14.1) to (ii-6-14.4), the structural formula (ii-6-15.1), the structural formulae (ii-6-16.1) to (ii-6-16.5), the structural formulae (ii-6-17.1) to (ii-6-17.4), the structural formulae (ii-6-18.1) to (ii-6-18.8), the structural formulae (ii-6-19.1) to (ii-6-19.4), the structural formulae (ii-6-20.1) to (ii-6-20.4), the structural formulae (ii-6-21.1) to (ii-6-21.4), the structural formulae (ii-6-22.1) to (ii-6-22.14), the structural formulae (ii-6-23.1) to (ii-6-23.10), the structural formulae (ii-6-24.1) and (ii-6-24.2), the structural formulae (ii-6-25.1) to (ii-6-25.5), the structural formulae (ii-6-26.1) and (ii-6-26.2), the structural formula (ii-6-27.1), the structural formulae (ii-6-28.1) to (ii-6-28.5), the structural formula (ii-6-29.1), the structural formula (ii-6-30.1), the structural formulae (ii-6-31.1) to (ii-6-31.5), the structural formula (ii-6-32.1), the structural formulae (ii-6-33.1) to (ii-6-33.4), the structural formulae (ii-6-34.1) to (ii-6-34.3), the structural formulae (ii-6-35.1) to (ii-6-35.4), the structural formulae (ii-6-36.1) to (ii-6-36.3), the structural formula (ii-7-1.1), the structural formulae (ii-8-1.1) to (ii-8-1.3), or the structural formulae (ii-8-2.1) to (ii-8-2.6).

The upper limit of the total amount of the compounds represented by the following formulae per 100% by mass of the liquid crystal composition is preferably 95% by mass or less, preferably 90% by mass or less, preferably 55% by mass or less, preferably 50% by mass or less, preferably 45% by mass or less, preferably 40% by mass or less, preferably 35% by mass or less, preferably 25% by mass or less, preferably 158 by mass or less, preferably 10% by mass or less, or preferably 58 by mass or less: the general formula (ii), the general formulae (ii-1) to (ii-8), the general formulae (ii-1-1) to (ii-1-4), the general formulae (ii-2-1) to (ii-2-14), the general formulae (ii-3-1) to (ii-3-22), the general formulae (ii-4-1) to (ii-4-30), the general formulae (ii-5-1) to (ii-5-8), the general formulae (ii-6-1) to (ii-6-36), the general formula (ii-7-1), the general formulae (ii-8-1) and (ii-8-2), the structural formulae (ii-1-1.1) to (ii-1-1.4), the structural formulae (ii-1-2.1) to (ii-1-2.6), the structural formulae (ii-1-3.1) to (ii-1-3.3), the structural formulae (ii-1-4.1) to (ii-1-4.3), the structural formulae (ii-2-1.1) to (ii-2-1.4), the structural formulae (ii-2-2.1) to (ii-2-2.10), the structural formulae (ii-2-3.1) to (ii-2-3.3), the structural formulae (ii-2-4.1) to (ii-2-4.8), the structural formulae (ii-2-5.1) to (ii-2-5.4), the structural formulae (ii-2-6.1) to (ii-2-6.4), the structural formulae (ii-2-7.1) to (ii-2-7.3), the structural formulae (ii-2-8.1) to (ii-2-8.3), the structural formulae (ii-2-9.1) to (ii-2-9.4), the structural formulae (ii-2-10.1) to (ii-2-10.4), the structural formulae (ii-2-11.1) to (ii-2-11.3), the structural formulae (ii-2-12.1) to (ii-2-12.3), the structural formulae (ii-2-13.1) to (ii-2-13.3), the structural formula (ii-2-14.1), the structural formulae (ii-3-1.1) to (ii-3-1.4), the structural formulae (ii-3-2.1) to (ii-3-2.4), the structural formulae (ii-3-3.1) to (ii-3-3.7), the structural formulae (ii-3-4.1) to (ii-3-4.5), the structural formulae (ii-3-5.1) to (ii-3-5.7), the structural formulae (ii-3-6.1) to (ii-3-6.3), the structural formulae (ii-3-7.1) to (ii-3-7.6), the structural formulae (ii-3-8.1) to (ii-3-8.3), the structural formulae (ii-3-9.1) to (ii-3-9.4), the structural formulae (ii-3-10.1) to (ii-3-10.3), the structural formulae (ii-3-11.1) to (ii-3-11.3), the structural formulae (ii-3-12.1) to (ii-3-12.3), the structural formulae (ii-3-13.1) to (ii-3-13.4), the structural formulae (ii-3-14.1) to (ii-3-14.3), the structural formulae (ii-3-15.1) to (ii-3-15.3), the structural formulae (ii-3-16.1) to (ii-3-16.3), the structural formulae (ii-3-17.1) to (ii-3-17.3), the structural formulae (ii-3-18.1) to (ii-3-18.3), the structural formulae (ii-3-19.1) to (ii-3-19.3), the structural formulae (ii-3-20.1) to (ii-3-20.3), the structural formulae (ii-3-21.1) to (ii-3-21.4), the structural formulae (ii-3-22.1) to (ii-3-22.3), the structural formulae (ii-4-1.1) to (ii-4-1.4), the structural formulae (ii-4-2.1) to (ii-4-2.4), the structural formulae (ii-4-3.1) to (ii-4-3.8), the structural formulae (ii-4-4.1) to (ii-4-4.5), the structural formula (ii-4-5.1), the structural formulae (ii-4-6.1) to (ii-4-6.5), the structural formula (ii-4-7.1), the structural formulae (ii-4-8.1) to (ii-4-8.4), the structural formulae (ii-4-9.1) to (ii-4-9.4), the structural formulae (ii-4-10.1) to (ii-4-10.4), the structural formulae (ii-4-11.1) to (ii-4-11.5), the structural formulae (ii-4-12.1) to (ii-4-12.5), the structural formulae (ii-4-13.1) to (ii-4-13.4), the structural formulae (ii-4-14.1) to (ii-4-14.4), the structural formulae (ii-4-15.1) to (ii-4-15.6), the structural formulae (ii-4-16.1) to (ii-4-16.3), the structural formulae (ii-4-17.1) to (ii-4-17.3), the structural formulae (ii-4-18.1) to (ii-4-18.4), the structural formulae (ii-4-19.1) to (ii-4-19.8), the structural formulae (ii-4-20.1) to (ii-4-20.4), the structural formulae (ii-4-21.1) to (ii-4-21.4), the structural formulae (ii-4-22.1) to (ii-4-22.3), the structural formulae (ii-4-23.1) to (ii-4-23.3), the structural formulae (ii-4-24.1) to (ii-4-24.3), the structural formulae (ii-4-25.1) to (ii-4-25.3), the structural formulae (ii-4-26.1) to (ii-4-26.3), the structural formulae (ii-4-27.1) and (ii-4-27.2), the structural formulae (ii-4-28.1) and (ii-4-28.2), the structural formulae (ii-4-29.1) to (ii-4-29.4), the structural formulae (ii-4-30.1) and (ii-4-30.2), the structural formulae (ii-5-1.1) to (ii-5-1.4), the structural formulae (ii-5-2.1) to (ii-5-2.4), the structural formula (ii-5-3.1), the structural formulae (ii-5-4.1) to (ii-5-4.3), the structural formulae (ii-5-5.1) to (ii-5-5.3), the structural formulae (ii-5-6.1) to (ii-5-6.3), the structural formulae (ii-5-7.1) to (ii-5-7.3), the structural formulae (ii-5-8.1) to (ii-5-8.3), the structural formulae (ii-6-1.1) to (ii-6-1.4), the structural formulae (ii-6-2.1) to (ii-6-2.4), the structural formulae (ii-6-3.1) to (ii-6-3.8), the structural formulae (ii-6-4.1) to (ii-6-4.4), the structural formulae (ii-6-5.1) to (ii-6-5.4), the structural formulae (ii-6-6.1) and (ii-6-6.2), the structural formulae (ii-6-7.1) to (ii-6-7.8), the structural formulae (ii-6-8.1) to (ii-6-8.9), the structural formulae (ii-6-9.1) to (ii-6-9.4), the structural formulae (ii-6-10.1) to (ii-6-10.4), the structural formulae (ii-6-11.1) to (ii-6-11.4), the structural formulae (ii-6-12.1) to (ii-6-12.4), the structural formulae (ii-6-13.1) to (ii-6-13.20), the structural formulae (ii-6-14.1) to (ii-6-14.4), the structural formula (ii-6-15.1), the structural formulae (ii-6-16.1) to (ii-6-16.5), the structural formulae (ii-6-17.1) to (ii-6-17.4), the structural formulae (ii-6-18.1) to (ii-6-18.8), the structural formulae (ii-6-19.1) to (ii-6-19.4), the structural formulae (ii-6-20.1) to (ii-6-20.4), the structural formulae (ii-6-21.1) to (ii-6-21.4), the structural formulae (ii-6-22.1) to (ii-6-22.14), the structural formulae (ii-6-23.1) to (ii-6-23.10), the structural formulae (ii-6-24.1) and (ii-6-24.2), the structural formulae (ii-6-25.1) to (ii-6-25.5), the structural formulae (ii-6-26.1) and (ii-6-26.2), the structural formula (ii-6-27.1), the structural formulae (ii-6-28.1) to (ii-6-28.5), the structural formula (ii-6-29.1), the structural formula (ii-6-30.1), the structural formulae (ii-6-31.1) to (ii-6-31.5), the structural formula (ii-6-32.1), the structural formulae (ii-6-33.1) to (ii-6-33.4), the structural formulae (ii-6-34.1) to (ii-6-34.3), the structural formulae (ii-6-35.1) to (ii-6-35.4), the structural formulae (ii-6-36.1) to (ii-6-36.3), the structural formula (ii-7-1.1), the structural formulae (ii-8-1.1) to (ii-8-1.3), or the structural formulae (ii-8-2.1) to (ii-8-2.6).

From the perspective of solubility, Δn, and/or Δεᵣ, the total content of the compounds represented by the following formulae per 100% by mass of the liquid crystal composition preferably ranges from 1% to 95% by mass, preferably 5% to 90% by mass, preferably 58 to 55% by mass, or preferably 10% to 45% by mass: the general formula (ii), the general formulae (ii-1) to (ii-8), the general formulae (ii-1-1) to (ii-1-4), the general formulae (ii-2-1) to (ii-2-14), the general formulae (ii-3-1) to (ii-3-22), the general formulae (ii-4-1) to (ii-4-30), the general formulae (ii-5-1) to (ii-5-8), the general formulae (ii-6-1) to (ii-6-36), the general formula (ii-7-1), the general formulae (ii-8-1) and (ii-8-2), the structural formulae (ii-1-1.1) to (ii-1-1.4), the structural formulae (ii-1-2.1) to (ii-1-2.6), the structural formulae (ii-1-3.1) to (ii-1-3.3), the structural formulae (ii-1-4.1) to (ii-1-4.3), the structural formulae (ii-2-1.1) to (ii-2-1.4), the structural formulae (ii-2-2.1) to (ii-2-2.10), the structural formulae (ii-2-3.1) to (ii-2-3.3), the structural formulae (ii-2-4.1) to (ii-2-4.8), the structural formulae (ii-2-5.1) to (ii-2-5.4), the structural formulae (ii-2-6.1) to (ii-2-6.4), the structural formulae (ii-2-7.1) to (ii-2-7.3), the structural formulae (ii-2-8.1) to (ii-2-8.3), the structural formulae (ii-2-9.1) to (ii-2-9.4), the structural formulae (ii-2-10.1) to (ii-2-10.4), the structural formulae (ii-2-11.1) to (ii-2-11.3), the structural formulae (ii-2-12.1) to (ii-2-12.3), the structural formulae (ii-2-13.1) to (ii-2-13.3), the structural formula (ii-2-14.1), the structural formulae (ii-3-1.1) to (ii-3-1.4), the structural formulae (ii-3-2.1) to (ii-3-2.4), the structural formulae (ii-3-3.1) to (ii-3-3.7), the structural formulae (ii-3-4.1) to (ii-3-4.5), the structural formulae (ii-3-5.1) to (ii-3-5.7), the structural formulae (ii-3-6.1) to (ii-3-6.3), the structural formulae (ii-3-7.1) to (ii-3-7.6), the structural formulae (ii-3-8.1) to (ii-3-8.3), the structural formulae (ii-3-9.1) to (ii-3-9.4), the structural formulae (ii-3-10.1) to (ii-3-10.3), the structural formulae (ii-3-11.1) to (ii-3-11.3), the structural formulae (ii-3-12.1) to (ii-3-12.3), the structural formulae (ii-3-13.1) to (ii-3-13.4), the structural formulae (ii-3-14.1) to (ii-3-14.3), the structural formulae (ii-3-15.1) to (ii-3-15.3), the structural formulae (ii-3-16.1) to (ii-3-16.3), the structural formulae (ii-3-17.1) to (ii-3-17.3), the structural formulae (ii-3-18.1) to (ii-3-18.3), the structural formulae (ii-3-19.1) to (ii-3-19.3), the structural formulae (ii-3-20.1) to (ii-3-20.3), the structural formulae (ii-3-21.1) to (ii-3-21.4), the structural formulae (ii-3-22.1) to (ii-3-22.3), the structural formulae (ii-4-1.1) to (ii-4-1.4), the structural formulae (ii-4-2.1) to (ii-4-2.4), the structural formulae (ii-4-3.1) to (ii-4-3.8), the structural formulae (ii-4-4.1) to (ii-4-4.5), the structural formula (ii-4-5.1), the structural formulae (ii-4-6.1) to (ii-4-6.5), the structural formula (ii-4-7.1), the structural formulae (ii-4-8.1) to (ii-4-8.4), the structural formulae (ii-4-9.1) to (ii-4-9.4), the structural formulae (ii-4-10.1) to (ii-4-10.4), the structural formulae (ii-4-11.1) to (ii-4-11.5), the structural formulae (ii-4-12.1) to (ii-4-12.5), the structural formulae (ii-4-13.1) to (ii-4-13.4), the structural formulae (ii-4-14.1) to (ii-4-14.4), the structural formulae (ii-4-15.1) to (ii-4-15.6), the structural formulae (ii-4-16.1) to (ii-4-16.3), the structural formulae (ii-4-17.1) to (ii-4-17.3), the structural formulae (ii-4-18.1) to (ii-4-18.4), the structural formulae (ii-4-19.1) to (ii-4-19.8), the structural formulae (ii-4-20.1) to (ii-4-20.4), the structural formulae (ii-4-21.1) to (ii-4-21.4), the structural formulae (ii-4-22.1) to (ii-4-22.3), the structural formulae (ii-4-23.1) to (ii-4-23.3), the structural formulae (ii-4-24.1) to (ii-4-24.3), the structural formulae (ii-4-25.1) to (ii-4-25.3), the structural formulae (ii-4-26.1) to (ii-4-26.3), the structural formulae (ii-4-27.1) and (ii-4-27.2), the structural formulae (ii-4-28.1) and (ii-4-28.2), the structural formulae (ii-4-29.1) to (ii-4-29.4), the structural formulae (ii-4-30.1) and (ii-4-30.2), the structural formulae (ii-5-1.1) to (ii-5-1.4), the structural formulae (ii-5-2.1) to (ii-5-2.4), the structural formula (ii-5-3.1), the structural formulae (ii-5-4.1) to (ii-5-4.3), the structural formulae (ii-5-5.1) to (ii-5-5.3), the structural formulae (ii-5-6.1) to (ii-5-6.3), the structural formulae (ii-5-7.1) to (ii-5-7.3), the structural formulae (ii-5-8.1) to (ii-5-8.3), the structural formulae (ii-6-1.1) to (ii-6-1.4), the structural formulae (ii-6-2.1) to (ii-6-2.4), the structural formulae (ii-6-3.1) to (ii-6-3.8), the structural formulae (ii-6-4.1) to (ii-6-4.4), the structural formulae (ii-6-5.1) to (ii-6-5.4), the structural formulae (ii-6-6.1) and (ii-6-6.2), the structural formulae (ii-6-7.1) to (ii-6-7.8), the structural formulae (ii-6-8.1) to (ii-6-8.9), the structural formulae (ii-6-9.1) to (ii-6-9.4), the structural formulae (ii-6-10.1) to (ii-6-10.4), the structural formulae (ii-6-11.1) to (ii-6-11.4), the structural formulae (ii-6-12.1) to (ii-6-12.4), the structural formulae (ii-6-13.1) to (ii-6-13.20), the structural formulae (ii-6-14.1) to (ii-6-14.4), the structural formula (ii-6-15.1), the structural formulae (ii-6-16.1) to (ii-6-16.5), the structural formulae (ii-6-17.1) to (ii-6-17.4), the structural formulae (ii-6-18.1) to (ii-6-18.8), the structural formulae (ii-6-19.1) to (ii-6-19.4), the structural formulae (ii-6-20.1) to (ii-6-20.4), the structural formulae (ii-6-21.1) to (ii-6-21.4), the structural formulae (ii-6-22.1) to (ii-6-22.14), the structural formulae (ii-6-23.1) to (ii-6-23.10), the structural formulae (ii-6-24.1) and (ii-6-24.2), the structural formulae (ii-6-25.1) to (ii-6-25.5), the structural formulae (ii-6-26.1) and (ii-6-26.2), the structural formula (ii-6-27.1), the structural formulae (ii-6-28.1) to (ii-6-28.5), the structural formula (ii-6-29.1), the structural formula (ii-6-30.1), the structural formulae (ii-6-31.1) to (ii-6-31.5), the structural formula (ii-6-32.1), the structural formulae (ii-6-33.1) to (ii-6-33.4), the structural formulae (ii-6-34.1) to (ii-6-34.3), the structural formulae (ii-6-35.1) to (ii-6-35.4), the structural formulae (ii-6-36.1) to (ii-6-36.3), the structural formula (ii-7-1.1), the structural formulae (ii-8-1.1) to (ii-8-1.3), or the structural formulae (ii-8-2.1) to (ii-8-2.6).

The compounds represented by the general formula (ii) (including subordinate concepts) can be synthesized by a known synthesis method.

### (Other Compounds)

From the perspective of Δn and/or Δεᵣ, a liquid crystal composition according to the present invention may further contain one or two or more compounds represented by the following general formula (vt) having at least one —C≡C— as a linking group.

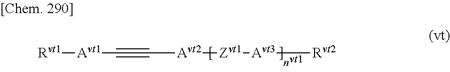

In the general formula (vt), R^{vt1} denotes a hydrogen atom or an alkyl group with 1 to 20 carbon atoms.

The alkyl group with 1 to 20 carbon atoms is a linear, branched, or cyclic alkyl group, preferably a linear alkyl group.

The number of carbon atoms in the alkyl group with 1 to 20 carbon atoms preferably ranges from 2 to 10 or preferably 2 to 6.

One or two or more —CH₂— groups in the alkyl group are each independently optionally substituted with —O—, —S—, —CO—, and/or —CS—.

One or two or more —CH₂—CH₂— groups in the alkyl group are each independently optionally substituted with —CO—O—, —O—CO—, —CO—S—, —S—CO—, —CO—NH—, —NH—CO—, —CH═CH—, —CF═CF—, and/or —C≡C—.

One or two or more hydrogen atoms in the alkyl group are each independently optionally substituted with a halogen atom.

The halogen atom may be a fluorine atom, a chlorine atom, a bromine atom, or an iodine atom.

When the alkyl group is substituted with a predetermined group, oxygen atoms are not directly bonded to each other.

From the perspective of the stability of the compound, sulfur atoms are preferably not directly bonded to each other, and/or an oxygen atom and a sulfur atom are preferably not directly bonded to each other.

For example, R^{vt1} can denote an alkoxy group with 1 to 19 carbon atoms by substituting one —CH₂— in the alkyl group with —O—.

The alkoxy group is a linear, branched, or cyclic alkoxy group, preferably a linear alkoxy group.

The number of carbon atoms in the alkoxy group preferably ranges from 2 to 10 or preferably 2 to 6.

R^{vt1} can denote an alkylsulfanyl group (alkylthio group) with 1 to 19 carbon atoms by substituting one —CH₂— in the alkyl group with —S—.

The alkylsulfanyl group is a linear, branched, or cyclic alkylsulfanyl group, preferably a linear alkylsulfanyl group.

The number of carbon atoms in the alkylsulfanyl group preferably ranges from 1 to 10 or preferably 1 to 6.

R^{vt1} can denote an alkenyl group with 2 to 20 carbon atoms by substituting one or two or more —CH₂—CH₂— groups in the alkyl group with —CH═CH—.

The alkenyl group is a linear, branched, or cyclic alkenyl group, preferably a linear alkenyl group.

The number of carbon atoms in the alkenyl group preferably ranges from 2 to 10 or preferably 2 to 6.

R^{vt1} can denote an alkynyl group with 2 to 20 carbon atoms by substituting one or two or more —CH₂—CH₂— groups in the alkyl group with —C≡C—.

The alkynyl group is a linear, branched, or cyclic alkynyl group, preferably a linear alkynyl group.

The number of carbon atoms in the alkynyl group preferably ranges from 2 to 10 or preferably 2 to 6.

R^{vt1} can denote an alkenyloxy group with 2 to 19 carbon atoms by substituting one —CH₂— in the alkyl group with —O— and substituting one or two or more —CH₂—CH₂— groups with —CH═CH—.

The alkenyloxy group is a linear, branched, or cyclic alkenyloxy group, preferably a linear alkenyloxy group.

The number of carbon atoms in the alkenyloxy group preferably ranges from 2 to 10 or preferably 2 to 6.

R^{vt1} can denote an alkyl halide group with 1 to 20 carbon atoms by substituting one or two or more hydrogen atoms in the alkyl group with a halogen atom.

The alkyl halide group is a linear, branched, or cyclic alkyl halide group, preferably a linear alkyl halide group.

The number of carbon atoms in the alkyl halide group preferably ranges from 2 to 10 or preferably 2 to 6.

R^{vt1} can denote an alkoxy halide group with 1 to 19 carbon atoms by substituting one —CH₂— in the alkyl group with —O— and substituting one or two or more hydrogen atoms in the alkyl group with a halogen atom.

The alkoxy halide group is a linear, branched, or cyclic alkoxy halide group, preferably a linear alkoxy halide group.

The number of carbon atoms in the alkoxy halide group preferably ranges from 2 to 10 or preferably 2 to 6.

Specific examples of the alkyl group with 1 to 20 carbon atoms (including substituted ones) in R^{vt1} include the groups represented by the formulae (R^{vt1}-1) to (R^{vt1}-36):

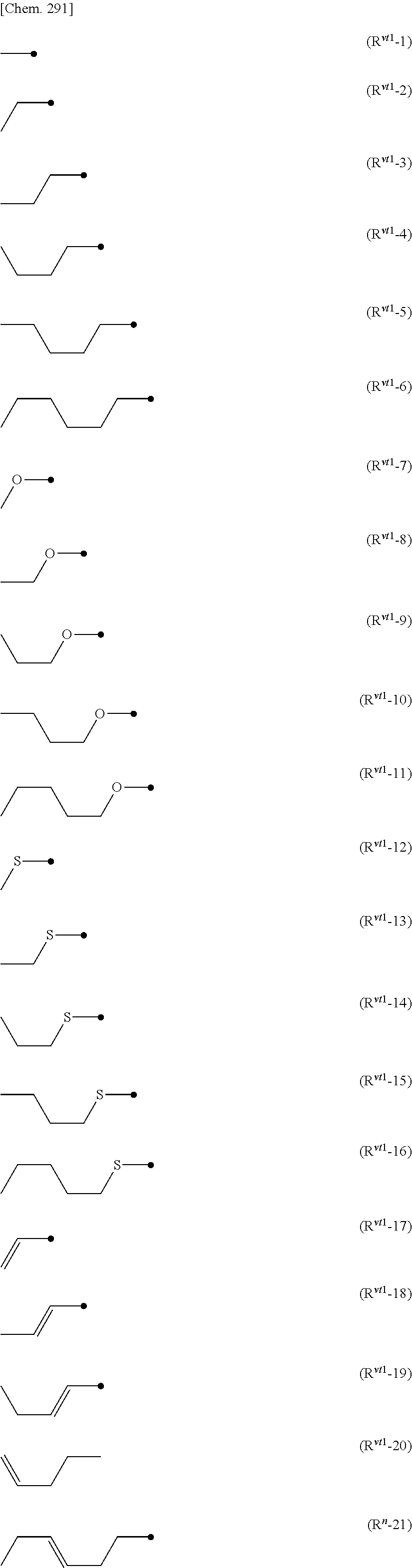

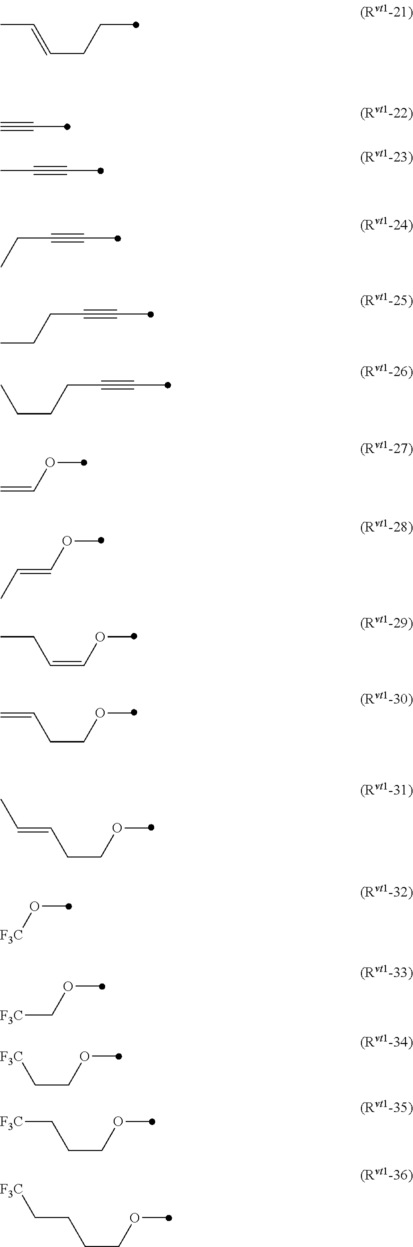

In the formulae (R^{vt1}-1) to (R^{vt1}-36), a black dot represents a bonding arm to A^{vt1}.

R^{vt1} is preferably an alkyl group with 1 to 12 carbon atoms when the reliability of the entire liquid crystal composition is regarded as important, and is preferably an alkenyl group with 2 to 8 carbon atoms when the decrease in viscosity of the entire liquid crystal composition is regarded as important.

When the ring structure to which R^{vt1} is bonded is a phenyl group (aromatic), a linear alkyl group with 1 to 5 carbon atoms, a linear alkoxy group with 1 to 4 carbon atoms, or an alkenyl group with 4 or 5 carbon atoms is preferred. When the ring structure to which R^{vt1} is bonded is a saturated ring structure, such as cyclohexane, pyran, or dioxane, a linear alkyl group with 1 to 5 carbon atoms, a linear alkoxy group with 1 to 4 carbon atoms, or a linear alkenyl group with 2 to 5 carbon atoms is preferred.

To stabilize a nematic phase, in R^{vt1}, the total number of carbon atoms and, if present, oxygen atoms is preferably 5 or less, and a straight chain is preferred.

From the perspective of solubility, R^{vt1} preferably denotes a linear alkyl group with 2 to 6 carbon atoms or a linear alkylsulfanyl group with 1 to 6 carbon atoms.

In the general formula (vt), R^{vt2} denotes any one of a hydrogen atom, a fluorine atom, a chlorine atom, a bromine atom, an iodine atom, a pentafluorosulfanyl group, a nitro group, a cyano group, an isocyano group, an amino group, a hydroxy group, a mercapto group, a methylamino group, a dimethylamino group, a diethylamino group, a diisopropylamino group, a trimethylsilyl group, a dimethylsilyl group, and an alkyl group with 1 to 20 carbon atoms.

The alkyl group with 1 to 20 carbon atoms is a linear, branched, or cyclic alkyl group, preferably a linear alkyl group.

The number of carbon atoms in the alkyl group preferably ranges from 2 to 10 or preferably 2 to 6.

One or two or more —CH₂— groups in the alkyl group are each independently optionally substituted with —O—, —S—, —CO—, and/or —CS—.

One or two or more —CH₂—CH₂— groups in the alkyl group are each independently optionally substituted with —CO—O—, —O—CO—, —CO—S—, —S—CO—, —CO—NH—, —NH—CO—, —CH═CH—, —CF═CF—, and/or —C≡C—.

One or two or more hydrogen atoms in the alkyl group are each independently optionally substituted with a halogen atom.

The halogen atom may be a fluorine atom, a chlorine atom, a bromine atom, or an iodine atom.

When the alkyl group is substituted with a predetermined group, oxygen atoms are not directly bonded to each other.

From the perspective of the stability of the compound, sulfur atoms are preferably not directly bonded to each other, and/or an oxygen atom and a sulfur atom are preferably not directly bonded to each other.

For example, R^{vt2} can denote an alkoxy group with 1 to 19 carbon atoms by substituting one —CH₂— in the alkyl group with —O—.

The alkoxy group is a linear, branched, or cyclic alkoxy group, preferably a linear alkoxy group.

The number of carbon atoms in the alkoxy group preferably ranges from 2 to 10 or preferably 2 to 6.

R^{vt2} can denote an alkylsulfanyl group (alkylthio group) with 1 to 19 carbon atoms by substituting one —CH₂— in the alkyl group with —S—.

The alkylsulfanyl group is a linear, branched, or cyclic alkylsulfanyl group, preferably a linear alkylsulfanyl group.

The number of carbon atoms in the alkylsulfanyl group preferably ranges from 1 to 10 or preferably 1 to 6.

R^{vt2} can denote an alkenyl group with 2 to 20 carbon atoms by substituting one or two or more —CH₂—CH₂— groups in the alkyl group with —CH═CH—.

The alkenyl group is a linear, branched, or cyclic alkenyl group, preferably a linear alkenyl group.

The number of carbon atoms in the alkenyl group preferably ranges from 2 to 10 or preferably 2 to 6.

R^{vt2} can denote an alkynyl group with 2 to 20 carbon atoms by substituting one or two or more —CH₂—CH₂— groups in the alkyl group with —C≡C—.

The alkynyl group is a linear, branched, or cyclic alkynyl group, preferably a linear alkynyl group.

The number of carbon atoms in the alkynyl group preferably ranges from 2 to 10 or preferably 2 to 6.

R^{vt2} can denote an alkenyloxy group with 2 to 19 carbon atoms by substituting one —CH₂— in the alkyl group with —O— and substituting one or two or more —CH₂—CH₂— groups with —CH═CH—.

The alkenyloxy group is a linear, branched, or cyclic alkenyloxy group, preferably a linear alkenyloxy group.

The number of carbon atoms in the alkenyloxy group preferably ranges from 2 to 10 or preferably 2 to 6.

R^{vt2} can denote an alkyl halide group with 1 to 20 carbon atoms by substituting one or two or more hydrogen atoms in the alkyl group with a halogen atom.

The alkyl halide group is a linear, branched, or cyclic alkyl halide group, preferably a linear alkyl halide group.

The number of carbon atoms in the alkyl halide group preferably ranges from 2 to 10 or preferably 2 to 6.

R^{vt2} can denote an alkoxy halide group with 1 to 19 carbon atoms by substituting one —CH₂— in the alkyl group with —O— and substituting one or two or more hydrogen atoms in the alkyl group with a halogen atom.

The alkoxy halide group is a linear, branched, or cyclic alkoxy halide group, preferably a linear alkoxy halide group.

The number of carbon atoms in the alkoxy halide group preferably ranges from 2 to 10 or preferably 2 to 6.

Specific examples of the alkyl group with 1 to 20 carbon atoms (including substituted ones) in R^{vt2} include the groups represented by the formulae (R^{vt2}-1) to (R^{vt2}-36):

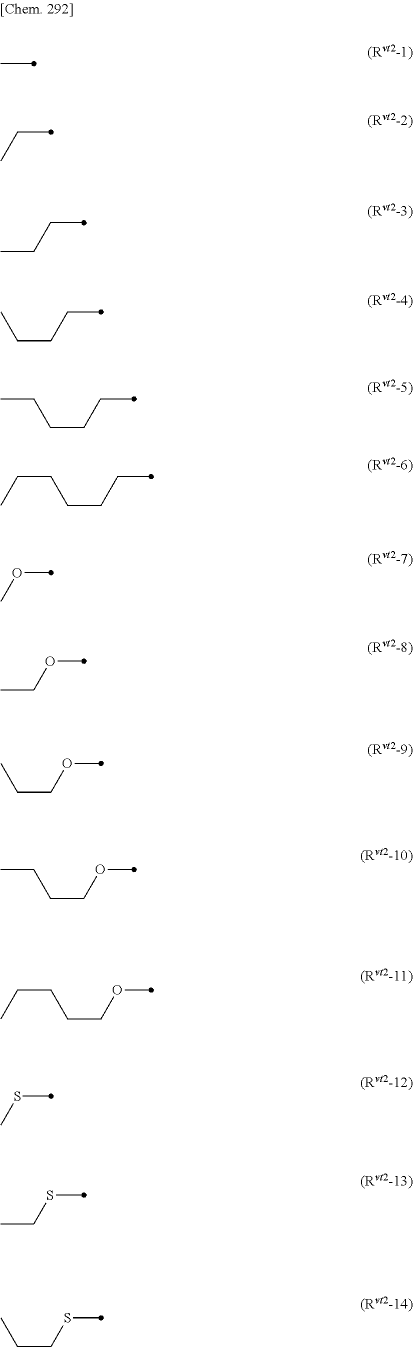

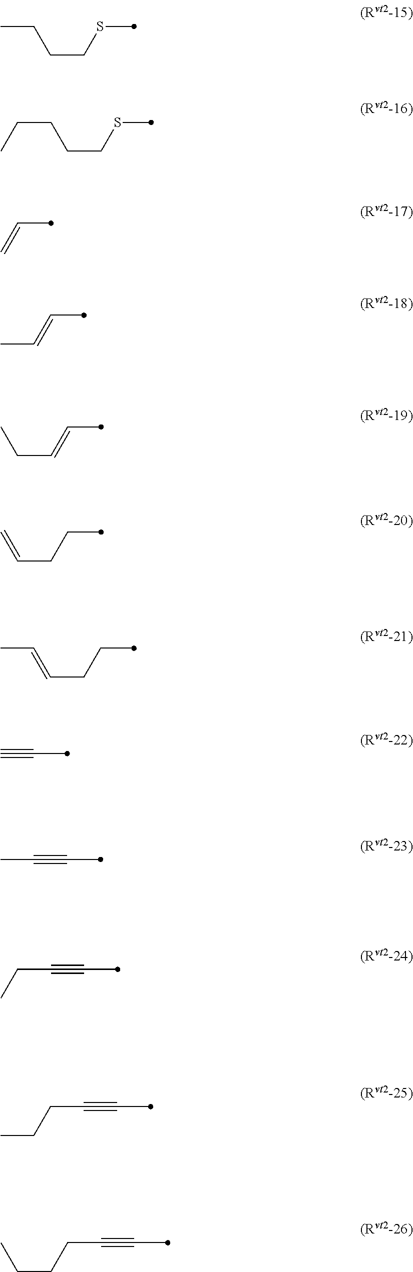

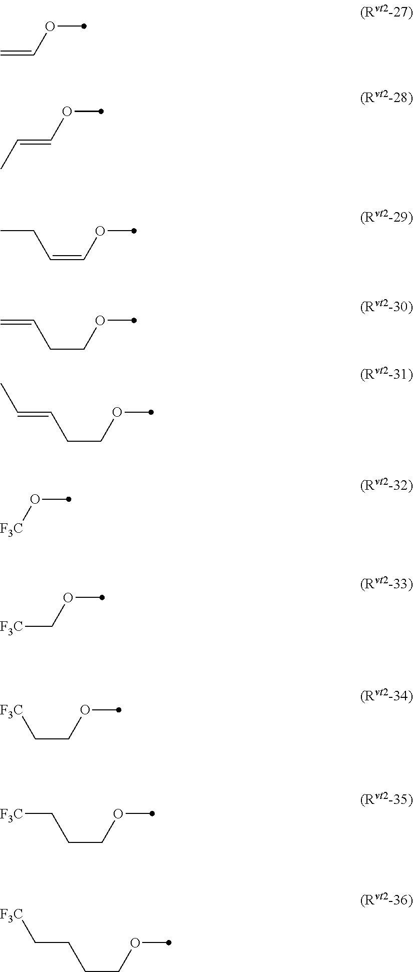

In the formulae (R^{vt2}-1) to (R^{vt2}-36), a black dot represents a bonding arm to A^{vt3}.

When the ring structure to which R^{vt2} is bonded is a phenyl group (aromatic), a linear alkyl group with 1 to 5 carbon atoms, a linear alkoxy group with 1 to 4 carbon atoms, or an alkenyl group with 4 or 5 carbon atoms is preferred. When the ring structure to which R^{vt2} is bonded is a saturated ring structure, such as cyclohexane, pyran, or dioxane, a linear alkyl group with 1 to 5 carbon atoms, a linear alkoxy group with 1 to 4 carbon atoms, or a linear alkenyl group with 2 to 5 carbon atoms is preferred.

To stabilize a nematic phase, in R^{vt2}, the total number of carbon atoms and, if present, oxygen atoms is preferably 5 or less, and a straight chain is preferred.

From the perspective of solubility, Δn, and/or Δεᵣ, R^{vt2} is preferably a fluorine atom, a cyano group, a linear alkyl group with 2 to 6 carbon atoms, a linear alkoxy group with 1 to 6 carbon atoms, or a linear alkylsulfanyl group with 1 to 6 carbon atoms.

In the general formula (vt), A^{vt1}, A^{vt2}, and A^{vt3} each independently denote any one of a hydrocarbon ring with 3 to 16 carbon atoms or a heterocycle with 3 to 16 carbon atoms.

More specifically, a hydrocarbon ring with 3 to 16 carbon atoms or a heterocycle with 3 to 16 carbon atoms preferably denotes a group selected from the group consisting of the groups (a), (b), (c), and (d):
(a) a 1,4-cyclohexylene group (one —CH₂— or two or more nonadjacent —CH₂— groups in this group are optionally substituted with —O— or —S—),
(b) a 1,4-phenylene group (one —CH═ or two or more —CH═ groups in this group are optionally substituted with —N═),
(c) a 1,4-cyclohexenylene group, a bicycle[2.2.2]octane-1,4-diyl group, a naphthalene-2,6-diyl group, a naphthalene-1,4-diyl group, a 1,2,3,4-tetrahydronaphthalene-2,6-diyl group, a 5,6,7,8-tetrahydronaphthalene-1,4-diyl group, a decahydronaphthalene-2,6-diyl group, an anthracene-2,6-diyl group, an anthracene-1,4-diyl group, an anthracene-9,10-diyl group, a phenanthrene-2,7-diyl group (one —CH═ or two or more —CH═ groups in the naphthalene-2,6-diyl group, the naphthalene-1,4-diyl group, the 1,2,3,4-tetrahydronaphthalene-2,6-diyl group, the 5,6,7,8-tetrahydronaphthalene-1,4-diyl group, the anthracene-2,6-diyl group, the anthracene-1,4-diyl group, the anthracene-9,10-diyl group, or the phenanthrene-2,7-diyl group are optionally substituted with —N═), and
(d) a thiophene-2,5-diyl group, a benzothiophene-2,5-diyl group, a benzothiophene-2,6-diyl group, a dibenzothiophene-3,7-diyl group, a dibenzothiophene-2,6-diyl group, a thieno[3,2-b]thiophene-2,5-diyl group (one —CH═ or two or more —CH═ groups in this group are optionally substituted with —N═).

One or two or more hydrogen atoms in A^{vt1}, A^{vt2}, and A^{vt3} are each independently optionally substituted with a substituent S^{vt1}.

The substituent S^{vt1} denotes any one of a fluorine atom, a chlorine atom, a bromine atom, an iodine atom, a pentafluorosulfanyl group, a nitro group, a cyano group, an isocyano group, an amino group, a hydroxy group, a mercapto group, a methylamino group, a dimethylamino group, a diethylamino group, a diisopropylamino group, a trimethylsilyl group, a dimethylsilyl group, a thioisocyano group, and an alkyl group with 1 to 20 carbon atoms.

The alkyl group is a linear, branched, or cyclic alkyl group, preferably a linear alkyl group.

The number of carbon atoms in the alkyl group preferably ranges from 2 to 10 or preferably 3 to 6.

One or two or more —CH₂— groups in the alkyl group are each independently optionally substituted with —O—, —S—, and/or —CO—.

One or two or more —CH₂—CH₂— groups in the alkyl group are each independently optionally substituted with —CH═CH—, —CF═CF—, —C≡C—, —CO—O—, —O—CO—, —CO—S—, —S—CO—, —CO—NH—, and/or —NH—CO—.

One or two or more hydrogen atoms in the alkyl group are each independently optionally substituted with a halogen atom.

The halogen atom may be a fluorine atom, a chlorine atom, a bromine atom, or an iodine atom.

When the alkyl group is substituted with a predetermined group, oxygen atoms are not directly bonded to each other.

From the perspective of the stability of the compound, sulfur atoms are preferably not directly bonded to each other, and/or an oxygen atom and a sulfur atom are preferably not directly bonded to each other.

The substituent S^{vt1} is preferably a fluorine atom or a linear alkyl group with 1 to 3 carbon atoms.

At least one of A^{vt1}, A^{vt2}, and A^{vt3} is preferably substituted with at least one substituent S^{vt1}.

A^{vt1} is preferably substituted with at least one substituent S^{vt1}.

A plurality of substituents S^{vt1}, if present, may be the same or different.

The substitution position of the substituent S^{vt1} in A^{vt1} is preferably any one of the following formulae (A^{vt1}-SP-1) to (A^{vt1}-SP-3):

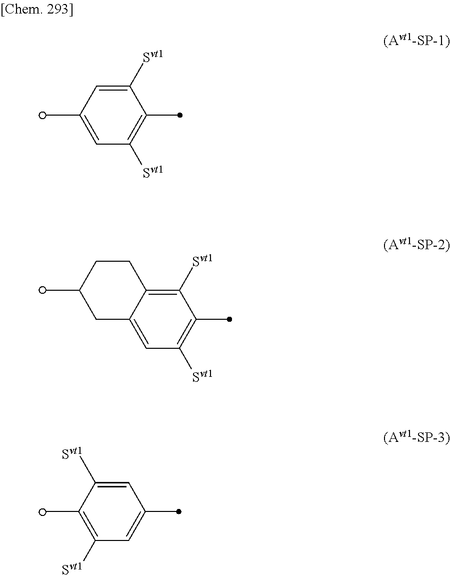

In the formulae (A^{vt1}-SP-1) to (A^{vt1}-SP-3), a white dot represents a bonding arm to R^{vt1}, and a black dot represents a bonding arm to —C≡C—.

The substitution position of the substituent S^{vt1} in A^{vt2} is preferably any one of the following formulae (A^{vt2}-SP-1) to (A^{vt2}-SP-7), preferably any one of the formulae (A^{vt2}-SP-1) to (A^{vt2}-SP-5) from the perspective of compatibility with another liquid crystal compound.

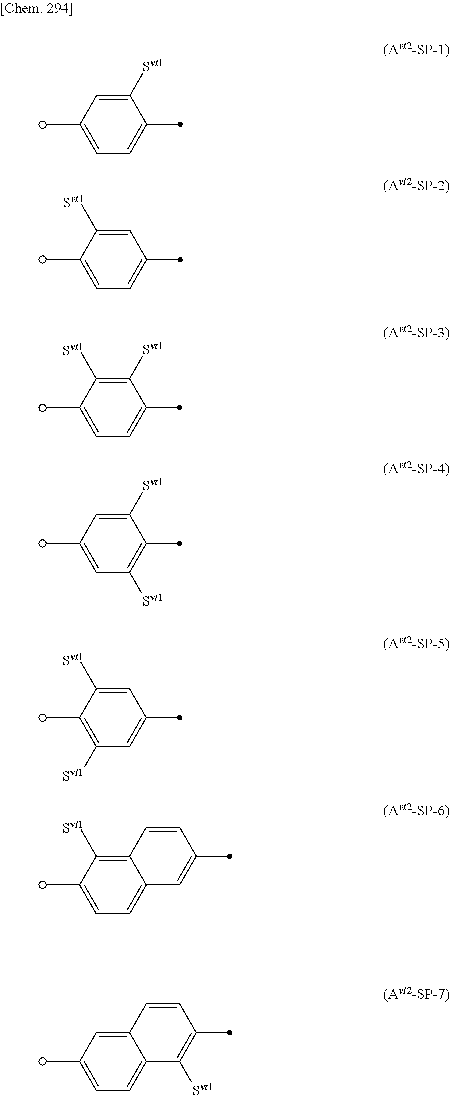

In the formulae (A^{vt2}-SP-1) to (A^{vt2}-SP-7), a white dot represents a bonding arm to —C≡C—, and a black dot represents a bonding arm to Z^{vt1}.

The substitution position of the substituent S^{vt3} in A^{vt3} is preferably any one of the following formulae (A^{vt3}-SP-1) to (A^{vt3}-SP-8), preferably any one of the following formulae (A^{vt3}-SP-1) to (A^{vt3}-SP-5) from the perspective of solubility.

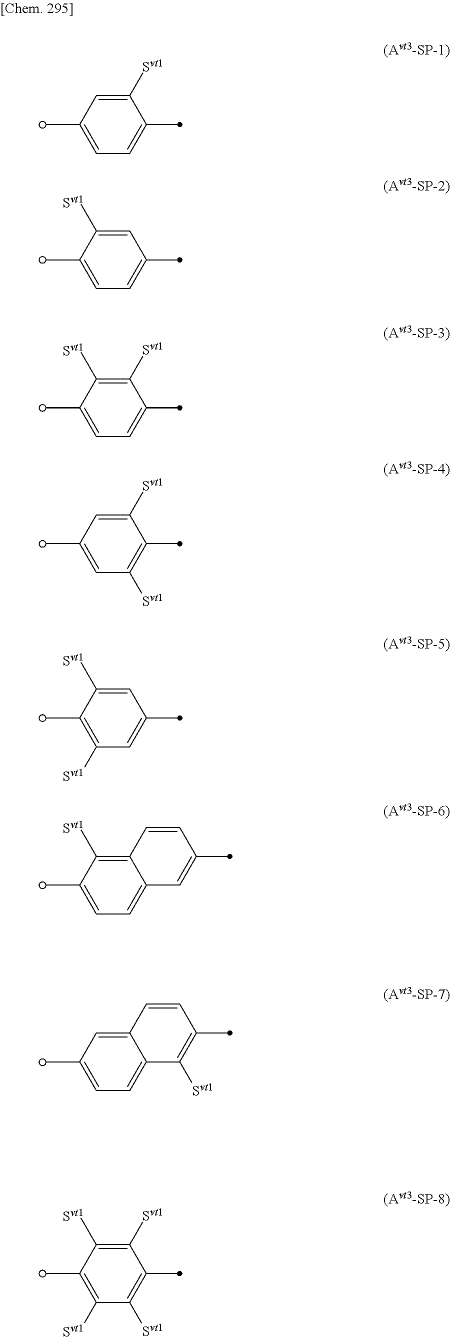

In the formulae (A^{vt3}-SP-1) to (A^{vt3}-SP-8), a white dot represents a bonding arm to Z^{vt1}, and a black dot represents a bonding arm to Z^{vt1} or R^{vt2}.

More specifically, A^{vt1} preferably denotes any one of the following formulae (A^{vt1}-1) to (A^{vt1}-5):

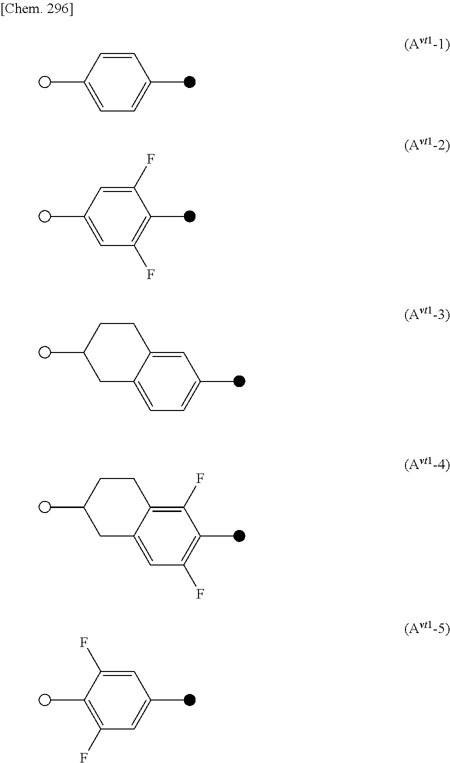

In the formulae (A^{vt1}-1) to (A^{vt1}-5), a white dot represents a bonding arm of R^{vt1}, and a black dot represents a bonding arm to —C≡C—.

More specifically, A^{vt2} preferably denotes any one of the following formulae (A^{vt2}-1) to (A^{vt2}-6):

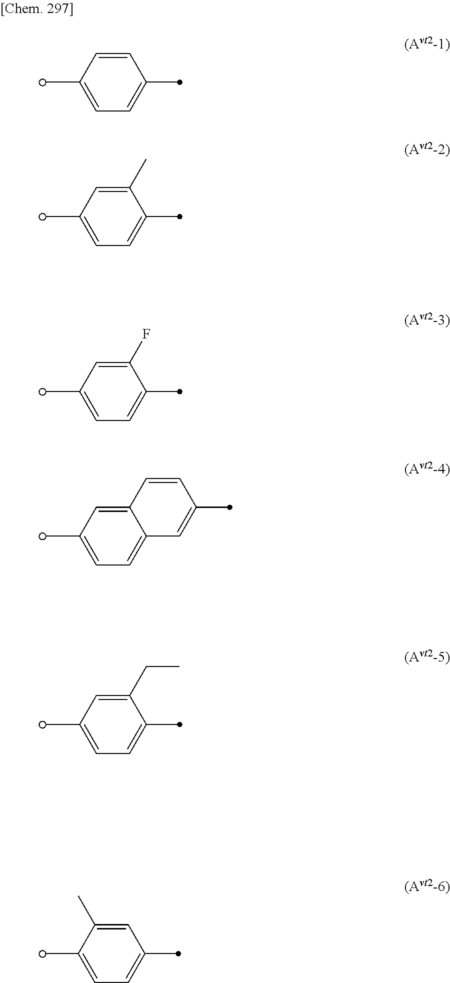

In the formulae (A^{vt2}-1) to (A^{vt2}-6), a white dot represents a bonding arm of —C≡C—, and a black dot represents a bonding arm to Z^{vt1}.

More specifically, A^{vt3} preferably denotes any one of the following formulae (A^{vt3}-1) to (A^{vt3}-5):

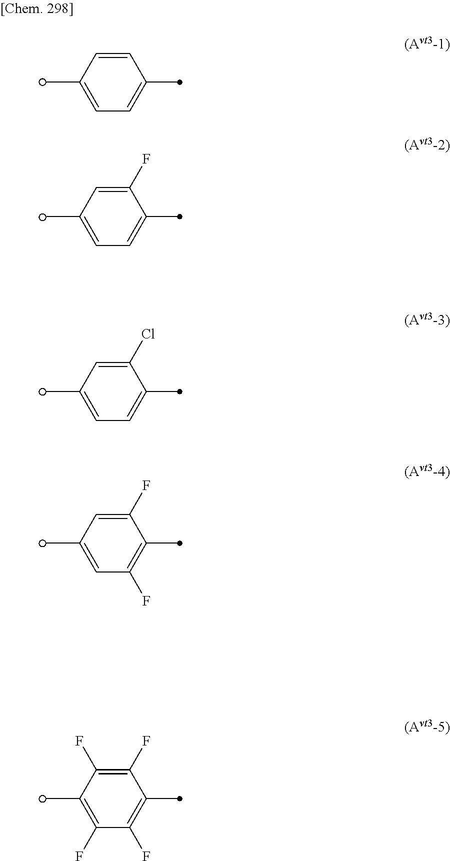

In the formulae (A^{vt3}-1) to (A^{vt3}-5), a white dot represents a bonding arm to Z^{vt1}, and a black dot represents a bonding arm to Z^{vt1} or R^{vt2}.

In the general formula (vt), Z^{vt1} each independently denotes any one of a single bond and an alkylene group with 1 to 20 carbon atoms.

The alkylene group is a linear, branched, or cyclic alkylene group, preferably a linear alkylene group.

The number of carbon atoms in the alkylene group preferably ranges from 2 to 10 or preferably 2 to 6.

One or two or more —CH₂— groups in the alkylene group are each independently optionally substituted with —O—, —CF₂—, and/or —CO—.

One or two or more —CH₂—CH₂— groups in the alkylene group are each independently optionally substituted with —CH₂—CH(CH₃)—, —CH(CH₃)—CH₂—, —CH═CH—, —CF═CF—, —CH═C(CH₃)—, —C(CH₃)═CH—, —CH═N—, —N═CH—, —N═N—, —C≡C—, —CO—O—, and/or —O—CO—.

When the alkylene group is substituted with a predetermined group, oxygen atoms are not directly bonded to each other.

Specific examples of the alkylene group with 2 to 20 carbon atoms (including substituted ones) include the groups represented by the formulae (Z^{vt1}-1) to (Z^{vt1}-24):

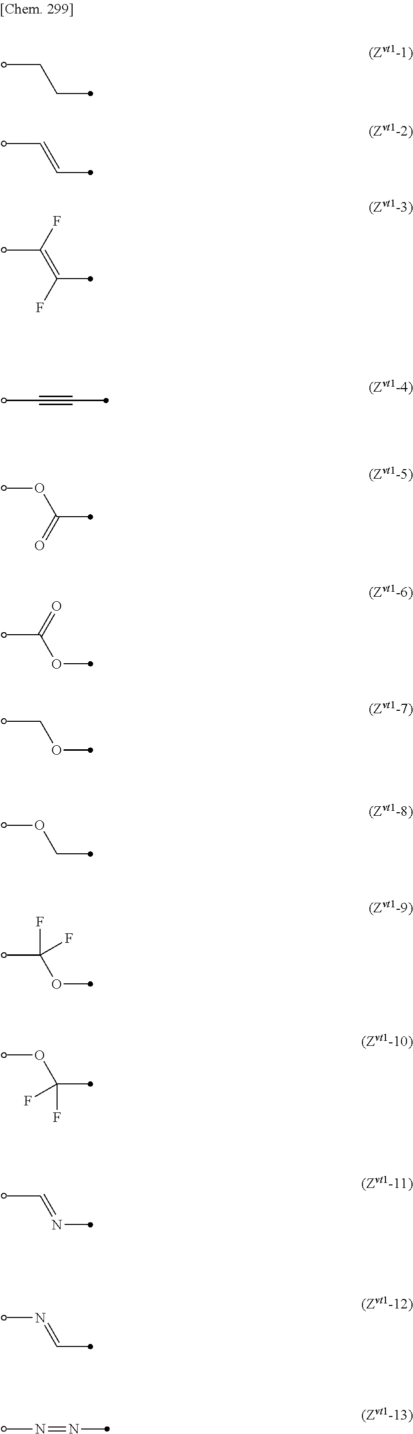

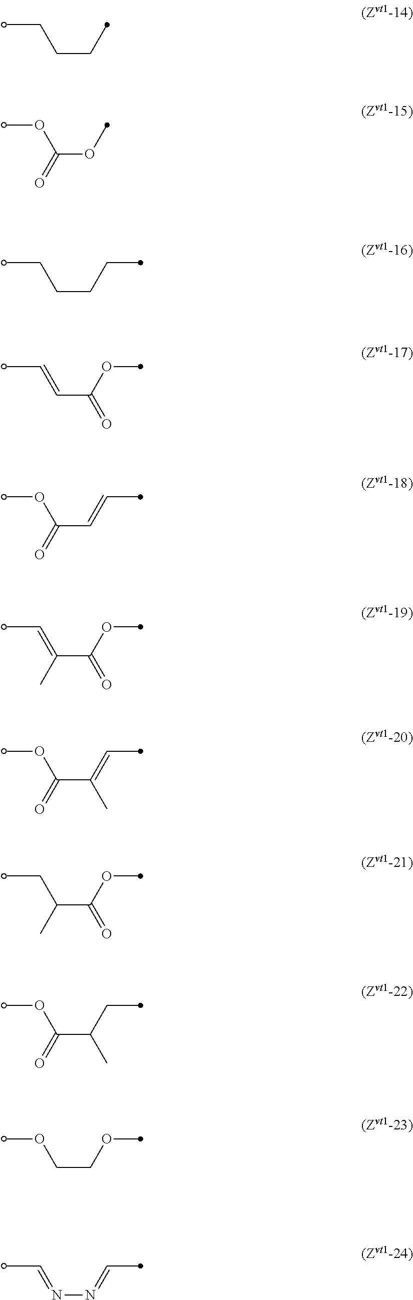

In the formulae (Z^{vt1}-1) to (Z^{vt1}-24), a white dot represents a bonding arm to A^{vt2} or A^{vt3}, and a black dot represents a bonding arm to A^{vt3}.

In the general formula (vt), n^{vt1} denotes an integer in the range of 1 to 3, preferably 1 or 2.

When n^{vt1} is 1, from the perspective of Δn and/or Δεᵣ, Z^{vt1} preferably denotes —C≡C—.

When n^{vt1} is 2 or 3, from the perspective of Δn and/or Δεᵣ, at least one Z^{vt1} preferably denotes —C≡C—.

In the general formula (vt), a plurality of A^{vt3}, if present, may be the same or different, and a plurality of Z^{vt1}, if present, may be the same or different.

Compounds represented by the general formula (vt) are preferably the compounds represented by the following general formula (vt-1):

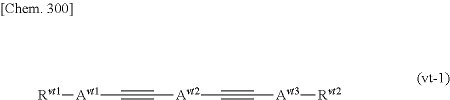

In the general formula (vt-1), R^{vt1}, R^{vt2}, A^{vt1}, A^{vt2}, and A^{vt3} denote the same as R^{vt1}, R^{vt2}, A^{vt1}, A^{vt2}, and A^{vt3}, respectively, in the general formula (vt).

Compounds represented by the general formula (vt-1) are preferably the compounds represented by the following general formulae (vt-1-1) to (vt-1-3):

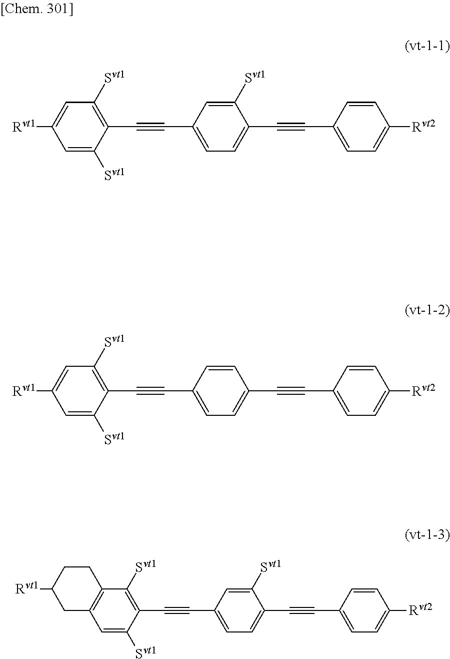

In the general formulae (vt-1-1) to (vt-1-3), R^{vt1}, R^{vt2}, and S^{vt1} each independently denote the same as R^{vt1}, R^{vt2}, and S^{vt1} in the general formula (vt).

Specific examples of the compounds represented by the general formula (vt-1-1) include the compounds represented by the following structural formulae (vt-1-1.1) to (vt-1-1.24):

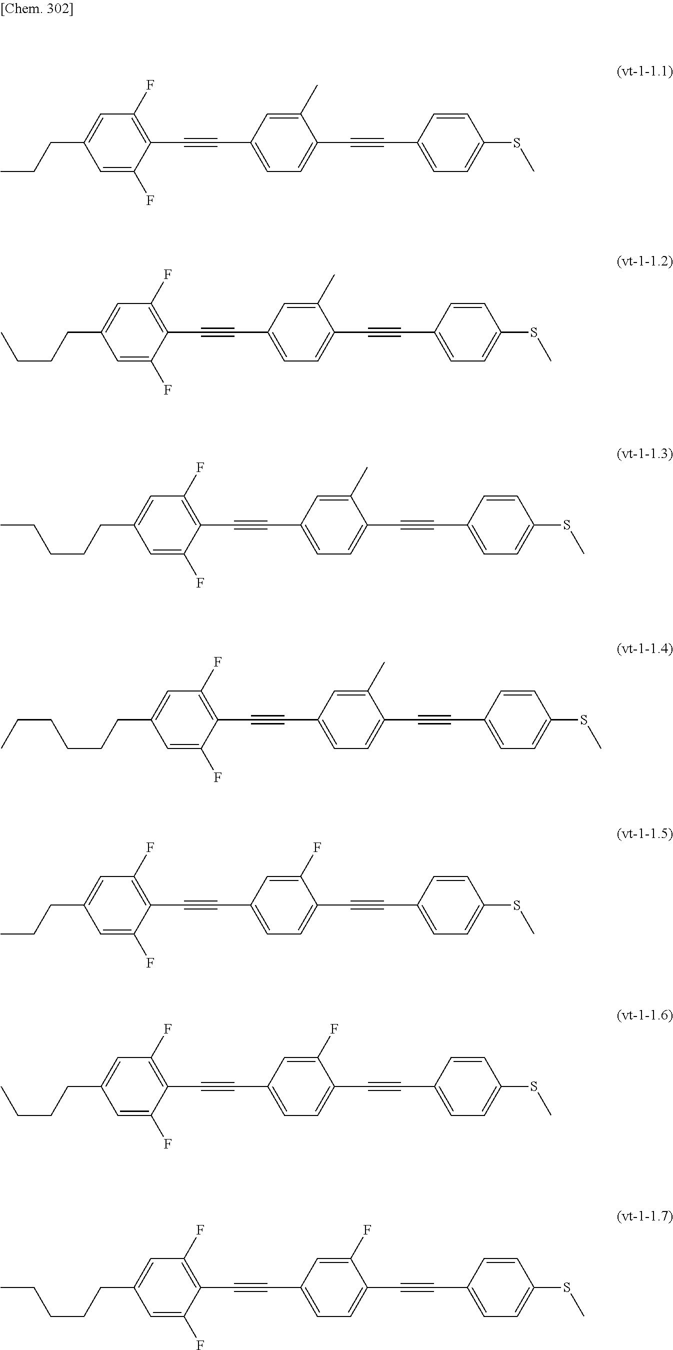

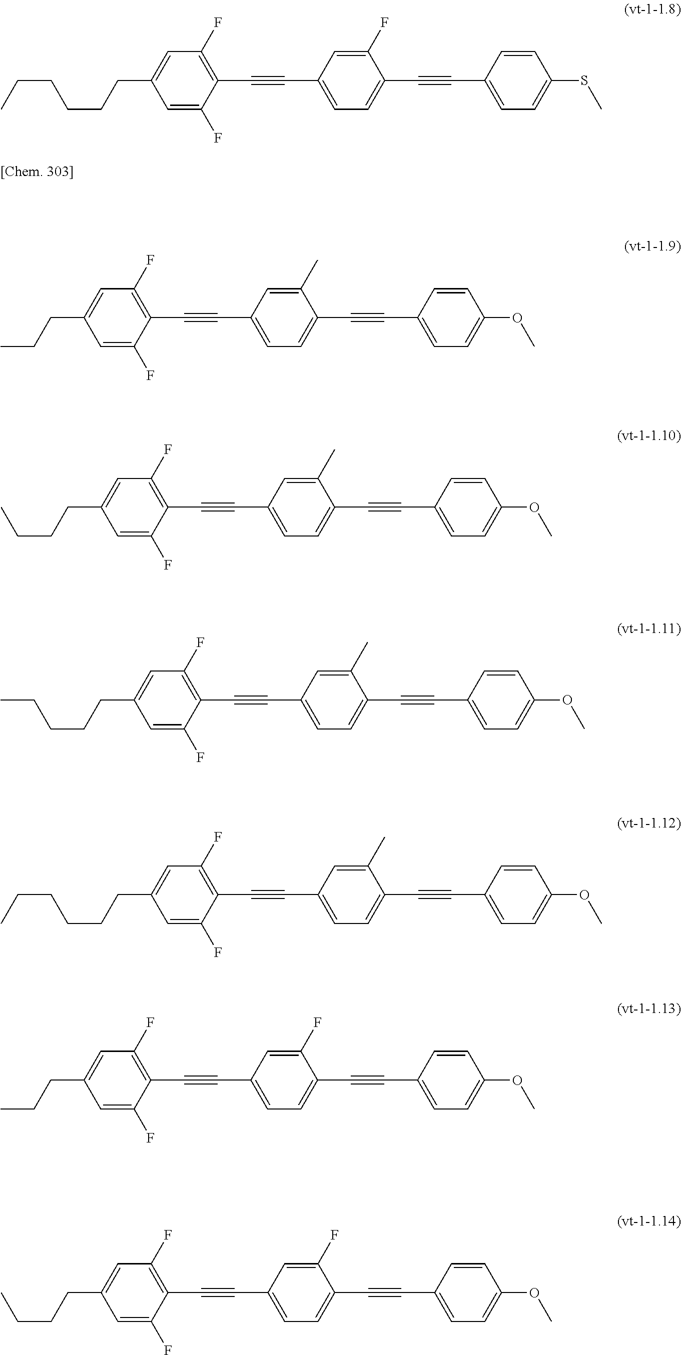

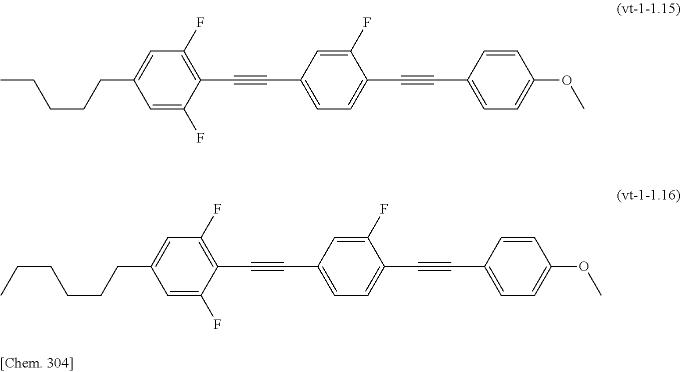

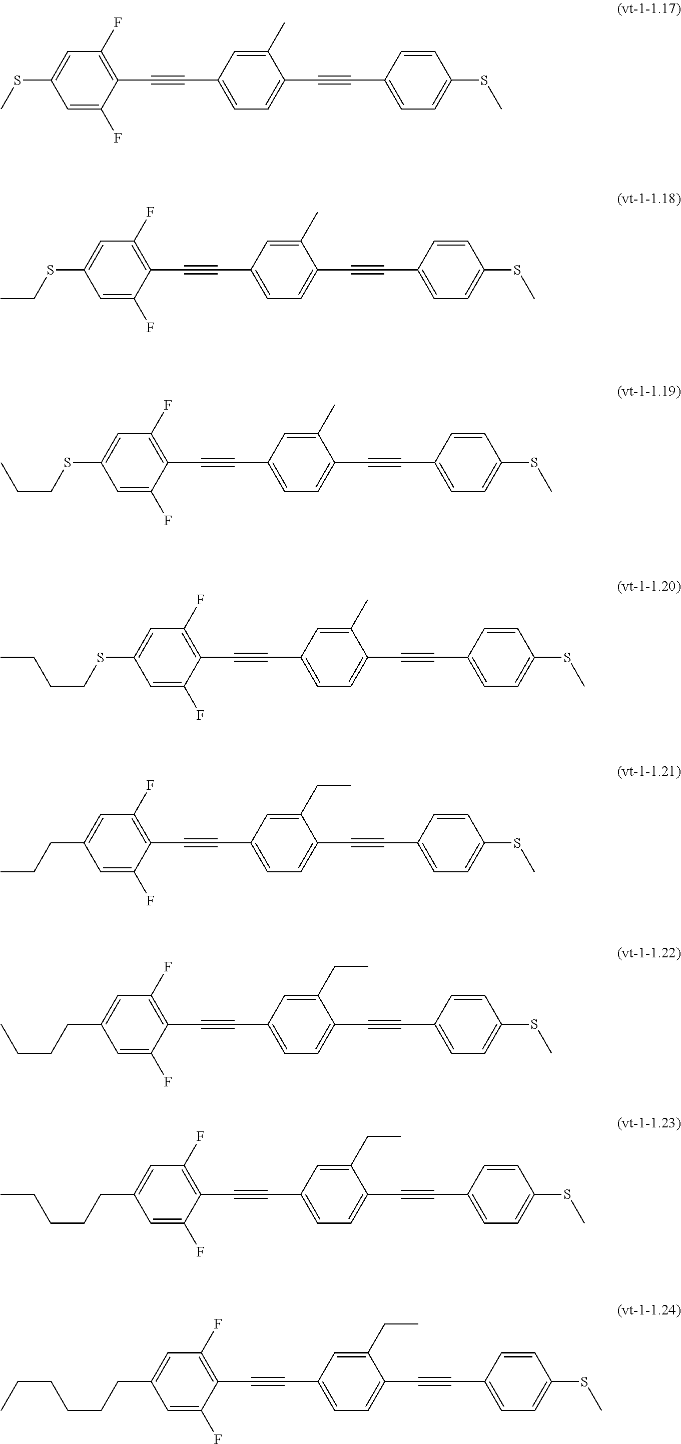

Specific examples of the compounds represented by the general formula (vt-1-2) include the compounds represented by the following structural formulae (vt-1-2.1) to (vt-1-2.8):

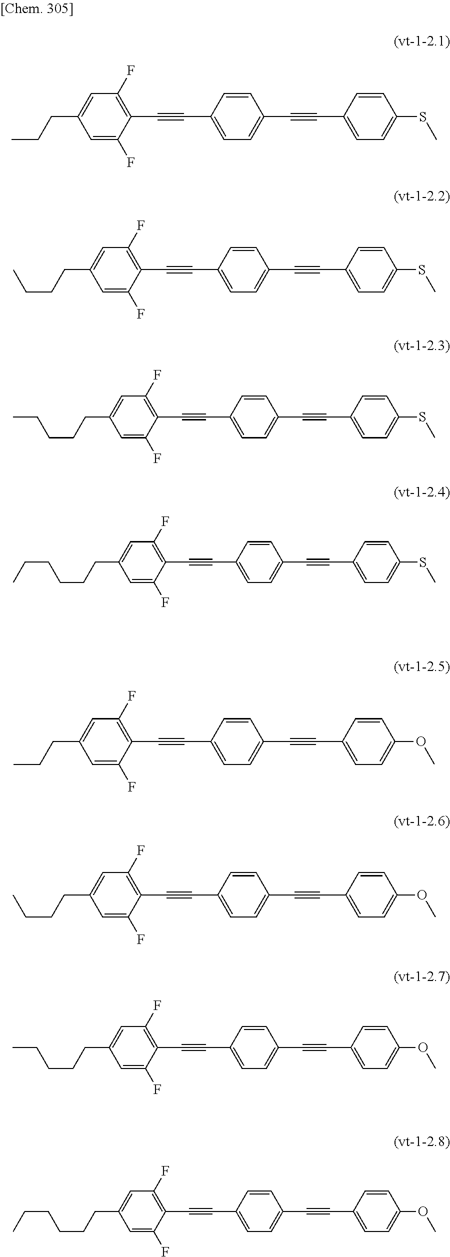

Specific examples of the compounds represented by the general formula (vt-1-3) include the compounds represented by the following structural formulae (vt-1-3.1) to (vt-1-3.4):

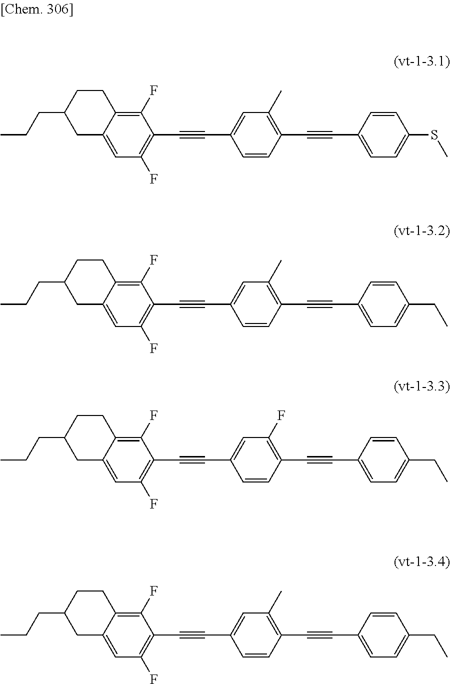

The number of types of compounds represented by the following formulae used in liquid crystal compositions is one or two or more, preferably one to five, preferably one to four, preferably one to three, preferably one or two, or preferably one: the general formula (vt), the general formula (vt-1), the general formulae (vt-1-1) to (vt-1-3), the structural formulae (vt-1-1.1) to (vt-1-1.24), the structural formulae (vt-1-2.1) to (vt-1-2.8), or the structural formulae (vt-1-3.1) to (vt-1-3.4).

The lower limit of the total amount of the compounds represented by the following formulae per 100% by mass of the liquid crystal composition is preferably 0.18 by mass or more, 0.5% by mass or more, or 18 by mass or more: the general formula (vt), the general formula (vt-1), the general formulae (vt-1-1) to (vt-1-3), the structural formulae (vt-1-1.1) to (vt-1-1.24), the structural formulae (vt-1-2.1) to (vt-1-2.8), or the structural formulae (vt-1-3.1) to (vt-1-3.4).

The upper limit of the total amount of the compounds represented by the following formulae per 100% by mass of the liquid crystal composition is preferably 25% by mass or less, preferably 20% by mass or less, or preferably 15% by mass or less: the general formula (vt), the general formula (vt-1), the general formulae (vt-1-1) to (vt-1-3), the structural formulae (vt-1-1.1) to (vt-1-1.24), the structural formulae (vt-1-2.1) to (vt-1-2.8), or the structural formulae (vt-1-3.1) to (vt-1-3.4).

From the perspective of solubility, Δn, and/or Δεᵣ, the total amount of the compounds represented by the following formulae per 100% by mass of the liquid crystal composition preferably ranges from 0.1% to 25% by mass, 0.5% to 20% by mass, or 1% to 15% by mass: the general formula (vt), the general formula (vt-1), the general formulae (vt-1-1) to (vt-1-3), the structural formulae (vt-1-1.1) to (vt-1-1.24), the structural formulae (vt-1-2.1) to (vt-1-2.8), or the structural formulae (vt-1-3.1) to (vt-1-3.4).

The compounds represented by the general formula (vt) (including subordinate concepts) can be synthesized by a known synthesis method.

From the perspective of solubility, a liquid crystal composition according to the present invention may further contain one or two or more compounds represented by the following general formulae (np-1) to (np-3):

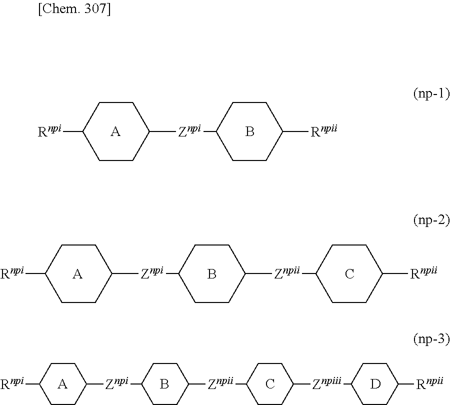

In the general formulae (np-1) to (np-3), R^{npi} and R^{npi1} each independently denote any one of an alkyl group with 1 to 20 carbon atoms and a halogen atom.

The alkyl group with 1 to 20 carbon atoms is a linear, branched, or cyclic alkyl group, preferably a linear alkyl group.

The number of carbon atoms in the alkyl group with 1 to 20 carbon atoms preferably ranges from 2 to 10 or preferably 2 to 6.

One or two or more —CH₂— groups in the alkyl group are each independently optionally substituted with —O—, —S—, —CO—, and/or —CS—.

One or two or more —CH₂—CH₂— groups in the alkyl group are each independently optionally substituted with —CO—O—, —O—CO—, —CO—S—, —S—CO—, —CO—NH—, —NH—CO—, —CH═CH—, —CF═CH—, —CH═CF—, —CF═CF—, and/or —C≡C—.

One or two or more hydrogen atoms in the alkyl group are each independently optionally substituted with a halogen atom.

The halogen atom may be a fluorine atom, a chlorine atom, a bromine atom, or an iodine atom.

When the alkyl group is substituted with a predetermined group, oxygen atoms are not directly bonded to each other.

From the perspective of the stability of the compound, sulfur atoms are preferably not directly bonded to each other, and/or an oxygen atom and a sulfur atom are preferably not directly bonded to each other.

For example, R^{npi} and R^{npi1} can denote an alkoxy group with 1 to 19 carbon atoms by substituting one —CH₂— in the alkyl group with —O—.

The alkoxy group is a linear, branched, or cyclic alkoxy group, preferably a linear alkoxy group.

The number of carbon atoms in the alkoxy group preferably ranges from 2 to 10 or preferably 2 to 6.

R^{npi} and R^{npi1} can denote an alkylsulfanyl group (thioalkyl group) with 1 to 19 carbon atoms by substituting one —CH₂— in the alkyl group with —S—.

The alkylsulfanyl group is a linear, branched, or cyclic alkylsulfanyl group, preferably a linear alkylsulfanyl group.

The number of carbon atoms in the alkylsulfanyl group preferably ranges from 2 to 10 or preferably 2 to 6.

R^{npi} and R^{npi1} can denote an alkenyl group with 2 to 20 carbon atoms by substituting one or two or more —CH₂—CH₂— groups in the alkyl group with —CH═CH—.

The alkenyl group is a linear, branched, or cyclic alkenyl group, preferably a linear alkenyl group.

The number of carbon atoms in the alkenyl group preferably ranges from 2 to 10 or preferably 2 to 6.

R^{npi} and R^{npi1} can denote an alkynyl group with 2 to 20 carbon atoms by substituting one or two or more —CH₂—CH₂— groups in the alkyl group with —C≡C—.

The alkynyl group is a linear, branched, or cyclic alkynyl group, preferably a linear alkynyl group.

The number of carbon atoms in the alkynyl group preferably ranges from 2 to 10 or preferably 2 to 6.

R^{npi} and R^{npi1} can denote an alkenyloxy group with 2 to 19 carbon atoms by substituting one —CH₂— in the alkyl group with —O— and substituting one or two or more —CH₂—CH₂— groups with —CH═CH—.

The alkenyloxy group is a linear, branched, or cyclic alkenyloxy group, preferably a linear alkenyloxy group.

The number of carbon atoms in the alkenyloxy group preferably ranges from 2 to 10 or preferably 2 to 6.

R^{npi} and R^{npi1} can denote an alkyl halide group with 1 to 20 carbon atoms by substituting one or two or more hydrogen atoms in the alkyl group with a halogen atom.

The alkyl halide group is a linear, branched, or cyclic alkyl halide group, preferably a linear alkyl halide group.

The number of carbon atoms in the alkyl halide group preferably ranges from 2 to 10 or preferably 2 to 6.

R^{npi} and R^{npi1} can denote an alkoxy halide group with 1 to 19 carbon atoms by substituting one —CH₂— in the alkyl group with —O— and substituting one or two or more hydrogen atoms in the alkyl group with a halogen atom.

The alkoxy halide group is a linear, branched, or cyclic alkoxy halide group, preferably a linear alkoxy halide group.

The number of carbon atoms in the alkoxy halide group preferably ranges from 2 to 10 or preferably 2 to 6.

Specific examples of the alkyl group with 1 to 20 carbon atoms (including substituted ones) in R^{npi} and R^{npi1} include the groups represented by the formulae (R^{npi}/ii-1) to (R^{npi}/ii-36):

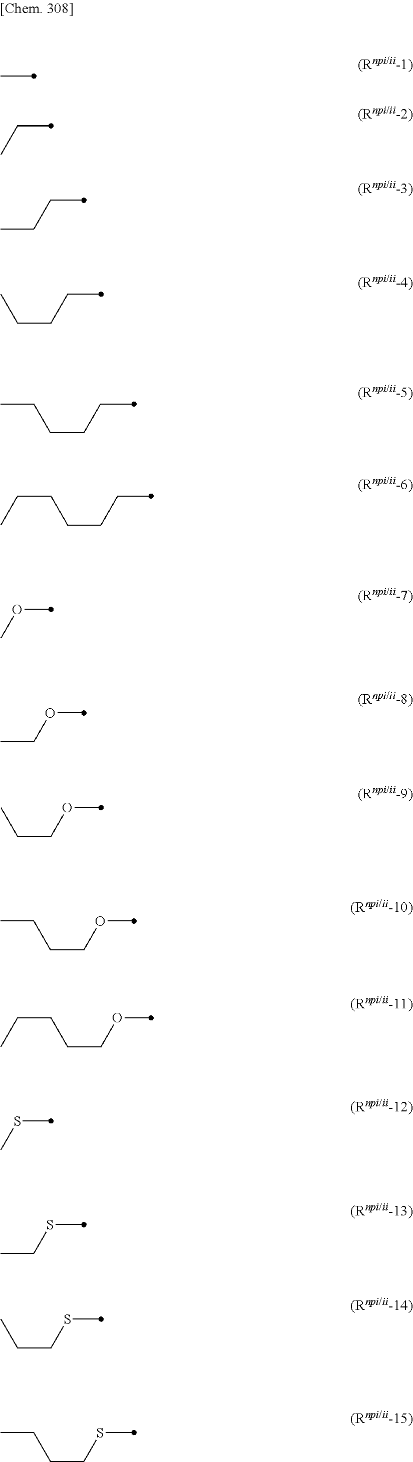

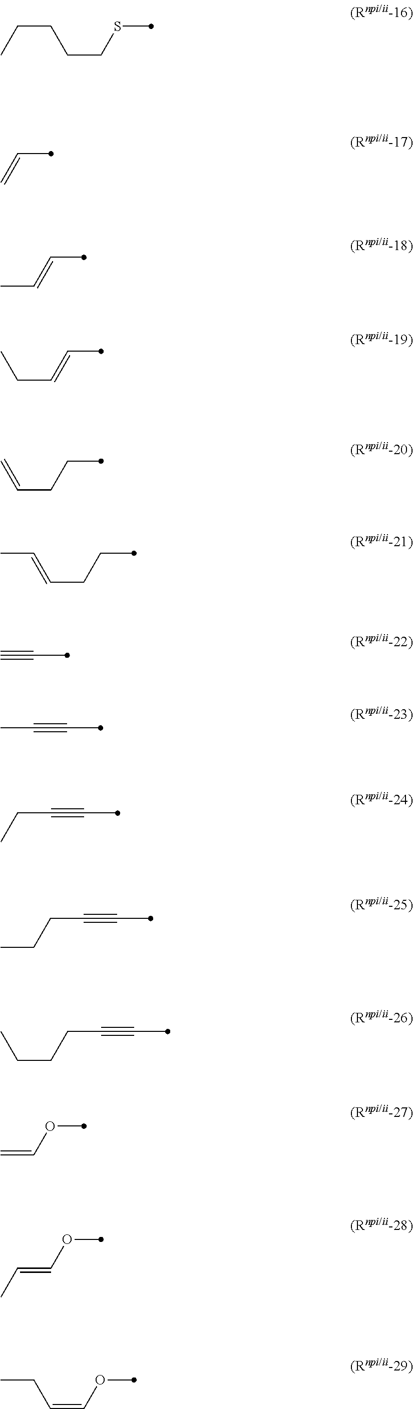

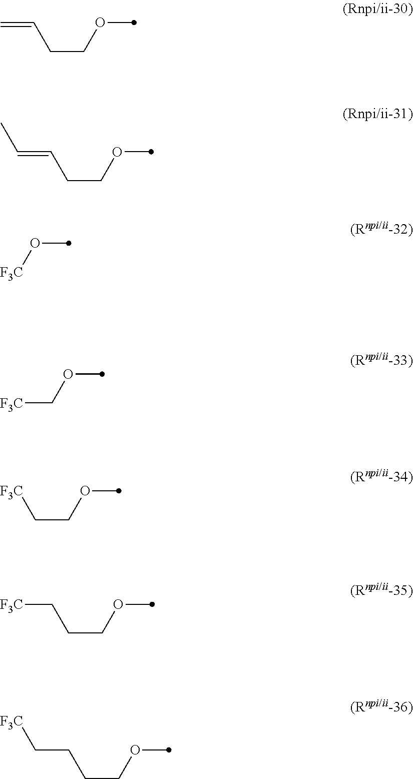

In the formulae (R^{npi}/ii-1) to (R^{npi}/ii-36), a black dot represents a bonding arm to a ring A, B, C, or D.

The halogen atom in R^{npi} and R^{npi1} may be a fluorine atom, a chlorine atom, a bromine atom, an iodine atom, or the like.

In the general formulae (np-1) to (np-3), the rings A, B, C, and D each independently denote a group selected from the group consisting of the groups (a), (b), (c), and (d):
(a) a 1,4-cyclohexylene group (one —CH₂— or two or more nonadjacent —CH₂— groups in this group are optionally substituted with —O—),
(b) a 1,4-phenylene group (one —CH═ or two or more —CH═ groups in this group are optionally substituted with —N═),
(c) a naphthalene-2,6-diyl group, a 1,2,3,4-tetrahydronaphthalene-2,6-diyl group, or a decahydronaphthalene-2,6-diyl group (one —CH═ or two or more —CH═ groups in the naphthalene-2,6-diyl group or the 1,2,3,4-tetrahydronaphthalene-2,6-diyl group are optionally substituted with —N═), and
(d) a 1,4-cyclohexenylene group, a 1,3-dioxane-trans-2,5-diyl group, a pyrimidine-2,5-diyl group, or a pyridine-2,5-diyl group.

One or two or more hydrogen atoms in the rings A, B, C, and D are each independently optionally substituted with a substituent S^{npi1}.

The substituent S^{npi1} denotes any one of a halogen atom, a cyano group, and an alkyl group with 1 to 20 carbon atoms.

The halogen atom may be a fluorine atom, a chlorine atom, a bromine atom, or an iodine atom, preferably a fluorine atom from the perspective of stability and safety.

The alkyl group with 1 to 20 carbon atoms is a linear, branched, or cyclic alkyl group, preferably a linear alkyl group.

The number of carbon atoms in the alkyl group with 1 to 20 carbon atoms preferably ranges from 2 to 10 or preferably 2 to 6.

One or two or more —CH₂— groups in the alkyl group are each independently optionally substituted with —O—, —S—, —CO—, and/or —CS—.

One or two or more —CH₂—CH₂— groups in the alkyl group are each independently optionally substituted with —CO—O—, —O—CO—, —CO—S—, —S—CO—, —CO—NH—, —NH—CO—, —CH═CH—, —CF═CF—, and/or —C≡C—.

One or two or more hydrogen atoms in the alkyl group are each independently optionally substituted with a halogen atom.

The halogen atom may be a fluorine atom, a chlorine atom, a bromine atom, or an iodine atom.

When the alkyl group is substituted with a predetermined group, oxygen atoms are not directly bonded to each other.

From the perspective of the stability of the compound, sulfur atoms are preferably not directly bonded to each other, and/or an oxygen atom and a sulfur atom are preferably not directly bonded to each other.

The substituent S^{npi1} is preferably a halogen atom, preferably a fluorine atom.

A plurality of substituents S^{npi1}, if present, may be the same or different.

The substitution position of the substituent S^{npi1} in the ring A is preferably represented by the following formula (A-SP-1):

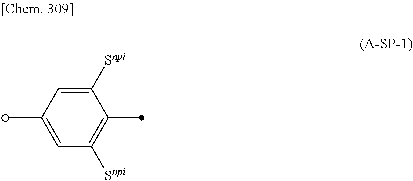

In the formula (A-SP-1), a white dot represents a bonding arm to R^{npi}, and a black dot represents a bonding arm to Z^{npi}.

More specifically, the ring A preferably denotes any one of the following formulae (A-1) to (A-3):

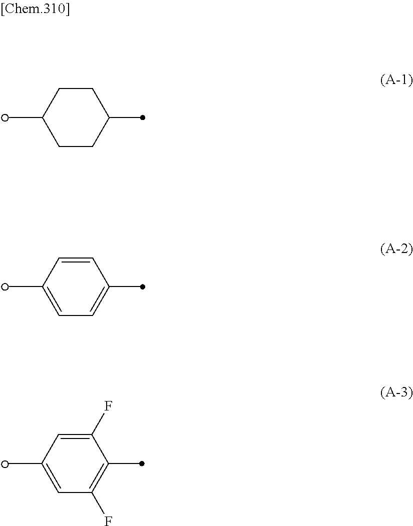

In the formulae (A-1) to (A-3), a white dot represents a bonding arm to R^{npi}, and a black dot represents a bonding arm to Z^{npi}.

More specifically, the ring B preferably denotes any one of the following formulae (B-1) and (B-2):

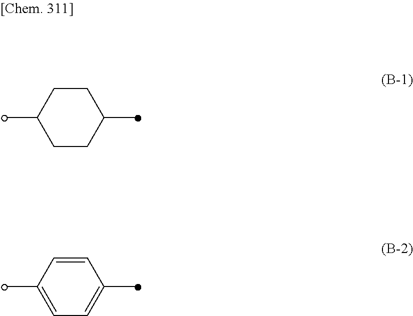

In the formulae (B-1) and (B-2), a white dot represents a bonding arm to Z^{npi}, and a black dot represents a bonding arm to R^{npi1} or Z^{npii}.

More specifically, the ring C preferably denotes any one of the following formulae (C-1) and (C-2):

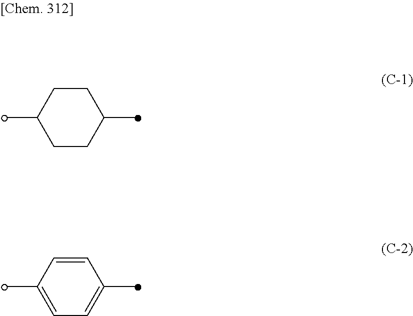

In the formulae (C-1) and (C-2), a white dot represents a bonding arm to Z^{npii}, and a black dot represents a bonding arm to R^{npi1} or Z^{npiii}.

In the general formulae (np-1) to (np-3), Z^{npi}, Z^{npii}, and Z^{npiii} each independently denote any one of a single bond and an alkylene group with 1 to 20 carbon atoms.

One or two or more —CH₂— groups in the alkylene group are each independently optionally substituted with —O—, —CF₂—, and/or —Co—.

One or two or more —CH₂—CH₂— groups in the alkylene group are each independently optionally substituted with —CH₂—CH(CH₃)—, —CH(CH₃)—CH₂—, —CH═CH—, —CF═CF—, —CH═C(CH₃)—, —C(CH₃)═CH—, —CH═N—, —N═CH—, —N═N—, —C≡C—, —CO—O—, and/or —O—CO—.

When the alkyl group with 1 to 10 carbon atoms is substituted with a predetermined group, oxygen atoms are not directly bonded to each other.

From the perspective of the stability of the compound, sulfur atoms are preferably not directly bonded to each other, and/or an oxygen atom and a sulfur atom are preferably not directly bonded to each other.

Specific examples of the alkylene group with 1 to 20 carbon atoms (including substituted ones) include the groups represented by the formulae (Z^{npi/ii/iii}-1) to (Z^{npi/ii/iii}-24):

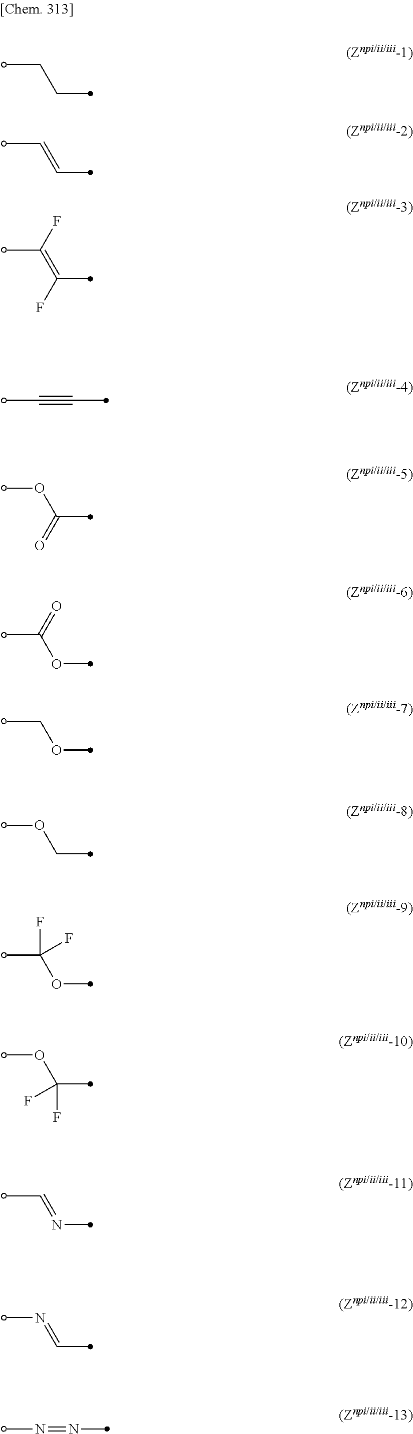

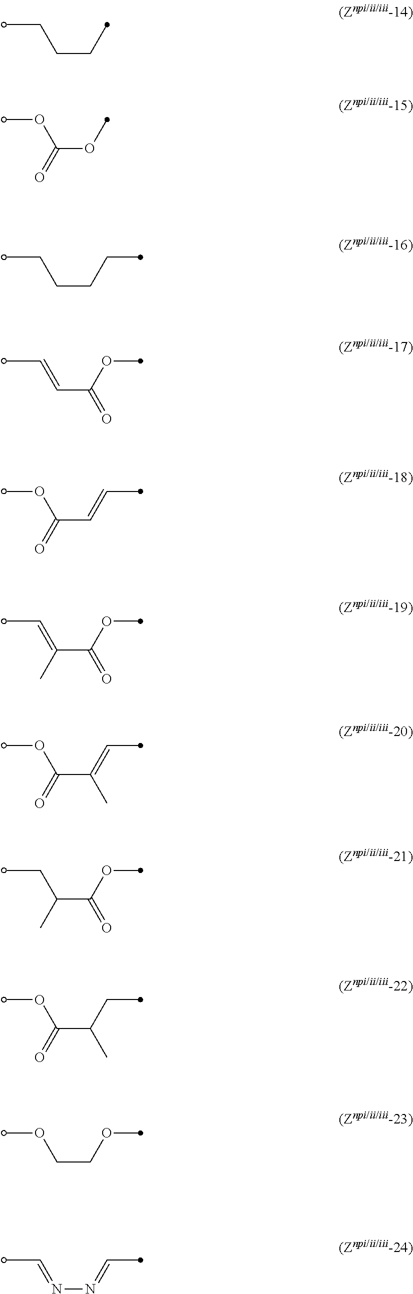

In the formulae (Z^{npi/ii/iii}-1) to (Z^{npi/ii/iii}-24), a white dot represents a bonding arm to the ring A, B, or C, and a black dot represents a bonding arm to the ring B, C, or D.

From the perspective of Δn and/or Δεᵣ, Z^{npi}, Z^{npii}, and Z^{npiii} each independently preferably denote any one of a single bond, —C≡C—, and —CO—O—.

Among the compounds represented by the general formulae (np-1) to (np-3), however, the compounds represented by the general formula (vt) (including subordinate concepts) are excluded.

Compounds represented by the general formula (np-2) are preferably the compounds represented by the following general formulae (np-2-1) to (np-2-3):

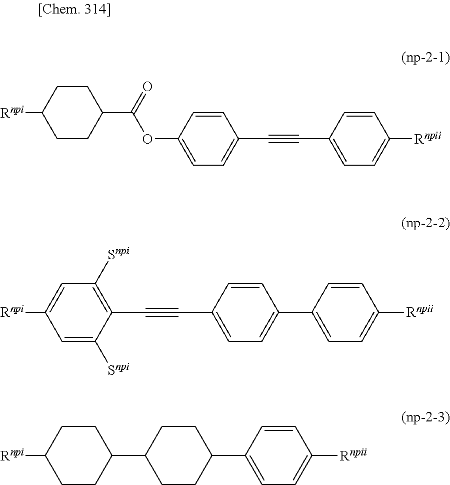

In the general formulae (np-2-1) to (np-2-3), Rapi, R^{npi1}, and S^{npi} denote the same as R^{npi}, R^{npi1}, and S^{npi}, respectively, in the general formulae (np-1) to (np-3).

Specific examples of the compounds represented by the general formula (np-2-1) include the compound represented by the following structural formula (np-2-1.1):

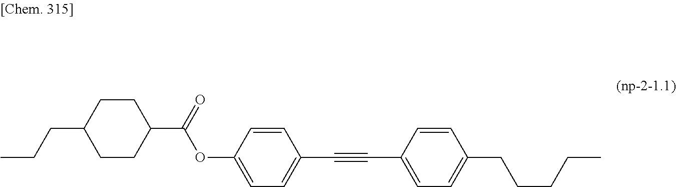

Specific examples of the compounds represented by the general formula (np-2-2) include the compounds represented by the following structural formulae (np-2-2.1) to (np-2-2.5):

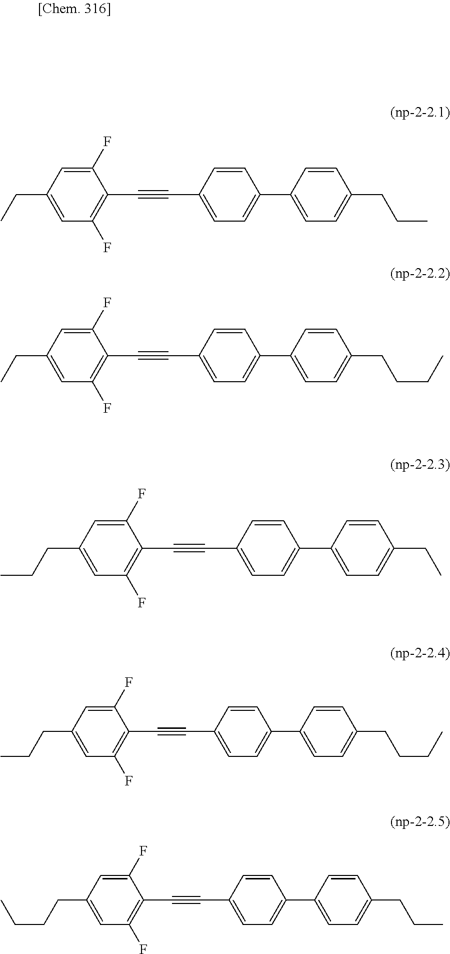

Specific examples of the compounds represented by the general formula (np-2-3) include the compounds represented by the following structural formulae (np-2-3.1) to (np-2-3.5):

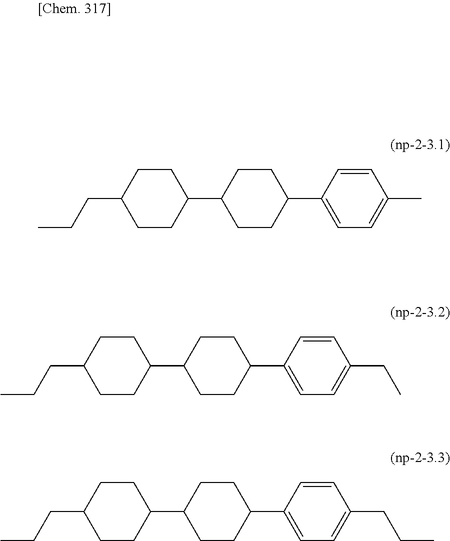

The number of types of compounds represented by the following formulae used in liquid crystal compositions is one or two or more, preferably one to ten, preferably one to eight, preferably one to six, preferably one to four, or preferably one or two: the general formulae (np-1) to (np-3), the general formulae (np-2-1) to (np-2-3), the structural formula (np-2-1.1), the structural formulae (np-2-2.1) to (np-2-2.5), or the structural formulae (np-2-3.1) to (np-2-3.3).

The lower limit of the total amount of the compounds represented by the following formulae per 100% by mass of the liquid crystal composition is preferably 0.1% by mass, preferably 0.5% by mass, or preferably 1% by mass: the general formulae (np-1) to (np-3), the general formulae (np-2-1) to (np-2-3), the structural formula (np-2-1.1), the structural formulae (np-2-2.1) to (np-2-2.5), or the structural formulae (np-2-3.1) to (np-2-3.3).

The upper limit of the total amount of the compounds represented by the following formulae per 100% by mass of the liquid crystal composition is preferably 30% by mass, preferably 20% by mass, or preferably 15% by mass: the general formulae (np-1) to (np-3), the general formulae (np-2-1) to (np-2-3), the structural formula (np-2-1.1), the structural formulae (np-2-2.1) to (np-2-2.5), or the structural formulae (np-2-3.1) to (np-2-3.3).

From the perspective of solubility, Δn, and/or Δεᵣ, the total amount of the compounds represented by the following formulae per 100% by mass of the liquid crystal composition preferably ranges from 0.1% to 30% by mass, preferably 0.5% to 20% by mass, or preferably 1% to 15% by mass: the general formulae (np-1) to (np-3), the general formulae (np-2-1) to (np-2-3), the structural formula (np-2-1.1), the structural formulae (np-2-2.1) to (np-2-2.5), or the structural formulae (np-2-3.1) to (np-2-3.3).

The compounds (including subordinate concepts) represented by the general formulae (np-1) to (np-3) can be produced by a known method.

### (Liquid Crystal Composition)

A liquid crystal composition according to the present invention can be produced, for example, by mixing the compound represented by the general formula (i), the compound represented by the general formula (ii), another optional compound described above, and an additive.

The additive may be a stabilizer, a dye compound, a polymerizable compound, an azotolane compound, or the like

The stabilizer is, for example, a hydroquinone, a hydroquinone monoalkyl ether, a tert-butylcatechol, a pyrogallol, a thiophenol, a nitro compound, a β-naphthylamine, a β-naphthol, a nitroso compound, a hindered phenol, or a hindered amine.

The hindered phenol may be a hindered phenol antioxidant represented by one of the following structural formulae (XX-1) to (XX-3):

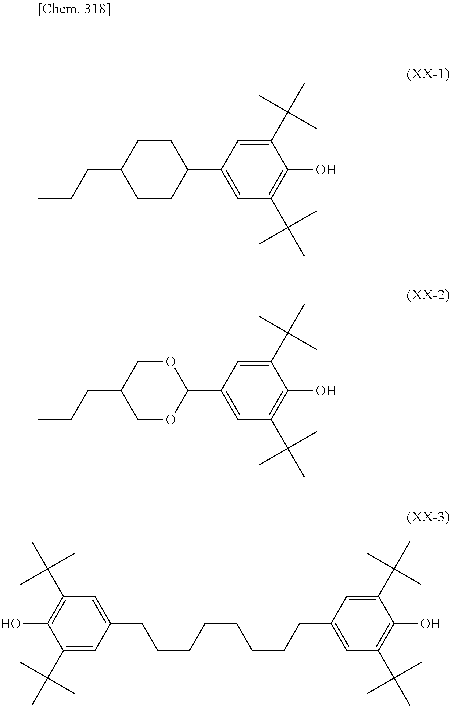 [SMILES: CCCC1CCC(c2cc(C(C)(C)C)c(O)c(C(C)(C)C)c2)CC1 ; CCCC1COC(c2cc(C(C)(C)C)c(O)c(C(C)(C)C)c2)OC1 ; CC(C)(C)c1cc(CCCCCCCCc2cc(C(C)(C)C)c(O)c(C(C)(C)C)c2)cc(C(C)(C)C)c1O]

The hindered amine may be a hindered amine light stabilizer represented by one of the following structural formulae (YY-1) and (YY-2):

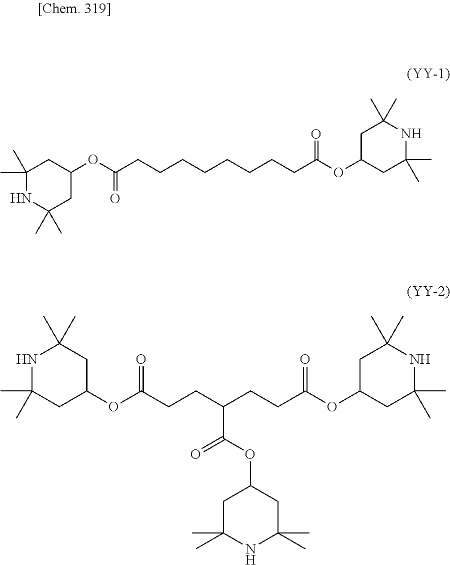

When a stabilizer is used, the number of types of stabilizers used in the liquid crystal composition is one or two or more, preferably one to ten, preferably one to eight, preferably one to six, preferably one to four, or preferably one or two.

When the stabilizer is used, the total stabilizer content per 100% by mass of the liquid crystal composition preferably ranges from 0.005% to 1% by mass, preferably 0.02% to 0.50% by mass, or preferably 0.03% to 0.35% by mass.

From the perspective of solubility, Δn, and/or Δεᵣ, the combination of compounds used in the liquid crystal composition is preferably:
1) a combination of a compound represented by the general formula (i) (including subordinate concepts) and a compound represented by the general formula (ii) (including subordinate concepts),
2) a combination of a compound represented by the general formula (i) (including subordinate concepts), a compound represented by the general formula (ii-2) (including subordinate concepts), a compound represented by the general formula (ii-3) (including subordinate concepts), a compound represented by the general formula (ii-4) (including subordinate concepts), and a compound represented by the general formula (ii-6) (including subordinate concepts),
3) a combination of a compound represented by the general formula (1-3) (including subordinate concepts), a compound represented by the general formula (ii-2) (including subordinate concepts), a compound represented by the general formula (ii-3) (including subordinate concepts), a compound represented by the general formula (ii-4) (including subordinate concepts), and a compound represented by the general formula (ii-6) (including subordinate concepts),
4) a combination of a compound represented by the general formula (1-2) (including subordinate concepts), a compound represented by the general formula (i-3) (including subordinate concepts), a compound represented by the general formula (1-4) (including subordinate concepts), a compound represented by the general formula (ii-2) (including subordinate concepts), a compound represented by the general formula (ii-3) (including subordinate concepts), a compound represented by the general formula (ii-4) (including subordinate concepts), and a compound represented by the general formula (ii-6) (including subordinate concepts), or
5) a combination of a compound represented by the general formula (1-3) (including subordinate concepts), a compound represented by the general formula (ii-1) (including subordinate concepts), a compound represented by the general formula (ii-2) (including subordinate concepts), a compound represented by the general formula (ii-3) (including subordinate concepts), and a compound represented by the general formula (ii-5) (including subordinate concepts).

### <Characteristic Values of Liquid Crystal Composition>

The liquid crystal phase upper limit temperature (Tₙᵢ) is a phase transition temperature of a liquid crystal composition from a nematic phase to an isotropic phase.

Tₙᵢ is measured by preparing a preparation in which a liquid crystal composition is disposed between a slide glass and a cover glass, followed by polarized light microscopy during heating on a hot stage.

It can also be measured by differential scanning calorimetry (DSC).

The unit is “C”.

With a higher Tₙᵢ, the nematic phase can be maintained even at high temperatures, and the driving temperature range can be widened.

The liquid crystal phase upper limit temperature (Tₙᵢ) of a liquid crystal composition according to the present invention can be appropriately set for the indoors or the inside of an automobile where the external temperature of a liquid crystal display element can be controlled, or for the outdoors, and, from the perspective of the driving temperature range, is preferably 100° C. or more, preferably 100° C. to 200° C., preferably 110° C. to 190° C., or preferably 150° C. to 180° C.

The liquid crystal phase lower limit temperature (Tn) is a phase transition temperature of a liquid crystal composition from another phase (a glass phase, a smectic phase, or a crystalline phase) to a nematic phase.

T_{→n} is measured by filling a glass capillary with a liquid crystal composition, immersing the liquid crystal composition in a refrigerant at −70° C. for a phase transition to another phase, and observing the liquid crystal composition while increasing the temperature.

It can also be measured by differential scanning calorimetry (DSC).

The unit is “° C.”.

With a lower T_{÷n}, the nematic phase can be maintained even at low temperatures, and the driving temperature range can therefore be widened.

The liquid crystal phase lower limit temperature (T_{→n}) of a liquid crystal composition according to the present invention is preferably 10° C. or less, preferably −70° C. to 0° C., or preferably −40° C. to −5° C., from the perspective of the driving temperature range.

Δn (refractive index anisotropy) correlates with Δn in the near-infrared region used in an optical sensor described later.

A higher Δn results in a higher phase modulation power of light with a target wavelength and is therefore particularly suitable for an optical sensor.

An at 25° C. and at 589 nm is determined with an Abbe refractometer from the difference (nₑ−nₒ) between the extraordinary refractive index (nₑ) and the ordinary refractive index (nₒ) of a liquid crystal composition.

Δn can also be determined with a phase difference measuring apparatus.

The phase difference Re, the thickness d of a liquid crystal layer, and Δn have the relationship Δn=Re/d.

A liquid crystal composition is injected into a glass cell with a polyimide alignment film, which has a cell gap (d) of approximately 3.0 μm and has been subjected to antiparallel rubbing treatment, and the in-plane Re is measured using a retardation film and an optical material inspection apparatus RETS-100 (manufactured by Otsuka Electronics Co., Ltd.).

The measurement is performed at a temperature of 25° C. and at 589 nm, and there is no unit.

Δn of a liquid crystal composition according to the present invention at 25° C. and at 589 nm is preferably 0.38 or more, preferably 0.38 to 0.65, preferably 0.40 to 0.60, or preferably 0.45 to 0.55, from the perspective of the phase modulation power of light of a wavelength.

The rotational viscosity (γᵢ) is a viscosity coefficient related to the rotation of a liquid crystal molecule.

γ₁ can be measured with LCM-2 (manufactured by Toyo Corporation) by filling a glass cell having a cell gap of approximately 10 μm with a liquid crystal composition and applying a voltage of 50 V.

A horizontal alignment cell is used for a liquid crystal composition with positive dielectric anisotropy, and a vertical alignment cell is used for a liquid crystal composition with negative dielectric anisotropy.

The measurement is performed at a temperature of 25° C., and the unit is mPa·s.

A liquid crystal composition with lower vi has a higher response speed and is suitable for any liquid crystal display element.

From the perspective of the response speed, a liquid crystal composition according to the present invention preferably has a rotational viscosity (γᵢ) in the range of 150 to 2000 mPa·s, preferably 200 to 1500 mPa·s, or preferably 250 to 1000 mPa·s, at 25° C.

Higher dielectric anisotropy in a high-frequency region results in a higher phase modulation power for a radio wave in a target frequency band and is particularly suitable for antenna applications.

In antenna applications, a lower dielectric loss tangent in a high-frequency region is preferred due to a smaller energy loss in a target frequency band.

In a liquid crystal composition according to the present invention, the dielectric anisotropy Aar and the average value tan δᵢₛₒ of dielectric loss tangent at 10 GHz were measured as representative characteristics in a high-frequency region.

Δεᵣ=(ε_{r//}−εr_{⊥}), and tan δᵢₛₒ=(2ε_{r⊥}tan δ_{⊥}+ε_{r//}tan δ_{//})/(2ε_{t⊥}+ε_{r//}).

“εᵣ” denotes a dielectric constant, “tan δ” denotes a dielectric loss tangent, the subscript “//” denotes a component in a direction parallel to the orientation direction of a liquid crystal, and the subscript “⊥” denotes a component in a direction perpendicular to the orientation direction of a liquid crystal.

Δεᵣ and tan δᵢₛₒ can be measured by the following method.

First, a liquid crystal composition is introduced into a capillary made of polytetrafluoroethylene (PTFE).

The capillary has an inner radius of 0.80 mm, an outer radius of 0.835 mm, and an effective length of 4.0 cm.

The capillary filled with the liquid crystal composition is introduced into the center of a cavity resonator with a resonance frequency of 10 GHZ (manufactured by EM labs, Inc.).

The cavity resonator has an external shape with a diameter of 30 mm and a width of 26 mm.

A signal is input, and the result of an output signal is recorded with a network analyzer (manufactured by Keysight Technologies).

The dielectric constant (εᵣ) and the loss angle (δ) at 10 GHz are determined using the difference between the resonance frequency or the like of the PTFE capillary without the liquid crystal composition and the resonance frequency or the like of the PTFE capillary filled with the liquid crystal composition.

The tangent of δ thus determined is the dielectric loss tangent (tan δ).

The resonance frequency or the like of the PTFE capillary filled with the liquid crystal composition is determined by the orientation control of liquid crystal molecules as the value of a characteristic component perpendicular to the orientation direction of the liquid crystal molecules and as the value of a characteristic component parallel to the orientation direction of the liquid crystal molecules.

A magnetic field of a permanent magnet or an electromagnet is used to arrange liquid crystal molecules in the vertical direction (perpendicular to the effective length direction) or in the parallel direction (parallel to the effective length direction) of the PTFE capillary.

The magnetic field has, for example, a distance of 45 mm between magnetic poles and a magnetic field strength of 0.23 tesla near the center.

The desired characteristic component is obtained by rotating the PTFE capillary filled with the liquid crystal composition in parallel or perpendicular to the magnetic field.

The measurement is performed at a temperature of 25° C., and both Δεᵣ and tan δᵢₛₒ have no units.

The Aer of a liquid crystal composition according to the present invention at 25° C. is preferably higher and, from the perspective of the phase modulation power in the GHz band, is preferably 0.90 or more, preferably 0.90 to 1.50, preferably 0.95 to 1.45, or preferably 1.00 to 1.40.

The tan δᵢₛₒ of a liquid crystal composition according to the present invention at 25° C. is preferably lower and, from the perspective of loss in the GHz band, is preferably 0.025 or less, preferably 0.001 to 0.025, preferably 0.003 to 0.020, preferably 0.005 to 0.017, preferably 0.007 to 0.015, preferably 0.008 to 0.013, or preferably 0.009 to 0.012.

### (Liquid Crystal Display Element, Sensor, Liquid Crystal Lens, Optical Communication Equipment, and Antenna)

A liquid crystal display element, a sensor, a liquid crystal lens, optical communication equipment, and an antenna using a liquid crystal composition according to the present invention are described below.

A liquid crystal display element according to the present invention is characterized by using the liquid crystal composition and is preferably driven by an active matrix system or a passive matrix system.

A liquid crystal display element according to the present invention is preferably a liquid crystal display element that reversibly switches permittivity by reversibly changing the orientation direction of liquid crystal molecules of the liquid crystal composition.

A sensor according to the present invention is characterized by using the liquid crystal composition and is, for example, in one embodiment, a range sensor utilizing electromagnetic waves, visible light, or infrared light, an infrared sensor utilizing a temperature change, a temperature sensor utilizing a reflected light wavelength change due to a pitch change of a cholesteric liquid crystal, a pressure sensor utilizing a reflected light wavelength change, an ultraviolet sensor utilizing a reflected light wavelength change due to a composition change, an electric sensor utilizing a temperature change due to the voltage or the electric current, a radiation sensor utilizing a temperature change associated with a track of radiation particles, an ultrasonic sensor utilizing a liquid crystal molecular arrangement change due to mechanical vibration of ultrasonic waves, or an electromagnetic field sensor utilizing a reflected light wavelength change due to a temperature change or a liquid crystal molecular arrangement change due to an electric field.

The range sensor is preferably used for light detection and ranging (LiDAR) using a light source.

The LiDAR is preferably used for an artificial satellite, an aircraft, an unmanned aerial vehicle (drone), an automobile, a railway, or a ship.

A LiDAR for an automobile is particularly preferably a LiDAR for a self-driving vehicle.

The light source is preferably an LED or a laser, preferably a laser.

The light used for LiDAR is preferably infrared light and preferably has a wavelength in the range of 800 to 2000 nm.

In particular, an infrared laser with a wavelength of 905 nm or 1550 nm is preferred.

An infrared laser with a wavelength of 905 nm is preferred when the cost of a photodetector to be used and the sensitivity in all weather are regarded as important, and an infrared laser with a wavelength of 1550 nm is preferred when the safety related to human vision is regarded as important.

A liquid crystal composition according to the present invention has a high Δn and can therefore provide a sensor with a high phase modulation power in visible light, infrared light, and electromagnetic wave regions and with high detectivity.

A liquid crystal lens according to the present invention is characterized by using the liquid crystal composition and includes, for example, in one embodiment, a first transparent electrode layer, a second transparent electrode layer, a liquid crystal layer containing the liquid crystal composition between the first transparent electrode layer and the second transparent electrode layer, an insulating layer between the second transparent electrode layer and the liquid crystal layer, and a high-resistance layer between the insulating layer and the liquid crystal layer.

A liquid crystal lens according to the present invention is utilized, for example, as a 2D or 3D switching lens, a focusing lens for a camera, or the like.

Optical communication equipment according to the present invention is characterized by using the liquid crystal composition, and is, for example, in one embodiment, a liquid crystal on silicon (LCOS) including a liquid crystal layer in which a liquid crystal constituting each of a plurality of pixels is two-dimensionally arranged on a reflective layer (electrode).

Optical communication equipment according to the present invention is used, for example, as a spatial phase modulator.

An antenna according to the present invention is characterized by using the liquid crystal composition.

More specifically, an antenna according to the present invention includes a first substrate with a plurality of slots, a second substrate facing the first substrate and having a power supply portion, a first dielectric layer between the first substrate and the second substrate, a plurality of patch electrodes arranged corresponding to the plurality of slots, a third substrate provided with the patch electrodes, and a liquid crystal layer between the first substrate and the third substrate, wherein the liquid crystal layer contains the liquid crystal composition described above.

A liquid crystal composition containing one or two or more compounds represented by the general formula (i) (including subordinate concepts) having a tetracyclic structure, —C≡C— as a linking group between the ring structures, and an isothiocyanate group (—NCS) can be used as a liquid crystal composition to provide an antenna that has a high Tₙᵢ, a high Δn, a high Δεᵣ, a low tan δᵢₛₒ, and high storage stability at room temperature and that therefore has high reliability against an external stimulus, such as heat.

This makes it possible to provide an antenna that enables greater phase control of a microwave or millimeter-wave electromagnetic wave.

An antenna according to the present invention preferably operates at a Ka band frequency, a K band frequency, or a Ku band frequency used for satellite communications.

An antenna according to the present invention preferably has a configuration in which a radial line slot array and a patch antenna array are combined.

As the structure of an antenna according to the present invention, for example, the matters described in International Publication No. WO 2021/157189 pamphlet or the like can be referred to and applied.

### EXAMPLES

Although the present invention is described in more detail in the following examples, the present invention is not limited to these examples.

A composition in the following examples and comparative examples contains compounds in the proportion shown in tables, and their respective contents are expressed in “% by mass”.

The following abbreviations are used to describe the compounds.

Unless otherwise specified, a compound that can have a cis form or a trans form is represented by a trans form.

### <Ring Structure>

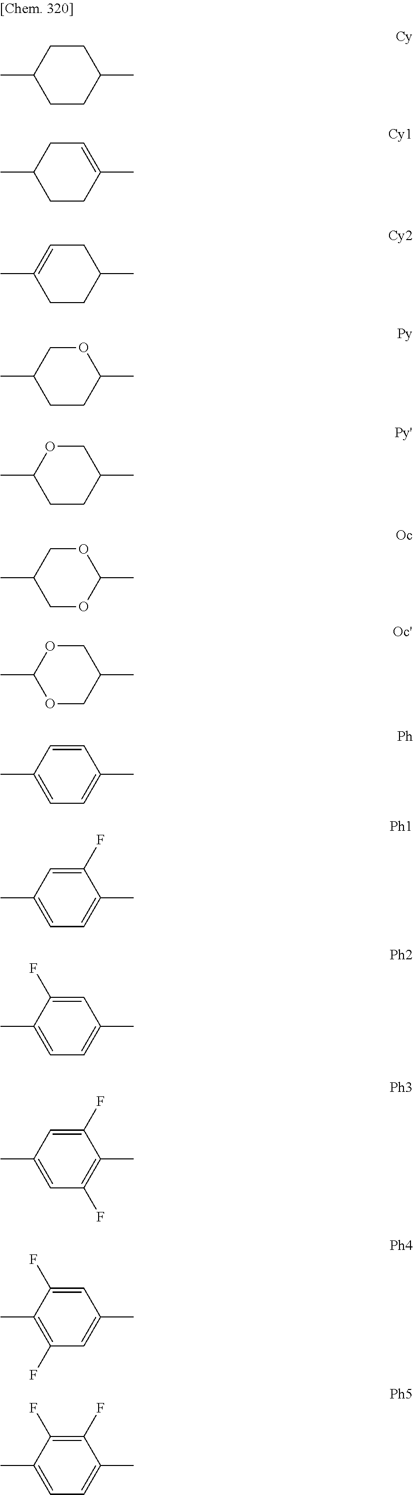

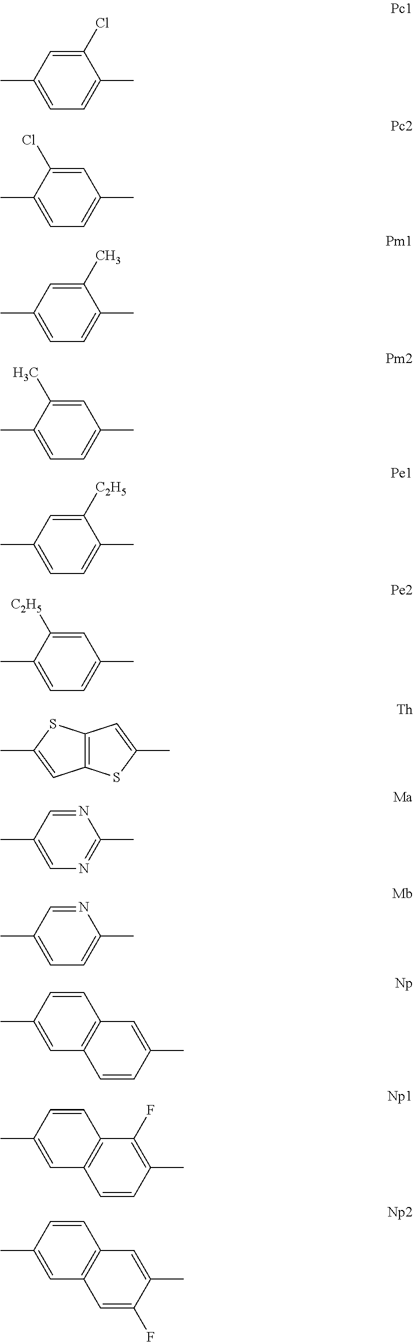

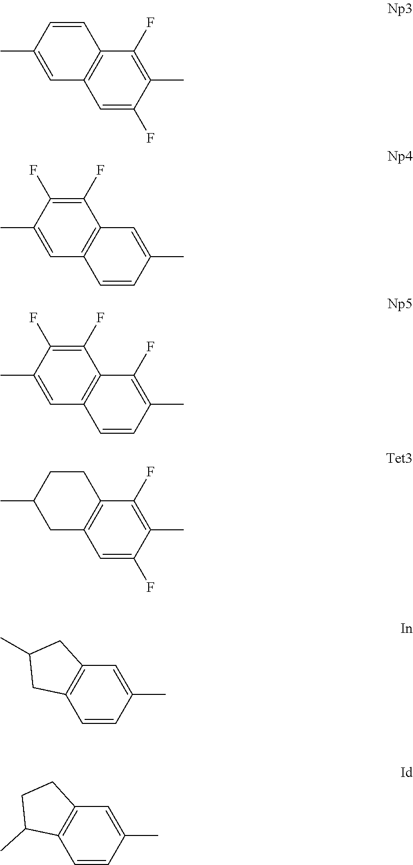

### <Terminal Structure>

TABLE 1

| Abbreviation | Chemical structure |
|---|---|
| -n | —CₙH₂ₙ₊₁ |
| n- | CₙH₂ₙ₊₁— |
| —On | —O—CₙH₂ₙ₊₁ |
| nO— | CₙH₂ₙ₊₁—O— |
| —Sn | —S—CₙH₂ₙ₊₁ |
| nS— | CₙH₂ₙ₊₁—S— |
| —V | —CH═CH₂ |
| V— | CH₂═CH— |
| —V1 | —CH═CH—CH₃ |
| 1V— | CH₃—CH═CH— |
| —2V | —CH₂—CH₂—CH═CH₂ |
| V2— | CH₂═CH—CH₂—CH₂— |
| —2V1 | —CH₂—CH₂—CH═CH—CH₃ |
| 1V2— | CH₃—CH═CH—CH₂—CH₂— |
| —OCF3 | —O—CF₃ |
| CF3O— | CF₃—O— |
| —H | —H |
| H— | H— |
| —CN | —CN |
| CN— | CN— |
| —NCS | —NCS |
| NCS— | NCS— |
| -(1)4 | —CH₂CH₂CH(CH₃)CH₃ |
| 4(1)- | CH₃CH(CH₃)CH₂CH₂— |

(n in the table denotes a natural number)

### <Linking Structure>

TABLE 2

| Abbreviation | Chemical structure |
|---|---|
| -n- | —CₙH₂ₙ— |
| -nO— | —CₙH₂ₙ—O— |
| —On- | —O—CₙH₂ₙ— |
| —COO— | —C(═O)—O— |
| —OCO— | —O—C(═O)— |
| —V— | —CH═CH— |
| -nV— | —CₙH₂ₙ—CH═CH— |
| —Vn- | —CH═CH—CₙH₂ₙ— |
| -T- | —C≡C— |
| —CF₂O— | —CF₂—O— |
| —OCF₂— | —O—CF₂— |
| -Az- | —N═N— |

(n in the table denotes a natural number)

### (Hindered Phenol Antioxidant)

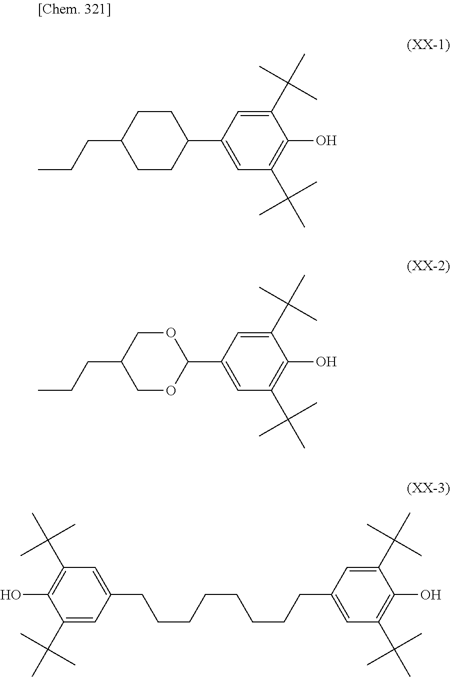 [SMILES: CCCC1CCC(c2cc(C(C)(C)C)c(O)c(C(C)(C)C)c2)CC1 ; CCCC1COC(c2cc(C(C)(C)C)c(O)c(C(C)(C)C)c2)OC1 ; CC(C)(C)c1cc(CCCCCCCCc2cc(C(C)(C)C)c(O)c(C(C)(C)C)c2)cc(C(C)(C)C)c1O]

### (Hindered Amine Light Stabilizer)

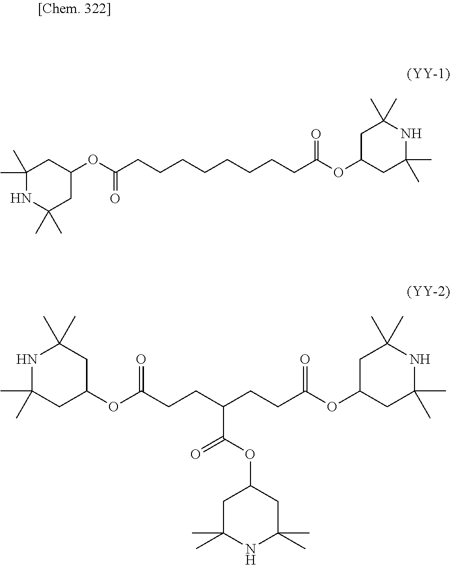

### (Preparation of Liquid Crystal Composition)

LC-A, LC-B, and LC-01 to LC-07 in Table 3 were prepared.

TABLE 3

| Table 3 | LC-A | LC-B | LC-01 | LC-02 | LC-03 | LC-04 | LC-05 | LC-06 | LC-07 |
|---|---|---|---|---|---|---|---|---|---|
| 3-T-Pm2-Ph1-Ph-T-Ph3-NCS | | | 5 | 5 | 5 | | 5 | | 10 |
| 4-T-Pm2-Ph1-Ph-T-Ph3-NCS | | | | 10 | 10 | | | | 10 |
| 5-T-Pm2-Ph1-Ph-T-Ph3-NCS | | | | | 5 | | | | |
| 3-T-Pm2-Ph2-Ph-T-Ph3-NCS | | | | | | 5 | | | |
| 4-T-Pm2-Ph2-Ph-T-Ph3-NCS | | | | | | 5 | | | |
| 4-T-Ph2-Pm2-T-Ph-Ph3-NCS | | | | | | | 5 | | |
| 4-T-Ph-T-Pm2-Ph-T-Ph3-NCS | | | | | | | 5 | | |
| 4-Ph-Ph-Ph-T-Ph3-NCS | | | | | | | | 5 | |
| 4-Pm2-Ph1-Ph-T-Ph3-NCS | | | | | | | | 5 | |
| 3-In-Ph-T-Ph3-NCS | | | | | | 10 | 5 | | |
| 4-In-T-Ph1-T-Ph3-NCS | | | | | | | 5 | | |
| 3-Tet3-T-Ph-T-Ph1-NCS | | | 7 | | | 7 | 7 | | |
| 4-T-Ph-T-Ph-Ph3-NCS | | | 5 | | | 5 | 5 | 5 | |
| 5-T-Ph-T-Ph-Ph3-NCS | | | | | 3 | | | | |
| 4-T-Ph-T-Ph1-Ph3-NCS | | | 5 | 5 | 5 | 5 | 5 | 5 | |
| 5-T-Ph-T-Ph1-Ph3-NCS | | | 5 | 5 | 7 | | | 5 | |
| 4-T-Ph-Ph2-T-Ph3-NCS | | | 10 | 10 | 5 | 10 | 10 | 10 | |
| 3-T-Pm2-Ph-T-Ph3-NCS | | | 5 | 5 | | 5 | 5 | 5 | |
| 4-T-Pm2-Ph-T-Ph3-NCS | | | 10 | 10 | 4 | 10 | 10 | 10 | |
| 5-T-Pm2-Ph-T-Ph3-NCS | | | 5 | 5 | | 5 | | 5 | |
| 4-T-Ph2-T-Ph-Ph3-NCS | | | 10 | 10 | 5 | | | 10 | |
| 4-T-Ph1-Ph-T-Ph3-NCS | | | | | 4 | | | | |
| 5-T-Ph1-Ph-T-Ph3-NCS | | | | | 7 | | | | |
| 5-Ph3-T-Ph-Ph3-NCS | | | | | 5 | | | | |
| 3-Cy-T-Ph-T-Ph3-NCS | | | 5 | 6 | 5 | 5 | 5 | 6 | |
| 4-Cy-T-Ph-T-Ph3-NCS | | | 5 | 6 | 5 | 5 | 5 | 6 | |
| 5-Cy-Ph-NCS | | 6 | | | | | | | 5 |
| 4-Ph-T-Pc1-NCS | | 11 | | | | | | | 9 |
| 40-Ph2-T-Ph-NCS | | 5 | | | | | | | 4 |
| 50-Ph2-T-Ph-NCS | | 5 | | | | | | | 4 |
| 5-Ph-T-Ph1-NCS | | 5 | | | | | | | 4 |
| 2-Ph-T-Ph3-NCS | | | 10 | 10 | 10 | 10 | | 10 | |
| 3-Ph-T-Ph3-NCS | 13 | | 13 | 8 | 15 | | 10 | 8 | |
| 4-Ph-T-Ph3-NCS | | | | | | 13 | | | |
| 5-Ph-T-Ph3-NCS | 11 | | | 5 | | | 13 | 5 | |
| 2-Cy-Ph-Ph3-NCS | | 12 | | | | | | | 10 |
| 4-Cy-Ph-Ph3-NCS | | 12 | | | | | | | 10 |
| 4-Cy-Ph-T-Ph1-NCS | | 16 | | | | | | | 12 |
| 5-Cy-Ph-T-Ph1-NCS | | 13 | | | | | | | 10 |
| 4-Cy-Ph-T-Ph3-NCS | 14 | | | | | | | | |
| 5-Cy-Ph-T-Ph3-NCS | 20 | | | | | | | | |
| CF30-Ph-Ph-Ph3-NCS | 24 | | | | | | | | |
| 4-Ph-Ph-T-Ph3-NCS | 6 | | | | | | | | |
| 5-Ph-Ph-T-Ph3-NCS | 12 | | | | | | | | |
| 5-Ph-Ph5-T-Ph1-NCS | | 15 | | | | | | | 12 |
| Total [mass %] | 100 | 100 | 100 | 100 | 100 | 100 | 100 | 100 | 100 |

### Examples 1 to 37 and Comparative Examples 1 and 2

Liquid crystal compositions described in Tables 4 to 9 were prepared from LC-A, LC-B, and LC-01 to LC-07, hindered phenol antioxidants (XX-1) to (XX-3), and hindered amine light stabilizers (YY-1) and (YY-2), and their physical properties were measured, and <Storage Test> was performed. Tables 4 to 9 show the results. Comparative Example 1 was crystallized at room temperature, and its high-frequency characteristics (Δεᵣ and tan δᵢₛₒ) were not measured.

### <Storage Test>

0.5 g of a liquid crystal composition was weighed in a 1-mL sample bottle (manufactured by Maruemu Corporation) and was degassed at 150 to 250 Pa for 10 minutes. It was then purged with dry nitrogen and was covered with a fitting lid. This was stored in a temperature-controlled thermostat (SH-241 manufactured by Espec Corp.) at 25° C. for 2 weeks, and the liquid crystal composition was visually inspected for crystallization every week.

TABLE 4

| | Comparative | Comparative | Example | Example | Example | Example | Example | Example | Example |
|---|---|---|---|---|---|---|---|---|---|
| Table 4 | example 1 | example 2 | 1 | 2 | 3 | 4 | 5 | 6 | 7 |
| Liquid crystal | LC-A | LC-B | LC-01 | LC-02 | LC-03 | LC-04 | LC-05 | LC-06 | LC-07 |
| composition | | | | | | | | | |
| Tₙᵢ [°C] | 156 | 150 | 170 | 170 | 171 | 175 | 184 | 165 | 174 |
| Δn | 0.413 | 0.368 | 0.502 | 0.500 | 0.495 | 0.497 | 0.507 | 0.496 | 0.408 |
| Δεᵣ | — | 1.091 | 1.356 | 1.355 | 1.347 | 1.350 | 1.365 | 1.348 | 1.137 |
| tanδᵢₛₒ | — | 0.019 | 0.011 | 0.011 | 0.012 | 0.011 | 0.013 | 0.012 | 0.014 |
| Storage | Crystallized | No | No | No | No | No | No | No | No |
| stability | at room | crystal- | crystal- | crystal- | crystal- | crystal- | crystal- | crystal- | crystal- |
| (25 °C) | temperature | lization | lization | lization | lization | lization | lization | lization | lization |
| | | for two | for two | for two | for two | for two | for two | for two | for two |
| | | weeks | weeks | weeks | weeks | weeks | weeks | weeks | weeks |

TABLE 5

| Table 5 | | Example 8 | Example 9 | Example 10 | Example 11 | Example 12 | Example 13 |
|---|---|---|---|---|---|---|---|
| Liquid crystal | LC-01 | 99.70 | 99.80 | 99.80 | 99.80 | 99.75 | 99.75 |
| composition | | | | | | | |
| [mass %] | | | | | | | |
| Additive | XX-1 | | | 0.20 | | | |
| [mass %] | XX-2 | | | | 0.20 | 0.20 | |
| | XX-3 | 0.30 | 0.15 | | | | 0.20 |
| | YY-1 | | | | | 0.05 | 0.05 |
| | YY-2 | | 0.05 | | | | |

| | | | | | | |
|---|---|---|---|---|---|---|
| Total [mass %] | 100.00 | 100.00 | 100.00 | 100.00 | 100.00 | 100.00 |
| Tₙᵢ [°C] | 169 | 169 | 169 | 169 | 169 | 169 |
| Δn | 0.501 | 0.501 | 0.501 | 0.501 | 0.501 | 0.501 |
| Δεᵣ | 1.356 | 1.356 | 1.356 | 1.356 | 1.356 | 1.356 |
| tanδᵢₛₒ | 0.011 | 0.011 | 0.011 | 0.011 | 0.011 | 0.011 |
| Storage | No | No | No | No | No | No |
| stability | crystallization | crystallization | crystallization | crystallization | crystallization | crystallization |
| (25 °C) | for two weeks | for two weeks | for two weeks | for two weeks | for two weeks | for two weeks |

TABLE 6

| Table 6 | | Example 14 | Example 15 | Example 16 | Example 17 | Example 18 | Example 19 |
|---|---|---|---|---|---|---|---|
| Liquid crystal | LC-02 | 99.70 | 99.80 | 99.80 | 99.80 | 99.75 | 99.75 |
| composition | | | | | | | |
| [mass %] | | | | | | | |
| Additive | XX-1 | | | 0.20 | | | |
| [mass %] | XX-2 | | | | 0.20 | 0.20 | |
| | XX-3 | 0.30 | 0.15 | | | | 0.20 |
| | YY-1 | | | | | 0.05 | 0.05 |
| | YY-2 | | 0.05 | | | | |

| | | | | | | |
|---|---|---|---|---|---|---|
| Total [mass %] | 100.00 | 100.00 | 100.00 | 100.00 | 100.00 | 100.00 |
| Tₙᵢ [°C] | 169 | 169 | 169 | 169 | 169 | 169 |
| Δn | 0.499 | 0.499 | 0.499 | 0.499 | 0.499 | 0.499 |
| Δεᵣ | 1.355 | 1.355 | 1.355 | 1.355 | 1.355 | 1.355 |
| tanδᵢₛₒ | 0.011 | 0.011 | 0.011 | 0.011 | 0.011 | 0.011 |
| Storage | No | No | No | No | No | No |
| stability | crystallization | crystallization | crystallization | crystallization | crystallization | crystallization |
| (25 °C) | for two weeks | for two weeks | for two weeks | for two weeks | for two weeks | for two weeks |

TABLE 7

| Table 7 | | Example 20 | Example 21 | Example 22 | Example 23 | Example 24 | Example 25 |
|---|---|---|---|---|---|---|---|
| Liquid crystal | LC-04 | 99.70 | 99.80 | 99.80 | 99.80 | 99.75 | 99.75 |
| composition | | | | | | | |
| [mass %] | | | | | | | |
| Additive | XX-1 | | | 0.20 | | | |
| [mass %] | XX-2 | | | | 0.20 | 0.20 | |
| | XX-3 | 0.30 | 0.15 | | | | 0.20 |
| | YY-1 | | | | | 0.05 | 0.05 |
| | YY-2 | | 0.05 | | | | |

| | | | | | | |
|---|---|---|---|---|---|---|
| Total [mass %] | 100.00 | 100.00 | 100.00 | 100.00 | 100.00 | 100.00 |
| Tₙᵢ [°C] | 174 | 174 | 174 | 174 | 174 | 174 |
| Δn | 0.496 | 0.496 | 0.496 | 0.496 | 0.496 | 0.496 |
| Δεᵣ | 1.350 | 1.350 | 1.350 | 1.350 | 1.350 | 1.350 |
| tanδᵢₛₒ | 0.011 | 0.011 | 0.011 | 0.011 | 0.011 | 0.011 |
| Storage | No | No | No | No | No | No |
| stability | crystallization | crystallization | crystallization | crystallization | crystallization | crystallization |
| (25 °C) | for two weeks | for two weeks | for two weeks | for two weeks | for two weeks | for two weeks |

TABLE 8

| Table 8 | | Example 26 | Example 27 | Example 28 | Example 29 | Example 30 | Example 31 |
|---|---|---|---|---|---|---|---|
| Liquid crystal | LC-06 | 99.70 | 99.80 | 99.80 | 99.80 | 99.75 | 99.75 |
| composition | | | | | | | |
| [mass %] | | | | | | | |
| Additive | XX-1 | | | 0.20 | | | |
| [mass %] | XX-2 | | | | 0.20 | 0.20 | |
| | XX-3 | 0.30 | 0.15 | | | | 0.20 |
| | YY-1 | | | | | 0.05 | 0.05 |
| | YY-2 | | 0.05 | | | | |

| | | | | | | |
|---|---|---|---|---|---|---|
| Total [mass %] | 100.00 | 100.00 | 100.00 | 100.00 | 100.00 | 100.00 |
| Tₙᵢ [°C] | 164 | 164 | 164 | 164 | 164 | 164 |
| Δn | 0.495 | 0.495 | 0.495 | 0.495 | 0.495 | 0.495 |
| Δεᵣ | 1.348 | 1.348 | 1.348 | 1.348 | 1.348 | 1.348 |
| tanδᵢₛₒ | 0.012 | 0.012 | 0.012 | 0.012 | 0.012 | 0.012 |
| Storage | No | No | No | No | No | No |
| stability | crystallization | crystallization | crystallization | crystallization | crystallization | crystallization |
| (25 °C) | for two weeks | for two weeks | for two weeks | for two weeks | for two weeks | for two weeks |

TABLE 9

| Table 9 | | Example 32 | Example 33 | Example 34 | Example 35 | Example 36 | Example 37 |
|---|---|---|---|---|---|---|---|
| Liquid crystal | LC-07 | 99.70 | 99.80 | 99.80 | 99.80 | 99.75 | 99.75 |
| composition | | | | | | | |
| [mass %] | | | | | | | |
| Additive | XX-1 | | | 0.20 | | | |
| [mass %] | XX-2 | | | | 0.20 | 0.20 | |
| | XX-3 | 0.30 | 0.15 | | | | 0.20 |
| | YY-1 | | | | | 0.05 | 0.05 |
| | YY-2 | | 0.05 | | | | |

| | | | | | | |
|---|---|---|---|---|---|---|
| Total [mass %] | 100.00 | 100.00 | 100.00 | 100.00 | 100.00 | 100.00 |
| Tₙᵢ [°C] | 173 | 173 | 173 | 173 | 173 | 173 |
| Δn | 0.407 | 0.407 | 0.407 | 0.407 | 0.407 | 0.407 |
| Δεᵣ | 1.137 | 1.137 | 1.137 | 1.137 | 1.137 | 1.137 |
| tanδᵢₛₒ | 0.014 | 0.014 | 0.014 | 0.014 | 0.014 | 0.014 |
| Storage | No | No | No | No | No | No |
| stability | crystallization | crystallization | crystallization | crystallization | crystallization | crystallization |
| (25 °C) | for two weeks | for two weeks | for two weeks | for two weeks | for two weeks | for two weeks |

Examples 1 to 7 showed that a liquid crystal composition using a compound represented by the general formula (i) had a high Tₙᵢ, a high Δn, a high Δεᵣ, a low tan δᵢₛₒ, and high storage stability at room temperature.

In particular, Examples 1, 2, and 5 had particularly high Δn and Δεᵣ.

On the other hand, Comparative Examples 1 and 2 showed that a liquid crystal composition not using a compound represented by the general formula (i) was crystallized at room temperature or had a low Δn of less than 0.38.

Furthermore, even when a hindered phenol antioxidant or a hindered amine light stabilizer was used in combination, Examples 8 to 37 showed a high Tₙᵢ, a high Δn, a high Δεᵣ, a low tan δᵢₛₒ, and high storage stability at room temperature.

The synthesis of a compound represented by the general formula (i) is described below.

### (Synthesis Example 1) Production of Compound Represented by Formula (I-1)

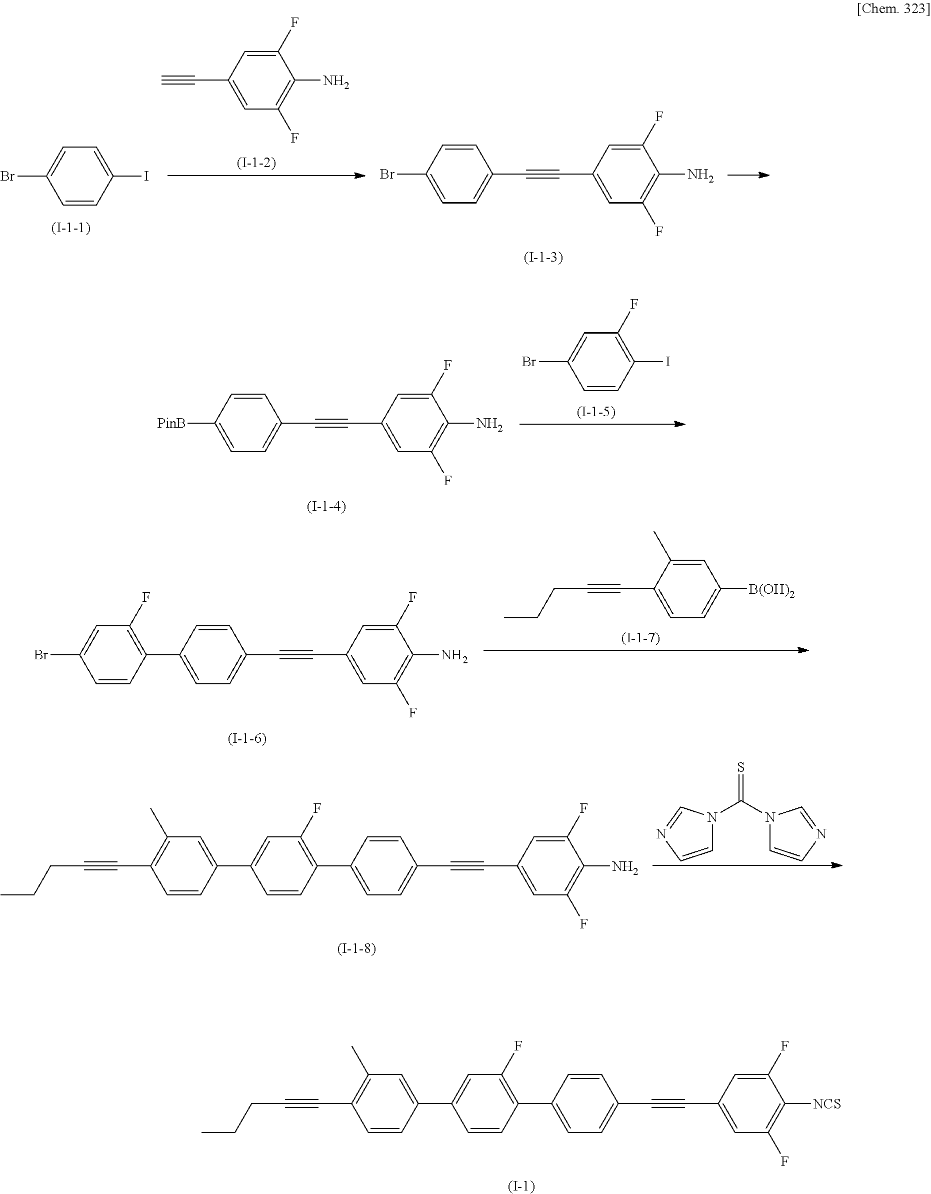

50 g of a compound represented by the formula (I-1-1), 1.4 g of copper (I) iodide, 2.5 g of bis(triphenylphosphine) palladium (II) dichloride, 53.7 g of triethylamine, and 125 mL of tetrahydrofuran were put into a reaction vessel in a nitrogen atmosphere at room temperature. Next, 30.0 g of a compound represented by the formula (I-1-2) dissolved in 125 mL of tetrahydrofuran was added dropwise while stirring at room temperature and was stirred at room temperature for 1 hour. After completion of the reaction, saturated aqueous ammonium chloride was poured into the reaction liquid, and extraction with toluene was performed. The organic layer was washed with saturated saline and was purified by column chromatography (amino silica gel, toluene/hexane=1/8 to 1/0) to produce 50.3 g of a compound represented by the formula (I-1-3).

Next, 42.0 g of the compound represented by the formula (I-1-3), 36.3 g of bis(pinacolato)diboron, 40.1 g of potassium acetate, 2.2 g of [1,1′-bis(diphenylphosphino)ferrocene] palladium (II) dichloride dichloromethane adduct, and 420 mL of dimethyl sulfoxide were added to a reaction vessel in a nitrogen atmosphere at room temperature and were stirred at 90° C. After completion of the reaction, saturated aqueous ammonium chloride was poured into the reaction liquid, and extraction with toluene was performed. The organic layer was washed with saturated saline and was purified by column chromatography (alumina silica gel, toluene) to produce 36.6 g of a compound represented by the formula (I-1-4).

Next, 6.3 g of a compound represented by the formula (I-1-5), 7.5 g of the compound represented by the formula (I-1-4), 0.1 g of bis(triphenylphosphine) palladium (II) dichloride, 4.3 g of sodium carbonate, 30 mL of tetrahydrofuran, and 20 mL of water were added to a reaction vessel in a nitrogen atmosphere at room temperature and were stirred at 75° C. for 2 hours. After completion of the reaction, the product was collected by filtration and was purified by column chromatography (amino silica gel, toluene/hexane=2/3 to 1/0) to produce 6.5 g of a compound represented by the formula (1-1-6).

Next, 6.5 g of the compound represented by the formula (I-1-6), 3.6 g of a compound represented by the formula (1-1-7), 0.1 g of bis(triphenylphosphine) palladium (II) dichloride, 4.3 g of sodium carbonate, 30 mL of tetrahydrofuran, and 20 mL of water were added to a reaction vessel in a nitrogen atmosphere at room temperature and were stirred at 75° C. for 2 hours. After completion of the reaction, the product was collected by filtration and was purified by column chromatography (amino silica gel, toluene/hexane=2/3 to 1/0) to produce 6 g of a compound represented by the formula (I-1-8).

Next, 6 g of the compound represented by the formula (I-1-8), 17 mL of dichloromethane, and 2.5 g of 1,1-thiocarbonyldiimidazole were added to a reaction vessel in a nitrogen atmosphere at room temperature and were stirred at room temperature. After completion of the reaction, the organic layer was washed with saturated saline and was purified by column chromatography (silica gel, toluene) and subsequent recrystallization (toluene/hexane=1/1) to produce 5 g of a compound represented by the formula (I-1).

MS (EI): m/z=521

### (Synthesis Example 2) Production of Compound Represented by Formula (I-2)

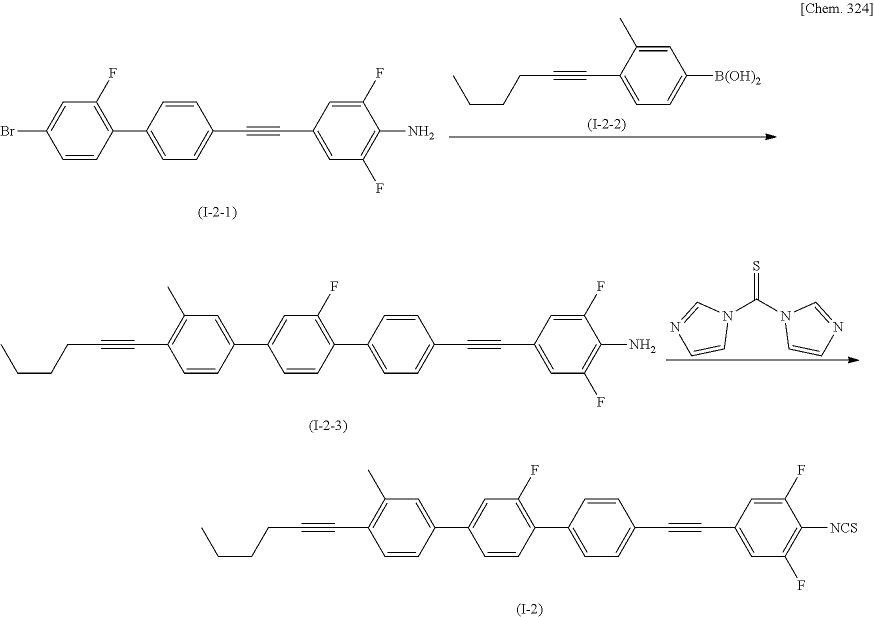

6.5 g of a compound represented by the formula (I-2-1), 3.7 g of a compound represented by the formula (1-2-2), 0.1 g of bis(triphenylphosphine) palladium (II) dichloride, 4.3 g of sodium carbonate, 30 ml of tetrahydrofuran, and 20 ml of water were added to a reaction vessel in a nitrogen atmosphere at room temperature and were stirred at 75° C. for 2 hours. After completion of the reaction, the product was collected by filtration and was purified by column chromatography (amino silica gel, toluene/hexane=2/3 to 1/0) to produce 6.2 g of a compound represented by the formula (I-2-3).

Next, 6.2 g of the compound represented by the formula (I-2-3), 17 mL of dichloromethane, and 2.6 g of 1,1-thiocarbonyldiimidazole were added to a reaction vessel in a nitrogen atmosphere at room temperature and were stirred at room temperature. After completion of the reaction, the organic layer was washed with saturated saline and was purified by column chromatography (silica gel, toluene) and subsequent recrystallization (toluene/hexane=1/1) to produce 5.3 g of a compound represented by the formula (1-2).

MS (EI): m/z=535

### (Synthesis Example 3) Production of Compound Represented by Formula (I-3)

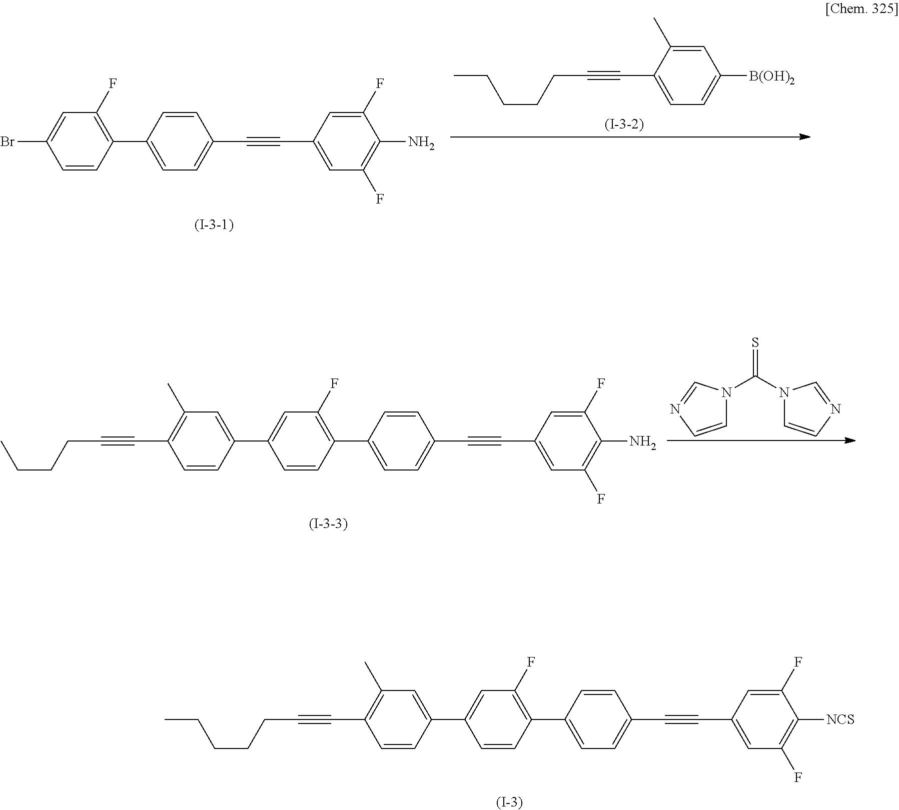

6.5 g of a compound represented by the formula (1-3-1), 3.8 g of the compound represented by the formula (1-3-2), 0.1 g of bis(triphenylphosphine) palladium (II) dichloride, 4.3 g of sodium carbonate, 30 mL of tetrahydrofuran, and 20 mL of water were added to a reaction vessel in a nitrogen atmosphere at room temperature and were stirred at 75° C. for 2 hours. After completion of the reaction, the product was collected by filtration and was purified by column chromatography (amino silica gel, toluene/hexane=2/3 to 1/0) to produce 6.3 g of a compound represented by the formula (I-3-3).

Next, 6.3 g of the compound represented by the formula (I-3-3), 17 mL of dichloromethane, and 2.6 g of 1,1-thiocarbonyldiimidazole were added to a reaction vessel in a nitrogen atmosphere at room temperature and were stirred at room temperature. After completion of the reaction, the organic layer was washed with saturated saline and was purified by column chromatography (silica gel, toluene) and subsequent recrystallization (toluene/hexane=1/1) to produce 5.5 g of a compound represented by the formula (I-3).

MS (EI): m/z=549

### (Synthesis Example 4) Production of Compound Represented by Formula (I-4)

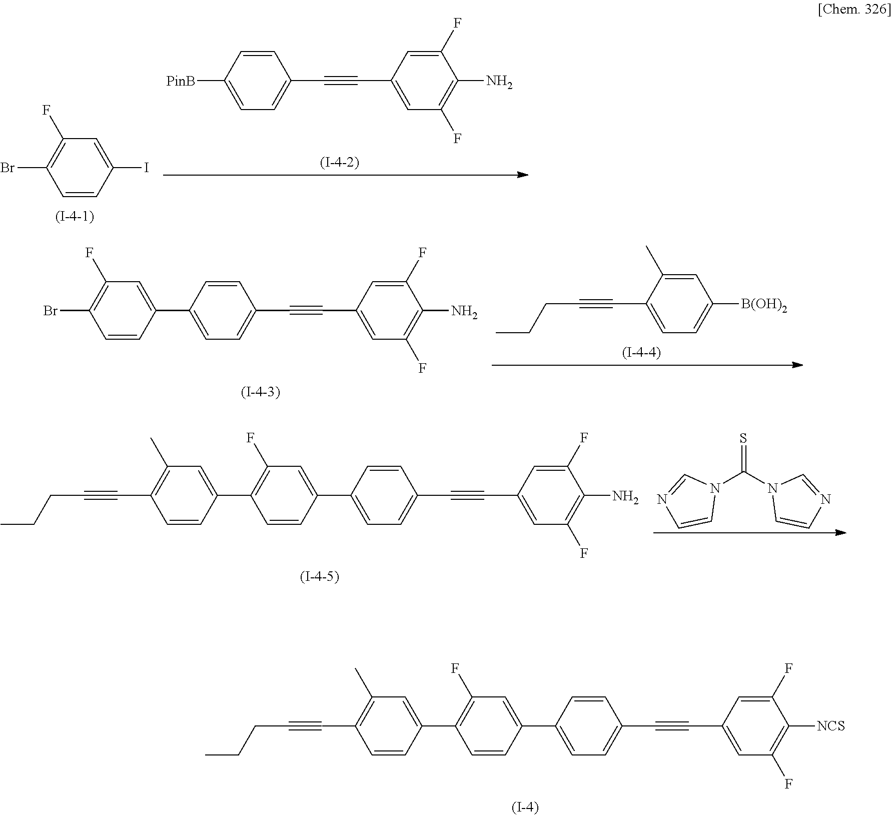

6.3 g of a compound represented by the formula (I-4-1), 7.5 g of the compound represented by the formula (I-4-2), 0.1 g of bis(triphenylphosphine) palladium (II) dichloride, 4.3 g of sodium carbonate, 30 ml of tetrahydrofuran, and 20 ml of water were added to a reaction vessel in a nitrogen atmosphere at room temperature and were stirred at 75° C. for 2 hours. After completion of the reaction, the product was collected by filtration and was purified by column chromatography (amino silica gel, toluene/hexane=2/3 to 1/0) to produce 6.8 g of a compound represented by the formula (I-4-3).

Next, 6.8 g of the compound represented by the formula (I-4-3), 3.5 g of a compound represented by the formula (I-4-4), 0.1 g of bis(triphenylphosphine) palladium (II) dichloride, 4.3 g of sodium carbonate, 30 mL of tetrahydrofuran, and 20 mL of water were added to a reaction vessel in a nitrogen atmosphere at room temperature and were stirred at 75° C. for 2 hours. After completion of the reaction, the product was collected by filtration and was purified by column chromatography (amino silica gel, toluene/hexane=2/3 to 1/0) to produce 6.5 g of a compound represented by the formula (I-4-5).

Next, 6.5 g of the compound represented by the formula (I-4-5), 17 mL of dichloromethane, and 2.4 g of 1,1-thiocarbonyldiimidazole were added to a reaction vessel in a nitrogen atmosphere at room temperature and were stirred at room temperature. After completion of the reaction, the organic layer was washed with saturated saline and was purified by column chromatography (silica gel, toluene) and subsequent recrystallization (toluene/hexane=1/1) to produce 5.5 g of a compound represented by the formula (I-4).

MS (EI): m/z=521

### (Synthesis Example 5) Production of Compound Represented by Formula (I-5)

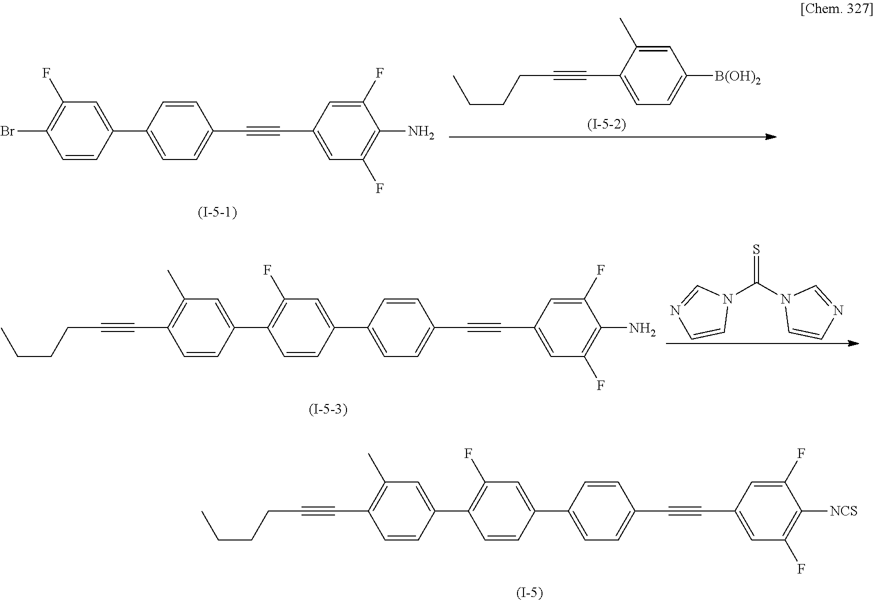

6.8 g of the compound represented by the formula (I-5-1), 3.7 g of a compound represented by the formula (I-5-2), 0.1 g of bis(triphenylphosphine) palladium (II) dichloride, 4.3 g of sodium carbonate, 30 mL of tetrahydrofuran, and 20 mL of water were added to a reaction vessel in a nitrogen atmosphere at room temperature and were stirred at 75° C. for 2 hours. After completion of the reaction, the product was collected by filtration and was purified by column chromatography (amino silica gel, toluene/hexane=2/3 to 1/0) to produce 6.8 g of a compound represented by the formula (1-5-3).

Next, 6.8 g of the compound represented by the formula (I-5-3), 17 mL of dichloromethane, and 2.7 g of 1,1-thiocarbonyldiimidazole were added to a reaction vessel in a nitrogen atmosphere at room temperature and were stirred at room temperature. After completion of the reaction, the organic layer was washed with saturated saline and was purified by column chromatography (silica gel, toluene) and subsequent recrystallization (toluene/hexane=1/1) to produce 6 g of a compound represented by the formula (I-5).

MS (EI): m/z=535

### (Synthesis Example 6) Production of Compound Represented by Formula (I-6)

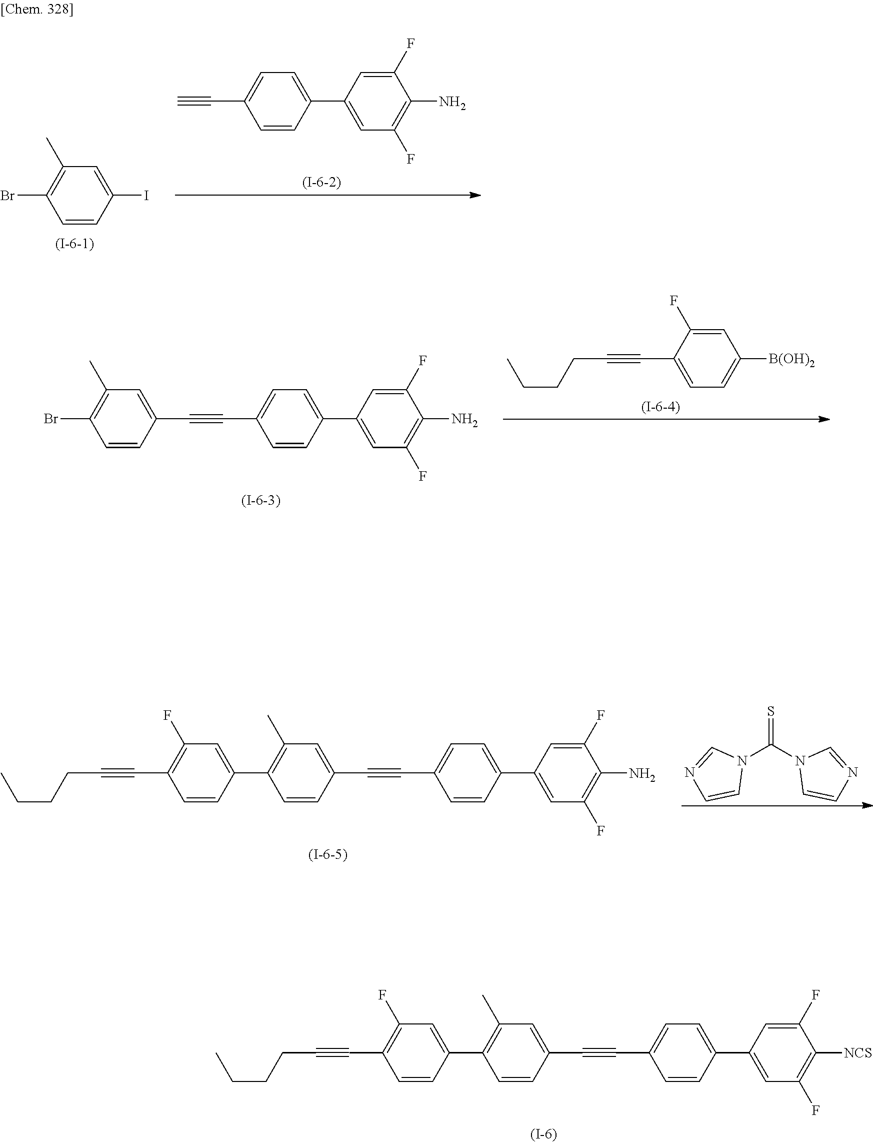

10 g of a compound represented by the formula (I-6-1), 0.25 g of copper (I) iodide, 1 g of bis(triphenylphosphine) palladium (II) dichloride, 25 g of triethylamine, and 50 mL of tetrahydrofuran were put into a reaction vessel in a nitrogen atmosphere at room temperature. Next, 8.3 g of a compound represented by the formula (I-6-2) dissolved in 25 mL of tetrahydrofuran was added dropwise while stirring at room temperature and was stirred at room temperature for 1 hour. After completion of the reaction, saturated aqueous ammonium chloride was poured into the reaction liquid, and extraction with toluene was performed. The organic layer was washed with saturated saline and was purified by column chromatography (amino silica gel, toluene/hexane=1/8 to 1/0) to produce 10.5 g of a compound represented by the formula (I-6-3).

Next, 10.5 g of the compound represented by the formula (I-6-3), 6.0 g of a compound represented by the formula (I-6-4), 0.2 g of bis(triphenylphosphine) palladium (II) dichloride, 6.5 g of sodium carbonate, 50 mL of tetrahydrofuran, and 25 mL of water were added to a reaction vessel in a nitrogen atmosphere at room temperature and were stirred at 75° C. for 2 hours. After completion of the reaction, the product was collected by filtration and was purified by column chromatography (amino silica gel, toluene/hexane=2/3 to 1/0) to produce 10.2 g of a compound represented by the formula (I-6-5).

Next, 10.2 g of the compound represented by the formula (I-6-5), 30 mL of dichloromethane, and 4.4 g of 1,1-thiocarbonyldiimidazole were added to a reaction vessel in a nitrogen atmosphere at room temperature and were stirred at room temperature. After completion of the reaction, the organic layer was washed with saturated saline and was purified by column chromatography (silica gel, toluene) and subsequent recrystallization (toluene/hexane=1/1) to produce 8.7 g of a compound represented by the formula (I-6).

MS (EI): m/z=535

### (Synthesis Example 7) Production of Compound Represented by Formula (I-7)

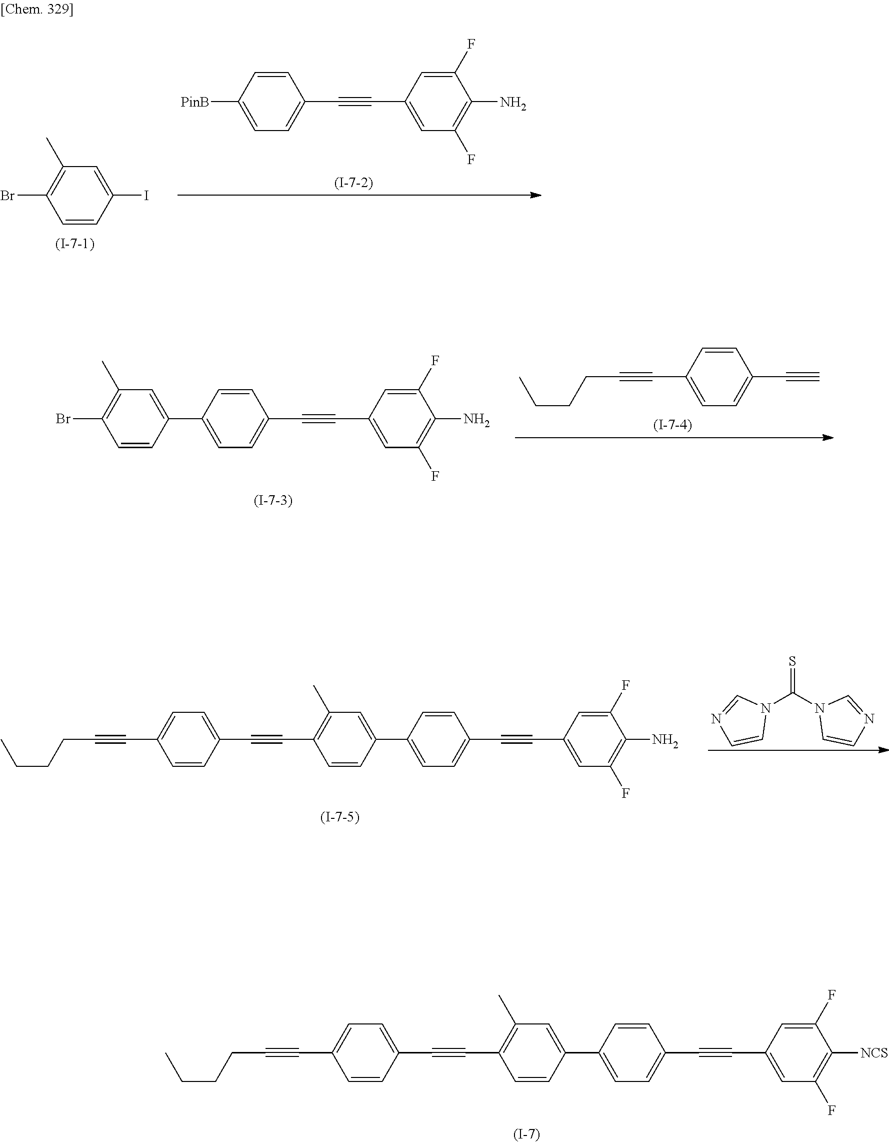

10 g of a compound represented by the formula (1-7-1), 7.7 g of a compound represented by the formula (1-7-2), 0.2 g of bis(triphenylphosphine) palladium (II) dichloride, 7 g of sodium carbonate, 50 mL of tetrahydrofuran, and 25 mL of water were added to a reaction vessel in a nitrogen atmosphere at room temperature and were stirred at 75° C. for 2 hours. After completion of the reaction, the product was collected by filtration and was purified by column chromatography (amino silica gel, toluene/hexane=2/3 to 1/0) to produce 10.5 g of a compound represented by the formula (1-7-3).

Next, 10.5 g of the compound represented by the formula (I-7-3), 0.2 g of copper (I) iodide, 0.4 g of bis(triphenylphosphine) palladium (II) dichloride, 25 g of triethylamine, and 50 mL of tetrahydrofuran were put into a reaction vessel in a nitrogen atmosphere at room temperature. Next, 7 g of a compound represented by the formula (I-7-4) dissolved in 15 mL of tetrahydrofuran was added dropwise while stirring at room temperature and was stirred at room temperature for 1 hour. After completion of the reaction, saturated aqueous ammonium chloride was poured into the reaction liquid, and extraction with toluene was performed. The organic layer was washed with saturated saline and was purified by column chromatography (amino silica gel, toluene/hexane=1/8 to 1/0) to produce 10.3 g of a compound represented by the formula (I-7-5).

Next, 10.3 g of the compound represented by the formula (I-7-5), 25 mL of dichloromethane, and 4 g of 1,1-thiocarbonyldiimidazole were added to a reaction vessel in a nitrogen atmosphere at room temperature and were stirred at room temperature. After completion of the reaction, the organic layer was washed with saturated saline and was purified by column chromatography (silica gel, toluene) and subsequent recrystallization (toluene/hexane=1/1) to produce 5 g of a compound represented by the formula (I-7).

MS (EI): m/z=535

### (Synthesis Example 8) Production of Compound Represented by Formula (I-8)

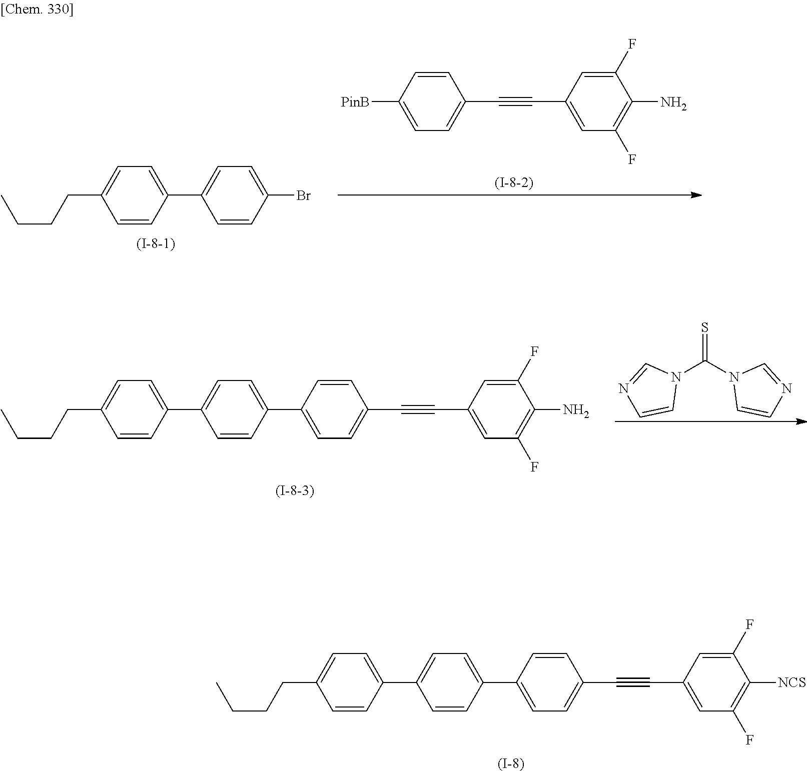

10 g of a compound represented by the formula (1-8-1), 7.9 g of a compound represented by the formula (1-8-2), 0.2 g of bis(triphenylphosphine) palladium (II) dichloride, 7 g of sodium carbonate, 50 mL of tetrahydrofuran, and 25 mL of water were added to a reaction vessel in a nitrogen atmosphere at room temperature and were stirred at 75° C. for 2 hours. After completion of the reaction, the product was collected by filtration and was purified by column chromatography (amino silica gel, toluene/hexane=2/3 to 1/0) to produce 10.4 g of a compound represented by the formula (1-8-3).

Next, 10.4 g of the compound represented by the formula (I-8-3), 25 mL of dichloromethane, and 5 g of 1,1-thiocarbonyldiimidazole were added to a reaction vessel in a nitrogen atmosphere at room temperature and were stirred at room temperature. After completion of the reaction, the organic layer was washed with saturated saline and was purified by column chromatography (silica gel, toluene) and subsequent recrystallization (toluene/hexane=1/1) to produce 9.8 g of a compound represented by the formula (I-8).

MS (EI): m/z=478

### (Synthesis Example 9) Production of Compound Represented by Formula (I-9)

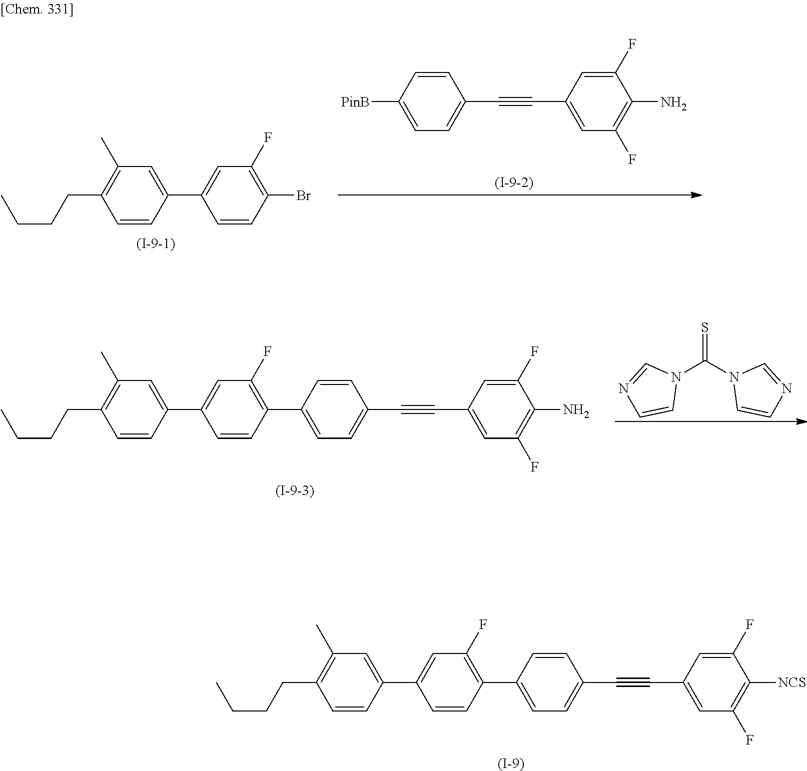

10 g of a compound represented by the formula (I-9-1), 7.8 g of a compound represented by the formula (I-9-2), 0.2 g of bis(triphenylphosphine) palladium (II) dichloride, 7 g of sodium carbonate, 50 mL of tetrahydrofuran, and 25 mL of water were added to a reaction vessel in a nitrogen atmosphere at room temperature and were stirred at 75° C. for 2 hours. After completion of the reaction, the product was collected by filtration and was purified by column chromatography (amino silica gel, toluene/hexane=2/3 to 1/0) to produce 10.7 g of a compound represented by the formula (I-9-3).

Next, 10.7 g of the compound represented by the formula (I-9-3), 25 mL of dichloromethane, and 4.4 g of 1,1-thiocarbonyldiimidazole were added to a reaction vessel in a nitrogen atmosphere at room temperature and were stirred at room temperature. After completion of the reaction, the organic layer was washed with saturated saline and was purified by column chromatography (silica gel, toluene) and subsequent recrystallization (toluene/hexane=1/1) to produce 10.5 g of a compound represented by the formula (I-9).

MS (EI): m/z=511

### INDUSTRIAL APPLICABILITY

A compound and a liquid crystal composition according to the present invention can be utilized in a liquid crystal display element, a sensor, a liquid crystal lens, optical communication equipment, and an antenna.

1. A liquid crystal composition comprising one or two or more compounds represented by the following general formula (i),
[Chem. 1]
Rⁱ¹-Aⁱ¹-Zⁱ¹-Aⁱ²-Zⁱ²-Aⁱ³-Zⁱ³-Aⁱ⁴-NCS (i)
(in the general formula (i),
Rⁱ¹ denotes a hydrogen atom or an alkyl group with 1 to 20 carbon atoms,
one or two or more —CH₂— groups in the alkyl group are each independently optionally substituted with —O—, —S—, —CO—, and/or —CS—,
one or two or more —CH₂—CH₂— groups in the alkyl group are each independently optionally substituted with —CO—O—, —O—CO—, —CO—S—, —S—CO—, —CO—NH—, —NH—CO—, —CH—CH—, —CF═CF—, and/or —C≡C—,
one or two or more hydrogen atoms in the alkyl group are each independently optionally substituted with a halogen atom,
provided that oxygen atoms are not directly bonded to each other,
Aⁱ¹, Aⁱ², Aⁱ³, and Aⁱ⁴ each independently denote any one of a hydrocarbon ring with 3 to 16 carbon atoms and a heterocycle with 3 to 16 carbon atoms,
one or two or more hydrogen atoms in Aⁱ¹, Aⁱ², Aⁱ³, and Aⁱ⁴ are each independently optionally substituted with a substituent Sⁱ¹,
the substituent Sⁱ¹ denotes any one of a fluorine atom, a chlorine atom, a bromine atom, an iodine atom, a pentafluorosulfanyl group, a nitro group, a cyano group, an isocyano group, an amino group, a hydroxy group, a mercapto group, a methylamino group, a dimethylamino group, a diethylamino group, a diisopropylamino group, a trimethylsilyl group, a dimethylsilyl group, a thioisocyano group, and an alkyl group with 1 to 20 carbon atoms,
one or two or more —CH₂— groups in the alkyl group are each independently optionally substituted with —O—, —S—, and/or —CO—,
one or two or more —CH₂—CH₂— groups in the alkyl group are each independently optionally substituted with —CH═CH—, —CF═CF—, —C≡C—, —CO—O—, —O—CO—, —CO—S—, —S—CO—, —CO—NH—, and/or —NH—CO—,
one or two or more hydrogen atoms in the alkyl group are each independently optionally substituted with a halogen atom,
provided that oxygen atoms are not directly bonded to each other,
a plurality of substituents Sⁱ¹, if present, may be the same or different, Zⁱ¹, Zⁱ², and Zⁱ³ each independently denote any one of a single bond and an alkylene group with 1 to 20 carbon atoms,
at least one of Zⁱ¹, Zⁱ², and Zⁱ³ denotes —C≡C—,
one or two or more —CH₂— groups in the alkylene group are each independently optionally substituted with —O—, —CF₂—, and/or —CO—, and
one or two or more —CH₂—CH₂— groups in the alkylene group are each independently optionally substituted with —CH₂—CH(CH₃)—, —CH(CH₃)—CH₂—, —CH═CH—, —CF—CF—, —CH═C(CH₃)—, —C(CH₃)═CH—, —CH═N—, —N═CH—, —N═N—, —C≡C—, —CO—O—, and/or —O—CO—,
provided that oxygen atoms are not directly bonded to each other).

2. The liquid crystal composition according to claim 1, wherein Rⁱ¹ denotes an alkynyl group with 2 to 20 carbon atoms by substituting one or two or more —CH₂—CH₂-groups in the alkyl group with —C≡C—.

3. The liquid crystal composition according to claim 1, wherein Zⁱ³ denotes —C≡C—.

4. The liquid crystal composition according to claim 1, further comprising one or two or more compounds represented by the following general formula (ii), 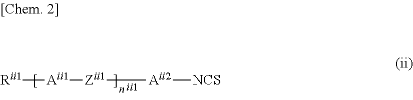
(in the general formula (ii),
Rⁱⁱ¹ each independently denotes an alkyl group with 1 to 20 carbon atoms,
one or two or more —CH₂— groups in the alkyl group are each independently optionally substituted with —O—, —S—, —CO—, and/or —CS—,
one or two or more —CH₂—CH₂— groups in the alkyl group are each independently optionally substituted with —CH═CH—, —CO—O—, —O—CO—, —CO—S—, —S—CO—, —CO—NH—, —NH—CO—, —CH═CH—, —CF═CF—, and/or —C≡C—,
one or two or more hydrogen atoms in the alkyl group are each independently optionally substituted with a halogen atom,
provided that oxygen atoms are not directly bonded to each other,
Aⁱⁱ¹ and Aⁱⁱ² each independently denote a group selected from the group consisting of the following groups (a), (b), (c), and (d):
(a) a 1,4-cyclohexylene group (one —CH₂— or two or more nonadjacent —CH₂-groups in this group are optionally substituted with —O— and/or —S—),
(b) a 1,4-phenylene group (one —CH═ or two or more —CH═ groups in this group are optionally substituted with —N═),
(c) a 1,4-cyclohexenylene group, a bicyclo[2.2.2]octane-1,4-diyl group, a naphthalene-2,6-diyl group, a naphthalene-1,4-diyl group, a 1,2,3,4-tetrahydronaphthalene-2,6-diyl group, a 5,6,7,8-tetrahydronaphthalene-1,4-diyl group, a decahydronaphthalene-2,6-diyl group, an anthracene-2,6-diyl group, an anthracene-1,4-diyl group, an anthracene-9,10-diyl group, a phenanthrene-2,7-diyl group, a 2,3-dihydroindene-2,5-diyl group, a 2,3-dihydroindene-1,5-diyl group, or a 2,3-dihydroindene-3,5-diyl group (one —CH═ or two or more —CH═ groups in the naphthalene-2,6-diyl group, the naphthalene-1,4-diyl group, the 1,2,3,4-tetrahydronaphthalene-2,6-diyl group, the 5,6,7,8-tetrahydronaphthalene-1,4-diyl group, the anthracene-2,6-diyl group, the anthracene-1,4-diyl group, the anthracene-9,10-diyl group, or the phenanthrene-2,7-diyl group are optionally substituted with —N═), and
(d) a thiophene-2,5-diyl group, a benzothiophene-2,5-diyl group, a benzothiophene-2,6-diyl group, a dibenzothiophene-3,7-diyl group, a dibenzothiophene-2,6-diyl group, a thieno[3,2-b]thiophene-2,5-diyl group, or a benzo[1,2-b: 4,5-b′]dithiophene-2,6-diyl group (one —CH═ or two or more —CH═ groups in this group are optionally substituted with —N═),
one or two or more hydrogen atoms in Aⁱⁱ¹ and Aⁱⁱ² are each independently optionally substituted with a substituent Sⁱⁱ¹,
the substituent Sⁱⁱ¹ denotes any one of a halogen atom, a pentafluorosulfanyl group, a nitro group, a cyano group, an isocyano group, an amino group, a hydroxy group, a mercapto group, a methylamino group, a dimethylamino group, a diethylamino group, a diisopropylamino group, a trimethylsilyl group, a dimethylsilyl group, a thioisocyano group, and an alkyl group with 1 to 20 carbon atoms,
one or two or more —CH₂— groups in the alkyl group are each independently optionally substituted with —O—, —S—, —CO—, and/or —CS—,
one or two or more —CH₂—CH₂— groups in the alkyl group are each independently optionally substituted with —CO—O—, —O—CO—, —CO—S—, —S—CO—, —CO—NH—, —NH—CO—, —CH═CH—, —CF═CF—, and/or —C≡C—,
one or two or more hydrogen atoms in the alkyl group are each independently optionally substituted with a halogen atom,
provided that oxygen atoms are not directly bonded to each other,
a plurality of substituents Sⁱⁱ¹, if present, may be the same or different,
Zⁱⁱ¹ denotes any one of a single bond and an alkylene group with 1 to 20 carbon atoms,
one or two or more —CH₂— groups in the alkylene group are each independently optionally substituted with —O—, —CF₂—, and/or —CO—, and
one or two or more —CH₂—CH₂— groups in the alkylene group are each independently optionally substituted with —CH₂—CH(CH₃)—, —CH(CH₃)—CH₂—, —CH—CH—, —CF═CF—, —CH═C(CH₃)—, —C(CH₃)—CH—, —CH═N—, —N═CH—, —N═N—, —C≡C—, —CO—O—, and/or —O—CO—,
provided that oxygen atoms are not directly bonded to each other,
nⁱⁱ¹ denotes an integer in the range of 1 to 4, and
a plurality of Aⁱⁱ¹, if present, may be the same or different, and a plurality of Zⁱ¹, if present, may be the same or different,
provided that the compounds represented by the general formula (i) are excluded).

5. The liquid crystal composition according to claim 4, wherein the compound represented by the general formula (ii) is selected from the group consisting of compounds represented by the following general formulae (ii-1) to (ii-8), 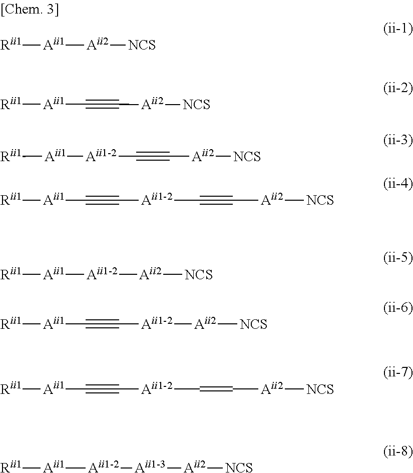
(in the general formulae (ii-1) to (ii-8),
Rⁱⁱ¹, Aⁱⁱ¹, and Aⁱⁱ² denote the same as Rⁱⁱ¹, Aⁱⁱ¹, and Aⁱⁱ², respectively, in the general formula (ii), and
Aⁱⁱ¹-2 and Aⁱⁱ¹-3 in the general formulae (ii-3) to (ii-8) are each independently defined in the same way as Aⁱ¹ in the general formula (ii)).

6. The liquid crystal composition according to claim 1, wherein Δn at 25° C. and at 589 nm is 0.38 or more.

7. A liquid crystal display element which used the liquid crystal composition according to claim 1.

8. The liquid crystal display element according to claim 7, wherein the liquid crystal display element is driven by an active matrix system or a passive matrix system.

9. A liquid crystal display element that reversibly switches permittivity by reversibly changing an orientation direction of liquid crystal molecules of the liquid crystal composition according to claim 1.

10. A sensor which used the liquid crystal composition according to claim 1.

11. A liquid crystal lens which used the liquid crystal composition according to claim 1.

12. An optical communication equipment which used the liquid crystal composition according to claim 1.

13. An antenna which used the liquid crystal composition according to claim 1.

14. The antenna according to claim 13, wherein the antenna comprises:
a first substrate with a plurality of slots;
a second substrate facing the first substrate and provided with a power supply portion;
a first dielectric layer provided between the first substrate and the second substrate;
a plurality of patch electrodes arranged corresponding to the plurality of slots;
a third substrate on which the patch electrodes are provided; and
a liquid crystal layer provided between the first substrate and the third substrate,
wherein the liquid crystal layer contains the liquid crystal composition according to claim 1.

15. A compound represented by the following general formula (i),
[Chem. 4]
Rⁱ¹-Aⁱ¹-Zⁱ¹-Aⁱ²-Zⁱ²-Aⁱ³-Zⁱ³-Aⁱ⁴-NCS (i)
(in the general formula (i),
Rⁱ¹ denotes a hydrogen atom or an alkyl group with 1 to 20 carbon atoms,
one or two or more —CH₂— groups in the alkyl group are each independently optionally substituted with —O—, —S—, —CO—, and/or —CS—,
one or two or more —CH₂—CH₂— groups in the alkyl group are each independently optionally substituted with —CO—O—, —O—CO—, —CO—S—, —S—CO—, —CO—NH—, —NH—CO—, —CH═CH—, —CF═CF—, and/or —C≡C—,
one or two or more hydrogen atoms in the alkyl group are each independently optionally substituted with a halogen atom,
provided that oxygen atoms are not directly bonded to each other,
Aⁱ¹, Aⁱ², Aⁱ³, and Aⁱ⁴ each independently denote any one of a hydrocarbon ring with 3 to 16 carbon atoms and a heterocycle with 3 to 16 carbon atoms,
one or two or more hydrogen atoms in Aⁱ¹, Aⁱ², Aⁱ³, and Aⁱ⁴ are each independently optionally substituted with a substituent Sⁱ¹,
the substituent Sⁱ¹ denotes any one of a fluorine atom, a chlorine atom, a bromine atom, an iodine atom, a pentafluorosulfanyl group, a nitro group, a cyano group, an isocyano group, an amino group, a hydroxy group, a mercapto group, a methylamino group, a dimethylamino group, a diethylamino group, a diisopropylamino group, a trimethylsilyl group, a dimethylsilyl group, a thioisocyano group, and an alkyl group with 1 to 20 carbon atoms,
one or two or more —CH₂— groups in the alkyl group are each independently optionally substituted with —O—, —S—, and/or —CO—,
one or two or more —CH₂—CH₂— groups in the alkyl group are each independently optionally substituted with —CH—CH—, —CF—CF—, —C≡C—, —CO—O—, —O—CO—, —CO—S—, —S—CO—, —CO—NH—, and/or —NH—CO—,
one or two or more hydrogen atoms in the alkyl group are each independently optionally substituted with a halogen atom,
provided that oxygen atoms are not directly bonded to each other,
a plurality of substituents Sⁱ¹, if present, may be the same or different, Zⁱ¹, Zⁱ², and Zⁱ³ each independently denote any one of a single bond and an alkylene group with 1 to 20 carbon atoms,
at least one of Zⁱ¹, Zⁱ², and Zⁱ³ denotes —C≡C—,
one or two or more —CH₂— groups in the alkylene group are each independently optionally substituted with —O—, —CF₂—, and/or —CO—, and
one or two or more —CH₂—CH₂— groups in the alkylene group are each independently optionally substituted with —CH₂—CH(CH₃)—, —CH(CH₃)—CH₂—, —CH—CH—, —CF═CF—, —CH═C(CH₃)—, —C(CH₃)—CH—, —CH═N—, —N═CH—, —N═N—, —C≡C—, —CO—O—, and/or —O—CO—,
provided that oxygen atoms are not directly bonded to each other).
